(19) United States
(12) Patent Application Publication
(10) Pub. No.: US 2009/0270989 A1
(43) Pub. Date: Oct. 29, 2009

(54) SPINAL IMPLANTS AND METHODS

(75) Inventors: E. Scott Conner, Santa Barbara, CA (US); Jay A. Lenker, Laguna Beach, CA (US); Khoi Nguyen, Murrieta, CA (US); Jeffrey J. Valko, San Clemente, CA (US); Matthew Scott Lake, Carlsbad, CA (US); Peter Gregory Davis, Dana Point, CA (US)

Correspondence Address: MCDERMOTT WILL & EMERY LLP, 18191 VON KARMAN AVE., SUITE 500, IRVINE, CA, 92612-7108, US

(73) Assignee: Magellan Spine Technologies, Inc., Irvine, CA (US)

(21) Appl. No.: 12/274,339

(22) Filed: Nov. 19, 2008

Related U.S. Application Data
(60) Provisional application No. 61/032,921, filed on Feb. 29, 2008.
(60) Provisional application No. 61/016,417, filed on Dec. 21, 2007.
(60) Provisional application No. 60/989,100, filed on Nov. 19, 2007.

(51) Int. Cl. A61F 2/44; A61M 29/00

(52) U.S. Cl. 623/17.16; 606/191

(57) ABSTRACT

Spinal implants are disclosed that can be used for annular repair, facet unloading, disc height preservation, disc decompression, or for sealing a portal through which a nucleus implant was placed. In some embodiments, an implant is placed within the intervertebral disc space, primarily within the region of the annulus fibrosus. In some embodiments, the implant is expandable. In some embodiments, the implant has a sealing tail structure comprising a tail flange and a linkage. In some embodiments, the sealing tail structure limits the extrusion or expulsion of disc material, either annulus fibrosus or nucleus, into the posterior region of the spine where it could impinge on nerves. In some embodiments, the tail structure is retained in place within the annulus fibrosus by means of an anchor. In some embodiments, the anchor is constructed from multiple components.

### CROSS-REFERENCE TO RELATED APPLICATIONS

This application claims priority to U.S. Provisional Patent App. No. 61/032,921, filed on Feb. 29, 2008, which in turn claims priority to U.S. Provisional Patent App. No. 61/016,417, filed on Dec. 21, 2007, which in turn claims priority to U.S. Provisional Patent App. No. 60/989,100, filed on Nov. 19, 2007, the entire contents of all of these applications are herein incorporated by reference.

### BACKGROUND

1. Field

The present disclosure relates to devices and methods for treating intervertebral discs using implants.

2. Description of the Related Art

The vertebral spine is the axis of the skeleton upon which all of the body parts “hang,” or are supported. In humans, the normal spine has seven cervical, twelve thoracic, and five lumbar segments. Functionally each segment can be thought of as comprising an intervertebral disc, sandwiched between two vertebral bodies. The lumbar segments sit upon a sacrum, which then attaches to a pelvis, in turn supported by hip and leg bones. The bony vertebral bodies of the spine are separated by intervertebral discs, which act as joints, but allow known degrees of flexion, extension, lateral bending and axial rotation.

Each intervertebral disc serves as a mechanical cushion between the vertebral bones, permitting controlled motions within vertebral segments of the axial skeleton. For example, FIG. 4 illustrates a healthy intervertebral disc 30 and adjacent vertebrae 32. A spinal nerve 34 extends along the spine posteriorly thereof.

The normal disc is a unique, mixed structure, comprised of three component tissues: The nucleus pulposus (“nucleus”), the annulus fibrosus (“annulus”), and two opposing vertebral end plates. The two vertebral end plates are each composed of thin cartilage overlying a thin layer of hard, cortical bone which attaches to the spongy, richly vascular, cancellous bone of the vertebral body. The end plates thus serve to attach adjacent vertebrae to the disc. In other words, a transitional zone is created by the end plates between the malleable disc and the bony vertebrae.

The annulus of the disc is a tough, outer fibrous ring that binds together adjacent vertebrae. This fibrous portion is generally about 10 to 15 millimeters (“mm”) in height and about 15 to 20-mm in thickness, although in diseased discs these dimensions may be diminished. The fibers of the annulus consist of 15 to 20 overlapping multiple plies, and are inserted into the superior and inferior vertebral bodies at roughly a 30-degree angle in both directions. This configuration particularly resists torsion, as about half of the angulated fibers will tighten when the vertebrae rotate in either direction, relative to each other. The laminated plies are less firmly attached to each other.

Immersed within the annulus, within the intervertebral disc space, is the nucleus pulposus. The annulus and opposing end plates maintain a relative position of the nucleus in what can be defined as a nucleus cavity. The healthy nucleus is largely a gel-like substance, comprising poly-mucosaccharides having high water content, and similar to air in a tire, serves to keep the annulus tight yet flexible. The nucleus-gel moves slightly within the annulus when force is exerted on the adjacent vertebrae with bending, lifting, etc. The nucleus is capable of absorbing water and generating varying amounts of pressure within the intervertebral disc. As a person ages, intervertebral discs, especially those of the lumbar spine, tend to increasingly lose the distinction between annulus and nucleus. The annulus tissue, comprising circumferentially disposed fibrous tissue, tends to migrate inward taking up space formerly occupied by nucleus. The demarcation between annulus and nucleus becomes progressively undefined. Previously nuclear tissue becomes annulus tissue with the decreasing amount of nucleus tissue being constrained increasingly radially inward within the intervertebral disc. The ability of an aged lumbar intervertebral disc to retain water is diminished relative to the disc of a younger person.

Under certain circumstances, an annulus defect (or annulotomy) can arise that requires surgical attention. These annulus defects can be naturally occurring, the result of injury, surgically created, or a combination thereof. A naturally occurring annulus defect is typically the result of trauma or a disease process, and may lead to a disc herniation. FIG. 5 illustrates a herniated disc 36. A disc herniation occurs when the annulus fibers are weakened or torn and the inner tissue of the nucleus becomes permanently bulged, distended, or extruded out of its normal, internal annular confines. The mass of a herniated or “slipped” nucleus 38 can compress a spinal nerve 40, resulting in leg pain, loss of muscle control, or even paralysis.

Where the naturally occurring annulus defect is relatively minor and/or little or no nucleus tissue has escaped from the nucleus cavity, satisfactory healing of the annulus may be achieved by immobilizing the patient for an extended period of time. However, many patients require surgery (microdiscectomy) to remove the herniated portion of the disc. FIG. 6 illustrates a disc from which a portion has been removed through a microdiscectomy procedure. After the traditional microdiscectomy, loss of disc space height may also occur because degenerated disc nucleus is removed as part of the surgical procedure. Loss of disc space height can also be a source of continued or new lumbar spine generated pain.

Further, a more problematic annulus defect concern arises in the realm of annulotomies encountered as part of a surgical procedure performed on the disc space. Alternatively, with discal degeneration, the nucleus loses its water binding ability and deflates, as though the air had been let out of a tire. Subsequently, the height of the nucleus decreases, causing the annulus to buckle in areas where the laminated plies are loosely bonded. As these overlapping laminated plies of the annulus begin to buckle and separate, either circumferential or radial annular tears can occur, which may contribute to persistent and disabling back pain. Adjacent, ancillary spinal facet joints can also be forced into an overriding position, which can create additional back pain.

In many cases, to alleviate pain from degenerated or herniated discs, the nucleus is removed and the two adjacent vertebrae surgically fused together. While this treatment can alleviate the pain, all discal motion is lost in the fused segment. Ultimately, this procedure places greater stress on the discs adjacent the fused segment as they compensate for the lack of motion, perhaps leading to premature degeneration of those adjacent discs.

Regardless of whether the annulus defect occurs naturally or as part of a surgical procedure, an effective device and method for repairing such defects, while at the same time providing for dynamic stability of the motion segment, would be of great benefit to sufferers of herniated discs and annulus defects.

### SUMMARY

A more desirable solution entails replacing, in part or as a whole, the damaged nucleus with a suitable prosthesis having the ability to complement the normal height and motion of the disc while stimulating, at least in part, natural disc physiology. Disclosed embodiments of the present spinal implants and methods of providing dynamic stability to the spine have several features, no single one of which is solely responsible for their desirable attributes. Without limiting the scope of these spinal implants and methods as expressed by the claims that follow, their more prominent features will now be discussed briefly. After considering this discussion, and particularly after reading the section entitled “Detailed Description,” one will understand how the features of the disclosed embodiments provide advantages, which include, inter alia, the capability to repair annular defects and stabilize adjacent motion segments of the spine without substantially diminishing the range of motion of the spine, simplicity of structure and implantation, and a low likelihood that the implant will migrate from the implantation site.

The implant can be fabricated from materials such as biocompatible metals such as titanium, stainless steel, or cobalt nickel alloys, or it can comprise biocompatible polymers such as polyetheretherketone, polyester, and polysulfone. The implant can further comprise biodegradable/erodable materials such as polylactic acid, polyglycolic acid, sugars, collagen, and the like. The axially elongate structure can comprise rigid materials or it can be compressible to assist with the maintenance of spine mobility.

In some embodiments, the implant can be suited for a population of patients who have pain from an unruptured hernia (bulge) that can be decompressed by implanting a distraction device separating the vertebrae enough to pull the bulge in and relieving the disc of axial compression, and perhaps allowing the disc to re-hydrate. The decompression feature of the device can assist in preventing future herniation. In some embodiments, the implant can further serve as a stabilizer for the spine since it can be configured to apply support uniformly from left to right. Further, the implant can preserve some motion in the spine since the spine can still hinge forward or backward about the device to at least some extent. The axially elongate implant can serve as this distraction device. The location of the implant can be at the center of flexion-extension and the implant can serve as a barrier against re-herniation along the entire length of the internal posterior wall of the annulus. In some embodiments, a single implant can be placed to separate, or distract, the vertebrae. In some embodiments, a plurality of implants can be placed to separate the vertebrae. In certain embodiments, two implants can be placed, one on each side of the posterior portion of the spine, to stabilize the spine laterally and to provide one or more of the functions of decompression, vertebral distraction, facet unloading, nerve decompression, and disc height preservation or restoration. In some embodiments, the implants can have their longitudinal axes oriented generally laterally with regard to the anatomic axis of the spine. In some embodiments, the implants can have their longitudinal axes oriented generally in the approximate anterior or posterior direction. In certain embodiments, the implants can have their longitudinal axes oriented radially with respect to the geometric center of the intervertebral disc. In some embodiments, these devices can provide for motion preservation of the spine segment within which the devices are implanted. In certain embodiments, the implants can partially or totally restrict motion within that segment. In some embodiments, the implants can be used in conjunction with spinal fusion procedures to maintain early postoperative stability of spinal support. In certain embodiments, the implant can reside totally within the outer boundary of the annulus of the intervertebral disc. In some embodiments, the implant can reside with a portion of its structure external to the outer boundary of the intervertebral disc annulus. In some embodiments, the decompression devices are placed using a posterior access. In some embodiments, the decompression devices are placed using posteriolateral access. In some embodiments, the decompression devices are placed using anterior or anteriolateral access.

With each embodiment, an implant procedure can also be provided. The implant procedure can comprise preparation steps including, but not limited to, magnetic resonance imaging of the affected region, computer aided tomography imaging of the affected region, placement of a trocar at the correct location under fluoroscopy, advancement of nested, staged, or expanding access sheaths into the target location, monitoring under fluoroscopy, and monitoring under direct vision such as through a surgical operating microscope.

The implant procedure can include steps including tunneling through the facets using burrs or Rongeurs to carefully remove the minimum material necessary for access. The implant procedure can include the steps of moving nerves aside and protecting nerves from damage. The implant procedure can include the steps of removing herniated disc material using grasping, scraping, or scooping instruments placed through the sheath. The implant procedure can include, without limitation, the use of lip sizers, the use of lip reamers, the use of implant reamers, the use of trial units to determine appropriate implant fit, the use of distracting instrumentation, the use of annulus coring tools, the use of implant delivery tools, and the like.

In some embodiments, the devices and procedures described herein are configured to secure a plug or seal to a defect in the annulus of an intervertebral disc. Those intervertebral discs exhibiting herniation and requiring repair may have non-discreet delineation between the nucleus and the annulus tissue. There may be little or no clearly defined nucleus. There may be no inner boundary of the annulus against which an implant can be secured. The annulus may be highly degenerated and incapable of supporting sutures or other attachments which could otherwise be able to provide some fixation for an implant. These conditions are more likely than not to occur in patients requiring a plug in an annular defect. The devices described herein are configured to be constrained by the vertebrae, the end plates of the vertebrae, or by an intact annulus. These devices do not require that any nucleus be present within the intervertebral disc.

In some embodiments, the devices described herein are configured for support of spinal fusion procedures. In other embodiments, the devices described herein are configured for annular repair of an intervertebral disc. In other embodiments, the devices described herein are configured for support or treatment of scoliosis. The scoliosis-targeted implants can be asymmetric lordotic implants. In other embodiments, the devices described herein are configured for disc decompression, facet unloading, height preservation, or height restoration. The devices described herein can be used in embodiments that preserve spinal motion along at least one axis. The motion preserving devices can be configured to provide dynamic stability to the spine.

In some or all of the embodiments disclosed herein, the implant devices can be used and/or implanted within a vertebral body, such as for the treatment of compression fractures. A compression fracture occurs when a normal vertebral body of a spine has collapsed or compressed from its original anatomical size. Typically, these vertebrae fail at an anterior cortical wall causing a wedge shaped collapse of the vertebra. Fractures can be painful for the patient typically causing a reduced quality of life. These fractures can be repaired by the insertion, into the vertebral body, of certain embodiments of the spinal implants disclosed herein, to reinforce the fractured bone, alleviate associated pain, and to prevent further vertebral collapse.

In some embodiments, the devices described herein can be configured for placement using posterior approaches. In other embodiments, the devices described herein can be configured for lateral approaches. In some embodiments, the devices described herein can be configured for percutaneous or minimally invasive approaches. In some embodiments, the devices described herein can be configured for trans-foramenal approaches.

In some embodiments, reamers are described for use in removing or modifying tissue within the annulus or adjacent vertebrae. In some embodiments, the reamers are expandable. These expandable reamers comprise a first unexpanded state dimension in the reaming head. The expandable reamers also comprise a second dimension in the reaming head that is larger than the corresponding dimension in the first, unexpanded state. In some embodiments, the reaming head can unfurl or unfold to create the second, larger dimension. In other embodiments, the reaming head can comprise a blade that hinges outward in response to control forces exerted at the proximal end of the device. In other embodiments, the reaming head, generally located at or near the distal end of the reamer or reaming instrument, is expanded by forcing a central wedge therethrough, causing a collet-like structure to expand in the reaming head.

In some embodiments, implants are provided that can be placed through lateral, or posterior-lateral approaches. These implants can be unitary in construction or the implants can comprise a plurality of components. These implants, which in some embodiments comprise axially elongate structures, can be configured to comprise a first, unexpanded state and a second expanded state, wherein the expansion occurs in a direction generally normal or lateral to the longitudinal axis of the implant. The expandable implants that run generally in the lateral direction from left to right, or right to left, can expand by means including but not limited to, swellable components, by means of spring loaded components, by means of insertion of cores that force expansion of the exterior, by means or rotating a cam, or the like.

In some embodiments, implants placed using a lateral, posterior-lateral, trans-foramenal or other similar approach can be guided into place using a delivery system. The delivery system can comprise a catheter, trocar, port, guidewire, or the like. The delivery system can comprise a pre-curved or adjustable curve configuration. Adjustability, shape change, or curving can be accomplished using shape memory means, spring-loaded means, or steering means, wherein the steering means are controlled from the proximal end of the delivery system.

In some embodiments, instruments are disclosed for distracting the vertebrae, vertebral lips, intervertebral disc opening, or the like. The distraction instruments can be applied through an open surgical incision, or they can be applied through a minimally invasive approach such as port access. The distraction instruments generally comprise an axially elongate shaft, a handle, and distraction components that distract using approaches such as reverse pliers, a rotating cam, an expandable collet, or the like. In some embodiments, the force to cause distraction is applied by squeezing opposing grips or pulling a trigger or lever at the proximal end of the device with the force being delivered along the length of the axially elongate instrument by means of linkages, shafts, or the like. In other embodiments, the distraction force can be applied by rotating an element at the proximal end of the instrument which causes the entire instrument, or a part thereof, to rotate at the distal end. In yet other embodiments, the distraction at the distal end can be generated with mechanical advantage by operably connecting the distracting jaws or elements to a jackscrew, lever, threaded rod, or the like.

In certain embodiments, an implant is provided for maintaining a height between adjacent vertebrae. The implant includes an expandable member comprising an inflation port, the expandable member configured to expand between adjacent vertebrae of a patient upon inflation of the expandable member through the inflation port. When implanted in the patient and expanded, the expandable member fills a portion of the intervertebral disc space between the adjacent vertebrae and maintains a height between the vertebrae.

In certain embodiments, when implanted in the patient and expanded, the expandable member exerts a bias force on the adjacent vertebrae. In certain embodiments, the implant further includes a lumen extending through the implant, and at least one injection port fluidly connected to the lumen. The at least one injection port is configured to permit passage of an injectable material from outside the implant into the lumen and into the intervertebral disc space. In certain embodiments, the expandable member is sized and shaped to be inserted through a defect in the annulus fibrosus of an intervertebral disc between the adjacent vertebrae. In certain embodiments, at least a portion of the expandable member is compressible by the adjacent vertebrae. In certain embodiments, the expandable member includes a swellable polymer. In certain embodiments, the expandable member includes a balloon. In certain embodiments, the implant is part of an implant system that also includes a fluid reservoir in fluid communication with the expandable member and configured to expand the expandable member in response to a flow of fluid from the reservoir to the expandable member. In certain embodiments of the implant system, when implanted in the patient, the fluid reservoir and the implant reside in the intervertebral disc space, and upon compression by the adjacent vertebrae, the fluid reservoir transfers fluid to the expandable member.

In certain embodiments, an implant is provided for maintaining a height between adjacent vertebrae. The implant includes an expandable member comprising a shape memory material, the expandable member changing from an unexpanded configuration to an expanded configuration in response to an activation energy. When implanted in the patient and expanded between adjacent vertebrae in response to the activation energy, the expandable member fills a portion of the intervertebral disc space between the adjacent vertebrae and maintains a height between the vertebrae.

In certain embodiments, an implant is provided for maintaining a height between adjacent vertebrae. The implant includes an expandable member, sized and shaped to be positioned between the adjacent vertebrae, and an expander member configured to couple to the expandable member and to expand the expandable member radially when the expander member moves axially with respect to the expandable member. Radial expansion of the expandable member is effective to anchor the implant between the adjacent vertebrae. In certain embodiments, the expandable member and the expander member are sized and shaped to be inserted through a defect in the annulus fibrosus of an intervertebral disc between the adjacent vertebrae. In certain embodiments, the expandable member has a lumen within it, and the expander member moves axially within the lumen. In certain embodiments, the expandable member includes a screw thread, and the expander member moves axially within the lumen when the expander member is rotated. In certain embodiments, the expandable member includes a screw configured to foreshorten at least a portion of the implant, while effecting radial expansion of the expandable member. In certain embodiments, the expandable member includes a wedge, located within a lumen of the implant, the wedge configured to expand radially the expandable member as the wedge is moved within the lumen.

In certain embodiments, an implant is provided for maintaining a height between adjacent vertebrae. The implant includes a head, comprising a central portion and an expandable member, wherein the expandable member is radially disposed around at least part of the central portion. When implanted in the patient, the expandable member resides within the intervertebral disc space and exerts an outward bias force on the adjacent vertebrae, resulting in anchoring of the implant within the intervertebral disc space. The central portion is configured to move axially with respect to the expandable member.

In certain embodiments, when the expandable member is compressed by the adjacent vertebrae, the central portion moves axially with respect to the expandable member. In certain embodiments, the at least one expandable member is self-expanding. In certain embodiments, the central portion includes a groove, configured to receive a portion of the expandable member. In certain embodiments, the expandable member is sized and shaped to be inserted through a defect in an intervertebral disc between the adjacent vertebrae.

In certain embodiments, an implant is provided for implantation between adjacent vertebrae. The implant includes an first expandable member, and a second expandable member in fluid communication with the first expandable member and configured to expand the first expandable member in response to a flow of fluid from the second expandable member toward the first expandable member. When the first and second expandable members are implanted in the intervertebral disc space between the adjacent vertebrae, and the first expandable member is expanded, the first expandable member fills a portion of the intervertebral disc space between the adjacent vertebrae. When the first expandable member is compressed by the adjacent vertebrae, fluid flows from the first expandable member toward the second expandable member, resulting in expansion of the second expandable member. In certain embodiments, the first expandable member includes a fluid reservoir.

In certain embodiments, a method is provided for maintaining a height between the adjacent vertebrae. The method includes providing an implant having a head in an unexpanded state, inserting the head into the intervertebral disc space of the patient, and, after the inserting, expanding the head from the unexpanded state to an expanded state until the head substantially engages tissue in the intervertebral disc space. The implant also includes after the expanding, a portion of the implant maintains a height between the adjacent vertebrae.

In certain embodiments, the method further includes inflating the expandable member to expand the expandable member. In certain embodiments of the method, the engaged tissue includes at least one of the vertebrae. In certain embodiments, a method is provided for maintaining a height between adjacent vertebrae or otherwise treating a spinal disorder. The method includes providing an implant having an expandable member fluidly coupled to a fluid reservoir, positioning the expandable member and the fluid reservoir in the intervertebral disc space between the adjacent vertebrae, and expanding the expandable member by delivering fluid toward the expandable member from the fluid reservoir, thereby exerting a force within the intervertebral disc space.

In certain embodiments, the method further includes delivering fluid toward the fluid reservoir from the expandable member in response to compression of the expandable member by the adjacent vertebrae.

A method is provided for maintaining a height between adjacent vertebrae. The method includes placing an implant into an intervertebral disc space between two adjacent vertebrae, and actuating an adjustment member of the implant, thereby radially expanding at least a portion of an expandable member of the implant. When radially expanded, the expandable member maintains the implant substantially in place between the adjacent vertebrae and prevents expulsion of the implant from the intervertebral disc space.

In certain embodiments of the method, the placing includes inserting the implant through a defect in the annulus fibrosus of an intervertebral disc between the adjacent vertebrae. In certain embodiments of the method, the placing includes positioning the implant entirely within the annulus fibrosus of an intervertebral disc between the adjacent vertebrae.

In certain embodiments, an implant is provided for at least one of (i) treating an annular defect in an intervertebral disc between two adjacent vertebrae of a patient, and (ii) maintaining a height between the adjacent vertebrae. The implant includes an expandable anchor, configured to be expanded between the adjacent vertebrae, and a tail portion, coupled to the expandable anchor. When implanted in the patient and expanded, the expandable anchor fills a portion of the intervertebral disc space and maintains a height between the vertebrae. When the expandable anchor is implanted and expanded between the adjacent vertebrae, the tail portion forms a barrier effective to prevent substantial expulsion of material from the intervertebral disc space.

In certain embodiments, the implant further includes a lumen extending through at least one of the expandable anchor and the tail portion, and at least one injection port fluidly connected to the lumen, wherein the at least one injection port is configured to permit passage of an injectable material from outside the implant into the lumen. In certain embodiments, the tail portion includes a flange that, at least in part, forms the barrier. In certain embodiments, the tail portion includes a flange and a coupling member, the coupling member is configured to couple the tail flange to the expandable anchor, and the barrier is formed at least in part by the coupling member. In certain embodiments, the coupling portion includes a surface structure that promotes tissue ingrowth. In certain embodiments, the coupling portion includes a material that promotes tissue ingrowth. In certain embodiments, when the tail portion is implanted and forms the barrier, the tail portion contacts an outer surface of the intervertebral disc.

In certain embodiments, at least a portion of the expandable member is compressible by the adjacent vertebrae. In certain embodiments, the expandable anchor includes an inflation port, configured for inflation of the anchor to expand it. In certain embodiments, when implanted in the patient and expanded, the expandable anchor exerts a bias force on the adjacent vertebrae. In certain embodiments, the expandable anchor is sized and shaped to be inserted through the annular defect. In certain embodiments, the expandable anchor includes a swellable polymer. In certain embodiments, the tail portion is expandable. In certain embodiments, the tail portion includes a swellable polymer. In certain embodiments, the expandable anchor includes a balloon. In certain embodiments, the expandable anchor includes a shape memory material that changes from an unexpanded configuration to an expanded configuration in response to an activation energy.

In certain embodiments, the implant is included in an implant system. The implant system also includes a fluid reservoir in fluid communication with the expandable anchor and configured to expand the expandable anchor in response to flow of fluid from the reservoir to the expandable anchor. In certain embodiments of the implant system includes, when implanted in the patient, the fluid reservoir and the implant reside in the intervertebral disc space, and upon compression by the adjacent vertebrae, the fluid reservoir transfers fluid to the expandable anchor.

In certain embodiments, an implant system is provided for at least one of (i) treating an annular defect in an intervertebral disc between two adjacent vertebrae of a patient, and (ii) maintaining a height between the adjacent vertebrae. The implant system includes an implant, including a head, a tail portion, and a coupling member that couples the head and tail portion. The tail portion is configured to expand laterally relative to a longitudinal axis of the implant. The implant system also includes an adjustment member that couples to the implant and moves the tail portion from an unexpanded configuration to an expanded configuration. When the implant is implanted in the patient, and when the tail portion is in the expanded configuration, the head resides between the adjacent vertebrae, and the tail portion forms a barrier effective to limit expulsion of intervertebral disc material from the intervertebral disc space.

In certain embodiments of the implant system, the adjustment member is configured to remain coupled to the implant, and to remain implanted in the patient, after the implant is implanted in the patient. In certain embodiments, the implant system includes, wherein the tail portion includes at least one hinge, and the tail portion expands by movement at the at least one hinge. In certain embodiments, the implant system includes, wherein the tail portion includes a gear, and the tail portion expands by movement of the gear. In certain embodiments of the implant system, the head is expandable from a first configuration to a second configuration. In certain embodiments, the implant system further includes a locking mechanism coupled to the tail portion, configured to maintain the tail portion in the expanded configuration.

In certain embodiments, an implant is provided for at least one of (i) treating an annular defect in an intervertebral disc between two adjacent vertebrae of a patient, and (ii) maintaining a height between the adjacent vertebrae. The implant includes a head, sized and shaped to be placed between the adjacent vertebrae, wherein the head is positionable within the intervertebral disc space in a first collapsed state and expandable within the intervertebral disc space to engage tissue in the intervertebral disc space. The implant also includes a tail portion. When the head is positioned between the two adjacent vertebrae, the tail portion contacts an outer surface of the intervertebral disc and forms a barrier that prevents substantial expulsion of material from within the disc past the barrier. The implant also includes a coupling member that couples the tail portion to the head. The tail portion is advanceable along the coupling member toward the head. The coupling member is configured to fix the tail portion in a position relative to the head, such that the tail portion contacts the outer surface of the disc when the head is positioned within the intervertebral disc space.

In certain embodiments, when the head is positioned between the adjacent vertebrae, at least one of the tail portion and the coupling member maintains a height between the adjacent vertebrae. In certain embodiments, when the head is positioned between the two adjacent vertebrae, the head engages at least one of the adjacent vertebrae. In certain embodiments, the coupling member includes a screw thread, and the tail portion is rotatably advanceable along the coupling member. In certain embodiments, the tail portion is expandable. In certain embodiments, the tail portion includes a flange that, at least in part, forms the barrier. In certain embodiments, the tail portion includes a flange and a coupling member, the coupling member is configured to couple the tail flange to the expandable anchor, and the barrier is formed at least in part by the coupling member.

In certain embodiments, an implant is provided for at least one of (i) treating an annular defect in an intervertebral disc between two adjacent vertebrae of a patient, and (ii) maintaining a height between the adjacent vertebrae. The implant includes an expandable anchor sized and shaped to be positioned between the adjacent vertebrae, and a tail portion. The implant also includes an expander member coupled to the tail portion and configured to expand the expandable anchor radially when the expander member moves axially with respect to the expandable anchor. Radial expansion of the expandable anchor is effective to anchor the implant between the adjacent vertebrae. When implanted in the patient, the tail portion is configured to form a barrier effective to prevent substantial expulsion of material from the intervertebral disc, when the expandable anchor is radially expanded between the adjacent vertebrae.

In certain embodiments, the expandable anchor is sized and shaped to be inserted through the annular defect. In certain embodiments, the expandable anchor has a lumen within it, and the expander member moves axially within the lumen. In certain embodiments, the expandable anchor includes a screw thread, and the expander member moves axially within the lumen when the expander member is rotated.

In certain embodiments, an implant is provided for at least one of (i) treating an annular defect in an intervertebral disc between two adjacent vertebrae of a patient, and (ii) maintaining a height between the adjacent vertebrae. The implant includes a head, comprising a central portion and an expandable anchor, wherein the expandable anchor is radially disposed around at least part of the central portion. The implant also includes a tail portion coupled to the head. When implanted in the patient, the expandable anchor resides within the intervertebral disc space and exerts an outward bias force on the adjacent vertebrae, resulting in anchoring of the implant within the intervertebral disc space. When the head is anchored within the intervertebral disc space, the tail portion forms a barrier effective to prevent substantial expulsion of material from the intervertebral disc. The central portion is configured to move axially with respect to the expandable anchor.

In certain embodiments, when the expandable anchor is compressed by the adjacent vertebrae, the central portion moves axially with respect to the expandable anchor. In certain embodiments, when the expandable anchor is compressed by the adjacent vertebrae, the central portion moves axially with respect to the expandable anchor, resulting in the tail portion moving closer to the expandable anchor. In certain embodiments, the expandable anchor is self-expanding. In certain embodiments, the central portion includes a groove, configured to receive a portion of the expandable anchor. In certain embodiments, the expandable anchor is sized and shaped to be inserted through the annular defect.

In certain embodiments, a method is provided for at least one of (i) treating an annular defect in an intervertebral disc between two adjacent vertebrae of a patient, and (ii) maintaining a height between the adjacent vertebrae. The method includes inserting an implant, having an anchor coupled to a tail portion, into the intervertebral disc space of the patient until the tail portion forms a barrier effective to prevent substantial expulsion of material from the intervertebral disc. The method also includes expanding the anchor within the intervertebral disc space while the anchor remains coupled to the tail portion.

In certain embodiments, a method is provided for at least one of (i) treating an annular defect in an intervertebral disc between two adjacent vertebrae of a patient, and (ii) maintaining a height between the adjacent vertebrae. The method includes providing an implant, having a head coupled to a tail portion, the head being in an unexpanded state, inserting the head into the intervertebral disc space of the patient, and, after the inserting, expanding the head from the unexpanded state to an expanded state until the head substantially engages tissue in the intervertebral disc space. The method also includes advancing the tail portion toward the head until the tail flange is in contact with an outer surface of the intervertebral disc.

In certain embodiments, a method is provided for treating an annular defect in an intervertebral disc between two adjacent vertebrae of a patient. The method includes inserting, through the defect, an implant having an expandable anchor that is coupled to both a tail portion and a fluid reservoir, until the expandable anchor and the fluid reservoir are positioned in the intervertebral disc space between the adjacent vertebrae, and the tail flange contacts an outer surface of the disc and forms a barrier at the defect that prevents substantial expulsion of material from the disc. The method also includes expanding the expandable anchor by delivering fluid toward the expandable anchor from the fluid reservoir.

In certain embodiments, the method further includes delivering fluid toward the fluid reservoir from the expandable member in response to compression of the expandable member by the adjacent vertebrae.

In certain embodiments, a method is provided for treating an annular defect in an intervertebral disc between two adjacent vertebrae of a patient. The method includes inserting an implant into the defect, the implant comprising a tail portion and a swellable polymer, such that the implant is effectively anchored between the adjacent vertebrae. The method also includes activating the swellable polymer such that a space between the implant and a body structure of the patient is substantially occupied. The method also includes, with the tail portion, forming a barrier effective to prevent substantial expulsion of material from the intervertebral disc.

In certain embodiments of the method, while the tail portion acts as the barrier effective to prevent substantial expulsion of material from the intervertebral disc, the tail portion contacts an outer surface of the intervertebral disc.

In certain embodiments, an implant is provided for at least one of (i) treating an annular defect in an intervertebral disc between adjacent vertebrae of a patient, and (ii) maintaining a height between the adjacent vertebrae. The implant includes a head portion, sized and shaped to be positioned within the intervertebral disc space between the adjacent vertebrae and configured to engage tissue in the intervertebral disc space, a tail portion. The implant also includes a coupling member that couples the tail portion to the head portion. When the head portion is positioned between the adjacent vertebrae, the tail portion contacts a surface of the annulus fibrosus of the intervertebral disc and forms a barrier that prevents substantial expulsion of material from within the disc past the barrier.

In certain embodiments, the coupling member is configured to allow the tail portion to move relative to the anchor. In certain embodiments, when the head portion is positioned between the adjacent vertebrae, at least one of the tail portion and the coupling member maintains a height between the adjacent vertebrae. In certain embodiments, the head portion is configured to engage at least one of the adjacent vertebrae. In certain embodiments, the coupling member is releasably coupled to at least one of the head portion and the tail portion. In certain embodiments, the barrier is formed, at least in part, by the coupling member. In certain embodiments, the a head portion includes at least one bone compaction opening. In certain embodiments, the a head portion includes a plurality of slits disposed about a perimeter of the head portion. In certain embodiments, the tail portion includes a swellable polymer configured, when hydrated, to substantially fill a space between the adjacent vertebrae. In certain embodiments, the head portion includes a plurality of components, cooperatively assembled and engaged to form a substantially contiguous structure.

In certain embodiments, the head portion is moveable from a first configuration to a second configuration, wherein the first configuration is configured to permit placement of the implant within the intervertebral disc space. The second configuration is configured to fix the implant in place within the intervertebral disc space following implantation. In certain embodiments, the implant further includes a lumen extending through at least one of the head portion and the tail portion, and at least one injection port fluidly connected to the lumen, wherein the at least one injection port is configured to permit passage of an injectable material from outside the implant into the lumen. In certain embodiments, the coupling member includes a flexible tether. In certain embodiments, the head portion and the tail portion interact so as to preserve substantially a normal physiological range of motion of the adjacent vertebrae after implantation of the implant in the intervertebral disc space.

In certain embodiments, at least one of the head portion and tail portion is configured to unload compressive forces exerted on spinal facets. In certain embodiments, at least one of the head portion and tail portion is configured to decompress impinged spinal nerves upon implantation of the implant. In certain embodiments, the head portion includes a plurality of anchor units, configured to be placed sequentially between the adjacent vertebrae, the plurality of units forming a resultant anchor that lodges between the adjacent vertebrae. In certain embodiments, the head portion includes a layer of bone growth factor on at least a portion of an outer surface. In certain embodiments, the tail portion is advanceable along the coupling member toward the head portion. In certain embodiments, the coupling member includes a screw thread, and the tail portion is rotatably advanceable along the coupling member. In certain embodiments, at least a portion of the head portion is configured to be embedded through an endplate of, and into, at least one of the adjacent vertebrae. In certain embodiments, at least a portion of the head portion is configured to be embedded into each of the adjacent vertebrae.

In certain embodiments, the head portion includes at least one screw, configured to be embedded into at least one of the adjacent vertebrae. In certain embodiments, the head portion includes at least one of a hook and a barb, configured to be embedded into at least one of the adjacent vertebrae. In certain embodiments, the head portion includes at least one spike, configured to be embedded into at least one of the adjacent vertebrae. In certain embodiments, the head portion includes no more than one spike, configured to be embedded into either a superior or an inferior vertebra. In certain embodiments, the head portion includes a spike, wherein the spike includes a flexible shaft having column strength and tensile strength such that the spike can be advanced from the tail flange area and deflect either superiorly or inferiorly to embed within either of the adjacent vertebrae. In certain embodiments, the coupling member is configured to fix the tail portion in a position relative to the head portion. In certain embodiments, at least one of the coupling member and the tail portion includes a ratchet, configured to fix the tail portion in a position relative to the head portion. In certain embodiments, the coupling member threadably engages the tail portion to fix the tail portion in a position relative to the head portion. In certain embodiments, the coupling member locks with the tail portion to fix the tail portion in a position relative to the head portion. In certain embodiments, the at least one coupling member further includes a bias member configured to provide a force that maintains effective contact between the tail portion and the surface of the disc. In certain embodiments, the bias member pulls the head portion toward the tail portion to assist in the preventing substantial expulsion of material from within the disc.

In certain embodiments of the implant, the head portion has a height and a width that are each substantially transverse to a long axis of the head portion, wherein the height and the width are such that, when the head is in a first rotational position with respect to the long axis, the head portion passes into the intervertebral disc space as the head portion is advanced between the adjacent vertebrae. Furthermore, when the head portion is in the intervertebral disc space and is rotated into a second rotational position with respect to the long axis, the head portion engages tissue in intervertebral disc space, substantially conforming to a height of a region of the intervertebral disc space to the height of the head portion. In certain such embodiments, wherein the height and the width are such that, when the head is in the first rotational position with respect to the long axis, the head portion passes into the intervertebral disc space as the head portion is advanced substantially along the long axis between the adjacent vertebrae. In certain embodiments, an angle of rotation between the first rotational position and the second rotational position is about 90°. In certain embodiments, the engaged tissue in the intervertebral disc space includes at least one of the adjacent vertebrae. In certain embodiments, after the head portion is rotated into the second rotational position, a portion of the implant maintains a height between the adjacent vertebrae. In certain embodiments, the implant further includes a lumen extending through at least one of the head portion and the tail portion. The implant also includes at least one injection port fluidly connected to the lumen, wherein the at least one injection port is configured to permit passage of an injectable material from outside the implant into the lumen.

In certain embodiments, an implant is provided for at least one of (i) treating an annular defect in an intervertebral disc between adjacent vertebrae of a patient, and (ii) maintaining a height between the adjacent vertebrae. The implant includes a spacer, sized and shaped to be positioned within the intervertebral disc space between the adjacent vertebrae to engage at least one of the adjacent vertebrae. When the implant is positioned between the adjacent vertebrae, a portion of the implant engages tissue in intervertebral disc space and forms a barrier that prevents substantial expulsion of material from within the disc past the barrier, wherein the spacer has a height and a width that are each substantially transverse to a long axis of the spacer. The height and the width are such that, when the spacer is in a first rotational position with respect to the long axis, the spacer passes into the intervertebral disc space as the spacer is advanced substantially along the long axis between the adjacent vertebrae. When the spacer is in the intervertebral disc space and is rotated into a second rotational position with respect to the long axis, the spacer engages tissue in intervertebral disc space, substantially conforming a height of a region of the intervertebral disc space to the height of the spacer.

In certain embodiments, an angle of rotation between the first rotational position and the second rotational position is about 90°. In certain embodiments, the engaged tissue in the intervertebral disc space includes at least one of the adjacent vertebrae. In certain embodiments, after the spacer is rotated into the second rotational position, a portion of the implant maintains a height between the adjacent vertebrae.

In certain embodiments, an implant is provided for at least one of (i) treating an annular defect in an intervertebral disc between two adjacent vertebrae of a patient, and (ii) maintaining a height between the adjacent vertebrae. The implant includes an anchoring member, configured to be positioned in the intervertebral disc space between the adjacent vertebrae, a portion of the anchoring member being configured to engage tissue in the intervertebral disc space. The implant also includes a tail portion, coupled to the at least one anchoring member, such that when the portion is embedded into the at least one of the adjacent vertebrae, the tail portion contacts a surface of the annulus fibrosus of the intervertebral disc and forms a barrier that prevents substantial expulsion of material from the disc past the tail portion. The implant also includes at least one coupling member that couples the anchoring member to the tail portion and fixes the tail portion in a position relative to the head, such that the tail portion contacts the surface of the disc.

In certain embodiments, when the anchoring member is positioned between the adjacent vertebrae, at least one of the tail portion and the at least one coupling member maintains a height between the adjacent vertebrae. In certain embodiments, the anchoring member is configured to engage at least one of the adjacent vertebrae. In certain embodiments, the portion of the anchoring member is configured to embed into each of the two adjacent vertebrae. In certain embodiments, the portion of the anchoring member is includes at least one of a spike, a hook, and a barb. In certain embodiments, the at least one coupling member further includes a bias member configured to provide a force that maintains effective contact between the tail portion and the surface of the disc.

In certain embodiments, an implant is provided for at least one of (i) treating an annular defect in an intervertebral disc between adjacent vertebrae of a patient, and (ii) maintaining a height between the adjacent vertebrae. The implant includes a tail portion, configured to form a barrier effective to prevent expulsion of material from an intervertebral disc. The implant also includes a head portion, coupled to the tail portion. The head portion is configured to transform from an uncoiled configuration to a coiled configuration in the intervertebral disc space. When the implant is positioned between the adjacent vertebrae, when the tail portion engages the annulus fibrosus of the intervertebral disc, and when the head portion has been transformed from the uncoiled configuration to the coiled configuration in the intervertebral disc space, the implant is anchored at the intervertebral disc. In certain embodiments, the head portion includes a shape memory portion, configured to transform from the uncoiled configuration to the coiled configuration in response to an activation energy.

In certain embodiments, an implant is provided for at least one of (i) treating an annular defect in an intervertebral disc between adjacent vertebrae of a patient, and (ii) maintaining a height between the adjacent vertebrae. The implant includes a tail portion. The implant also includes an anchor head, configured to engage a tissue within the intervertebral disc space, the anchor head comprising a plurality of anchor members. The implant also includes at least one bias member, coupling at least one of the anchor members to the tail portion and providing a force exerted by the at least one of the anchor members engaging with the tissue. When the implant is positioned between the adjacent vertebrae and the at least one anchor head is engaged with the tissue, the tail portion forms a barrier effective to prevent substantial expulsion of material from within the disc past the barrier.

In certain embodiments, when the anchor head is positioned between the adjacent vertebrae, at least one of the tail portion and the bias member maintains a height between the adjacent vertebrae. In certain embodiments, the anchor head is configured to engage at least one of the adjacent vertebrae. In certain embodiments, the bias member includes a spring.

In certain embodiments, a spinal implant is provided for at least one of (i) treating an annular defect in an intervertebral disc between adjacent vertebrae of a patient, and (ii) maintaining a height between the adjacent vertebrae. The implant includes a first elongate guide member, having a proximal portion and a distal portion, a second elongate guide member, having a proximal portion and a distal portion. The implant also includes a barrier member that is configured to extend from the first to the second guide member, wherein the proximal portion of the first guide member is configured to be anchored to a first location on an outer surface of a first vertebrae, and the distal portion of the first guide member is configured to be anchored to a second location on an outer surface of the first vertebrae. The proximal portion of the second guide member is configured to be anchored to a first location on an outer surface of a second vertebrae adjacent the first vertebrae, and the distal portion of the second guide member is configured to be anchored to a second location on an outer surface of the second vertebrae. The barrier member is movable between an unextended configuration and an extended configuration, when the first guide member and second guide member are anchored to their respective first and second vertebrae. When the barrier member in the extended configuration and spans from the first guide member to the second guide member, the barrier member forms a barrier effective to prevent substantial expulsion of material from within the disc past the barrier.

In certain embodiments, the extendable barrier member is configured to extend within the intervertebral disc. In certain embodiments, the extendable barrier member is configured to unfurl when moved from the unextended configuration to the extended configuration. In certain embodiments, the implant further includes a plurality of anchor members, configured to anchor the first guide member and second guide to the first and second vertebrae, respectively.

In certain embodiments, a spinal implant is provided for at least one of (i) treating an annular defect in an intervertebral disc between adjacent vertebrae of a patient, and (ii) maintaining a height between the adjacent vertebrae. The implant includes a head portion, configured to anchor the implant within an intervertebral disc located between adjacent vertebrae, and a tail portion, coupled to the head portion. The implant also includes at least one anchor member, the at least one anchor member configured to be directed into a tissue adjacent to an intervertebral disc. In certain embodiments, the tail portion is configured to contact an outer surface of the intervertebral disc. The at least one anchor member is coupled to the head portion, and is configured to move from a first configuration to a second configuration, and to engage the tissue when in the second configuration. The implant also includes a retainer member, configured to maintain the at least one anchor member in the first configuration until the implant is positioned in the disc. The implant also includes an anchor release member, configured to release the at least one anchor member from the retainer member, such that the at least one anchor member transforms from the first configuration to the second configuration. When the implant is positioned in the disc, at least one vertebrae is engaged by at least one anchor member, and the tail portion substantially contacts an outer surface of the intervertebral disc, forming a barrier effective to prevent substantial expulsion of material from within the disc past the barrier. In certain embodiments, the at least one anchor member includes a shape memory material, configured to transform from the first configuration to the second configuration in response to an activation energy. In certain embodiments, the retainer member slidably releases the at least one anchor member. In certain embodiments, the retainer member threadably releases the at least one anchor member.

In certain embodiments, an implant is provided for at least one of (i) treating an annular defect in an intervertebral disc between adjacent vertebrae of a patient, and (ii) maintaining a height between the adjacent vertebrae. The implant includes a body, a tail portion, coupled to the body. The implant also includes at least one anchor port, each anchor port having an anchor entry and an anchor exit, wherein each anchor port forms a lumen passing through the tail portion and the body. Each anchor port is configured to direct an anchor into a tissue adjacent to the intervertebral disc.

In certain embodiments, each anchor port further includes an anchor coupler effective to couple the anchor to the anchor port. In certain embodiments, the tissue includes a vertebra. In certain embodiments, the anchor is configured to thread into the tissue. In certain embodiments, at least one anchor port defines a path that is at least partially curved. In certain embodiments, the tail portion includes a flange and a coupling member, wherein the flange is configured to prevent the substantial expulsion of material, and wherein the coupling member is configured to couple the flange to the body, and wherein the barrier is formed at least in part by the flange and the body.

In certain embodiments, an implant is provided for at least one of (i) treating an annular defect in an intervertebral disc between adjacent vertebrae of a patient, and (ii) maintaining a height between the adjacent vertebrae. The implant includes a head, a tail portion, a bias member, configured to couple the head and tail portion in tension. The implant also includes a collapsible tail, between the head and tail portion, wherein the collapsible tail further includes a lumen, configured to admit the bias member. The collapsible tail is further configured to permit axial movement of the tail portion relative to the head in response to the tension, while limiting tissue encroachment into the bias member.

In certain embodiments, a method is provided for at least one of (i) treating an annular defect in an intervertebral disc between adjacent vertebrae of a patient, and (ii) maintaining a height between the adjacent vertebrae. The method includes inserting an implant, having a tail portion comprising a swellable polymer, into the intervertebral disc space of the patient until the tail portion forms a barrier effective to prevent substantial expulsion of material from the intervertebral disc. hydrating the swellable polymer until the swellable polymer fills a substantial space between the adjacent vertebrae.

In certain embodiments, a method is provided for at least one of (i) treating an annular defect in an intervertebral disc between adjacent vertebrae of a patient, and (ii) maintaining a height between the adjacent vertebrae. The method includes inserting an implant, having a head portion, a tail portion and an injection port, into the intervertebral disc space of the patient until the tail portion forms a barrier effective to prevent substantial expulsion of material from the intervertebral disc. The injection port forms a lumen passing through the tail portion and the head portion. directing an injectable material into a tissue adjacent to the intervertebral disc.

In certain embodiments, a method is provided for at least one of (i) treating an annular defect in an intervertebral disc between adjacent vertebrae of a patient, and (ii) maintaining a height between the adjacent vertebrae. The method includes inserting an implant, having a head portion coupled to a tail portion by a coupling member, into the intervertebral disc space of the patient. The method also includes advancing the tail portion along the coupling member toward the head portion until the tail portion forms a barrier effective to prevent substantial expulsion of material from the intervertebral disc.

In certain embodiments of the method, the tail portion is rotatably advanced along the coupling member.

In certain embodiments, a method is provided for at least one of (i) treating an annular defect in an intervertebral disc between adjacent vertebrae of a patient, and (ii) maintaining a height between the adjacent vertebrae. The method includes inserting a first guide member, having a proximal end and a distal end, at least partially within the intervertebral disc space between the adjacent vertebrae, inserting a second guide member, having a proximal end and a distal end, within the intervertebral disc space, anchoring the proximal end of the first guide member to a first location on an outer surface of a first vertebrae of the adjacent vertebrae, anchoring the distal end of the first guide member to a second location on an outer surface of the first vertebrae, anchoring the proximal end of the second guide member to a first location on an outer surface of a second vertebrae of the adjacent vertebrae. The method also includes anchoring the distal end of the second guide member to a second location on an outer surface of the second vertebrae, coupling an extendable barrier member, in an unextended configuration, to each of the first guide member and second guide member. The method also includes transforming the extendable barrier member from the unextended configuration to an extended configuration. When in the extended configuration, the extendable barrier member forms a barrier effective to prevent substantial expulsion of material from within the disc past the barrier.

In certain embodiments of the method, transforming the extendable barrier member from the unextended configuration to the extended configuration includes unfurling the extendable barrier member. In certain embodiments, the method further includes anchoring the first guide member to the first vertebrae using an anchor member. The implant also includes anchoring the second guide member to the second vertebrae using an anchor member.

In certain embodiments, a method is provided for at least one of (i) treating an annular defect in an intervertebral disc between adjacent vertebrae of a patient, and (ii) maintaining a height between the adjacent vertebrae. The method includes inserting, between the adjacent vertebrae, an implant comprising, a body, a tail portion, and an anchor port, wherein the anchor port includes an anchor entry and an anchor exit connected by a lumen passing through the tail portion and the body. The method also includes directing an anchor through the anchor entry and into a tissue adjacent to the intervertebral disc. In certain embodiments, the method further includes coupling the anchor to the anchor port. In certain embodiments of the method, the directing the anchor into the tissue includes threading the anchor into the tissue.

In certain embodiments, a method is provided for at least one of (i) treating an annular defect in an intervertebral disc between adjacent vertebrae of a patient, and (ii) maintaining a height between the adjacent vertebrae. The method includes inserting, between the adjacent vertebrae, an implant in a first configuration, the implant comprising an anchor head, a tail portion, and a bias member, wherein the anchor head includes a bias member coupled to at least one of a plurality of anchor members. The method also includes transforming the implant from the first configuration to a second configuration by activating the bias member, thereby producing a force that results in engagement of the tissue by the at least one anchor head. In certain embodiments of the method, the bias member includes a tubular spring coupled to the plurality of anchor members.

In certain embodiments, a method is provided for at least one of (i) treating an annular defect in an intervertebral disc between adjacent vertebrae of a patient, and (ii) maintaining a height between the adjacent vertebrae. The method includes inserting a portion of an implant, in a first configuration, through an opening in the intervertebral disc and into the intervertebral disc space between the adjacent vertebrae, transforming the portion, in the intervertebral disc space, from the first configuration to a second configuration that substantially inhibits the portion from exiting the intervertebral disc space through the opening, and engaging another portion of the implant with the disc, such that the other portion forms a barrier effective to prevent substantial expulsion of material from the disc.

In certain embodiments of the method, the transforming includes rotating the portion in the intervertebral disc space. In certain embodiments of the method, the transforming includes transforming a shape memory material in the portion.

In certain embodiments, a method is provided for at least one of (i) treating an annular defect in an intervertebral disc between adjacent vertebrae of a patient, and (ii) maintaining a height between the adjacent vertebrae. The method includes providing an implant having a head portion coupled to a tail portion, wherein the head portion has a long axis, inserting the head portion, in substantially a first rotational position with respect to the long axis, into the intervertebral disc space between the adjacent vertebrae, and when the head portion is in the intervertebral disc space, rotating at least the head portion from the first rotational position to a second rotational position with respect to the long axis, thereby engaging at least one of the adjacent vertebrae with the head portion. The method also includes engaging the disc with the tail portion, such that the tail portion forms a barrier effective to prevent substantial expulsion of material from the disc.

In certain embodiments of the method, the rotating includes rotating at least the head portion about 90°. In certain embodiments, the method further includes injecting a substance through a lumen in the implant from outside the spine, through the lumen, and into the intervertebral disc space. In certain embodiments of the method, the inserting includes advancing the head portion in a direction substantially along the long axis into the intervertebral disc space.

In certain embodiments, a method is provided for at least one of (i) treating an annular defect in an intervertebral disc between adjacent vertebrae of a patient, and (ii) maintaining a height between the adjacent vertebrae. The method includes providing an implant having a long axis, inserting the implant, in substantially a first rotational position with respect to the long axis, into the intervertebral disc space between the adjacent vertebrae, and when the implant is at least partially in the intervertebral disc space, rotating the implant from the first rotational position to a second rotational position with respect to the long axis, thereby engaging tissue in the intervertebral disc space with the implant. The method also includes engaging the disc with the implant so as to form a barrier effective to prevent substantial expulsion of material from the disc.

In certain embodiments of the method, the rotating includes rotating the implant about 90°. In certain embodiments of the method, engaged tissue in the intervertebral disc space includes at least one of the adjacent vertebrae. In certain embodiments of the method, after the rotating, a portion of the implant maintains a height between the adjacent vertebrae. In certain embodiments, the method further includes injecting a substance through a lumen in the implant from outside the spine, through the lumen, and into the intervertebral disc space. In certain embodiments of the method, the inserting includes advancing the implant in a direction substantially along the long axis into the intervertebral disc space.

A spinal implant system, for at least one of (i) treating a defect in the annulus fibrosus of an intervertebral disc between adjacent vertebrae of a patient, and (ii) maintaining a separation between the adjacent vertebrae. The implant includes a spacer, configured to be inserted into an intervertebral disc space and comprising a lumen. The implant also includes a dilator, configured to be slidably received into the lumen. When the spacer is positioned between the adjacent vertebrae and the dilator is received into the lumen, the spacer expands from a first configuration to a second configuration and secures the implant in the intervertebral disc space. In certain embodiments of the spinal implant system, the spacer is sized and shaped to be inserted through a defect in the annulus fibrosus of the intervertebral disc. In certain embodiments of the spinal implant system, the spacer is elongate, such that when the implant is secured in the intervertebral disc space, the spacer spans from one lateral half of the intervertebral disc space to the opposite lateral half of the intervertebral disc space. In certain embodiments, the spinal implant system also includes a lock that locks the spacer in the second configuration. In certain embodiments, the spinal implant system also includes a lock that locks the dilator in the spacer. In certain embodiments of the spinal implant system, the dilator includes a region that interacts with the spacer to result in at least one of locking the dilator in the spacer and limiting axial movement of the dilator within the spacer. In certain embodiments of the spinal implant system, an end of the spacer has a flared opening into the lumen, to ease insertion of the dilator into the opening. In certain embodiments, the spinal implant system also includes a guidewire configured to be received in the lumen. In certain embodiments, the spinal implant system also includes a pusher, advanceable along the guidewire so as to push the dilator along the guidewire into the lumen. In certain embodiments, when the spacer expands from the first configuration to the second configuration, the spacer expands primarily in an inferior-superior direction with respect to the adjacent vertebrae. In certain embodiments of the spinal implant system, as the dilator is moved axially within the lumen, at least one of an amount and a direction of expansion of the spacer is controllable by a cross-sectional geometry of the dilator. In certain embodiments of the spinal implant system, the spacer expands when the dilator is rotatably introduced into the spacer. In certain embodiments of the spinal implant system, the dilator is sectioned to allow for removal of a portion of the dilator while another portion of the dilator remains in the spacer.

In certain embodiments, an implant is provided for at least one of (i) treating an annular defect in an intervertebral disc between adjacent vertebrae of a patient, and (ii) maintaining a height between the adjacent vertebrae. The implant includes a first implant portion. The implant also includes a second implant portion, wherein the first and second implant portions are configured to be inserted serially into the intervertebral disc space between the adjacent vertebrae. The first and second implant portions are configured to couple to each other within the intervertebral disc space, thereby forming at least part of the implant, upon or after their insertion into the intervertebral disc space. When the implant is positioned between the adjacent vertebrae, the implant engages tissue in the intervertebral disc space, and forms a barrier that prevents substantial expulsion of material from within the disc past the barrier. In certain embodiments, the implant is configured to engage at least one of the adjacent vertebrae. In certain embodiments, the first and the second implant portions couple to form substantially the entire implant.

In certain embodiments, the first implant portion includes a first head portion and a first tail portion. the second implant portion includes a second head portion and a second tail portion, wherein the first head portion and the second head portion couple to form a combined head portion, wherein the first tail portion and the second tail portion couple to form a combined tail portion. When the implant is positioned between the adjacent vertebrae, the combined head portion resides within the intervertebral disc space and engages tissue in the intervertebral disc space, and the combined tail portion contacts a surface of the annulus fibrosus of the intervertebral disc and forms a barrier that prevents substantial expulsion of material from within the disc past the barrier. In certain embodiments, when the combined head portion is positioned between the adjacent vertebrae, the combined tail portion maintains a height between the adjacent vertebrae. In certain embodiments, the combined head portion is configured to engage at least one of the adjacent vertebrae. In certain embodiments, the first and the second implant portions each comprise about half of a mass of the implant. In certain embodiments, when the first and the second implant portions are coupled, the first implant portion at least partially surrounds the second implant portion. In certain embodiments, when the first implant portion and the second implant portions are coupled, they interdigitate with each other. In certain embodiments, the implant further includes a lock configured substantially to prevent separation of the first and second implant portions, once coupled. In certain embodiments, after the implant is positioned between the adjacent vertebrae, a portion of the implant resides within the intervertebral disc space, and another portion of the implant resides outside the intervertebral disc space.

In certain embodiments, a system is provided for use in placing a spinal implant at a site of an opening in an intervertebral disc at an intervertebral disc space. The implant includes a first portion of a spinal implant, a second portion of a spinal implant, wherein the first and second portions of the spinal implant are configured to couple to form a barrier at the opening. The system includes an elongate guide member, configured to be inserted at least partially into the opening and to permit advancement of the first and second portions, along the guide member, from outside the spine into the intervertebral disc space. When the first and second portions are serially advanced along the guide member through the opening and into the intervertebral disc space, and first and second portions couple, the resulting barrier is effective to prevent substantial expulsion of material from the intervertebral disc past the barrier.

In certain embodiments, the guide member slidably engages the first and second portions, and the advancement includes sliding. In certain embodiments, the implant system also includes an implant stop, coupled to the guide member and configured to limit advancement of at least one of the first portion and the second portion into the intervertebral disc space.

In certain embodiments, a spinal implant is provided for at least one of (i) treating a defect in the annulus fibrosus of an intervertebral disc between adjacent vertebrae of a patient, and (ii) maintaining a separation between the adjacent vertebrae. The implant includes a plurality of anchor subunits, each of the plurality of anchor subunits configured to be serially inserted into the intervertebral disc space between the adjacent vertebrae, wherein the anchor subunits are assemblable to form an anchor body after insertion into the intervertebral disc space. When the implant is in position in the patient, the anchor body resides between the adjacent vertebrae, and a portion of the implant engages tissue in the intervertebral disc space, thereby anchoring the implant in the intervertebral disc space. In certain embodiments, the anchor body is configured to engage at least one of the adjacent vertebrae. In certain embodiments, each of the plurality of anchor subunits configured to be slidably inserted along a delivery member into the intervertebral disc space. In certain embodiments, an implant system is provided including the implant, and a delivery member comprising an elongate body that includes at least one of a rod, a wire, and a rail. In certain embodiments, each of the plurality of anchor subunits is coupled to at least another of the anchor subunits. In certain embodiments, at least one of the plurality of anchor subunits is substantially ellipsoidal in shape. In certain embodiments, at least one of the plurality of anchor subunits is lockably coupled to another of the anchor subunits. In certain embodiments, the anchor subunits are assemblable end to end to form the anchor body. In certain embodiments, the anchor subunits are assemblable in a radial array to form the anchor body, each of the anchor subunits extending away from a longitudinal axis of the anchor body. In certain embodiments, the anchor subunits are assemblable in a bunch configuration to form the anchor body. In certain embodiments, the implant is included in an implant system that also includes a delivery member, comprising an elongate body selected from the group consisting of a rod and a wire. In certain embodiments, the implant further includes a first retainer member, coupled to a proximal portion of the anchor body. The implant also includes a second retainer member, coupled to a distal portion of the anchor body at the distal end. When the implant is in position in the patient, the anchor body resides between the adjacent vertebrae, and at least one of the first and second retainer members engages the annulus fibrosus, thereby anchoring the implant in the intervertebral disc space. In certain embodiments, the implant is configured such that, when in position in the patient, the anchor body resides between the adjacent vertebrae, and each of the first and second retainer members engages the annulus fibrosus.

In certain embodiments, the implant is configured such that, when in position in the patient, the anchor body resides between the adjacent vertebrae, and at least one of the first and second retainer members contacts an outer surface of the disc and forms a barrier effective to prevent substantial expulsion of material from the disc. In certain embodiments, the implant is configured such that, when in position in the patient, the anchor body resides between the adjacent vertebrae, and each of the first and second retainer members contacts an outer surface of the disc and forms a barrier effective to prevent substantial expulsion of material from the disc. In certain embodiments, the implant further includes a tail portion, coupled to the anchor body. When the implant is in position in the patient, the tail portion engages the annulus fibrosus of the disc to form a barrier effective to prevent substantial expulsion of material from the disc. In certain embodiments, the tail portion includes a flange.

In certain embodiments, the tail portion includes a flange and a coupling member, wherein the coupling member couples the flange to the anchor body, and wherein the barrier is formed at least in part by the coupling member. In certain embodiments, the implant further includes a connecting member connected to at least one of the anchor subunits, configured such that when a tension is applied to the connecting member, the plurality of anchor subunits assembles into the anchor body.

In certain embodiments, the implant further includes a tail portion, coupled to the anchor body. When the implant is in position in the patient, the tail portion engages the annulus fibrosus of the disc to form a barrier effective to prevent substantial expulsion of material from the disc, wherein the connecting member couples the tail portion to the anchor body and is configured to apply a force on the tail portion effective to maintain contact between the tail portion and the surface of the disc, when the implant is positioned in the patient's spine. In certain embodiments, the anchor body has an aggregate maximum cross-sectional dimension greater than a maximum cross-sectional dimension of any of the plurality of anchor body subunits.

In certain embodiments, a spinal implant is provided for at least one of (i) treating a defect in the annulus fibrosus of an intervertebral disc between adjacent vertebrae of a patient, and (ii) maintaining a separation between the adjacent vertebrae. The implant includes an anchor body that, when positioned between adjacent vertebrae in a spine, is configured to anchor the implant between the adjacent vertebrae and to flex under an axial loading force imposed on the spine. Flexibility of the anchor body is provided by at least one slit in the anchor body. In certain embodiments, the implant further includes a lumen extending through the implant. The implant also includes at least one injection port fluidly connected to the lumen, wherein the at least one injection port is configured to permit passage of an injectable material from outside the implant into the lumen and into the intervertebral disc space. In certain embodiments, the at least one slit has a cross-section having at least two limbs that are transverse to each other.

In certain embodiments, a spinal implant is provided for at least one of (i) treating a defect in the annulus fibrosus of an intervertebral disc between adjacent vertebrae of a patient, and (ii) maintaining a separation between the adjacent vertebrae. The implant includes a plurality of anchor subunits, each of the plurality of anchor subunits configured to be inserted into an intervertebral disc space between the adjacent vertebrae, wherein each of the plurality of anchor subunits slidably interlocks with an adjacent anchor subunit, wherein the plurality of anchor subunits assembles as an elongate anchor body having a proximal end and a distal end. The implant also includes a retainer member at the proximal end that engages the intervertebral disc.

In certain embodiments, at least one of the anchor subunits further includes an opening configured to permit ingrowth of tissue.

In certain embodiments, a spinal implant is provided for at least one of (i) treating a defect in the annulus fibrosus of an intervertebral disc between adjacent vertebrae of a patient, and (ii) maintaining a separation between the adjacent vertebrae. The implant includes a body configured to be inserted into the intervertebral disc space between the adjacent vertebrae, a plurality of anchor elements coupled to the body, configured to engage at least one tissue within or adjacent to the intervertebral disc. The implant also includes at least one bias element, effective to apply a force to at least one of the anchor elements, such that the at least one of the anchor elements engages the at least one tissue, resulting in securement of the implant at the at least one tissue.

In certain embodiments, when the anchor elements are engaged with the at least one tissue, the body forms a barrier effective to prevent substantial expulsion of material from the intervertebral disc. In certain embodiments, the implant further includes a lumen extending through the implant. The implant also includes at least one injection port fluidly connected to the lumen, wherein the at least one injection port is configured to permit passage of an injectable material from outside the implant into the lumen and into the intervertebral disc space. In certain embodiments, when the anchor elements are engaged with the at least one tissue, at least one of the anchor elements engages with at least one of the adjacent vertebrae. In certain embodiments, at least one of the anchor elements includes an arcuate portion. In certain embodiments, when the at least one of the anchor elements engages the at least one tissue, the at least one of the anchor elements moves slidably with respect to, and protrudes from, the body. In certain embodiments, each of the plurality of anchor elements provides a bias force effective to engage the at least one tissue. In certain embodiments, the at least one bias element includes a spring. In certain embodiments, the implant further includes an actuator that moves axially with respect to the body, thereby resulting in at least one of the anchor elements moving outwardly from the body to engage the at least one tissue. In certain embodiments, as the actuator is rotated about a long axis, the actuator moves axially along the long axis, thereby resulting in at least one of the anchor elements moving outwardly from the body to engage the at least one tissue. 6 In certain embodiments, the implant further includes a restraint that maintains at least one of the anchor elements in a first configuration until the implant is placed in the intervertebral disc space, the restraint is manipulable to permit the at least one of the anchor elements to move to a second configuration to engage the at least one tissue. In certain embodiments, the restraint includes a removable sheath. In certain embodiments, at least one of (i) the at least one bias element and (ii) at least one of the plurality of anchor elements includes a shape memory material, configured to change the anchor element from a first configuration to a second configuration in response to an activation energy.

In certain embodiments, a spinal implant is provided for at least one of (i) treating a defect in the annulus fibrosus of an intervertebral disc between adjacent vertebrae of a patient, and (ii) maintaining a separation between the adjacent vertebrae. The implant includes an elongate body having first and second ends and a length therebetween, the body configured to extend through an intervertebral disc, from a first area of the annulus fibrosus of the disc to a second area of the annulus. The implant also includes first and second end plates, located at respective ends of the body, at least one of the end plates being attachable to the body after at least a portion of the body is placed into the disc, such that the endplates each contact an outer surface of the annulus when they are attached to the body and when the body extends through and within the disc, wherein the elongate body has a cross-section that is wider in one dimension than another, such that rotation of the elongate body within the intervertebral disc permits adjustment of a height between the adjacent vertebrae.

In certain embodiments, the elongate body has a cross-section that varies along the length of the body, such that axial motion of the body within the intervertebral disc permits adjustment of a height between the adjacent vertebrae. In certain embodiments, the implant further includes a lumen extending through at least one of the end plates, permitting advancement of the implant along a guidewire. In certain embodiments, the elongate body includes a plurality of elongate slats that each extend between the end plates. In certain embodiments, the elongate body is configured to expand in a cross-sectional dimension by movement of at least one of the slats away from another of the slates. In certain embodiments, when the endplates each contact an outer surface of the annulus and are attached to the body, and when the body is positioned to extend through the disc, at least one of the end plates forms a barrier effective to prevent substantial expulsion of material from the intervertebral disc. In certain embodiments, the body is self-expanding. In certain embodiments, the body includes a shape memory material configured to expand in response to an activation energy.

In certain embodiments, a spinal implant is provided for at least one of (i) treating a defect in the annulus fibrosus of an intervertebral disc between adjacent vertebrae of a patient, and (ii) maintaining a separation between the adjacent vertebrae. The implant includes an elongate member having a lumen, the elongate member having first and second ends, the elongate member configured to extend through an intervertebral disc, from a first area of the annulus fibrosus of the disc to a second area of the annulus, and includes an injection port in fluid communication with the lumen and opening at or near the first end. The implant also includes at least one port in the elongate member, configured to permit movement of a substance from within the lumen, through the port, and into a space adjacent to the implant, a fixation member, coupled to the elongate member and passing through the lumen, such that when the elongate member is positioned in the intervertebral disc, the fixation member engages the annulus at a region closer to the second end of the elongate member than to the first end, resulting in fixation of the implant within the intervertebral disc. In certain embodiments, the fixation member includes a screw. In certain embodiments, the at least one port includes a plurality of ports arrayed along the elongate member.

In certain embodiments, a method is provided for at least one of (i) treating a defect in an intervertebral disc between adjacent vertebrae, and (ii) maintaining a separation between adjacent vertebrae. The method includes positioning a spacer in an intervertebral disc space between the adjacent vertebrae, and inserting a dilator into a lumen in the spacer, thereby expanding the spacer from a first configuration to a second configuration and thereby securing the implant in the intervertebral disc space.

In certain embodiments of the method, the positioning includes inserting the spacer through a defect in the annulus fibrosus of an intervertebral disc between the adjacent vertebrae. In certain embodiments of the method, the positioning includes inserting the spacer transversely, from one lateral aspect of the intervertebral disc space toward an opposite lateral aspect of the intervertebral disc space. In certain embodiments, the method further includes locking the spacer in the second configuration. In certain embodiments, the method further includes locking the dilator in the spacer, such that the spacer is in the second configuration after the locking. In certain embodiments, the method further includes interacting the dilator with the spacer to result in at least one of locking the dilator in the spacer and limiting axial movement of the dilator within the spacer. In certain embodiments, the method further includes inserting a guidewire into the lumen. In certain embodiments, the method also includes advancing a pusher along the guidewire, thereby pushing the dilator into the lumen and expanding the spacer. In certain embodiments, the method further includes entering, with a guidewire, into the intervertebral disc at a first location, exiting, with the guidewire, from the intervertebral disc at a second location, and advancing the spacer along the guidewire into the intervertebral disc space. In certain embodiments, the method also includes advancing the dilator along the guidewire into the lumen, thereby expanding the spacer. In certain embodiments, the inserting results in the spacer expanding primarily in an inferior-superior direction with respect to the adjacent vertebrae as the spacer expands from the first configuration to the second configuration. In certain embodiments, the method further includes moving the dilator axially within the lumen. In certain embodiments, the method further includes controlling at least one of an amount and a direction of expansion of the spacer based on a cross-sectional geometry of the dilator.

In certain embodiments, a method is provided for at least one of (i) treating a defect in an intervertebral disc between adjacent vertebrae, and (ii) maintaining a separation between adjacent vertebrae. The method includes inserting a first anchor subunit into an intervertebral disc space between the adjacent vertebrae, while or after inserting a second anchor subunit in the intervertebral disc space, slidably interlocking the first and second anchor subunits within the intervertebral disc space, such that the interlocked first and second anchor subunits form an anchor body that resides in the intervertebral disc space. The method also includes securing a proximal region of the anchor body at the annulus fibrosus of the intervertebral disc.

In certain embodiments of the method, the anchor body is elongate. In certain embodiments, the method further includes forming a barrier with the proximal region, effective to prevent substantial expulsion of material from the disc past the barrier. In certain embodiments of the method, the securing includes contacting an outer surface of the disc with a proximal part of the anchor body. In certain embodiments of the method, the inserting of the second anchor subunit results in maintaining a separation between the adjacent vertebrae by the anchor body.

In certain embodiments, a method is provided for at least one of (i) treating a defect in an intervertebral disc in an intervertebral disc space, and (ii) maintaining a separation between adjacent vertebrae. The method includes serially inserting a plurality of anchor subunits into an opening in the intervertebral disc, each of the anchor subunits being couplable to at least another of the anchor subunits, and arranging the plurality of anchor subunits in the intervertebral disc space to form an anchor body that is at least part of an implant, the anchor body configured such that it is inhibited from exiting the intervertebral disc space through the opening. The method also includes anchoring the implant in the intervertebral disc space.

In certain embodiments, the method further includes engaging the implant with the annulus fibrosus of the intervertebral disc, thereby forming a barrier effective to prevent substantial expulsion of material from the disc past the barrier. In certain embodiments, the method further includes locking the anchor body to inhibit movement of the plurality of anchor subunits. In certain embodiments, the method further includes coupling each of the anchor subunits to at least another of the anchor subunits. In certain embodiments of the method, the anchor subunits assemble end to end to form the anchor body. In certain embodiments of the method, the anchor subunits assemble in a radial array to form the anchor body, each of the anchor subunits extending away from a longitudinal axis of the anchor body. In certain embodiments of the method, the anchor subunits assemble in a bunch configuration to form the anchor body.

In certain embodiments, a method is provided for at least one of (i) treating an annular defect in an intervertebral disc between adjacent vertebrae of a patient, and (ii) maintaining a height between the adjacent vertebrae. The method includes inserting a first implant portion into the intervertebral disc space between the adjacent vertebrae. The method also includes after the inserting the first implant portion, inserting a second implant portion into the intervertebral disc space between the adjacent vertebrae, coupling the first implant portion with the second implant portion after their insertion into the intervertebral disc space, thereby forming at least part of the implant, engaging at least one of the adjacent vertebrae with the implant. The method also includes forming a barrier by engaging the disc with the implant, such that the barrier prevents substantial expulsion of material from within the disc past the barrier.

In certain embodiments of the method, the coupling of the first and the second implant portions forms substantially the entire implant. In certain embodiments of the method, the coupling includes at least partially surrounding one of the implant portions with the other of the implant portions. In certain embodiments of the method, the coupling includes interdigitating one of the implant portions with the other of the implant portions. In certain embodiments, the method further includes locking the first and second implant portions together, once coupled.

In certain embodiments, a method is provided for at least one of (i) treating an annular defect in an intervertebral disc between adjacent vertebrae of a patient, and (ii) maintaining a height between the adjacent vertebrae. The method includes inserting a first implant portion comprising a first head portion and a first tail portion between the adjacent vertebrae, and inserting a second implant portion comprising a second head portion and a second tail portion between the adjacent vertebrae. The method also includes coupling the first implant portion and the second implant portion. When the first and second implant portions are coupled between the adjacent vertebrae, the first tail portion and the second tail portion form a combined tail portion that contacts a surface of the intervertebral disc and form a barrier that prevents substantial expulsion of material from within the disc past the first and second tail portions. When the first and second implant portions are coupled between the adjacent vertebrae, the first head portion and the second head portion form a combined head portion that engages tissue at or near the intervertebral disc. In certain embodiments, the method further includes locking the first implant portion with the second implant portion.

In certain embodiments, a method is provided for at least one of (i) treating a defect in an intervertebral disc between adjacent vertebrae, and (ii) maintaining a separation between adjacent vertebrae. The method includes providing an elongate member, comprising (i) a lumen extending from a first end to a second end of the elongate member, and (ii) a fixation member that extends within the lumen and beyond the second end, inserting the elongate member into an intervertebral disc space between the adjacent vertebrae, such that the elongate member extends through the intervertebral disc, from a first area of the annulus fibrosus of the disc to a second area of the annulus, injecting a substance into the lumen from a point at or near the first end, such that substance moves from within the lumen, through at least one opening in the elongate member, and into the intervertebral disc space, manipulating the fixation member to secure the implant at the annulus at a region closer to the second end of the elongate member than to the first end, resulting in fixation of the implant within the intervertebral disc.

In certain embodiments of the method, the manipulating includes rotating the fixation member. In certain embodiments of the method, the substance includes at least one of a pharmaceutical agent, a gel, a swellable polymer, a paste, and a glue. In certain embodiments, a method is provided for maintaining a height between adjacent vertebrae of a patient. The method includes inserting an implant between the adjacent vertebrae, after the inserting, and with a movable portion of the implant, penetrating an endplate of at least one of the adjacent vertebrae, thereby securing the implant between the adjacent vertebrae.

In certain embodiments of the method, the inserting is performed through a minimally invasive surgical opening in the skin of the patient. In certain embodiments of the method, the anchor member includes is a screw. In certain embodiments of the method, the anchor member includes at least one of a hook and a barb.

In certain embodiments, a method is provided for at least one of (i) treating an annular defect in an intervertebral disc between adjacent vertebrae of a patient, and (ii) maintaining a height between the adjacent vertebrae. The method includes inserting an implant comprising a head portion and a tail portion, into the intervertebral disc space of the patient, wherein the head portion includes a plurality of anchor members, and directing, into a tissue of or adjacent to the intervertebral disc, the plurality of anchor members.

In certain embodiments of the method, the directing, into the tissue adjacent to the intervertebral disc, includes moving each of the plurality of anchor members from a first configuration to a second configuration. In certain embodiments of the method, the moving each of the plurality of anchor members from the first configuration to the second configuration includes releasing at least one of the plurality of anchor members from a retainer member configured to maintain the plurality of anchors in the first configuration. In certain embodiments of the method, the releasing the at least one of the plurality of anchor members from the retainer member includes slidably releasing an anchor release member configured to release the at least one of the plurality of anchor members from the retainer member. In certain embodiments of the method, the releasing the at least one of the plurality of anchor members from the retainer member includes threadably releasing an anchor release member. In certain embodiments of the method, the plurality of anchor members comprise a shape memory material, and wherein the moving each of the plurality of anchor members from the first configuration to the second configuration includes activating the shape memory material using an activation energy.

In certain embodiments disclosed herein, a reamer, for use in preparing a tissue at a surgical site, comprises a cutting system, comprises a handle; a first shaft, having proximal and distal portions, the proximal portion of the first shaft coupled to the handle; a first cutting member, coupled to the distal portion of the first shaft; and a limiter, coupled to the cutting system and configured to limit a depth of penetration of the reamer into the surgical site during preparation of the tissue.

In certain embodiments disclosed herein, the reamer further comprises a second cutting member; and the first cutting member and the second cutting member form an assembly, configured to expand from a first configuration, having a first cross-sectional dimension, to a second configuration, having a second cross-sectional dimension larger than the first cross-sectional dimension. In certain embodiments disclosed herein, the assembly comprises a tapered distal end to assist entry into an aperture in annulus fibrosus of an intervertebral disc. In certain embodiments disclosed herein, the reamer further comprises a tapered nose cone at a distal end of the reamer, the nose cone configured to distract adjacent vertebrae. In certain embodiments disclosed herein, in a reamer for use in preparing an intervertebral disc of a mammal to receive a spinal implant, the assembly in the first configuration is configured for insertion into an opening in the annulus of the intervertebral disc; and the assembly in the second configuration is configured for cutting tissue from within the intervertebral disc space.

In certain embodiments disclosed herein, the assembly changes from the first configuration to the second configuration in response to movement of the handle with respect to the first shaft. In certain embodiments disclosed herein, the reamer the movement comprises axial movement of the handle with respect to the first shaft. In certain embodiments disclosed herein, the movement comprises rotational movement of the handle with respect to the first shaft. In certain embodiments disclosed herein, at least one of the first and second cutting members comprises at least one cutting edge, comprises at least one of a straight cutting edge and a helical cutting edge. In certain embodiments disclosed herein, the reamer further comprises a second shaft, having proximal and distal portions, the proximal portion of the second shaft coupled to the handle; and the second cutting member is coupled to the distal portion of the second shaft.

In certain embodiments disclosed herein, the second shaft is spring biased away from the first shaft at a distal portion of the second shaft. In certain embodiments disclosed herein, the reamer further comprises a slider that at least partially surrounds the first and second shafts; at least one of the first and second shafts are slidable within the slider; and the assembly changes from the first configuration to the second configuration in response to movement of the slider with respect to the handle.

In certain embodiments disclosed herein, at least a portion of the first shaft is housed within a longitudinal cavity of the second shaft. In certain embodiments disclosed herein, the second shaft comprises a cutout portion extending along a length of the second shaft, such that, as the distal portion of the second shaft moves away from the first shaft due to the spring bias, at least a portion of the first shaft extends away from the second shaft through the cutout portion. In certain embodiments disclosed herein, the reamer further comprises a retainer, coupled to the first cutting member; and a slot in the second cutting member, the retainer extending into the slot; wherein a movement of the second cutting member with respect to the first cutting member in response to the spring bias is limited by a limitation of movement of the retainer in the slot.

In certain embodiments disclosed herein, at least a portion of the first shaft is housed within a longitudinal cavity of the second shaft; the first and second shafts rotate about a longitudinal axis; and an axial motion of the second shaft with respect to the first shaft, substantially along the longitudinal axis, results in a secondary rotation of the second cutting member about a different axis than the longitudinal axis and results in the assembly changing from the second configuration to the first configuration. In certain embodiments disclosed herein, rotation of the handle causes at least one of the assembly to lock in the second configuration. In certain embodiments disclosed herein, the handle comprises a first handle portion and the second handle portion, and the secondary rotation of the second cutting member occurs upon movement of the first handle portion with respect to the second handle portion.

In certain embodiments disclosed herein, a method for preparing an intervertebral disc to receive a spinal implant comprises providing a reamer, the reamer comprising a handle; a first shaft, having proximal and distal portions, the proximal portion of the first shaft coupled to the handle; a first cutting member, coupled to the distal portion of the first shaft; and a second cutting member; the first cutting member and the second cutting member form an assembly that has a primary rotation about a f axis of the shaft. The method further comprises inserting the assembly, in a first configuration having a first cross-sectional dimension, into an opening in an intervertebral disc space; in the intervertebral disc space, expanding the assembly from the first configuration to a second configuration having a second cross-sectional dimension larger than the first cross-sectional dimension; and using the first and the second cutting members, cutting tissue in the intervertebral disc space with the assembly in the second configuration.

In certain embodiments disclosed herein, the method further comprises limiting a depth of penetration of the reamer with a limiter coupled to the reamer. In certain embodiments disclosed herein, the method further comprises increasing a distance between distal ends of the first shaft and the second shaft by moving a coupling member that couples the first shaft to the second shaft. In certain embodiments disclosed herein, the method further comprises increasing a distance between the first cutting member and the second cutting member by removing a coupling member configured to couple the first shaft to the second shaft. In certain embodiments disclosed herein, the method further comprises moving the second shaft within a longitudinal cavity of the first shaft, thereby resulting in (i) a secondary rotation of the second cutting member, about a different axis than the longitudinal axis, and (ii) the assembly changing from the second configuration to the first configuration. In certain embodiments disclosed herein, the method further comprises locking the assembly in the second configuration by rotating a portion of the handle.

In certain embodiments disclosed herein, a spiral reamer, for use in preparing a tissue at a surgical site, comprises an attachment portion, configured for attachment to a rotatable device; and a cutting member, coupled to the attachment portion, comprises an elongate strip, wound at least partially in a coil, the strip having a free end at an outer aspect of the coil; wherein rotation of the cutting member at a tissue results in cutting of the tissue by the free end.

In certain embodiments disclosed herein, rotation of the cutting member results in at least a partial unwinding of the coil, resulting in expansion of a cross-sectional dimension of the coil, for cutting of the tissue. In certain embodiments disclosed herein, the spiral reamer further comprises at least one cutting element disposed in or on the strip, wherein the cutting element comprises at least one of an opening in the strip, a burr, and a spike.

In certain embodiments disclosed herein, a method for preparing an intervertebral disc and delivering a spinal implant to the disc, comprises forming an opening in the skin of a patient; with an instrument, inserting a reamer through the opening and into an intervertebral disc space between adjacent vertebrae of the patient; cutting tissue at the intervertebral disc pace with the reamer; withdrawing the instrument from the patient; and closing the opening in the skin, leaving the reamer at least partially in the intervertebral space, such that the reamer (a) forms a barrier effective to prevent substantial expulsion of material from the intervertebral disc space, or (b) maintains a height between the adjacent vertebrae, or both (a) and (b).

In certain embodiments disclosed herein, a distractor, for use in increasing the space between adjacent vertebrae, comprises an upper handle comprises an upper jaw; a lower handle, coupled to the upper handle about a pivot, comprises a lower jaw; and a ratchet engagement at a proximal end of the lower handle; and a ratchet member, coupled to a proximal portion of the upper handle, comprises a plurality of teeth; wherein the ratchet engagement couples to the ratchet member at least one of the plurality of teeth.

In certain embodiments disclosed herein, the distractor further comprises a bias spring, coupled to at least one of the upper handle and the lower handle, configured to assist in increasing a distance between the proximal ends of the upper handle and the lower handle.

In certain embodiments disclosed herein, a method for increasing the space between adjacent vertebrae, comprises providing a distractor, the distractor comprises an upper handle comprises an upper jaw; a lower handle, coupled to the upper handle about a pivot, comprises a lower jaw; and a ratchet engagement at a proximal end of the lower handle; and a ratchet member, coupled to a proximal portion of the upper handle; wherein the ratchet engagement adjustably couples to the ratchet member; inserting at least a portion of the upper jaw and a portion of the lower jaw into the intervertebral disc space; increasing the distance between the upper and the lower jaw and moving the ratchet engagement from a first position to a second position, thereby increasing a height of intervertebral disc space.

In certain embodiments disclosed herein, an implant delivery system, for placing a spinal implant at a site of an opening in an intervertebral disc, comprises a spinal implant, configured to be inserted into an intervertebral disc space; an elongate member; an implant coupler disposed at a distal end of the elongate member and configured to releasably engage the spinal implant; wherein the implant coupler comprises a sheath that slides around the implant and retracts proximally when the coupler releases the implant into the intervertebral disc space.

In certain embodiments disclosed herein, the device is configured to rotate the spinal implant after the implant is placed in the intervertebral disc space, to engage the implant with tissue at the intervertebral disc space.

In certain embodiments disclosed herein, an implant sizing kit, for sizing and placing a spinal implant at a site of an intervertebral disc, comprises a spinal implant, configured to be inserted into an intervertebral disc space; and an elongate sizing member, having an end portion that is substantially elliptical, with a major axis and a minor axis, in cross section; wherein the sizing member is configured to determine a height of the intervertebral disc space using a length of the minor axis; wherein the sizing member is further configured to distract the adjacent vertebrae to a height of approximately a length of the major axis, when the end portion is within the intervertebral disc space, by rotation of the end portion within the intervertebral disc space.

In certain embodiments disclosed herein, a sizing kit, for use in selecting a size of a spinal implant to be implanted in an intervertebral disc space, comprises a plurality of head portions, of varying sizes, each of the plurality of head portions sized and shaped to be placed between adjacent vertebrae; and a tail portion, configured to be coupled to at least one of the plurality of head portions; wherein, when at least one of the plurality of head portions is positioned between the two adjacent vertebrae, and the tail portion is coupled to the at least one of the plurality of head portions, the tail portion contacts a surface of an intervertebral disc located between the two adjacent vertebrae and forms a barrier that substantially prevents expulsion of material from within the disc past the barrier portion.

In certain embodiments disclosed herein, a method for selecting a size of a spinal implant to be implanted at a site of a defect in an intervertebral disc between adjacent vertebrae, comprises providing a plurality of head portions of varying sizes, at least one of the plurality of head portions sized and shaped to be placed between the adjacent vertebrae; inserting the at least one head portion from the plurality of head portions into the intervertebral disc space; positioning the at least one head portion between the adjacent vertebrae; and coupling a tail portion to the at least one head portion such that the tail portion contacts a surface of an intervertebral disc and forms a barrier that substantially prevents expulsion of material from within the intervertebral disc past the barrier portion.

In certain embodiments disclosed herein, a trial unit kit, for use in preparing an intervertebral disc for placement of a spinal implant, comprises a spinal implant, configured to be inserted into an intervertebral disc space between adjacent vertebrae; and a trial unit; comprises elongate member, comprises an end portion having a cross-sectional profile that is substantially identical to a cross-sectional profile of the implant; wherein the trial unit is configured to be inserted at least partially into the intervertebral disc space for at least one of sizing the intervertebral disc space, determining a depth of a space in the intervertebral disc space, arranging tissue in the intervertebral disc space, and distraction of the adjacent vertebrae.

In certain embodiments disclosed herein, a method for preparing a vertebral lip to receive a spinal implant, comprises providing a trial unit comprises a handle; a shaft, coupled to the handle; a head portion, coupled to the shaft; and a tail portion, configured to limit the depth of penetration of the trial unit during preparation of an implant site; creating an intervertebral disc space; and inserting the head portion into the intervertebral disc space.

For purposes of summarizing the disclosure, certain aspects, advantages and novel features are described herein. It is to be understood that not necessarily all such advantages may be achieved in accordance with any particular embodiment of the disclosure. Thus, for example, the disclosure can be embodied or carried out in a manner that achieves one advantage or group of advantages as taught herein without necessarily achieving other advantages as may be taught or suggested herein.

### BRIEF DESCRIPTION OF THE DRAWINGS

FIG. 1 is a front perspective view of an embodiment of the present spinal implants.

FIG. 2 is a front elevational view of the spinal implant of FIG. 1.

FIG. 3 is a right-side elevational view of the spinal implant of FIG. 1.

FIG. 4 is a right-side elevational view of a normal intervertebral disc, the adjacent vertebrae and a spinal nerve.

FIG. 5 is a right-side elevational view of a herniated intervertebral disc, the adjacent vertebrae and a spinal nerve.

FIG. 6 is a right-side elevational view of the disc of FIG. 5 after a microdiscectomy procedure.

FIG. 7 is a right-side elevational view of the disc of FIG. 6 and the implant of FIG. 1.

FIG. 8 is a right-side elevational view of the disc and the implant of FIG. 7, showing the implant implanted within the disc.

FIG. 9 is a right-side elevational view of the disc of FIG. 6 and an embodiment of a reaming tool that may be used during a procedure to implant the implant of FIG. 1.

FIG. 10 is a right-side elevational view of the disc of FIG. 9 after the reaming step, and a countersinking tool that may be used during a procedure to implant the implant of FIG. 1.

FIG. 11 is a right-side elevational view of the disc of FIG. 10 after the countersinking step, and a sizing tool that may be used during a procedure to implant the implant of FIG. 1.

FIG. 12 is a right-side elevational view of the disc of FIG. 11 after the sizing step, and a trial implant that may be used during a procedure to implant the implant of FIG. 1.

FIG. 13 is a right-side elevational view of the disc of FIG. 12 and the implant of FIG. 1, showing the implant implanted within the disc.

FIG. 14 is a front perspective view of another embodiment of the present spinal implants.

FIG. 15 is a front elevational view of the spinal implant of FIG. 14.

FIG. 16 is a right-side elevational view of the spinal implant of FIG. 14.

FIG. 17 is a front perspective view of an embodiment of the present spinal implants.

FIG. 18 is a front elevational view of the spinal implant of FIG. 17.

FIG. 19 is a right-side elevational view of the spinal implant of FIG. 17.

FIG. 20 is a front perspective view of an embodiment of the present spinal implants.

FIG. 21 is a front elevational view of the spinal implant of FIG. 20.

FIG. 22 is a right-side elevational view of the spinal implant of FIG. 20.

FIG. 23 is a front perspective view of an embodiment of a reaming tool that may be used during a procedure to implant the present implants.

FIG. 24 is a right-side elevational view of the reaming tool of FIG. 23.

FIG. 25 is a front perspective view of an embodiment of a countersinking tool that may be used during a procedure to implant the present implants.

FIG. 26 is a right-side elevational view of the countersinking tool of FIG. 25.

FIG. 27 is a front perspective view of an embodiment of a sizing tool that may be used during a procedure to implant the present implants.

FIG. 28 is a right-side elevational view of the sizing tool of FIG. 27.

FIG. 29 is a front perspective view of an embodiment of a trial implant that may be used during a procedure to implant the present implants.

FIG. 30 is a right-side elevational view of the trial implant of FIG. 29.

FIGS. 31(A)-(B) illustrate a front perspective view of a hollow spinal implant with bone compaction holes (A) and the device implanted within the disc (B).

FIGS. 32(A)-(C) illustrate a front perspective view of a hollow splined spinal implant with (A and C), and the device implanted within the disc (B).

FIGS. 33(A)-(C) illustrate a front perspective view of a splined spinal implant with a solid surface (A and C), and the device implanted within the disc (B).

FIGS. 34(A)-(B) illustrate a front perspective view of a threaded spinal implant with (A), and the device implanted within the disc (B).

FIGS. 35(A)-(B) illustrate a front perspective view of a barbed spinal implant with (A), and the device implanted within the disc (B).

FIGS. 36(A)-(B) illustrate a front perspective view of a spinal implant a centrally located hole for placement of the implant with a guide wire (A), and the device implanted within the disc (B).

FIGS. 37(A)-(B) illustrate a front perspective view of a spinal implant with a centrally located hole for placement of the implant with a guide wire, and thin tail segment (A), and the device implanted within the disc (B).

FIG. 38 illustrates a front perspective view of a spinal implant with a threadable tailpiece.

FIG. 39 illustrates a front perspective view of a spinal implant with an insertable tailpiece.

FIG. 40 illustrates a front perspective view of a spinal implant with head and tail portions made from different materials.

FIGS. 41(A)-(E) are side views of spinal implants with variously shaped tail flanges, implanted within the disc.

FIG. 42A illustrates an embodiment of an annular implant comprising a tail flange, an anchor, and a tether.

FIG. 42B illustrates an embodiment of an annular implant comprising a tail flange, a tether, and a collapsible anchor having been inserted into an intervertebral disc, wherein the collapsible anchor has expanded.

FIGS. 43(A)-(B) illustrate side and front views respectfully of an embodiment of an annular implant comprising a tail flange, a tail, and an anchor, wherein the anchor comprises slots which permit resilient compression of the anchor.

FIG. 44 illustrates an embodiment of an annular implant comprising a tail flange, a tether system, and fasteners capable of embedment within the vertebrae.

FIG. 45A illustrates an embodiment of an annular implant comprising a two-part structure that can be assembled in place to minimize height requirements for insertion.

FIG. 45B illustrates the annular implant of FIG. 45A wherein the two pieces have been brought together and coupled.

FIG. 46A illustrates an embodiment of an annular implant comprising an expandable anchor coupled to a rivet-like structure, the exterior of which seals an annular defect.

FIG. 46B illustrates the annular implant of FIG. 46A wherein the anchoring structures have been expanded into nuclear tissue laterally and into bony or cartilaginous structures out of the plane of the illustration.

FIG. 47 illustrates an embodiment of an annular implant comprising a tail flange coupled to hook anchors secured within the intervertebral disc.

FIG. 48 illustrates an embodiment of an annular implant comprising an artificial nucleus and a tail flange coupled together, wherein the artificial nucleus is an expandable sac filled with gel, liquid or other material.

FIG. 49A illustrates a longitudinal cross-sectional view of an annular implant comprising a core element, a tail flange having active retraction properties, and a compressed circumferential coil.

FIG. 49B illustrates a longitudinal cross-sectional view of an annular implant comprising a core element, a tail flange having active retraction properties, and an expanded circumferential coil.

FIGS. 50(A)-(B) illustrate an embodiment of an annular implant comprising a tail flange, and an axially elongate body comprising flat wire spring elements and polymeric bone seat elements.

FIG. 51A illustrates side view of an embodiment of an annular implant comprising a tail flange, a tail, an anchor body, and a pinwheel spring lock secured to the anchor body.

FIG. 51B illustrates a lateral cross-section of the annular implant of FIG. 51A wherein the pinwheel spring is compressed within a circumferential groove in the anchor body.

FIG. 51C illustrates a lateral cross-section of the annular implant of FIG. 51A wherein the pinwheel spring has expanded.

FIG. 52 illustrates an embodiment of an annular implant comprising a tail flange, a tail, and a hollow anchor body further comprising spring-loaded locking pins.

FIGS. 53(A)-(D) illustrate embodiments of an annular implant comprising an anchor body and spring loaded hooks.

FIGS. 54(A)-(C) illustrate views of an embodiment of an annular implant comprising a tail flange, an axially elongate body, and a plurality of radially outwardly deformable anchoring members.

FIG. 55A illustrates an embodiment of a tail configuration for an annular implant wherein the tail is coated with a thin layer of dried water-swellable hydrophilic hydrogel capable of volumetric expansion.

FIG. 55B illustrates the tail configuration of FIG. 55A wherein the hydrophilic hydrogel has absorbed water and has swollen to an increased volume.

FIG. 56 illustrates an embodiment of an annular implant comprising a tail flange, a tail, an anchoring body, and a plurality of spring-loaded hooks affixed thereto.

FIGS. 57(A)-(B) illustrate side and front views of an embodiment of an annular implant comprising spring elements cut from a tube and polymeric bone seat elements.

FIGS. 58(A)-(B) illustrate an embodiment of an annular implant comprising a tail flange, an axially elongate body, a split collet hook system, and a central wedge that can be advanced under mechanical advantage to expand and lock the collet hooks.

FIG. 59 illustrates an embodiment of an annular implant comprising an exterior patch and an interiorly projecting plug, wherein the exterior patch can comprise hooks or bond anchors for externally attaching to the vertebrae.

FIG. 60A illustrates an embodiment of an annular implant comprising an axially elongate rod advanced transversely through an intervertebral disc to prevent outflow of disc material through a posteriorly directed annular defect.

FIG. 60B illustrates an embodiment of an annular implant comprising a tail flange coupled to a self-tunneling coil structure that can be inserted into the core of the intervertebral disc.

FIG. 61 illustrates an embodiment of an annular implant comprising a tail flange, a tail, an anchor body, and bone growth materials affixed to either a cranially or caudally facing portion of the anchor body.

FIGS. 62(A)-(C) illustrate an embodiment of an annular implant comprising a multi-piece, assemble in place construction wherein an anchor is advanced into the annular defect and rotated 90° to maximally engage the vertebrae, following which a tail structure is affixed thereto.

FIG. 63A illustrates an embodiment of a collapsed annular implant comprising a tail flange, a tail, an inflatable anchor, and a filling port in the tail flange.

FIG. 63B illustrates an embodiment of an expanded annular implant comprising a tail flange, a tail, and an inflatable anchor, wherein the inflatable anchor has been filled with polymeric material through a port in the tail flange.

FIG. 64A illustrates an embodiment of an annular implant comprising a tail flange and a tail, where the tail can comprise a lumen or channel leading from the proximal side of the tail to an exit point near the distal end but on the radially outwardly directed surface of the tail. Anchoring fasteners can be passed through the channels and embedded within the vertebrae.

FIG. 64B illustrates the annular implant of FIG. 64A, where the anchoring fasteners have been inserted into the channels, deflected laterally, and are embedded in the vertebrae.

FIG. 65 illustrates an embodiment of an annular implant comprising a resilient polymeric anchor, tail, and tail flange.

FIG. 66 illustrates an embodiment of an annular implant comprising a resilient polymeric anchor affixed to a rigid tail and rigid tail flange.

FIG. 67A illustrates an annular defect in an intervertebral disc, wherein the defect has been prepared by reaming.

FIG. 67B illustrates the annular defect of FIG. 67A wherein a first piece and a second piece of an embodiment of a multi-piece implant have been inserted into the defect.

FIG. 67C illustrates the annular defect of FIG. 67A wherein a third piece of an embodiment of a multi-part implant is inserted into the defect, following which the first part can be drawn against the second and third pieces to complete assembly, following which the insertion tool has been disconnected leaving the three-part implant in place.

FIG. 68 illustrates a tail configuration for an embodiment of an implant adapted for closure of an annular defect in an intervertebral disc, wherein the tail can be spring biased toward the anchoring body of the implant.

FIG. 69A illustrates a tail configuration for an embodiment of an implant adapted for closure of an annular defect in an intervertebral disc, wherein the tail can be radially expandable using an accordion mechanism.

FIG. 69B illustrates a tail configuration for an embodiment of an implant adapted for closure of an annular defect in an intervertebral disc, wherein the tail can be radially expandable by rotating plates outward.

FIG. 69C illustrates a tail configuration for an embodiment of an implant adapted for closure of an annular defect in an intervertebral disc, wherein the tail can be radially expandable outward by a jackscrew.

FIGS. 70(A)-(B) illustrate embodiments of an annular implant comprising an expandable braid or mesh anchor and a tail flange, wherein reduction in the distance between the two ends of the braid can result in radial expansion of the expandable braid.

FIG. 71A illustrates a lateral view of an intervertebral disc with an annular defect, having been reamed to accommodate an embodiment of an annular implant.

FIG. 71B illustrates a lateral view of an intervertebral disc with an annular defect, wherein an embodiment of implant has been inserted into the annular defect such that the implant can be turned sideways to minimize its profile between the two vertebrae.

FIG. 71C illustrates the implant of FIG. 71B having been rotated 90 degrees to maximize the profile of an anchoring portion within the intervertebral disc.

FIG. 72A illustrates the implant of FIG. 60A wherein the implant comprises a straight cylindrical interconnecting member between two end plates to secure the implant in the patient's tissue.

FIG. 72B illustrates the implant of FIG. 72A, wherein the implant comprises a ribbon-like interconnecting member between two end plates.

FIG. 72C illustrates the implant of FIG. 72A wherein the implant comprises an interconnecting member that has variable diameter or thickness.

FIG. 72D illustrates the implant of FIG. 72A wherein the implant comprises multiple interconnecting members between two end plates and further wherein the interconnecting members are elastomeric and optionally expandable.

FIG. 73 illustrates a side view of an embodiment of a lip reamer.

FIG. 74A illustrates a side view of an embodiment of a delivery system, in partial breakaway view, for an annular implant.

FIG. 74B illustrates a side view of an embodiment of a delivery system for an annular implant, wherein the delivery system is capable of imparting rotational forces to the implant.

FIG. 75A illustrates a side view of an embodiment of a reamer for an annular implant.

FIG. 75B illustrates a face on view of an embodiment of a four flute reamer bit.

FIG. 76A illustrates a side view of an embodiment of a trial unit for an annular implant.

FIG. 76B illustrates a side view of an embodiment of a lip sizer for an annular implant.

FIG. 77(A)-(C) are side views of embodiments of spinal implants comprising a head portion and tail portion coupled by a flexible tether.

FIG. 77D is a view of an embodiment of an implant like those in FIG. 77(A)-(C), implanted in a disc.

FIG. 78 is a coronal view of an embodiment of a spinal implant as shown in FIG. 77-C, implanted in a spine.

FIG. 79(A)-(B) illustrate embodiments of spinal implants without tapered segments.

FIGS. 79(C)-(D) illustrate the implants of FIG. 79(A)-(B) implanted within the disc.

FIG. 80A illustrates a perspective view of a spinal implant device with a portion of the implant comprising bone-compaction holes.

FIG. 80B illustrates a front view of the implant shown in FIG. 80A.

FIG. 80C illustrates a side view of the implant of FIG. 80A implanted within the disc.

FIG. 81A illustrates a perspective view of an embodiment of a compliant spinal implant device comprising a split.

FIG. 81B illustrates a front view of the implant of FIG. 81 A.

FIG. 81C illustrates a side view of the implant of FIG. 81A implanted within the disc.

FIG. 82 illustrates a perspective view of an embodiment of a compliant spinal implant device that also comprises bone-compaction holes on one portion of the device.

FIG. 83A illustrates a perspective view of embodiments of compliant spinal implant devices comprising a head portion and including bone compaction holes.

FIG. 83B illustrates a perspective view of embodiments of compliant spinal implant devices comprising a head portion and lacking bone compaction holes.

FIG. 84A illustrates a side view of an embodiment of an annular implant, comprising a plurality of inner lumens configured to receive flexible anchors, at a site of a defect in an intervertebral disc.

FIG. 84B illustrates a side view of the annular implant of FIG. 84A, where flexible anchors have been inserted and forced into bone adjacent to the anchoring head.

FIG. 85A illustrates a side cross-sectional view of an embodiment of an annular implant with expandable members configured to expand close to the proximal end of the implant, the expandable members being shown in their compressed, unexpanded state.

FIG. 85B illustrates a front view of the implant of FIG. 85A wherein the expandable members have been released and are expanded radially outward.

FIG. 85C illustrates a side cross-sectional view of the expanded implant of FIG. 85B.

FIG. 85D illustrates a side cross-sectional view of the implant of FIG. 85C implanted with a cross-sectional representation of an intervertebral disc sandwiched between two vertebrae.

FIG. 86A illustrates a side view of an embodiment of an annular implant comprising a plurality of discreet initial geometric shapes interconnected by a tether to a tail flange, the initial geometric shapes which are separately inserted into an annular defect of an intervertebral disc one at a time.

FIG. 86B illustrates a side view of an embodiment of an annular implant following insertion into an annular defect within an intervertebral disc, and further following tensioning of the tether to cause the initial geometric shapes to align and lock into a final geometric shape, which forms the anchor for a tail flange.

FIG. 87A illustrates a side cross-sectional view of an embodiment of an annular implant comprising a plurality of initial geometric forms that are separately inserted into an annular defect within an intervertebral disc, the initial geometric forms being constrained by a loop tether and a tail flange.

FIG. 87B illustrates a side cross-sectional view of the annular implant of FIG. 87A wherein the initial geometric forms have been drawn together and tightened by the tether and locked to the tail flange to form an anchor which holds the tail flange against the outside of the annular defect to seal the defect.

FIG. 88A illustrates a side view of an embodiment of an annular implant comprising a plurality of initial geometric hoops that are slidably interconnected by a semi-rigid or rigid rod and which separately can be inserted through an annular defect into an intervertebral disc.

FIG. 88B illustrates a side view of the annular implant of FIG. 88A wherein the initial geometric hoops have been drawn together and tightened to the tail flange to form a second geometric shape serving the purpose of anchoring the tail flange against the annular defect to seal the annular defect against re-herniation.

FIG. 89 illustrates a cross-sectional view of an intervertebral disc with an embodiment of implant placed across the entire posterior portion thereof for the purpose of sealing the degenerated portion of the annulus against future herniation, the implant comprising a plurality of articulating segments and two end caps.

FIG. 90A illustrates an oblique view of an embodiment of an annular implant in its small diameter, rolled up configuration, the annular implant configured to span the entire posterior portion of the intervertebral disc.

FIG. 90B illustrates the annular implant of FIG. 90A in its expanded, planar configuration, wherein the implant is affixed to connector wires or rods and spans the distance therebetween with a membrane.

FIG. 90C illustrates the annular implant of FIG. 90A having been inserted into an intervertebral disc and wherein the connector wires have also been placed through lumens in the implant.

FIG. 90D illustrates the annular implant of FIG. 90B in its expanded configuration, within the intervertebral disc of FIG. 90C, wherein the connector wires have been secured to the vertebrae by anchoring screws, and further wherein the expanded membrane between the two connector wires serves to prevent the migration of nucleus pulposus or degenerated disc annulus in the posterior direction.

FIG. 91A illustrates a top view of an embodiment of a vertebral body spacer suitable for stabilizing the spine wherein the vertebral body spacer is provided in two parts, and the first part has been inserted into a surgically created void in an intervertebral disc, wherein the disc is shown in cross-sectional view.

FIG. 91B illustrates the vertebral body spacer of FIG. 91A following insertion of the second part to form a complete vertebral body spacer implant.

FIG. 92A illustrates the two parts of the vertebral body spacer of FIGS. 91A and 91B looking from the proximal end toward the distal end so that the tail lateral dimensions, the interlocking T-Slot on the right part and the T-projection on the left part are visible.

FIG. 92B illustrates the two parts of the vertebral body spacer of FIG. 92A wherein the T-projection is fitted within the T-slot to prevent lateral relative movement of one part away from the other part and further wherein the top and bottom surfaces of the spacer are substantially parallel to each other.

FIG. 92C illustrates embodiments of the vertebral body spacer looking from the rear or proximal end toward the distal end of the spacer, wherein the top and bottom surfaces are non-parallel to each other and wherein the lateral interlocking between the two parts is accomplished by a dovetail slot and projection.

FIG. 93A illustrates a side view of the vertebral body spacer of FIGS. 91A and 91B, wherein the spacer is shown fully inserted within an intervertebral disc.

FIG. 93B illustrates the vertebral body spacer of FIG. 93A as illustrated from the proximal end looking distally and showing the spacer in general contact with the top and bottom vertebrae.

FIG. 94A illustrates a side view of a spine segment, taken in cross-section, including an upper vertebra, a lower vertebra, and an intervertebral disc, wherein the posterior region of the intervertebral disc has collapsed in height due to degradation and further wherein the posterior portion of the intervertebral disc annulus is bulging posteriorly.

FIG. 94B illustrates the spine segment of FIG. 94A following placement of an embodiment of an intervertebral implant configured to distract and restore the collapsed spacing of the vertebrae and further wherein the implant is secured to at least one of the vertebrae by threaded anchors.

FIG. 94C illustrates the spine segment of FIG. 94A following implantation of an embodiment of an intervertebral spacer configured to distract and restore the collapsed spacing of the vertebrae and further wherein the implant is secured in place by an anchor head trapped anterior to the natural undercut of the vertebral lips.

FIG. 94D illustrates the spine segment of FIG. 94A following placement of an embodiment of an intervertebral spacer implant configured to distract and restore the collapsed spacing of the vertebrae and to eliminate the herniation bulge of the annulus, wherein the implant is secured in place by having its anchor head trapped within a hollowed out region in the intervertebral disc as well as the vertebrae themselves.

FIG. 95A illustrates a single spine implant of FIG. 94C against a cross-sectional view taken perpendicular to the longitudinal axis of the intervertebral disc.

FIG. 95B illustrates two spinal spacer implants of the type illustrated in FIG. 94D against a cross-sectional view taken perpendicular to the longitudinal axis of the intervertebral disc.

FIG. 96A illustrates a side view of an embodiment of an expandable reamer comprising two decoupled, sprung cutter elements, wherein the cutter elements are expanded to form a reamer bit with a second, large dimension.

FIG. 96B illustrates a front view of an embodiment of an expandable reamer bit comprising two sprung cutter elements, wherein the reamer bit is expanded into its second, large dimension.

FIG. 96C illustrates a side view of the expandable reamer of FIG. 96A, wherein the reamer bit is in its first, unexpanded state with the cutter elements sprung to form a smaller profile.

FIG. 97A illustrates a side view of an expandable reamer comprising two hinged cutter elements wherein the cutter elements are opened to form a reamer bit with a second, larger size.

FIG. 97B illustrates a front view of the expandable reamer bit of FIG. 97A wherein the cutter elements are expanded to form a reamer bit in its second, larger size.

FIG. 97C illustrates a side view of the expandable reamer of FIG. 97A wherein the two hinged cutter elements have rotated to form a reamer bit with a first, smaller dimension.

FIG. 97D illustrates a front view of the expandable reamer bit of FIG. 97C in its first, smaller dimensional configuration.

FIG. 98A illustrates a side view of an embodiment of an expandable reamer comprising a plurality of cutter elements rotatable about an axis parallel to the axis of the handle in its second, expanded configuration.

FIG. 98B illustrates a front view of the expandable reamer bit of FIG. 98A wherein the cutter elements are rotated to form a reamer bit with a second, larger configuration.

FIG. 98C illustrates a side view of an expandable reamer of FIG. 98A wherein the cutter elements have been rotated about an axis parallel to the longitudinal axis of the handle to form a reamer bit with a first, smaller configuration.

FIG. 98D illustrates a front view of the expandable reamer of FIG. 98C wherein the cutter elements are rotated to form a reamer bit having a first smaller dimension.

FIG. 99A illustrates a cross-sectional view of an intervertebral disc wherein an embodiment of a collapsed, laterally disposed implant has been placed.

FIG. 99B illustrates a cross-sectional view of the intervertebral disc wherein the laterally disposed implant of FIG. 99A has been expanded by introduction of a central dilator element.

FIG. 100A illustrates a side view of an embodiment of a distraction instrument, its distraction jaws in a closed position, which comprises a reverse-action pliers mechanism to distract the vertebral lips.

FIG. 100B illustrates a side view of the distraction instrument of FIG. 100A wherein the distraction jaws are in their open position.

FIG. 101A illustrates an oblique view of an embodiment of a spiral reamer comprising a central gripping region and a double barred spiral.

FIG. 101B illustrates a side view of the spiral reamer of FIG. 101A.

FIG. 102A illustrates a front view of an embodiment of a spiral reamer comprising a central gripping region and a double barred spiral with retainer tabs.

FIG. 102B illustrates a side view of the spiral reamer of FIG. 102A.

FIG. 103A illustrates an embodiment of an intervertebral disc implant comprising a fixation spike on the superior side.

FIG. 103B illustrates an embodiment of an intervertebral disc implant comprising a fixation spike on the inferior side wherein the fixation spike further comprises a barb.

FIG. 104 illustrates a cross-sectional view of a spine segment with an embodiment of an intervertebral disc implant placed therein, further wherein the implant is being used as a port to inject material into the intervertebral disc.

### DETAILED DESCRIPTION

In general, embodiments of the present spinal implant comprise a head portion and a barrier portion. The head portion is configured for placement between adjacent vertebrae at the site of an annular defect. The head portion includes a buttress portion that when positioned in the intervertebral space, spans a distance between, and contacts, adjacent vertebrae. The head portion is effective as a spacer to maintain a desired separation distance between the adjacent vertebrae. References to the instrumentation and the implant may use the words proximal and distal. An instrument or implant can have a longitudinal axis with the position relative to the longitudinal axis defined using the words proximal and distal. As used herein, the distal portion of an instrument or implant is that portion closest to the patient and furthest from the surgeon. The proximal portion is that portion closest to the surgeon and furthest from the patient.

Coupled to the head portion is a barrier portion. The barrier portion has a width that is greater than the width of the annular defect. The barrier portion is configured to prevent substantial extrusion of nucleus pulposus from the intervertebral disc when the barrier portion is positioned to contact an out surface of the annulus fibrosis, and spans the width of the annular defect.

The barrier portion can be further understood as including a tail portion and a tail flange portion, as is illustrated in the accompanying figures. As discussed herein, in certain embodiments, a tail portion includes a tail flange portion.

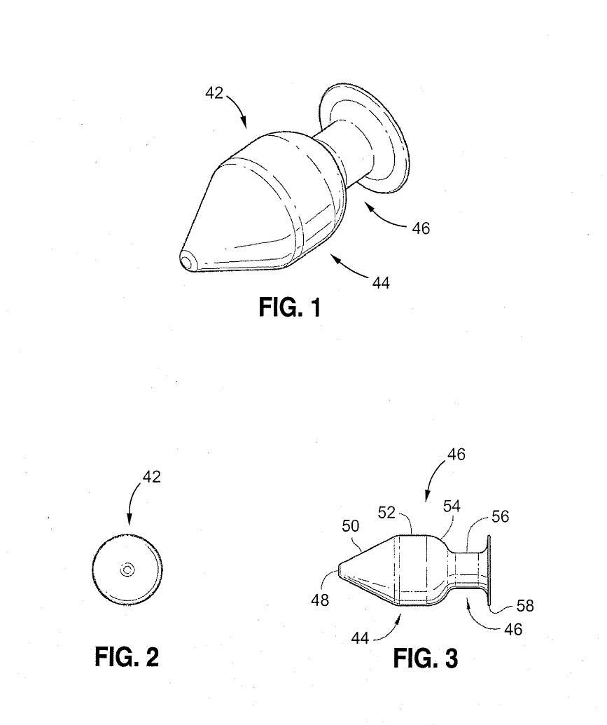

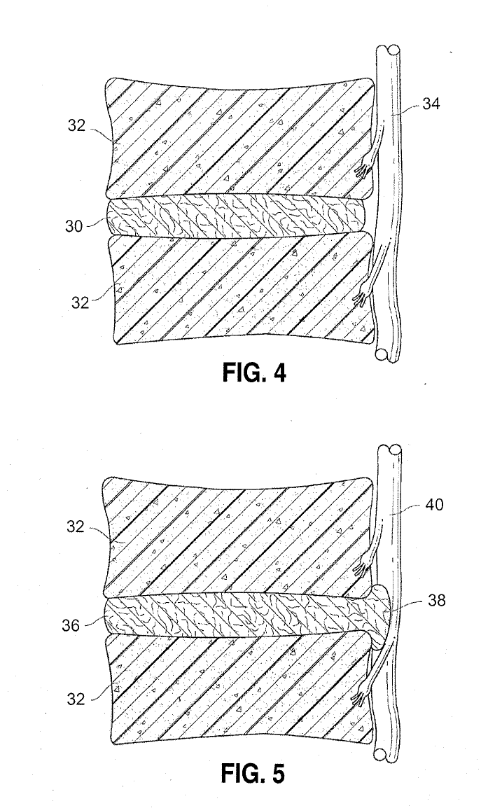

FIGS. 1-3 illustrate one embodiment of the present spinal implants. The implant 42 is shaped as a contoured plug having an enlarged head portion 44 and a relatively narrow tail portion 46 (FIG. 3). In the illustrated embodiment, cross-sections taken perpendicularly to a longitudinal axis of the implant 42 are substantially circular. However, the area of a given cross-section varies along the longitudinal axis.

With reference to FIG. 3, the head portion 44 includes a substantially flat nose 48 at a first end of a conical segment 50. The conical segment increases in height and cross-sectional area at a substantially constant rate from the nose to a first end of a large cylindrical segment 52. The large cylindrical segment extends at a constant height and cross-sectional area from the conical segment to a first end of a tapered segment 54. The tapered segment decreases in height and cross-sectional area at an increasing rate from the large cylindrical segment to a first end of a small cylindrical segment 56. The small cylindrical segment is substantially smaller in diameter than the large cylindrical segment, and extends at a constant height and cross-sectional area from the tapered segment to a tail flange 58. The tail flange flares outwardly from a minimum height and cross-sectional area at a second end of the small cylindrical segment to a maximum height and cross-sectional area at a second end of the implant 42. The maximum height of the tail flange is approximately equal to that of the large cylindrical segment.

The illustrated shape of the implant 42, including the relative dimensions of the segments 50, 52, 54, 56 and the flange 58, is merely one example. For example, cross-sections of the implant 42 taken along the longitudinal axis may be oval or elliptical or rectangular instead of circular. In addition, the ratio of the diameter of the small cylindrical segment 56 to the diameter of the large cylindrical segment 52 may be lesser or greater, for example. In addition, the implant 42 need not include the substantially cylindrical segments 52, 56. For example, the implant 42 may continue to taper from the nose 48 to the tapered segment 54, and the small cylindrical segment 56 may be reshaped to resemble adjoining tapered segments joined by a neck of a minimum diameter. Furthermore, the anatomy of annular defects and of vertebral end plates has wide variations. Accordingly, the implant 42 may be manufactured in a variety of shapes and sizes to fit different patients. A plurality of differently sized implants may, for example, be available as a kit to surgeons so that during an implantation procedure a surgeon can select the proper size implant from a range of size choices. FIGS. 14-22, described in more detail below, illustrate implants having sample alternative shapes and sizes.

In certain embodiments, the implant 42 is constructed of a durable, biocompatible material. For example, bone, polymer or metal may be used. Examples of suitable polymers include silicone, polyethylene, polypropylene, polyetheretherketone, polyetheretherketone resins, etc. In some embodiments, the material is non-compressible, so that the implant 42 can provide dynamic stability to the motion segment, as explained in detail below. In certain other embodiments, the material may be compressible. Suitable compressible materials for spinal implants include, but are not limited to, polyurethane, polycarbonate urethane, nitinol, stainless steel, cobalt nickel alloy, titanium, silicone elastomer, and the like.

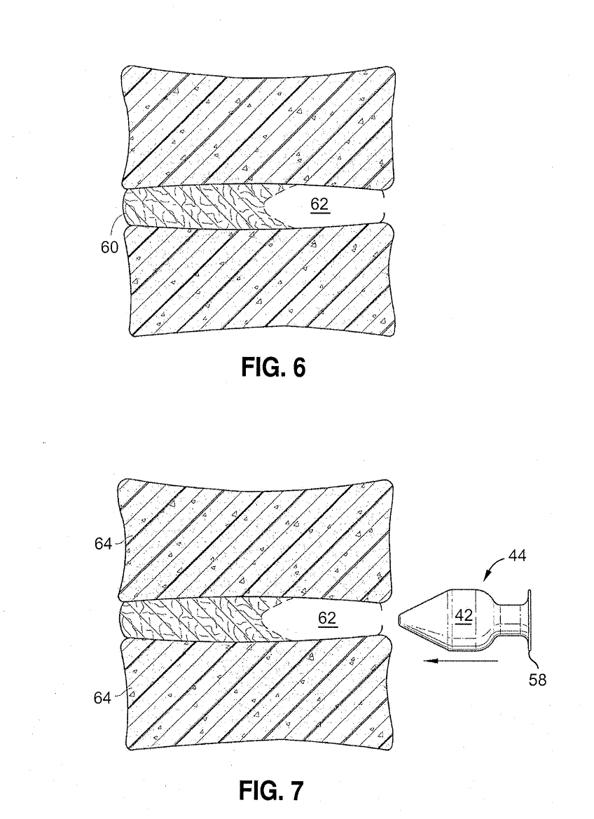

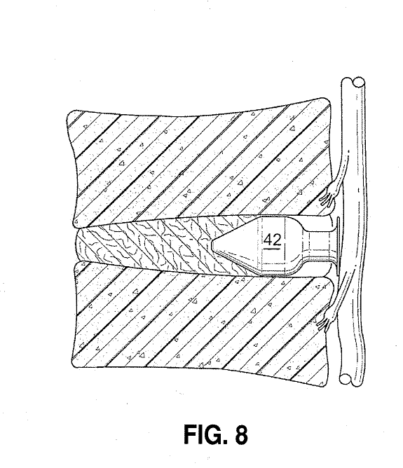

FIG. 6 illustrates an intervertebral disc 60 that has undergone a microdiscectomy procedure. A portion of the disc nucleus has been removed leaving a void 62. As shown in FIGS. 7 and 8, the implant 42 is adapted to be inserted between adjacent vertebrae 64 to fill the void 62. Once implanted, the contoured body of the implant 42, including the enlarged head portion 44 and the relatively narrow tail portion 46, may provide support to the adjacent vertebrae 64, resisting any tendency of these vertebrae to move closer to one another. However, in many cases the adjacent vertebrae 64 are not naturally shaped to provide mating engagement with the implant 42. As FIG. 8 shows, the implant 42 may sometimes be too large to fit within the intervertebral space, causing the adjacent vertebrae 64 to be forced apart.

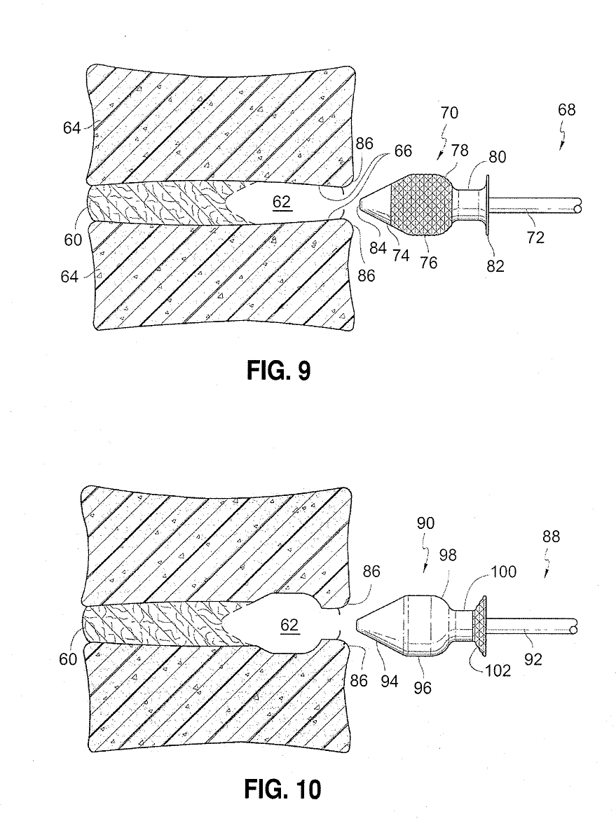

To avoid the ill-fitting engagement shown in FIG. 8, FIGS. 9-13 illustrate one embodiment of a method for implanting the implant 42 of FIGS. 1-3. In these figures, a portion of the intervertebral disc 60 has been removed through a microdiscectomy procedure. Before any disc material is removed, the implanting physician may visualize the implantation site using, for example, magnetic resonance imaging, or any other visualization technique. The visualization step allows the physician to determine what size and shape of implant is best suited to the procedure, which in turn allows the physician to determine what size and shape of tools to use during the procedure.

Before the implant 42 is introduced, the intervertebral space 62 and the adjacent vertebrae 64 may be prepared so that the implant 42 will fit properly. For example, each of the adjacent vertebrae 64 includes an end plate 66. In a healthy spine, these end plates abut the intervertebral discs. In the spine of FIGS. 9-13, these end plates will abut the implant 42 after it is implanted. Accordingly, the end plates may be shaped so that they have a mating or complementary fit with respect to the contoured implant 42 and assist the implant 42 in maintaining its desired position within the intervertebral space.

FIG. 9 illustrates one embodiment of a reaming tool 68 that is adapted to shape the end plates 66 of adjacent vertebrae 64. The reaming tool 68 includes a head portion 70 that extends from a distal end of a shaft 72. The head portion 70 and the shaft 72 may be formed integrally with one another, or the head portion 70 may be secured to the shaft 72 by any known means. In certain embodiments, the head portion and shaft are rigid, and may be made of a metal, for example. In the illustrated embodiment, the head portion is shaped substantially the same as the implant 42, and includes a conical segment 74, a large cylindrical segment 76, a tapered segment 78, a small cylindrical segment 80 and a tail flange 82. The illustrated size and shape of the head portion 70 is merely an example. However, it is advantageous for the head portion to be of similar size and shape to the implant that will ultimately be implanted in the intervertebral space 62 (whether that size and shape is the same as or different from the implant 42 of FIGS. 1-3).

At least a leading portion of the conical segment 74 includes a smooth outer surface. This smooth surface facilitates the entry of the head portion 70 into the intervertebral space 62, as described below. The small cylindrical segment 80 and tail flange 82 also each include a smooth outer surface. A trailing portion of the conical segment 74, the large cylindrical segment 76 and the tapered segment 78 each include a roughened surface. This surface may, for example, be knurled or burred. The roughened surface is adapted to remove bone from the vertebral end plates 66 in order to reshape the end plates so that they have a mating or complementary fit with respect to the contoured implant 42. In some embodiments, fewer or more segments of the head portion 70 can be roughened in order to provide desired capabilities for shaping the end plates 66.

To insert the head portion 70 into the intervertebral space 62, the surgeon positions the nose 84 of the head portion adjacent the extradiscal lips 86 on the adjacent vertebrae 64, as shown in FIG. 9. Then, applying digital pressure along the longitudinal axis of the shaft 72, the surgeon may push the head portion 70 into the void 62 between the adjacent vertebrae. Alternatively, the surgeon may strike a proximal end of the shaft 72 with a mallet to drive the head portion 70 into the void 62. The head portion 70 forces the adjacent vertebrae 64 apart as the head portion 70 penetrates into the void 62. Often, the adjacent vertebrae are resistant to being forced apart and significant force must be applied along the axis of the shaft 72 to force the head portion 70 into the void 62. The smooth surface at the leading end of the conical portion 74, which reduces friction between the head portion and the extradiscal lips 86, facilitates the entry of the head portion into the comparatively small void 62.

To remove material from the end plates 66, the surgeon rotates the shaft 72. The rotational force to the shaft may be applied directly by grasping the shaft with one's fingers, or by using a gripping instrument. Alternatively, a proximal end of the shaft may engage a powered drill, which may impart a rotational force to the shaft. The rotating shaft 72 rotates the head portion so that the roughened surfaces on the conical portion 74, the large cylindrical segment 76 and the tapered segment 78 scrape material from the end plates 66 of the adjacent vertebrae. The surgeon continues to remove bone material until the end plates achieve a desired surface contour to complement or mate with the implant 42, as shown in FIG. 10. The surgeon then removes the head portion 70 from the void 62 by applying digital pressure along the shaft 72, or by employing an instrument such as a slap hammer.

FIG. 10 illustrates one embodiment of a countersinking tool 88 that is adapted to shape the extradiscal lips 86 of adjacent vertebrae. A surgeon may use the countersinking tool in order to shape the extradiscal lips so that they more closely complement or mate with the tail flange 58 and prevent the implant 42 from being pushed into the intervertebral space 62.

The countersinking tool 88 includes a head portion 90 that extends from a distal end of a shaft 92. The head portion 90 and the shaft 92 may be formed integrally with one another, or the head portion 90 may be secured to the shaft 92 by any known means. In certain embodiments, the head portion and shaft are rigid, and may be made of a metal, for example. In the illustrated embodiment, the head portion is shaped substantially the same as the implant 42, and includes a conical segment 94, a large cylindrical segment 96, a tapered segment 98, a small cylindrical segment 100 and a tail flange 102. The illustrated size and shape of the head portion 90 is merely an example, and a variety of shapes and sizes may be used for this purpose.

The conical segment 94, large cylindrical segment 96, tapered segment 98, and small cylindrical segment 100 each include a smooth outer surface. The smooth surfaces facilitate the entry of the head portion 90 into the intervertebral space 62, as described above with respect to the reaming tool 68. The tail flange 102 includes a roughened surface. This surface may, for example, be knurled or burred. The roughened surface is adapted to remove bone from the extradiscal lips 86 in order to reshape the lips so that they provide a surface that complements or mates with the contoured implant 42.

In one embodiment of the method, the surgeon inserts the head portion 90 into the intervertebral space 62 in the same manner as described above with respect to the head portion 70. The head portion 90 fits within the void 62 such that the roughened surface on the tail flange 102 abuts the extradiscal lips 86. To remove material from the lips 86, the surgeon rotates the shaft 92. As with the reaming tool 68, the surgeon may impart a rotational force to the shaft 92 by grasping the shaft with one's fingers, a gripping instrument or a powered drill, for example. The rotating shaft 72 rotates the head portion so that the roughened surface on the tail flange 102 scrapes material from the lips 86. The surgeon continues to remove bone material until the end plates achieve a surface contour to complements or mates with the implant 42, as shown in FIG. 11. The surgeon then removes the head portion 90 from the void 62 in the same manner as described above with respect to the head portion 70.

In some embodiments, it may also be desirable to omit the step of countersinking the extradiscal lips. In these cases, the tail flange portion would abut the extradiscal lips, thus providing an effective barrier to prevent extrusion of material, in particular the nucleus pulposus, from the intervertebral disc space.

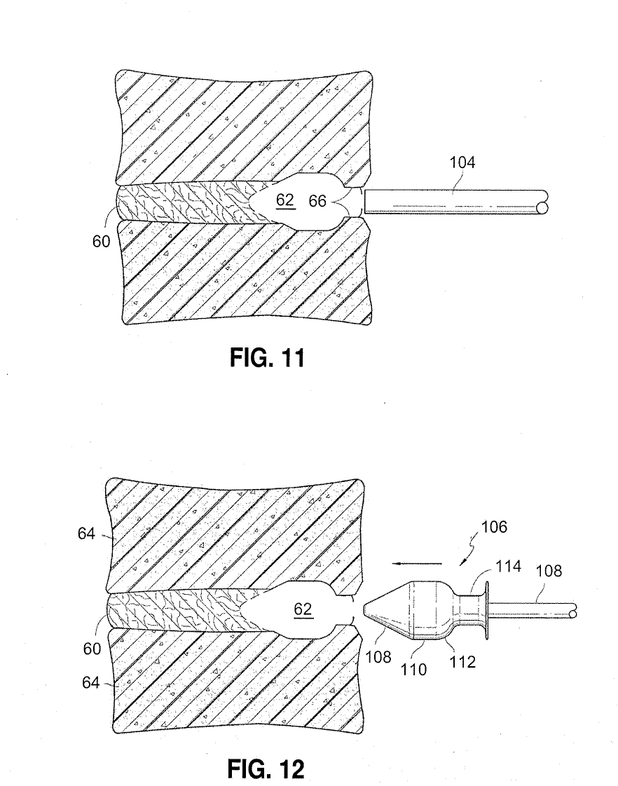

In certain embodiments, after the surgeon has shaped the vertebral end plates and extradiscal lips, he or she may use a sizing tool to measure the width of the opening between adjacent vertebral end plates 66. FIG. 11 illustrates one embodiment of a sizing tool 104. The tool comprises a cylindrical shaft of a known diameter. The surgeon may have several sizing tools of varying diameters close at hand during an implantation procedure. By attempting to insert sizing tools of increasing or decreasing diameters into the opening between adjacent vertebral end plates 66, the surgeon can measure the size of the opening. After measuring the distance between adjacent vertebral end plates 66, the surgeon will select the appropriate size of implant. He or she may begin with a trial implant, such as the implant 106 shown in FIG. 12.

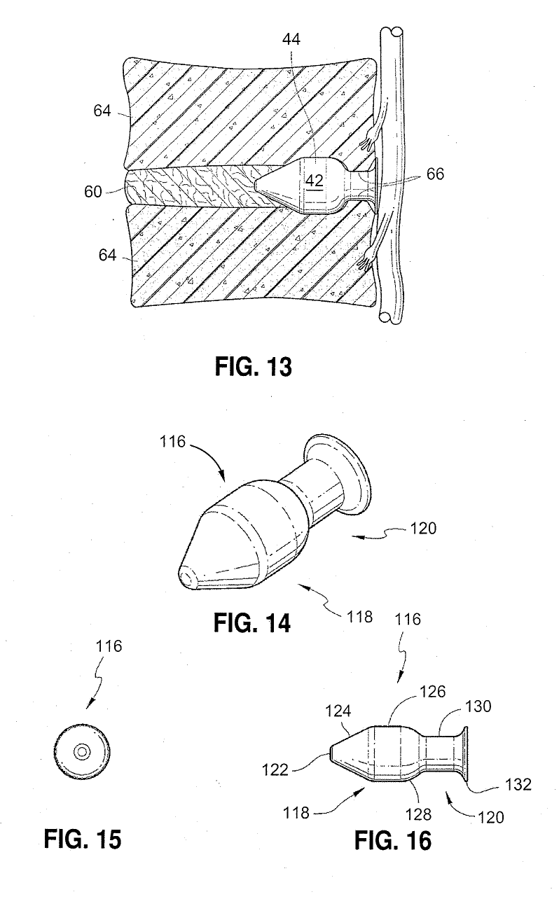

In the illustrated embodiment, the trial implant 106 is shaped exactly as the implant 42 of FIGS. 1-3, and is secured to the distal end of a shaft 108. The trial implant may be permanently or temporarily secured to the shaft. The surgeon may insert the trial implant 106 into the void 62 in the same manner as described above with respect to the head portions 70, 90. The smooth surface of the trial implant 106 facilitates its entry into the void 62. The conical portion 108 forces the vertebrae 64 apart as the surgeon advances the trial implant 108. Then, as the extradiscal lips pass over the large cylindrical segment 110 and reach the tapered segment 112, the vertebrae snap shut around the implant and the extradiscal lips come to rest around the small cylindrical segment 114. If the surgeon determines that the trial implant is the proper size to fit within the void, then he or she will withdraw the trial implant in the same manner as described above with respect to the head portions 70, 90. He or she will then select an implant that is the same size and shape as the trial implant 108, and insert the selected implant into the void 62, as shown in FIG. 13. The implant 42 may be temporarily secured to the distal end of a shaft (not shown), such that the insertion procedure is substantially the same as that described above with respect to the trial implant 108. If the implant is temporarily secured to the distal end of a shaft, it may engage the shaft through a threaded connection, for example. Once the implant is in place, the surgeon can then remove the shaft by unscrewing it from the implant.

The implant 42 advantageously stabilizes the region of the spine where it is implanted without substantially limiting the mobility of the region. Referring to FIGS. 3 and 13, it is seen that the conical segment 50, the large cylindrical segment 52, the tapered segment 54 and the small cylindrical segment 56 each abut and support the vertebral end plates 66, preventing the vertebrae 64 from moving closer to one another. Further, inter-engagement of the shaped end plates 66 and the tapered segment 54 resists any forces tending to push the implant 42 out of the intervertebral space, while inter-engagement of the tail flange 58 and the shaped extradiscal lips 86 resists any forces tending to push the implant 42 deeper into the intervertebral space. The border of the defect in the disc annulus (not visible in FIG. 13) comes to rest on the small cylindrical segment 56 and the tail flange 58, thus preventing any of nucleus pulposus from being squeezed out of the defect.

The implantation procedure described above can be performed using a guard device that would not be limited to preventing surrounding tissue from interfering with the procedure, but also protecting the surrounding tissue from damage. For example, a tubular guard (not shown) may be employed around the implantation site. The guard can prevent surrounding tissue from covering the implantation site, and prevent the implantation instruments from contacting the surrounding tissue.

In certain embodiments of the present methods, the spacing between adjacent vertebrae is maintained. Thus, the spacing between adjacent vertebrae after one of the present implants has been inserted therebetween is approximately the same as the spacing that existed between those same vertebrae prior to the implantation procedure. In such a method, it is unnecessary for the implanting physician to distract the vertebrae prior to introducing the implant. As described above, the increasing size of the conical segment and the large cylindrical segment of the implant temporarily distracts the vertebrae as it passes between the discal lips thereof, after which the vertebrae snap shut around the implant. In certain other embodiments of the present methods, however, it may be advantageous to increase the spacing of the adjacent vertebrae through the implantation procedure, so that the spacing between the adjacent vertebrae after the implant has been inserted therebetween is greater than the spacing that existed between those same vertebrae prior to the implantation procedure. In such embodiments, the implanting physician may distract the adjacent vertebrae prior to implanting the implant in order to achieve the desired spacing.

FIGS. 14-22 illustrate alternative embodiments of the present spinal implants. These alternative embodiments are adapted for use in spinal discs where the patient's anatomy is better suited to an implant having a different size and/or shape. For example, FIGS. 14-16 illustrate a spinal implant 116 having an enlarged head portion 118 and a relatively narrow tail portion 120 (FIG. 16). As in the implant 42 of FIGS. 1-3, the head portion 118 of the implant 116 of FIGS. 14-16 includes a substantially flat nose 122, a conical segment 124, a large cylindrical segment 126 and a tapered segment 128. The tail portion 120 includes a small cylindrical segment 130 and a tail flange 132. In comparing the embodiment of FIGS. 1-3 to the embodiment of FIGS. 14-16, the conical segment 50 is longer than the conical segment 124, and the large cylindrical segment 52 is wider in diameter than the large cylindrical segment 126. The tail flange 58 is also somewhat wider in diameter than the tail flange 132. Thus, the implant 116 of FIGS. 14-16 is adapted for implantation in an intervertebral disc having a relatively small diameter, or where it is advantageous for the implant 116 to penetrate a relatively short distance into the disc.

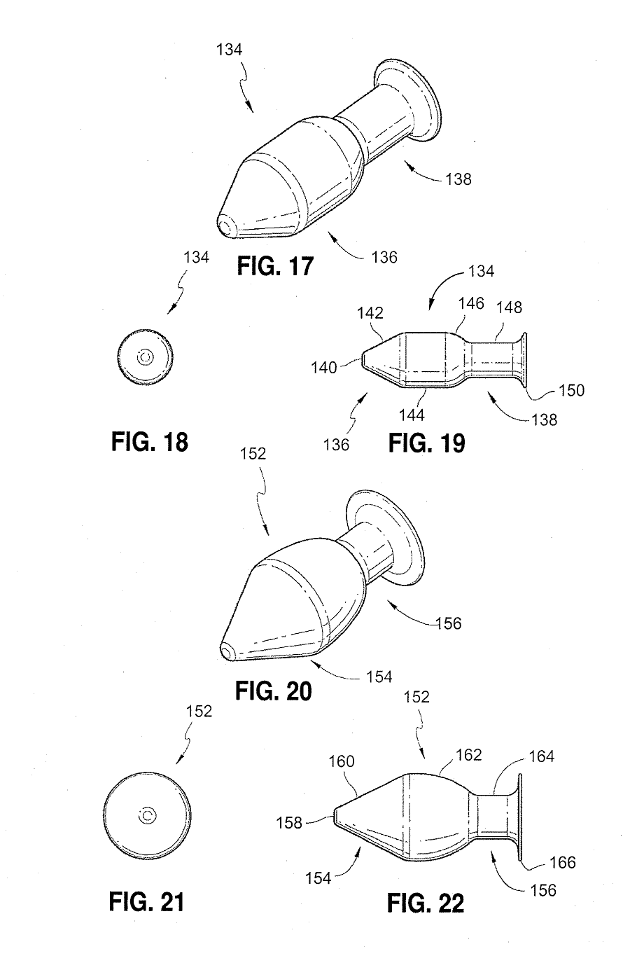

FIGS. 17-19 illustrate a spinal implant 134 having an enlarged head portion 136 and a relatively narrow tail portion 138 (FIG. 19). Cross-sections taken perpendicularly to a longitudinal axis of the implant are substantially circular, however, the area of a given cross-section varies along the longitudinal axis. As in the implants described above (and as with implants described herein and encompassed by the claims below), the cross-sectional shape of the implant 134 need not be circular, and could be, for example, elliptical or oval. Further, the cross-sectional shapes of the implants described herein may vary along the longitudinal axis.

The head portion 136 includes a substantially flat nose 140 at a first end of a conical segment 142. The conical segment increases in height and cross-sectional area at a substantially constant rate from the nose to a first end of a large cylindrical segment 144. The large cylindrical segment extends at a constant height and cross-sectional area from the conical segment to a first end of a tapered segment 146. The tapered segment decreases in height and cross-sectional area at an increasing rate from the large cylindrical segment to a first end of a small cylindrical segment 148. The small cylindrical segment is substantially smaller in height than the large cylindrical segment, and extends from the tapered segment to a tail flange 150. The tail flange flares outwardly from a minimum height and cross-sectional area at a second end of the small cylindrical segment to a maximum height and cross-sectional area at a second end of the implant 134. The maximum height of the tail flange may be approximately equal to that of the large cylindrical segment.

A comparison between the implant 116 of FIGS. 14-16 and the implant 134 of FIGS. 17-19 reveals that the implant 134 of FIGS. 17-19 has a longer large cylindrical segment 144 and a longer small cylindrical segment 148. The remaining segments in the implant 134 are substantially similar to their counterparts in the implant 116. The implant 134 of FIGS. 17-19 is thus adapted for implantation in an intervertebral disc where it is advantageous for the implant 134 to penetrate a greater distance into the disc as compared to the implant 116 of FIGS. 14-16.

FIGS. 20-22 illustrate a spinal implant 152 having a shape that is similar to the implant 42 of FIGS. 1-3. The implant 152 includes an enlarged head portion 154 and a relatively narrow tail portion 156 (FIG. 22). As in the implant 42 of FIGS. 1-3, the head portion 154 of the implant 152 of FIGS. 20-22 includes a substantially flat nose 158, a conical segment 160 and a tapered segment 162. However, the implant 152 does not include a large cylindrical segment. Instead, the conical segment directly adjoins the tapered segment, and the tapered segment tapers at a more gradual rate as compared to the tapered segment 54 of the implant 42 of FIGS. 1-3. The head portion 154 achieves a maximum height at the junction between the conical segment 160 and the tapered segment 162. This area of maximum height is adapted to provide stability to the adjacent vertebrae. As with the implant 42 of FIGS. 1-3, the tail portion 156 of the implant 152 of FIGS. 20-22 includes a small cylindrical segment 164 and a tail flange 166.

The relative dimensions shown in the figures are not limiting. For example, in FIG. 13 the implant 42 is illustrated as having certain dimensions relative to the dimensions of the vertebrae 64. In fact, the size of the implant relative to the vertebrae will be chosen based upon a variety of factors, including the patient's anatomy and the size of the annular defect to be repaired. In certain applications, the implant may be significantly smaller relative to the vertebrae, and may extend significantly less than halfway toward a vertical centerline of the intervertebral disc. In certain other applications, the implant may be significantly larger relative to the vertebrae, and may extend almost completely across the intervertebral disc.

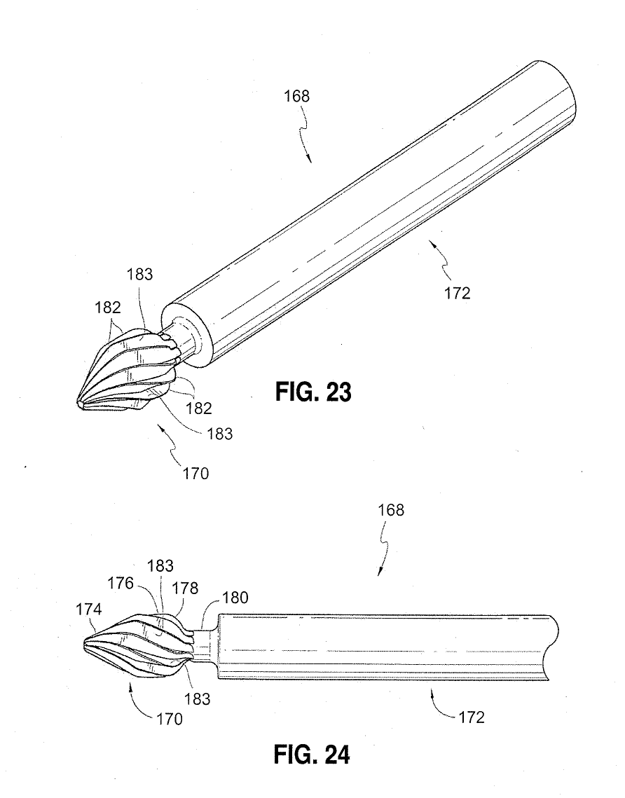

FIGS. 23 and 24 illustrate an alternative reaming tool 168 that may be used to shape the end plates of adjacent vertebrae. The reaming tool 168, which is similar to the reaming tool 68 described above and pictured in FIG. 9, includes a head portion 170 that extends from a distal end of a shaft 172. The head portion 170 and the shaft 172 may be formed integrally with one another, or the head portion 170 may be secured to the shaft 172 by any known means. In certain embodiments, the head portion 170 and shaft 172 are rigid, and may be made of a metal, for example. In the illustrated embodiment, the head portion 170 is shaped similarly to the implant 42, and includes a conical segment 174, a large cylindrical segment 176, a tapered segment 178 and a small cylindrical segment 180 (FIG. 24). The illustrated size and shape of the head portion 170 is merely an example. However, it is advantageous for the head portion 170 to be of similar size and shape to the implant that will ultimately be implanted in the intervertebral space (whether that size and shape is the same as or different from the implant 42 of FIGS. 1-3). In the illustrated embodiment, the shaft 172 has a greater width relative to the head portion 170 as compared to the reaming tool 68 described above, thereby making the reaming tool 168 easier to grip.

A plurality of curved blades 182 (FIG. 23) extend along the surfaces of the conical segment 174, the large cylindrical segment 176, the tapered segment 178 and the small cylindrical segment 180, giving the head portion 170 a scalloped surface. The blades 182 extend in a substantially helical pattern along a longitudinal axis of the head portion 170. Each pair of adjacent blades 182 is separated by a cavity 183. The blades 182 are adapted to remove bone from the vertebral end plates 66 in order to reshape the end plates so that they provide a surface that is complementary to the contoured implant 42. Operation of the reaming tool 168 is substantially identical to operation of the reaming tool 68 described above. The blades 182 scrape bone material away as the reaming tool 168 is rotated, and the cavities 183 provide a volume to entrain removed bone material.

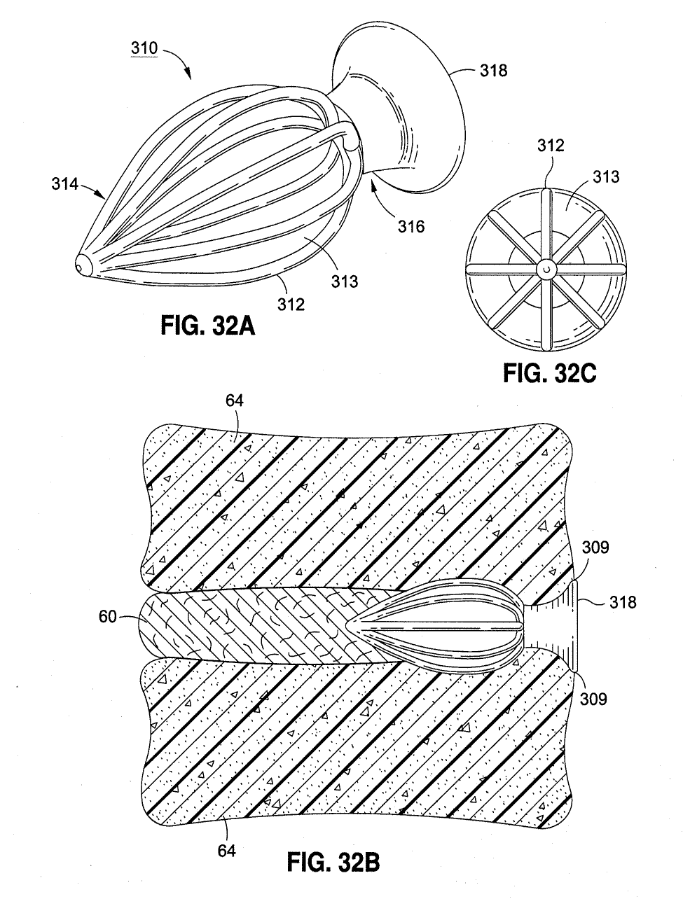

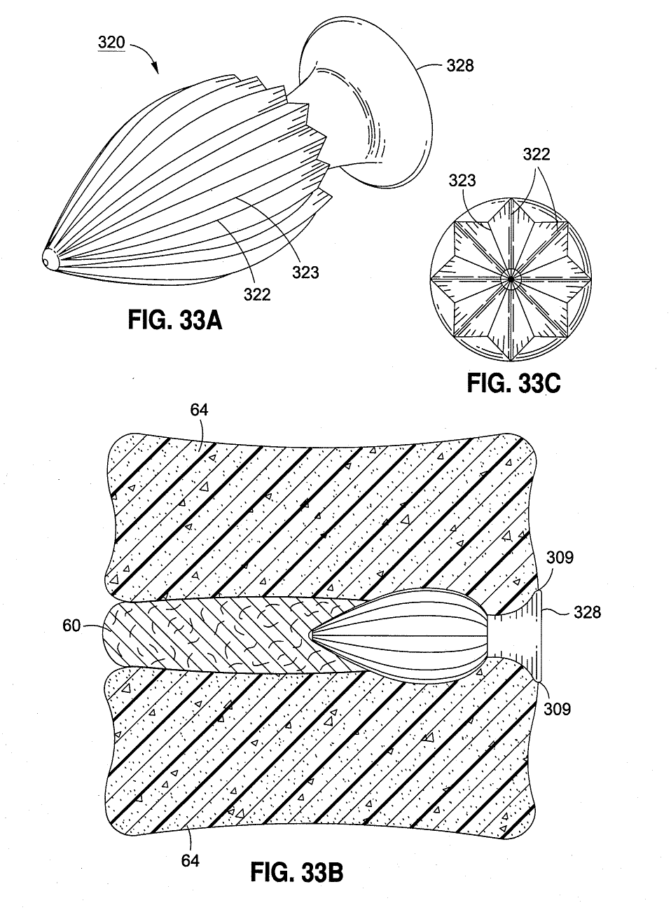

In certain embodiments, rather than having curved blades, the reaming tool 172 might be fashioned to provide a head portion 170 adapted to cut threads in the vertebral surfaces adjacent to the site of repair, analogous to a “tap” used in the mechanical arts to thread holes to receive bolts or screws. Providing a reaming tool with the ability to thread a repair site would provide a thread pattern that would substantially fit the pitch and depth of the threads included in an embodiment of the present spinal implant, for example that illustrated in FIG. 32A.

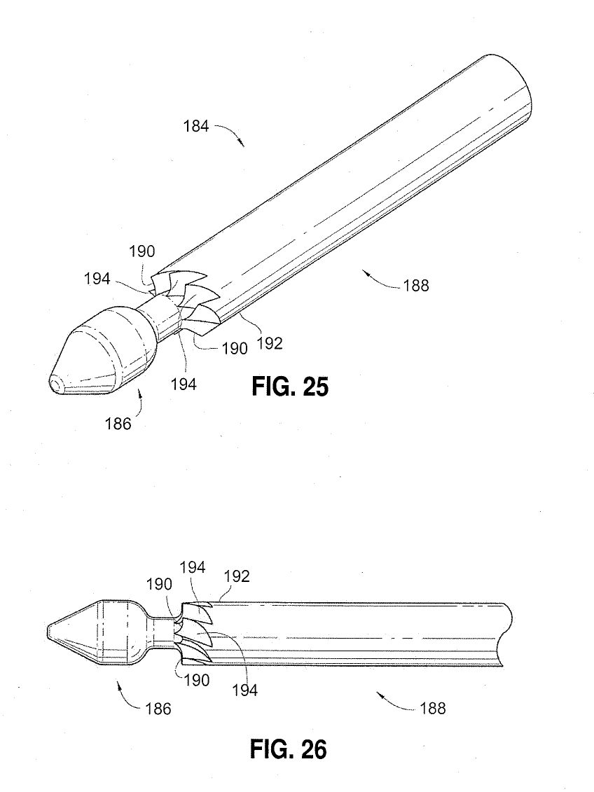

FIGS. 25 and 26 illustrate an alternative countersinking tool 184 that may be used to shape the extradiscal lips of adjacent vertebrae. The countersinking tool 184, which is similar to the countersinking tool 88 described above and pictured in FIG. 10, includes a head portion 186 that extends from a distal end of a shaft 188. The head portion 186 and the shaft 188 may be formed integrally with one another, or the head portion 186 may be secured to the shaft 188 by any known means. In certain embodiments, the head portion 186 and shaft 188 are rigid, and may be made of a metal, for example. In the illustrated embodiment, the head portion 186 is shaped similarly to the implant 42. The illustrated size and shape of the head portion 186 is merely an example. However, it is advantageous for the head portion 186 to be of similar size and shape to the implant that will ultimately be implanted in the intervertebral space (whether that size and shape is the same as or different from the implant 42 of FIGS. 1-3). In the illustrated embodiment, the shaft 188 has a greater width relative to the head portion 186 as compared to the countersinking tool 88 described above, thereby making the countersinking tool 184 easier to grip.

A plurality of curved blades 190 extends around a distal end 192 of the shaft 188, adjacent the head portion 186. An edge of each blade 190 faces the head portion 186, and each pair of adjacent blades 190 is separated by a wedge-shaped cavity 194. The blades 190 are adapted to remove bone from the extradiscal lips of adjacent vertebrae in order to reshape the vertebrae so that they provide a surface that is complementary to the contoured implant 42. Operation of the countersinking tool 184 is substantially identical to operation of the countersinking tool 88 described above. The blades 190 scrape bone material away as the countersinking tool 184 is rotated, and the cavities 194 provide a volume to entrain removed bone material.

In certain embodiments, the reaming tool may further comprise a stop to prevent the tool from penetrating into the intervertebral disc further than a desired distance. In some embodiments, the stop may comprise a flange on the shaft of the reaming tool that abuts the vertebrae when the tool has been inserted the desired distance.

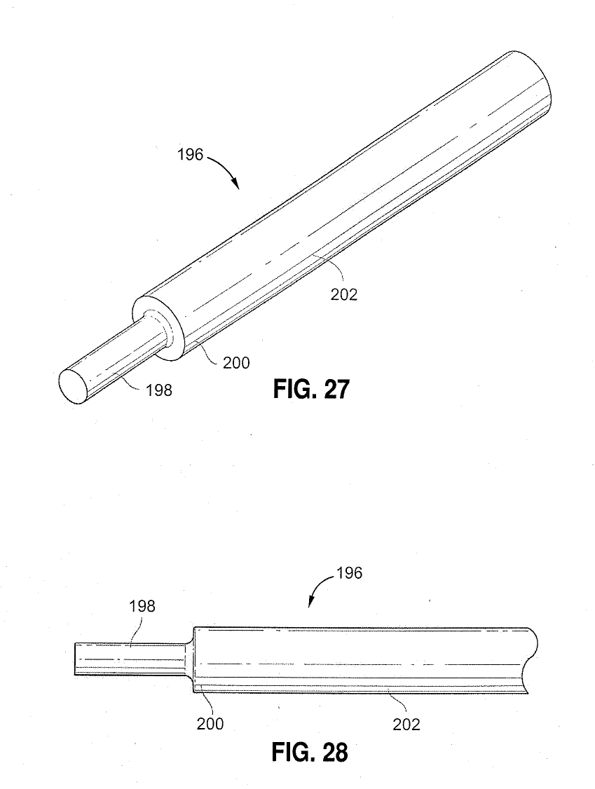

FIGS. 27 and 28 illustrate another embodiment of a sizing tool 196. The tool comprises a cylindrical shaft 198 of a known diameter that extends from a distal end 200 of a handle portion 202. Operation of the sizing tool 196 is substantially identical to operation of the sizing tool 104 described above. However, the sizing tool 196 of FIGS. 27 and 28 advantageously has a handle portion 202 that is wider than the cylindrical shaft 198, thereby making the sizing tool 196 easier to grip.

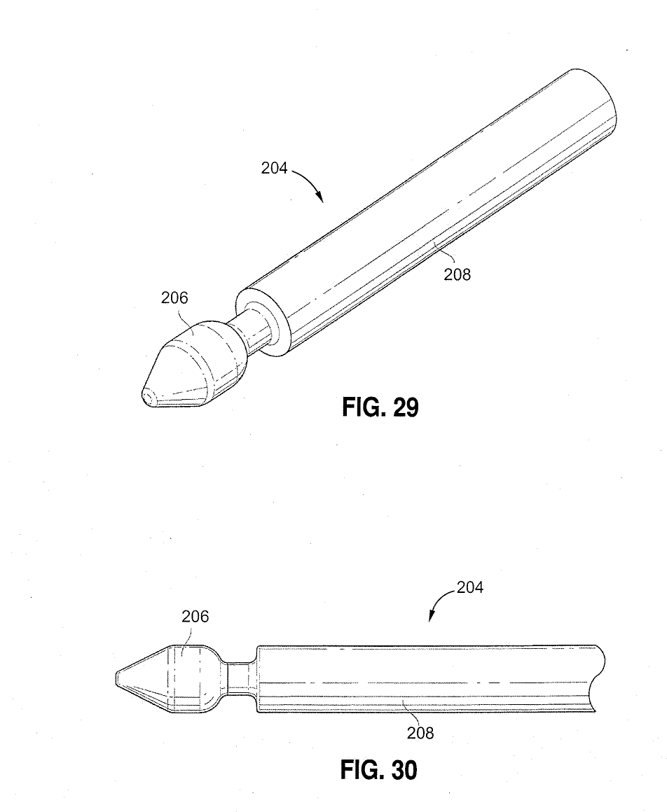

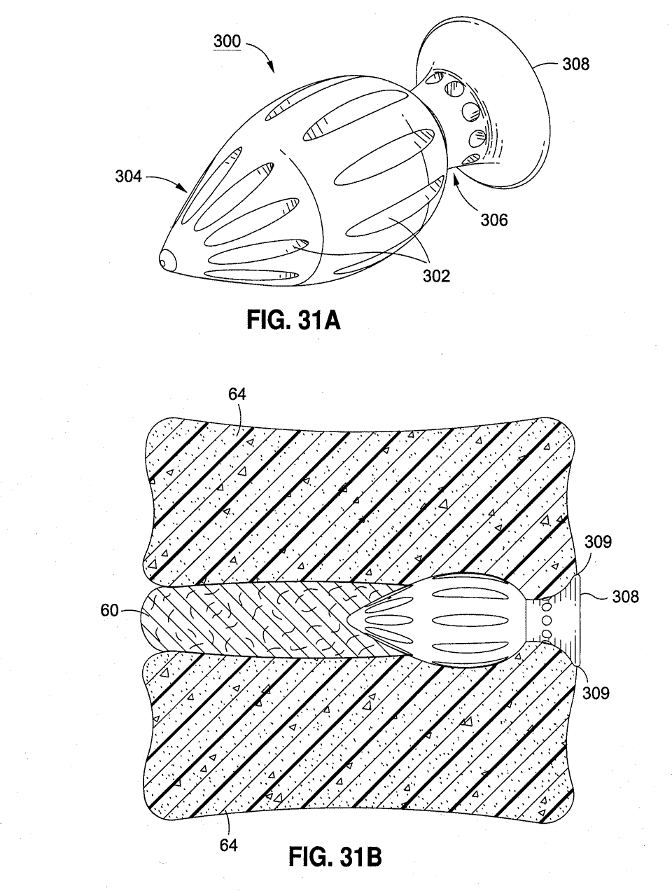

FIGS. 29 and 30 illustrate another embodiment of a trial implant 204. The trial implant 204, which comprises an implant portion 206 and a handle portion 208, is similar to the trial implant 106 described above. However, the trial implant 204 of FIGS. 29 and 30 advantageously has a wider handle portion 204, thereby making the trial implant 204 easier to grip.

In addition to the embodiments described above, a number of variations in the structure, shape or composition of the spinal implant are also possible and are intended to fall within the scope of the present disclosure.

For example, in certain embodiments, one of which is depicted in FIG. 31 A, the spinal implant 300 may be relatively hollow and may further comprise bone graft compaction holes 302. Either the head portion 304 and/or the tail portion 306 may be hollow, and either or both may include holes as desired. The compaction holes will permit spring back of vertebral bone into the implant, thus further securing the implant when it is placed in the intervertebral space between two adjacent vertebrae 64. As depicted in FIG. 35B, the tail flange 308 abuts the extradiscal lips 309 of adjacent vertebrae operative to limit or prevent extrusion of material such as nucleus pulposus from the intervertebral disc 60 when the barrier portion is positioned such that it contacts an outer surface of the annulus fibrosis and spans the width of the annular defect.

In some embodiments, one of which is depicted in FIG. 32A, the spinal implant 310 may include splines. The splines 312 may be spaced apart in a wire or basket-like configuration, the spaces between splines 314 providing access to the interior of the implant such that the implant is effectively hollow. In some embodiments, the material used to fashion the splines may be chosen to mimic the natural deformability of the annulus, while retaining sufficient rigidity to maintain a proper distance between the adjacent vertebrae 64, consistent with the spacer function provided by the head portion of the implant. The device may be constructed such that the head 314 alone is splined, the tail 318 alone is splined, or both the head and tail are splined. The tail flange 318 abuts the extradiscal lips 319 of adjacent vertebrae, operative to limit or prevent extrusion of material from the intervertebral disc 60 when the barrier portion is positioned such that it contacts an outer surface of the annulus fibrosis and spans the width of the annular defect.

In some embodiments, a splined implant may have a solid surface. For example, an implant 320 may be solid with a spline 322 and groove 323 pattern forming the surface of the implant as depicted in FIG. 33 A. Splined implants provide an advantage in that they will tend to resist rotation, which will serve to better secure the implant at the repair site as shown in FIG. 33B. As with other embodiments, the tail flange 328 abuts extradiscal lips 309 of adjacent vertebrae providing a barrier. Again, splines may be included on the head portion 324, the tail portion 326, or both the head and tail portion. The splines may be substantially aligned with the longitudinal axis of the implant, or alternatively, may have a rotational pitch imparted on them. Where the splines have a rotational pitch imparted on them, placement of the implant may be accomplished by a combined pushing and twisting motion.

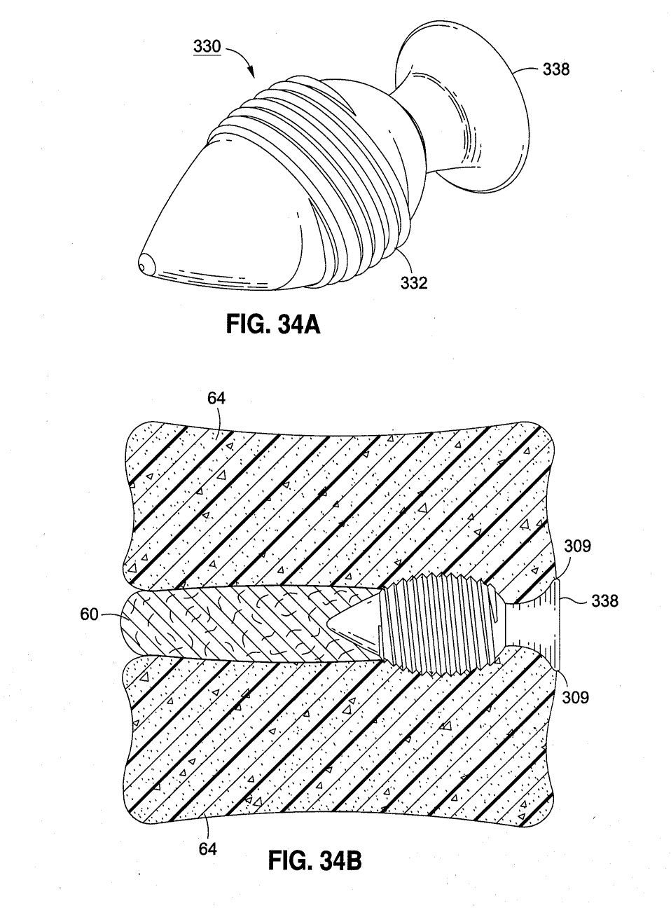

In some embodiments, the implant 330 may include a spiral “barb” 332 analogous to a screw thread, one of which is illustrated in FIG. 34A. In a spiral barb embodiment, placement and securing of the implant might also involve turning the implant such that the thread engages adjacent vertebrae 64 permitting the implant to be threaded into the intervertebral space. If desired the surface of adjacent vertebrae could be prepared by cutting a thread of substantially the same pitch as that on the implant head using a thread cutting tool, much like the typical method of tapping a hole in order to provide a means to engage a bolt as is well known in the mechanical arts. In this way, the implant could be more easily threaded into place, and a more secure fit would be obtained. Threading the implant into place further allows the tail flange 338 to be brought up snugly against the extradiscal lips 309 thus improving the barrier function of the implant, as is shown in FIG. 34B.

In some embodiments of the spinal implant 340, a plurality of substantially concentric barbs 342, one of which is shown in FIG. 35A, might be included. The orientation of the barbed ends could be biased either towards the front or rear of the spinal implant. Biasing of the barbs would provide an advantage in that barbs would better resist movement of the implant either in or out of the site of implantation, as is shown in FIG. 35B. Barbs may be provided either on the head portion, the barrier portion or both as desired. In certain embodiments, any number of barbs can be used and may be effective.

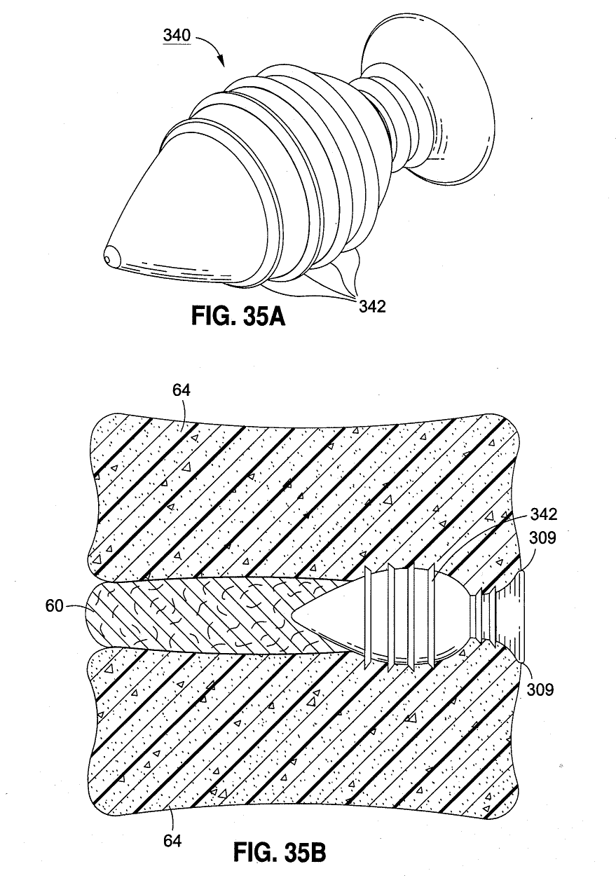

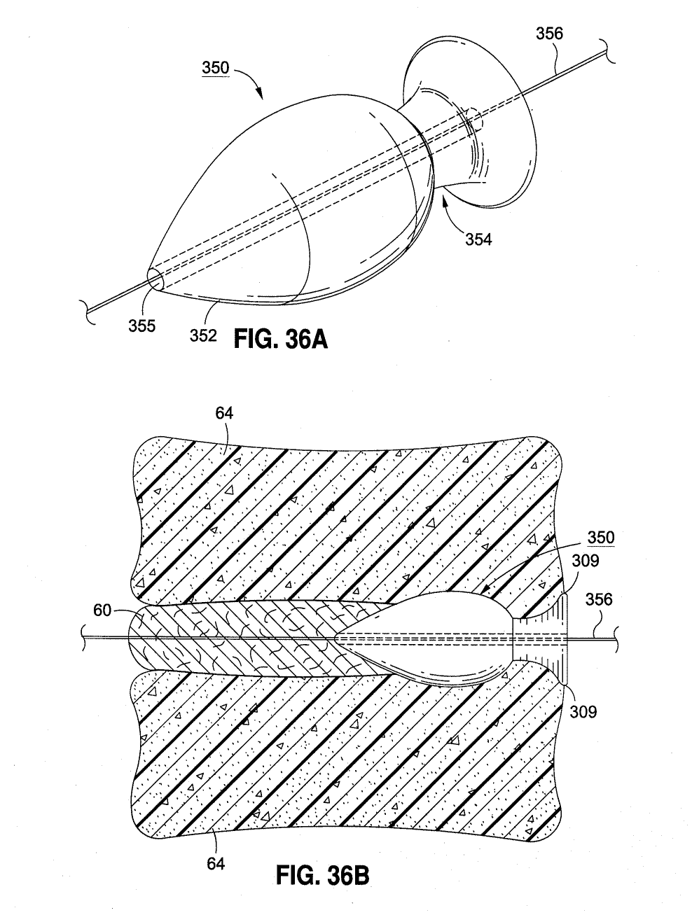

In some embodiments, one of which is illustrated in FIG. 36 the implant 350 comprises a head portion 352 and tail portion 354 with a lumen 355 extending through the spinal implant in a direction along a longitudinal axis of the spinal implant, the lumen being adapted to permit an elongate member to pass therethrough. In some embodiments, the elongate member comprises a guide wire 356. The guide wire provides the advantage of being able to re-locate the site for repair after first having identified the site with an endoscope or other similar minimally invasive device. Conveniently, in the course of repair surgery, for example using an endoscope or other minimally invasive method, the site of the desired repair may be marked with a guide wire that extends externally. Once the site for repair has been selected and marked, the implant can be fed onto the wire by passing the implant over the end of the wire outside the patient via the lumen 355. The implant may then be passed down the guide wire directly to the site to be repaired simply by sliding the implant along the wire.

In certain embodiments compatible with a guide wire, one of which is depicted in FIG. 37B, an implant 350 is shown with a relatively thin tail segment 354, the head and tail both including an axially located a lumen 355 extending through the spinal implant in a direction along a longitudinal axis of the spinal implant, the lumen being adapted to permit an elongate member to pass therethrough. In some embodiments, the elongate member comprises a guide wire 356. The tail flange 358 abuts the extradiscal lips 309 of adjacent vertebrae. The tail segment comprises a thin flexible material of sufficient tensile strength such that some radial movement is possible between the head and tail flange, but where the relative distance along the longitudinal axis between the two portions of the implant is maintained. Providing a thin and flexible tail segment would thus permit some movement of the head portion relative to the tail flange, potentially improving spinal mobility, without compromising either the anchoring and spacer functions of the head portion, or the barrier function of the implant.

As before, optionally providing a hole down the longitudinal axis of the implant would permit the use of a guide wire for locating the implant to the repair site using a minimally invasive method. The flexible tail portion will permit accommodation of some radial movement of the head portion relative to the tail portion, as might be expected with flexure of the spine, and thus would be operative to help maintain the tail flange 358 relatively in place with respect to the extradiscal lips 309 of adjacent vertebrae thus improving the barrier function of the tail flange.

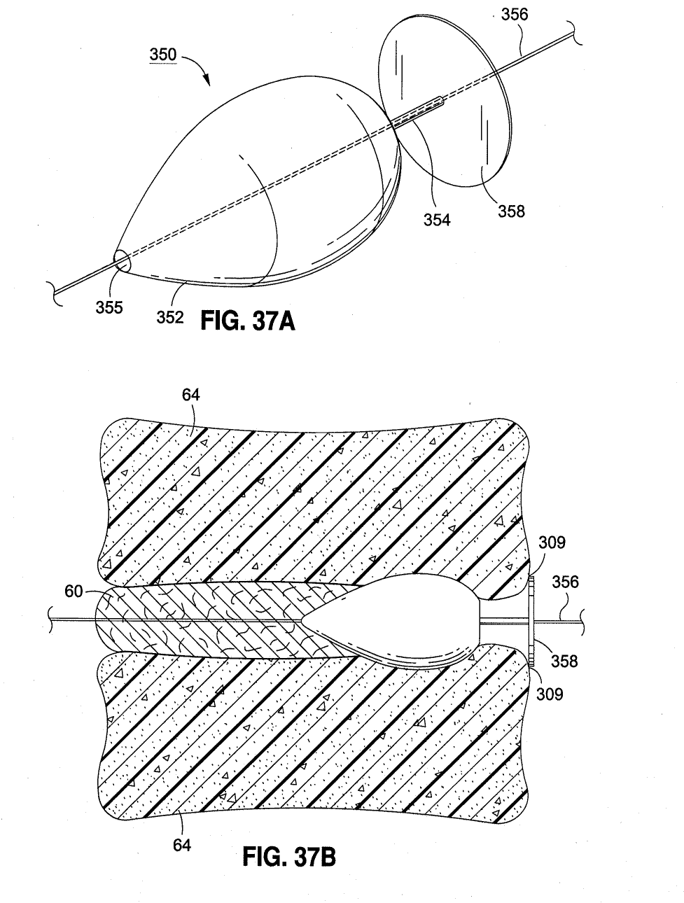

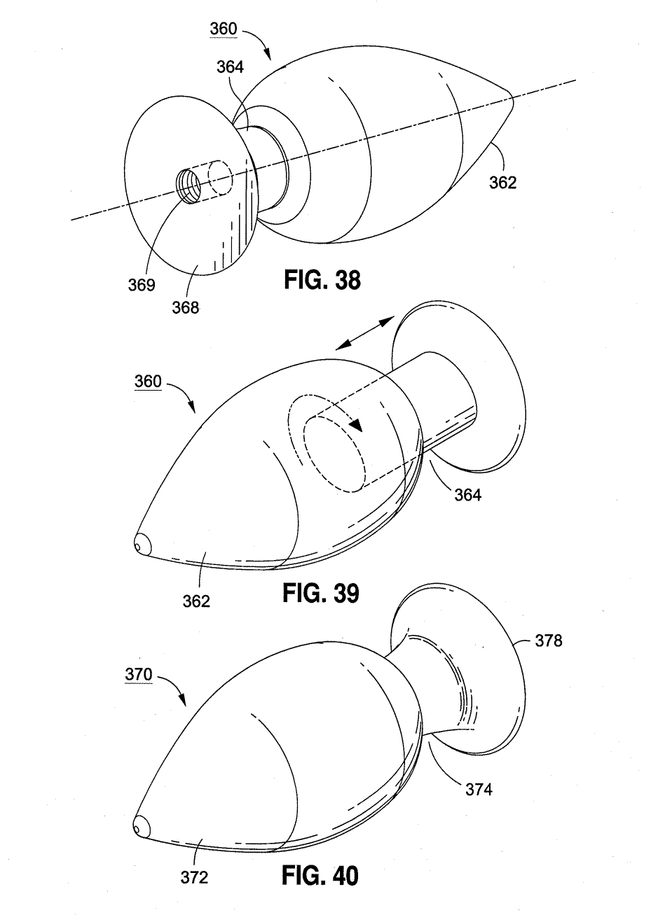

In some embodiments the spinal implant may comprises a plurality of components that are reversibly coupled, being assembled either prior to implantation, or as part of the implantation procedure, into the completed implant device. For example, FIGS. 38 and 39 depict an implant 360 comprising a head portion 362 into which a separate tail segment 364 or alternatively a separate tail flange 368 are reversibly coupled. For example, as shown in FIG. 38, the tail flange 368 could be separate from the tail segment 364 and head portion. In this instance, the tail flange would be threaded onto a bolt-like extension 369 that would extend from the tail segment 364. Alternatively, the tail segment and tail flange comprise a contiguous piece that engages a separate head portion as is shown in FIG. 39. In each of these cases, providing a mechanism for threading together the head and barrier portions provides a means for better securing the tail flange against the extradiscal lips of adjacent vertebrae, thus providing an improved barrier function to prevent extrusion of material, in particular the nucleus pulposus, from the intervertebral disc space. Although not illustrated, certain embodiments like those illustrated in FIGS. 38-39 could include a hole located substantially along the longitudinal axis in order to permit placement of the implant using a guide wire.

For embodiments of the present spinal implant comprising separate portions, the engagement means might be reversibly coupled by compatible threads. In some embodiments, the components of the spinal implant may be lockably coupled in order to prevent inadvertent separation after placement. For example, the head portion may be lockably couple to the barrier portion. In these cases there may be provided a twist-and-lock arrangement, or other similar means of lockably connecting the pieces.

An advantage is provided by reversibly coupled and lockably coupled embodiments in that the head portion may be placed in the prepared implantation site, and then the barrier portion subsequently coupled. It is a further advantage of such an arrangement that the tail flange will be brought into a very snug abutment relative to the extradiscal lips of adjacent vertebrae, thereby better securing and ensuring the stability of the implant. A variety of possible means with which to reversibly couple or lockably couple separate head and barrier portions are well known in the art and could include, without limitation, such means as threads, clips, spring-loaded ball bearing and groove combinations, biocompatible adhesives, or any other suitable means for connecting the two pieces in a secure fashion.

It is further realized that the various functional domains of the disclosed spinal implants need not be fashioned from a single material. As the head portion, tail segment and tail flange can perform different functions, there might be a potential advantage in fashioning these different functional domains of the implant from materials best suited to perform a particular function. For example, in some embodiments of the spinal implant 370, it may be desirable to provide a head portion 372 that is resilient and approximates the biomechanical properties of the native intervertebral disc. The tail segment 374 might be fashioned of a material that is more flexible to allow greater mobility of the spine without compromising the structural integrity provided by the implant. Likewise, the tail flange 378 may function better if it is made from a more rigid material that resists deformation in order to better carry out its barrier function, as in FIG. 40.

Thus, while the shape and design of the spinal implant may be varied, the various parts of each of these embodiments still perform the same basic functions. Namely, the head portion abuts and supports facing endplates of the first and second vertebral discs to aid in preventing collapse of the intervertebral disc while providing dynamic stability to the motion segment. The head portion further performs a spacer function, maintaining adjacent vertebrae at a relatively constant distance from each other, at least at the site of the herniation being repaired. The tail portion abuts and supports the facing endplates to aid in preventing collapse of the intervertebral disc while providing dynamic stability to the motion segment. In addition, the tail flange abuts the extradiscal lips of the first and second discs to prevent the implant from penetrating the disc beyond a certain pre-determined amount.

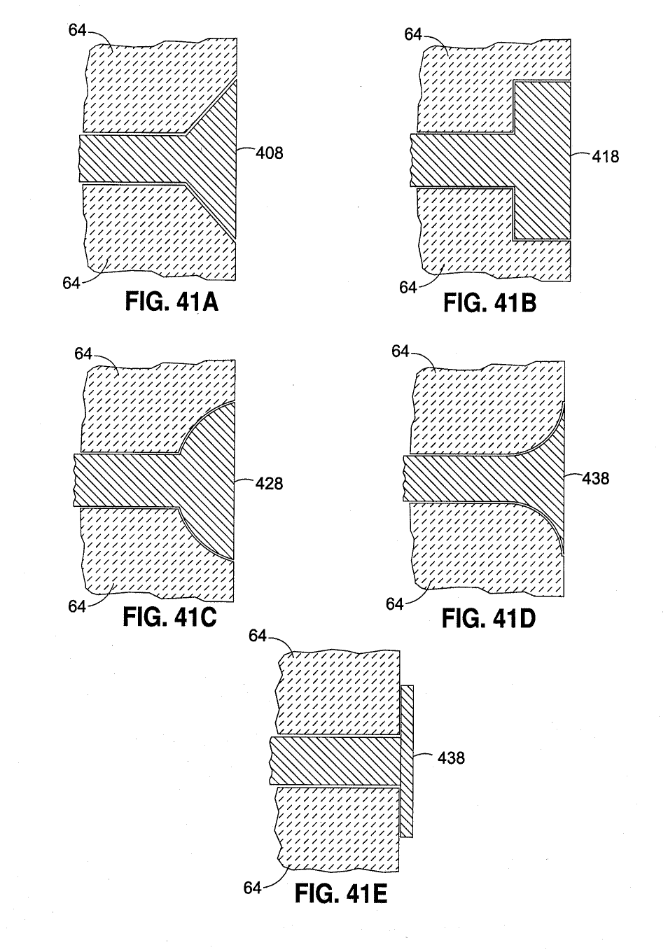

As described in certain embodiments above, methods of preparing the implantation site are also provided. To better secure the spinal implant in place, in certain embodiments it is desirable to ream the extradiscal lips of adjacent vertebrae in order to match the shape of the tail flange on the implant and to receive the implant device in a substantially complementary fit, i.e. countersinking. By doing this, the implant can be effectively countersunk into the adjacent vertebrae, thus limiting protrusion of the implant from the surface of the spine, without limiting its function. Some exemplary embodiments are shown in FIG. 41A-D, a variety of tail flange shapes are compatible with a countersinking method.

Alternatively, and as shown in FIG. 41E, the site may be prepared to receive the implant without countersinking. In either the countersunk or non-countersunk configurations, the tail flange still operates as an externally located barrier relative to the intervertebral disc to prevent loss of material, in particular nucleus pulposus from the interior of the disc.

Several possible general shapes are possible for the tail flange and countersunk region on the vertebrae. In one embodiment, FIG. 41 A, the tail flange 408 has a constant rate taper. In the embodiment illustrated in FIG. 41B, the tail flange 418 is not tapered but rather is relatively squared. In one embodiment, FIG. 41C, the tail flange 428 comprises a curved taper that is generally convex in shape, while in one embodiment, FIG. 41D, the tail flange 438 comprises a curved taper that is general concave in shape. In certain embodiments, the disclosed spinal implants are also compatible with a tail flange that is not countersunk, and which simply abuts the extradiscal lips of adjacent vertebrae, thereby providing an external barrier that prevents extrusion of material from within the intervertebral disc. The illustrated examples are included merely to illustrate some possibilities without intending to be limited to the precise shape and/or size depicted. Various degrees of taper or thickness of the tail flange are also possible.

While not essential for the functioning of the spinal implant, countersinking provides an advantage in that it permits better engagement of the tail flange and the adjacent intervertebral discs, as well as to better prevent inward movement of the implant. Additionally, countersinking permits for a substantially flush fit of the tail flange along the exterior surface of the discs, which may limit pressure on other anatomical structures in the vicinity of the repair site.

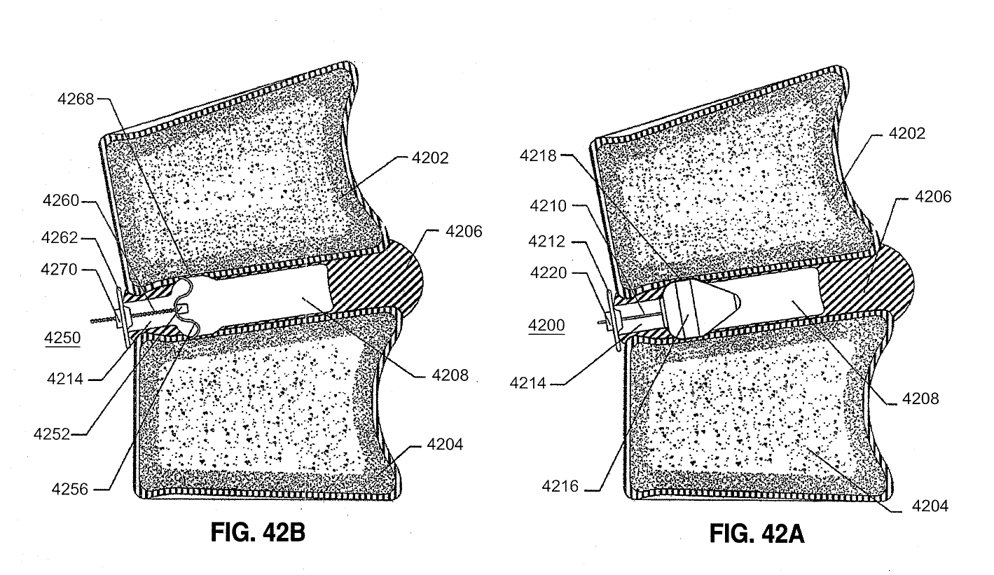

FIG. 42A illustrates an embodiment of an intervertebral disc implant 4200 configured to treat an annular defect, wherein the implant 4200 comprises an anchor head 4216, a tail flange 4212, a tail 4210, and a tail flange connector 4220. In the illustrated embodiment, the implant 4200 is shown implanted in a cross-section of a spine comprising an upper vertebra 4202, a lower vertebra 4204, a disc annulus 4206, a disc nucleus 4208, an annular defect 4214, and an anchor seating area 4218.

In the example shown, the anchor head 4216 is affixed to the tail 4210, which is, in turn, affixed at its proximal end to the tail flange connector 4220, which can be integral to or affixed to the tail flange 4212. The tail 4210 can be thin and/or flexible. The tail 4210 can be resilient or elastomeric but can be configured such that it will not stretch in length beyond a given predetermined limit. The construction of the tail 4210 can, for example, comprise materials such as, but not limited to, Kevlar, polyamide, polyamide, polyester, stainless steel, titanium, and nitinol, in the main structural element, while intermediate degrees of elasticity can be achieved using elastomers such as, but not limited to, silicone elastomer, thermoplastic elastomers, and coiled metal springs. The anchor head 4216 and the tail flange 4212 can be fabricated from materials such as, but not limited to, polyetheretherketone (PEEK), polycarbonate, polyurethane, silicone elastomer, polysulfone, polyester, titanium, nitinol, stainless steel, cobalt nickel alloy, or the like.

FIG. 42B illustrates an intervertebral disc implant 4250 configured to treat an annular defect wherein the implant 4250 comprises an expandable hook anchor head 4256, a tail flange 4262, a ratchet tail 4260, an anchor connector 4252, and a tail flange connector 4270. The implant 4250 can be implanted in a cross-section of a spine comprising an upper vertebra 4202, a lower vertebra 4204, a disc annulus 4206, a disc nucleus 4208, an annular defect 4214, and an anchor seating area 4218.

In the illustrated example, the anchor head 4256 is affixed to the anchor connector 4252, which is affixed to the ratchet tail 4260. The ratchet tail 4260 is constrained to move longitudinally within the tail flange connector 4270. The tail flange 4262 is affixed to the tail flange connector 4270. The ratchet tail 4260 comprises a plurality of bumps, the bumps further comprising one-way ramps on the proximal end of the bumps and vertical or overhang or undercut surfaces on the distal end of the bumps, so that the tail flange connector 4270 and tail flange 4262 can be advanced distally over the ratchet tail 4260 but not release proximally.

The anchor head 4256 can be configured to be elastomeric so that it can be folded or otherwise collapsed during insertion, and then opened up or otherwise expanded following insertion so that its edges dig into and reduce the risk that the implant 4250 will be expelled proximally from the annulus 4214. The anchor head can be fabricated from materials such as, but not limited to, nitinol, stainless steel, titanium, cobalt nickel alloys, and the like. The anchor head can be self-expanding, or can be expanded according to any method known to those of skill in the art, including, without limitation, inflation by a balloon, insertion of fluids such as by a syringe, and activation of a shape memory material.

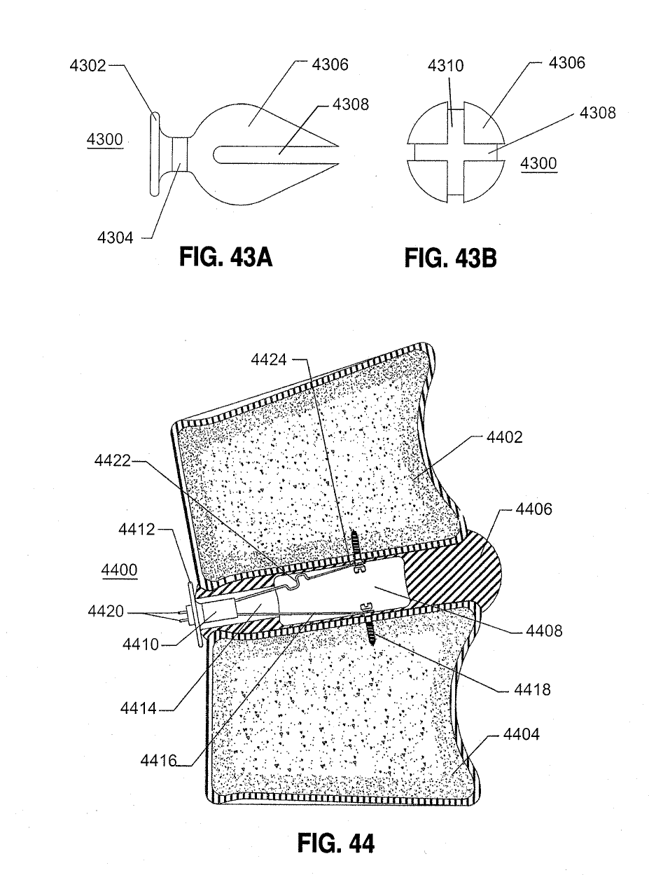

FIG. 43A illustrates a side view of an intervertebral disc implant 4300 comprising an anchor body 4306 further comprising one or more horizontal slots 4308, a tail flange 4302, and a tail 4304. The slots 4308 are cut into the body 4306 to generate a cantilever spring configuration within the body 4306. The cantilever spring configuration can be used to promote expansion of the body 4306, which in turn can be further expanded and heat-set to generate a larger profile that is compressible for insertion into an annular defect. The anchor body 4306 can be fabricated from materials such as, but not limited to, PEEK, polyurethane, polysulfone, titanium, nitinol, stainless steel, cobalt nickel alloy, polycarbonate, and the like.

FIG. 43B illustrates a front view of an intervertebral disc implant 4300 comprising a body 4306, horizontal slot 4308, and vertical slot 4310. The number of slots 4308 and 4310 can vary between 2 and 20, depending on the size of the implant 4300 and strength of the materials used in fabricating the anchor body 4306. The diameter of the anchor body 4306 can range from about 3-mm and about 25-mm and in some embodiments will range between about 4-mm and about 15-mm. This size range is appropriate for the embodiments of implant heads or anchor bodies as described herein.

FIG. 44 illustrates an intervertebral disc implant 4400 configured to treat an annular defect 4414 wherein the implant 4400 comprises a tail flange 4412, one or more anchor wires 4416, one or more anchor fasteners 4418, one or more fastener couplers 4424, a plurality of anchor wire extensions 4420, and a tail flange connector 4410. The anchor wires 4416 can further comprise spring element 4422. The implant 4400 is shown implanted in a cross-section of a spine comprising an upper vertebra 4402, a lower vertebra 4404, a disc annulus 4406, a disc nucleus 4408, and the annular defect 4414.

The tail flange 4412 is affixed to the tail flange connector 4410. The anchor wires 4416 are adjustably affixed within the tail flange connector 4410 and the amount of excess anchor wires or wire extensions 4420 can be adjusted and then trimmed to snug the tail flange 4412 against the annulus 4406. The anchor wires 4416 are affixed to the fasteners 4418 by the fastener couplers 4424. The fasteners 4418 can be screws, rivets, nails, hooks, cleats, or the like and are positively embedded within the upper vertebra 4402 and the lower vertebra 4404 via an open or minimally invasive surgical procedure. The spring element 4422 can be integral to or affixed to one or more of the anchor wires 4416. The anchor wires 4416 can be fabricated from materials such as, but not limited to, polyamide, polyamide, polyester, stainless steel, nitinol, titanium, and the like.

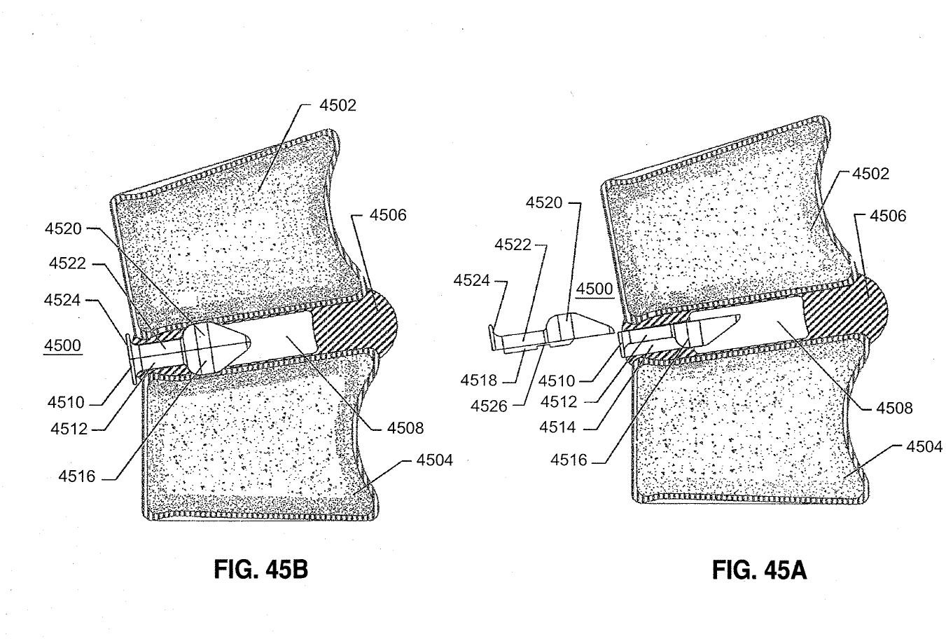

FIG. 45A illustrates a side view of a two-piece intervertebral disc implant 4500 configured to treat an annular defect 4514, wherein the implant 4500 comprises a first anchor head 4516, a first tail flange 4510, a first tail 4512, and a coupler slot (not shown). The implant 4500 further comprises a second anchor head 4520, a second tail 4522 and a second tail flange 4524. In the illustrated example, the implant 4500 is shown implanted in a cross-section of a spine comprising an upper vertebra 4502, a lower vertebra 4504, a disc annulus 4506, a disc nucleus 4508, and the annular defect 4514. The second part of the implant 4500 comprises the second anchor head 4520, the second tail 4522, and the second tail flange 4524 further comprising the dovetail projection 4518 and the locking slot 4526, which engage corresponding structures, a dovetail groove (not shown), and a spring lock projection (not shown), in the first tail 4512, the first anchor head 4516, and the first tail flange 4510 to prevent, respectively, lateral separation and axial separation of the two halves, once they are assembled, as shown in FIG. 45B.

The implant 4500 can be fabricated from materials such as, but not limited to, PEEK, polysulfone, stainless steel, titanium, cobalt nickel alloy, polyurethane, and the like. The length of the tail from the distal end of the tail flange 4524 and 4510 to the maximum diameter of the anchor head 4516, 4520 can range from about 3-mm to about 25-mm, and in some embodiments can range from about 4-mm to about 15-mm. The dovetail projection 4518 can be configured to comprise a wedge shape such as a trapezoid, a T-shaped cross-sectional projection, a circular or oval cross-section, or any other suitable undercut design which prevents separation of the two halves of the implant. The dovetail groove or slot (not shown) on the first part conveniently has a shape that corresponds to the dovetail projection 4518, but with a slightly larger size, to accommodate precise linear movement without binding.

FIG. 45B illustrates the vertebral segment from FIG. 45A comprising the upper vertebra 4502, the lower vertebra 4504, the disc annulus 4506, the disc nucleus 4508, and the annular defect (not shown). The two-piece implant 4500 has been assembled in place within the annular defect 4514. The first anchor head 4516 is longitudinally aligned with the second anchor head 4520 to form a large diameter anchoring structure that effectively resists expulsion from the annular defect 4514. The implant 4500 also comprises the first tail 4512 and the second tail 4522 as well as the first tail flange 4510 and the second tail flange 4524, which are longitudinally aligned. The coupler (not shown) is irreversibly engaged so that the two pieces will not separate from each other.

The coupler can be configured as a spring projection within the dovetail groove, or slot, which remains retracted under force by the dovetail projection 4518 but which can spring out into the locking slot 4526 to prevent the two parts from separating. The spring can be a leaf spring integrally formed in the plastic or it can be a separate spring and lock assembly affixed to the first part of the implant 4500.

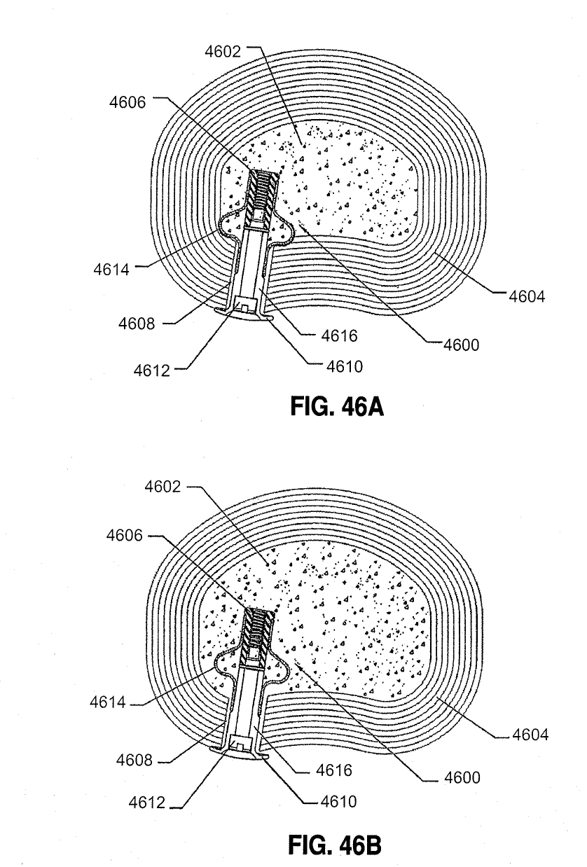

FIG. 46A illustrates an annular implant 4600 in place within an intervertebral disc annulus 4604 and nucleus 4602. The implant 4600 comprises a tail flange 4610, an adjusting screw 4612, a tail 4616, a distal support 4606, and an expandable anchor 4614. In the illustrated example, the implant 4600 is expanded within the nucleus 4602 such that anchors 4614 are expanded into the vertebrae and end plates to secure the implant 4600 and prevent expulsion. The tail flange 4610 and tail 4616 are configured to plug the defect 4608 in the annulus 4604. The line of demarcation between annulus 4604 and nucleus 4602 has been depicted as distinct in FIG. 46A even though in vivo that is generally not the case. While the anchor 4614 can anchor within healthy annulus 4604, patients needing an annular defect plug 4600 generally do not have healthy enough annulus 4604 to permit effective anchoring the implant 4600. Thus, in some embodiments, the anchor 4614 is configured to expand caudally and cranially to engage the vertebrae, vertebral end plates, and similar hard structures (not shown).

In FIG. 46A, the tail flange 4610 is shown affixed to the tail plug 4616. The adjusting screw 4612 is configured to rotate within, and be radially and longitudinally constrained by, the tail plug 4616. The distal support 4606 is constrained to move longitudinally but not rotate relative to the tail 4616. Thus, the tail 4616 and the distal support 4606 telescope relative to each other, the relative position being controlled by the adjusting screw 4612. The distal support 4606 and the tail 4616 comprise features that constrain the ends of the anchoring structure 4614 and capture the anchoring structure 4614 to limit axial or radial migration.

When the adjusting screw 4612 is turned to compress the distance between the tail 4616 and the distal support 4606, the anchoring structure 4614 compresses in length and expands in diameter, in regions where it is slotted to permit such movement. Conversely, turning the adjusting screw 4612 in the other direction results in the tail 4616 moving away from the distal support 4606, which results in lengthening the anchoring structure 4614, and reducing its diameter. The anchoring structure can comprise a longitudinally slotted tube, a series of bars or wires, and the like. The anchoring structure 4614 can be shape set from, for example, nitinol, in its fully expanded configuration so that axial stretching of the ends of the anchoring structure 4614 can cause it to lengthen and constrict radially. The nitinol can be martensite, superelastic and austenitic, or it can have shape memory characteristics that are affected by heating or cooling.

FIG. 46B illustrates the annular implant 4600 of FIG. 46A, with the anchors 4614 expanded completely within nuclear tissue 4602. The anchors 4614 project caudally and cranially to engage bony or cartilaginous end plates or vertebrae, rather than soft tissue such as annulus 4604 or nucleus 4602, which may be compromised or unable to provide adequate support an implant.

As illustrated, the implant 4600 is shown with the anchoring structure 4614 expanded inside what appears to be nucleus. However, this expansion is not for the purpose of anchoring. The anchoring function is provided by expansion of the anchoring structure 4614 in the cranial or caudal direction, resulting in embedding within the bony structures of the vertebrae or the vertebral end plates.

Note that it is very often the case that there will be no nucleus in which to expand an implant or anchor. The annulus may extend, in whole or in part, to the center of the intervertebral disc. Furthermore, the annulus can be structurally compromised and unable to effectively restrain any of the implants described herein. Thus, anchoring methodologies need to be directed toward the bony structures or vertebrae, or the very hard cartilaginous material adjacent thereto.

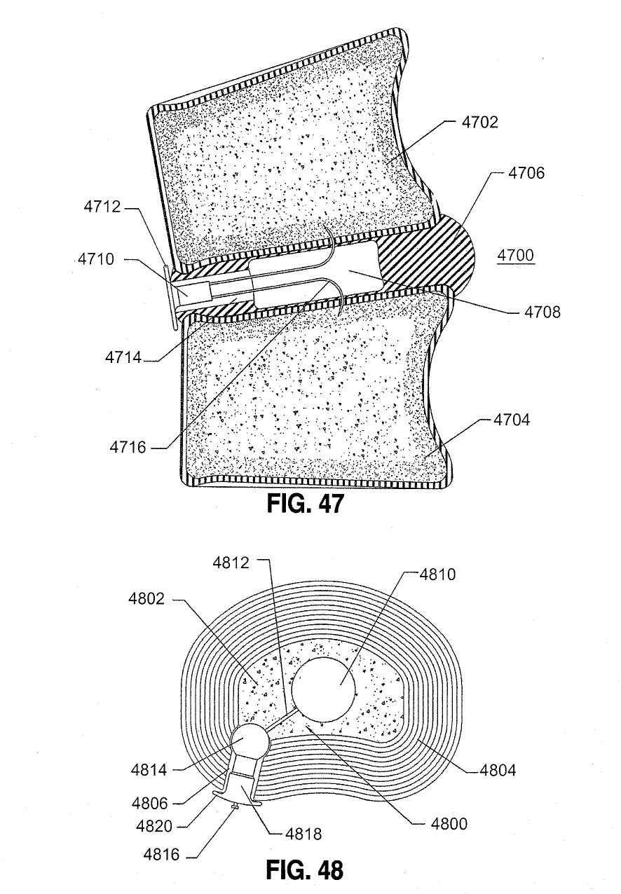

FIG. 47 illustrates an annular implant 4700 introduced to treat an annular defect 4714 in a disc annulus 4706. The implant 4700 comprises a tail flange 4712, a tail 4710, and a plurality of anchors 4716, which are engaged into the vertebral bony structures 4702 and 4704. The annulus 4706 surrounds a nucleus 4708.

The anchors 4716 can be configured to become embedded within the cartilaginous or bony structures of the vertebral anatomy such as the upper vertebra 4702 or the lower vertebra 4704. In some embodiments, the anchors 4716 are sharpened to improve their ability to embed. The anchors 4716 can be shielded or bent straight for insertion, and then released to form the illustrated curvature, which progressively becomes more embedded with time and physiologic compression. The anchors 4716 can be configured at the ends of tethers as in the illustrated embodiment. The anchors 4716 can be fabricated from metals such as, but not limited to, nitinol, stainless steel, tantalum, titanium, cobalt nickel alloy, and the like.

FIG. 48 illustrates an annular implant 4800 comprising a tail 4818, a tail flange 4820, an expandable anchor 4814, a nuclear compression reservoir 4810, a pressure transfer line 4812, and a fluid fill port 4816. In the illustrated example, the implant 4800 is shown implanted in a defect 4806 in an annulus 4804 surrounding a nucleus 4802 for the purpose of closing the defect and preventing re-herniation.

As shown in the illustrated example, the tail 4818 is affixed to the tail flange 4820, which is affixed to the expandable anchor 4814. An inner volume of the nuclear compression reservoir 4810 is operably connected to an inner lumen of the pressure transfer line 4812, which is operably connected to an inner volume of the expandable anchor 4814. The inner volume of the expandable anchor 4814 is operably connected to an inner lumen of the fluid fill port 4816.

The annular implant 4800 can be configured so that compression of the nuclear compression reservoir 4810, which would normally occur with spinal compression, fluid pressure buildup, or flexion, can pressurize fluid in the pressure transfer line 4812 and pressurize the expandable anchor 4814, improving the seating of the anchor 4814, and preventing expulsion of the implant 4800. The nuclear compression reservoir 4810, the pressure transfer line 4812, and the expandable anchor 4814 can be fabricated from materials such as, but not limited to, polyurethane, polycarbonate urethane, silicone elastomer, and the like. These structures can further be reinforced with an embedded mesh or coil fabricated from polyester, polyamide, polyamide, stainless steel, or the like. Fluids suitable for filling the system of the implant 4800 include, but are not limited to, silicone oil, water, hydrogel, and the like. The tail flange 4820 and the tail 4818 can be fabricated from materials as described elsewhere herein. The fluid fill port 4816 is beneficially of the self-sealing type and can comprise a manual shutoff valve or other structures such as a duckbill valve, hemostasis valve, Tuohy-Borst valve, and the like.

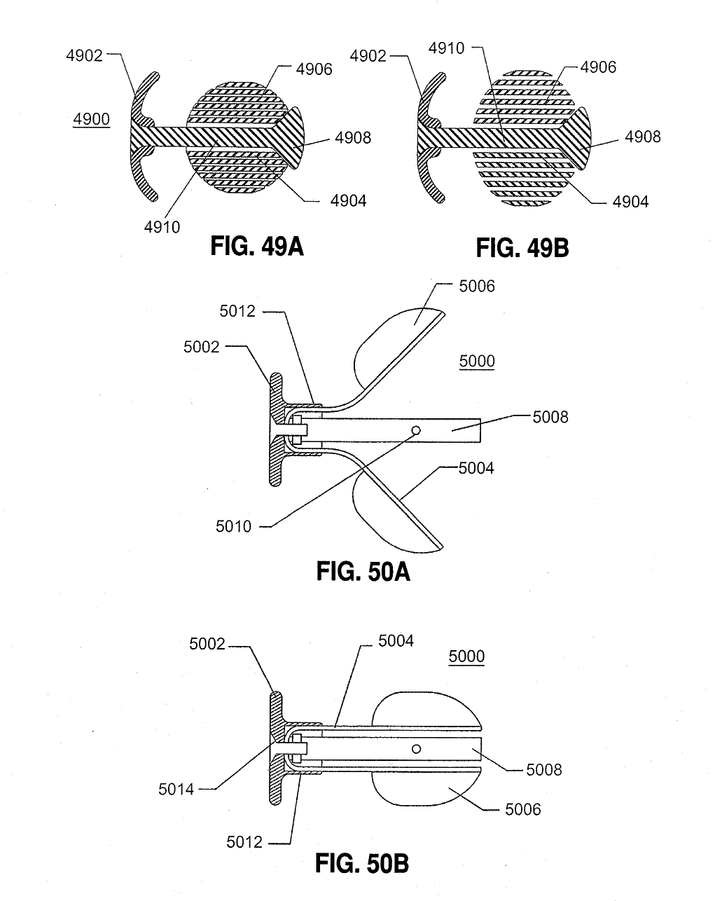

FIG. 49A illustrates a longitudinal cross-section of an expandable annular implant 4900 comprising a tail flange 4902, a body 4910, a distal ramp 4908, and a radially compressed coil spring anchor 4906, further comprising an innermost member 4904. The amount of spring force of the coil spring anchor 4906 can be set to substantially match, for example, the spring resiliency of the annulus (not shown) or it can be set to a higher force level.

In the illustrated example, the tail flange 4902 is affixed to the body 4910, which is affixed to, or integral to, the distal ramp 4908. The body 4910 is constrained to move axially within the innermost member 4904. The coil spring anchor 4906 is constrained by its innermost member 4904 to rest against the distal ramp 4908, and can expand radially outward to fill available volume. The coils spring anchor 4906 can be fabricated from cobalt nickel alloy, titanium, stainless steel, nitinol, or the like. The tail flange 4902 can be fabricated from PEEK or other materials identified herein. The body 4910 and the distal ramp 4908 can be fabricated from the same materials as the tail flange 4902 or the coil spring anchor 4906.

FIG. 49B illustrates a longitudinal cross-section of the annular implant 4900 of FIG. 49A wherein the coil spring anchor 4906 has expanded radially expanded. The distal ramp 4908 is configured such that should the innermost member 4904 expand, the anchor can move proximally thus allowing the tail flange 4902 to move proximally away from the annulus. This situation can occur when the disc is under relaxed conditions so pressures within the intervertebral disc are minimal. When compression occurs, the innermost member 4904 is compressed against the ramp 4908 forcing the body 4910 and the tail flange 4902 to move distally toward the annulus so that the tail flange 4902 is snug against the annulus of the intervertebral disc (not shown) when needed most, i.e., at high intradiscal pressure.

Any of a variety of restraining members can be used to restrain the annular implant 4900 in the radially constrained configuration illustrated in FIG. 49A. For example, a sheath substantially wrapped around a circumference of an outer surface of the coil spring anchor 4906 may be used while the annular implant 4900 is inserted into an intervertebral disc space, and thereafter the sheath may be removed in order to transform the annular implant 4900 from the radially constrained configuration to the radially expanded configuration illustrated in FIG. 49B.

FIG. 50A illustrates an annular implant 5000 comprising a tail flange 5002, a tail assembly 5012, a plurality of laterally projecting spring elements 5008, a plurality of vertically projecting spring elements 5004, and a plurality of vertebral engaging anchors 5006. For clarity in the illustration, the anchor 5006 is shown not affixed to spring element 5008. An attachment mechanism 5010 is shown on spring 5008. The spring elements 5004 are shown deflected outward in FIG. 50A. The tail assembly 5012 can comprise an introducer attachment feature 5014, which permits releasable connection between the implant 5000 and an introducer (not shown).

As shown in the illustration, the tail flange 5002 is affixed to the tail assembly 5012. The laterally projecting spring elements 5008 and the vertically projecting spring elements 5004 are affixed to the tail assembly 5012. The vertebral engaging anchors 5006 are affixed to the ends of the laterally and vertically projecting spring elements 5008 and 5004 by attachment mechanisms 5010. The attachment mechanisms 5010 can comprise holes drilled in the spring elements 5008 and 5004, to permit bonding by insert molding or attachment using fasteners such as screws, bolts, rivets, and the like. The spring elements 5008 and 5004 can be fabricated from materials such as, but not limited to, nitinol, cobalt nickel alloy, stainless steel, and the like. The spring elements 5008 and 5004 can be shape-set superelastic or shape-memory nitinol that are pre-formed in the outward configuration as shown in FIG. 50A. The thickness of the spring elements 5008 and 5004 can range from about 0.002 to about 0.030 inches and in some embodiments between about 0.010 and about 0.025 inches.

Conveniently, the spring elements 5008 and 5004 can be configured to have substantially the same spring constant as that of the intervertebral disc annulus. The vertebral engaging anchors 5006 can be fabricated from materials such as PEEK, which has similar hardness as that of the vertebrae. The anchors 5006 can be rounded, squared, or sharpened to positively engage the vertebrae (not shown). The number of spring elements 5008 and 5004 can range from two to 20 depending on the size of the implant and the material from which the components are fabricated. The spring elements 5008 and 5004 can be fabricated from flat wire.

FIG. 50B illustrates the implant 5000 of FIG. 50A wherein the spring elements 5004 have been compressed radially inward to generate a minimum diameter configuration. The anchors 5006 subtend the smallest possible cross-sectional area in FIG. 50B suitable for insertion into an annular defect.

Any of a variety of restraining members can be used to restrain the annular implant 5000 in the minimum diameter configuration illustrated in FIG. 50B. For example, a sheath substantially wrapped around a circumference of an outer surface of the annular implant 5000 may be used while the annular implant 5000 is inserted into an intervertebral disc space, and thereafter the sheath may be removed in order to transform the annular implant 5000 from the minimum diameter configuration to the configuration having a greater diameter, as illustrated in FIG. 50A.

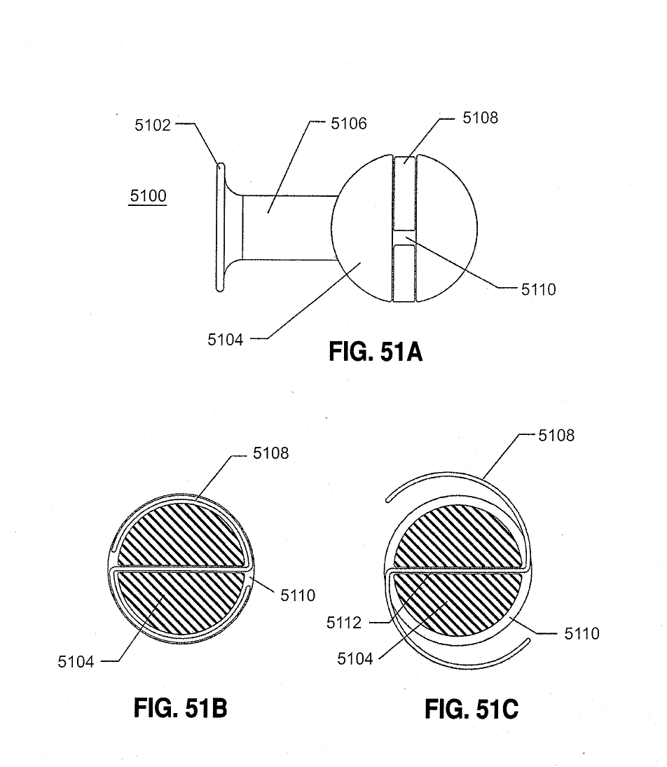

FIG. 51A illustrates a side view of an annular implant comprising a tail flange 5102, a tail 5106, a head 5104, a groove 5110, and a spiral spring anchor 5108. In the illustrated embodiment, the tail flange 5102 is shown affixed to the tail 5106, which is in turn affixed to the head 5104. The spring anchors 5108 are affixed, at a central point to the head 5104. The spring anchors 5108 can be compressed into the circumferential groove 5110, which is integral to the head 5104, such as with the use of a restraining member, e.g., a removable sheath (not shown). The head 5104, the tail 5106, and the tail flange 5102 can be fabricated from PEEK or other biocompatible materials as described herein. The spiral spring anchor 5108 can be fabricated from the same materials as described for the spring elements 5008 and 5004 of the embodiment illustrated in FIGS. 50A and 50B. In some embodiments, the spiral spring anchor 5108 can be tipped with polymeric materials such as PEEK to provide a non-traumatic bone contact surface, or they can be left bare.

FIG. 51B illustrates a lateral cross-sectional view of the head 5104 at the level of the spiral spring anchor 5108. The spiral spring anchor is illustrated within the groove 5110 fully compressed inward in a configuration suitable for insertion into the annulus of an intervertebral disc.

FIG. 51C illustrates a lateral cross-sectional view of the head 5104 of the implant 5100 of FIG. 51A. The spiral spring anchor 5108 is illustrated expanded radially outward to engage structures or tissue within the intervertebral disc. The spiral spring 5108 is shown with two projections and is affixed to the head 5104 by being threaded through a slot 5112 in the head 5104.

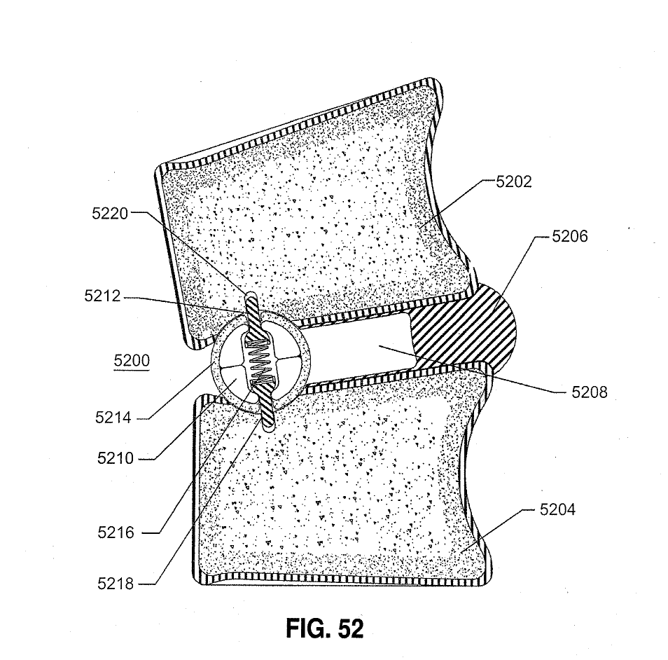

FIG. 52 illustrates a side cross-sectional view of an intervertebral disc comprising an annulus 5206, a nucleus 5208, and vertebrae, 5202 and 5204, wherein an implant 5200 has been inserted into a defect in the annulus 5206. The implant 5200 comprises a body 5210, a soft exterior layer 5214, a plurality of anchor pins 5212, and one or more spring bias elements 5216. The implant 5200 is illustrated placed within a reamed depression 5218 in the upper vertebra 5202 and a depression 5220 in the lower vertebra 5204. The depressions 5218 and 5220 are shown further comprising slots or recesses within which the pins 5212 project to secure the implant 5200 from expulsion.

The body 5210 can be fabricated in two or more pieces and then joined by welding, bonding, fastening, or the like. The spring bias elements 5216 are inserted into features within the body 5210 along with the anchor pins 5212, which are configured to be restrained at a certain limit of radial projection within the body 5210, such as with the use of a restraining member, e.g., a removable sheath (not shown). The soft exterior layer 5214 can be coated over the completed body 5210. The soft exterior layer 5214 can be fabricated from materials such as, but not limited to, silicone elastomer, polyurethane, polycarbonate urethane, thermoplastic elastomer, hydrogel, and the like. The body 5210 can be fabricated from PEEK, or other polymer or biocompatible metal. The anchor pins 5212 can be fabricated from metals such as stainless steel, titanium, tantalum, cobalt nickel alloy, and the like, or they can be fabricated from relatively hard polymers such as, but not limited to, PEEK, polysulfone, polyester, and the like.

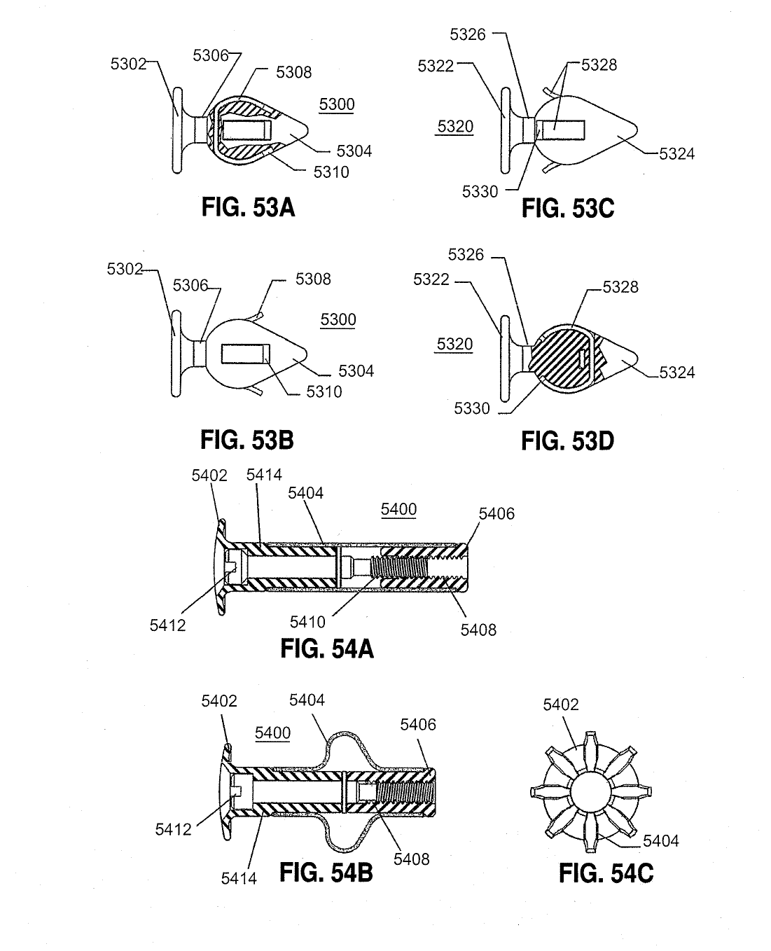

FIG. 53A illustrates a partial breakaway side view of an annular implant 5300 comprising a tail flange 5302, a tail 5306, a body 5304, slots 5310 and a plurality of spring anchors 5308. The spring anchors 5308 are illustrated compressed against the body 5304 into the grooves 5310 such that the implant 5300 can be inserted into an annulus. The grooves 5310 and the spring anchors 5308 are oriented so that they are constrained toward the tail end of the head and open outward toward the head end of the implant 5300. As shown in the illustrated example, the tail flange 5302 is affixed or in some embodiments integral to the tail 5306. The body 5304 can also be affixed, or in some embodiments integral, to the tail 5306. The grooves 5310 can be integral to the body 5304. The spring anchors 5308 can be affixed to the body 5304 at a central region but are free at their ends to be biased away from the body 5304 along substantially the length of their exposed outer surface. The materials used in construction of the implant 5300 can be the same as those used in construction of the implant 5000 shown in FIGS. 50A and 50B.

FIG. 53B illustrates a side view of the annular implant 5300 of FIG. 53A wherein the spring anchors 5308 have moved to their relaxed or neutral state out of the grooves 5310 such that the spring anchors 5308 can engage vertebral structures (not shown) to reduce the risk of expulsion of the implant 5300.

The amount of projection of the spring anchors 5308 out of the grooves 5310, when in their unconstrained state, can vary between about 0.5-mm and about 10-mm. The number of spring anchors 5308 can vary between 2 and 20, and the geometry, size, and materials will determine the optimum number of spring anchors 5308. The spring anchors 5308 can have bare metal ends, or they can be tipped with polymeric masses that offer the potential of reduced tissue trauma. The polymeric masses (not shown) can be fabricated from PEEK, polysulfone, polyester, or the like, and can be insert-molded, bonded, welded, ultrasonically welded, or pinned, or otherwise fastened to the spring anchors 5308. In some embodiments, the polymeric masses can be configured to be recessed within the body 5304, when in their retracted state.

FIG. 53C illustrates a side view of an embodiment of an annular implant 5320 comprising a tail flange 5322, a tail 5326, a head 5324, a plurality of grooves 5330, and a plurality of spring anchors 5328. In the illustrated example, the spring anchors 5320 and grooves 5330 are oriented so that the spring anchors 5328 are constrained or affixed to the head 5324 toward the head end of the implant 5320 and open toward the tail 5326 end of the implant 5320. The spring anchors 5328 are shown sprung outward in their relaxed or neutral state such that they can engage tissue and prevent expulsion of the implant 5320.

The tail flange 5322 can be affixed, or integral to, the tail 5326. The body 5324 can be affixed, or integral to, the tail 5326. The grooves 5330 are integral to the body 5324. The spring anchors 5328 are affixed to the body 5324 at a central region, but are free at their ends to be biased away from the body 5324 along substantially the length of their exposed outer surface. The materials used in construction of the implant 5320, as well as general overall dimensions, are the same as those used in construction of the implant 5000 shown in FIGS. 50A and 50B. In certain embodiments, the spring anchors 5328 can be restrained using a restraining member, e.g., a removable sheath (not shown).

FIG. 53D illustrates a partial breakaway side view of the implant 5320 of FIG. 53C wherein the spring anchors 5328 are compressed inward into the grooves 5330 in the head 5324 in a configuration suitable for insertion into an annular defect.

The amount of projection of the spring anchors 5328 out of the grooves 5330, when in their unconstrained state, can vary between about 0.5-mm and about 10-mm. The slots 5330 that run through the body 5324 from one side to the other can comprise fasteners or other bonding agents affix the spring anchors 5328 firmly to the body 5324. The proximally oriented opening of the spring elements 5328 allows for the implant 5320 to be inserted into a disc annulus but prevents expulsion, or withdrawal, of the implant 5320 from the annulus (not shown). In some embodiments, the spring elements 5328 can comprise bare ends, as illustrated. In some embodiments, the spring elements 5328 can be tipped with large footprint structures (not shown), for example fabricated from polymeric materials such as PEEK, polysulfone, polycarbonate, polyester, and the like, which limit trauma of surrounding tissues.

FIG. 54A illustrates an annular implant 5400 comprising a tail flange 5402, a threaded adjustment screw 5412, a tail 5414, a plurality of expandable anchor elements 5404, and a compression head 5406, further comprising an internal thread 5408. In the illustrated example, the expandable anchor elements 5404 are shown in their radially compressed configuration having a minimum profile suitable for insertion into an annular defect (not shown).

As shown in the illustration, the tail flange 5402 can be affixed to the tail 5414. The adjustment screw 5412 can rotate within, and be radially and longitudinally constrained by, the tail 5414. The compression head 5406 is constrained to move longitudinally but not rotate relative to the tail 5414. Thus, the tail 4616 and the distal compression head 5406 telescope relative to each other, the position being controlled by the adjustment screw 5412. The compression head 5406 and the tail 5414 comprise features that constrain the ends of the anchor elements 5404 and capture the anchor elements 5404 from migrating axially or radially. When the adjustment screw 5412 is turned to compress the distance between the tail 5414 and the compression head 5406, the anchor elements 5404 compress in length and expand in diameter in regions where it is slotted to permit such movement. Conversely, turning the adjustment screw 5412 in an opposite direction causes the tail 5414 to move away from the compression head 5406, lengthening the anchor elements 5404 and reducing its diameter. The anchor elements 5404 can be a longitudinally slotted tube, a series of bars or wires, or the like. The anchor elements 5404 can be shape-set from, for example, nitinol, in its fully expanded configuration so that axial stretching of the ends of the anchor elements 5404 can cause it to axially lengthen and constrict radially. The nitinol can be martensite, superelastic and austenitic, or it can have shape memory characteristics that are affected by heating or cooling.

FIG. 54B illustrates the anchor implant 5400 of FIG. 54A wherein the adjustment screw 5412 has been fully screwed into the threads 5408 of the compression head 5406 resulting in an outward radial deformation of the expandable anchors 5404 to subtend a maximum profile suitable for restraining the implant 5400 from expulsion from an intervertebral disc.

In some embodiments, the anchor elements 5404 are configured to expand to a maximum diameter of between 1.1 and 5 times their unexpanded diameter. The anchor elements 5404 can be configured to expand with various longitudinal cross-sectional shapes. In an illustrated example, the space between the proximal end of the compression head 5406 and the distal end of the tail 5414 has been reduced to a minimum distance, as shown in FIG. 54B. The outside of the tail 5414, the compression head 5406, or both, can be coated with a dried, hydrophilic, water-swellable hydrogel that is configured to increase in volume upon exposure to moisture in the body, effectively filling space interior to the expandable anchors 5404.

FIG. 54C illustrates a face-on lateral view looking toward the tail flange 5402 showing the lateral configuration of the expandable anchors 5404. The expandable anchors 5404 are configured, in this embodiment, with eight elements 5405 circumferentially disposed about the implant 5400. The number of anchor elements 5404 can range from one to 50, being practically limited by the ability to divide the material of the anchor elements 5404 into separate structures. The greater the number of anchor elements, the less prone the implant 5400 will be to reorient itself within the annulus in response to externally applied forces, for example, vertebral compression.

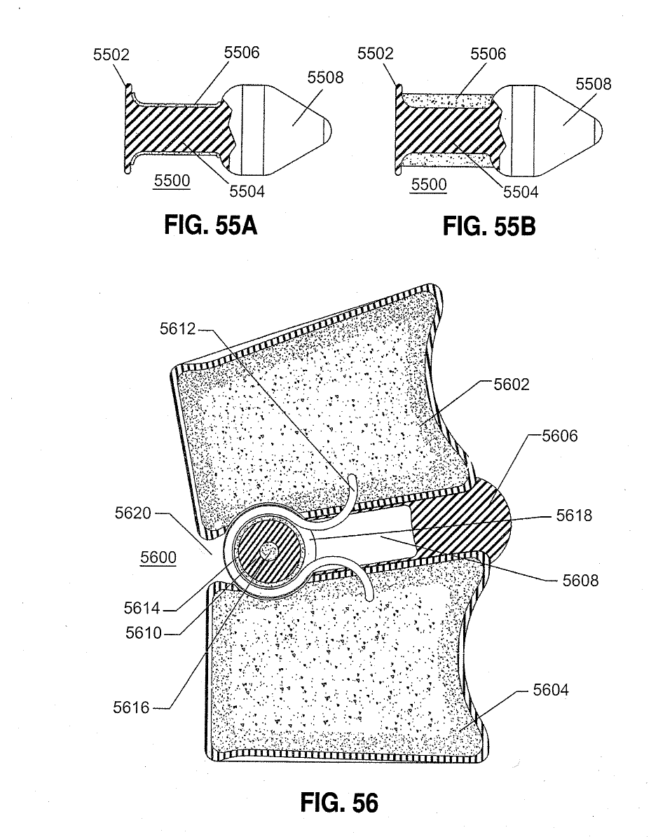

FIG. 55A illustrates an annular implant 5500 comprising a tail flange 5502, a tail 5504, an anchor head 5508, and a layer of dried hydrophilic hydrogel 5506 affixed to the tail 5504. This hydrophilic hydrogel embodiment can be applied to any of the embodiments for an annular repair plug disclosed herein to improve the sealing characteristics of the tail.

The tail flange 5502 can be affixed or integral to the tail 5504. The tail 5504 can be integral to, or affixed to, the anchor head 5508. The water-swellable layer of hydrophilic hydrogel 5506 can be applied in its dry formulation to the tail 5504 or it can be applied wet to at least some degree, and then be dried to minimize its volume.

FIG. 55B illustrates the annular implant of FIG. 55A wherein the swellable hydrophilic hydrogel 5506 has absorbed water and has swollen to increase its volume. Suitable water-swellable hydrogel materials include, but are not limited to, polyethylene glycol and polyHEMA, polymethyl cellulose, and the like. Swelling ratios between wet and dry materials ranging from about 2:1 to about 10:1 are achievable with these materials. The volume increase of the hydrogel 5506 assists with sealing of the tail 5504 within an annular defect (not shown) in an intervertebral disc.

The hydrogel 5506 can be applied to the tail 5504, as illustrated, or it can be applied to the distal end of the tail flange 5502, or to the exterior surface of the anchor head 5508. The exterior surfaces of the tail 5504, the anchor head 5508, or the tail flange 5502 can be configured with dimples, holes, villi, or other structures (not shown) to improve mechanical adherence of the hydrogel 5506 to the implant 5500.

FIG. 56 illustrates an annular implant 5600 for closing a defect 5620 in the annulus 5606 of an intervertebral disc. The implant 5600 comprises a body core 5616, a body main support 5610, a soft polymeric body surround 5614, a groove 5618, and a spring loaded hook 5612. The implant 5600 is configured to reside within space reamed out of the upper vertebra 5602 and lower vertebra 5604. The implant 5600 is configured to prevent the escape of nucleus material 5608 from the intervertebral disc through the defect 5620.

The body core 5616 can be fabricated from polymeric materials or it can be a hollowed out area within the body main support 5610. The body main support 5610 can be fabricated from PEEK, polycarbonate, polysulfone, polyester, and the like. The spring loaded hook 5612 is affixed to the body main support 5610 and can further reside within the groove 5618. The soft polymeric body surround 5416 can be a soft elastomer such as, but not limited to, hydrogel, silicone elastomer, thermoplastic elastomer, polyurethane, polycarbonate urethane, and the like.

The thickness of the soft polymeric body surround 5416 can range from about 0.25-mm to about 10-mm or more, or in some embodiments between about 1-mm and about 5-mm. The anchors 5612 can be configured to become embedded in both the upper vertebra 5602 and lower vertebra 5604. The anchors 5612 can be fashioned sharp and stiff enough to resist expulsion due to forces generated within the nucleus 5608 of the intervertebral disc. In some embodiments, the spring-loaded hooks, or anchors 5612, can be compressed inward for implantation or insertion, such as with the use of a restraining member, e.g., a removable sheath (not shown). Conveniently, the annular defect can be reamed to create a region of undercut in which the implant 5600 rests, effective to both seal the annular defect 5620 and assist with anchoring. In some embodiments, the main body support 5610 can be fabricated from elastomeric polymeric material that permits some compression, allowing the implant 5600 to retain its fit within the annulus 5618.

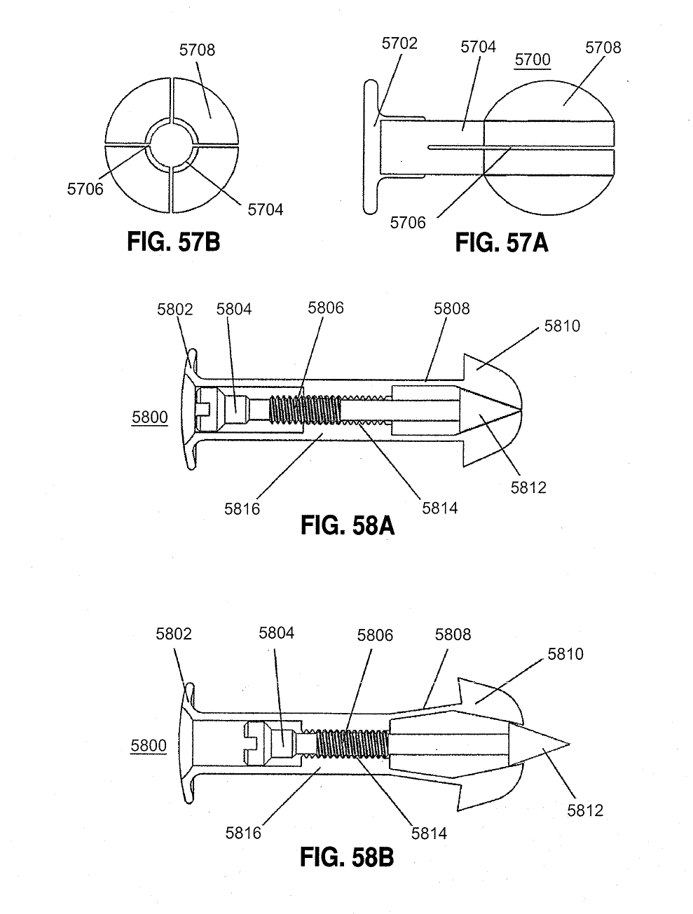

FIG. 57A illustrates a side view of an annular implant 5700 comprising a tail 5702, a tubular spring 5704, and further comprising a plurality of longitudinal slots or cuts 5706, and a plurality of anchors 5708. The implant 5700 can further comprise an optional elastomeric casing (not shown) to limit contact of the interior of the tubular spring 5704 with tissue.

This implant 5700 can be similar in function to the implant 5000 of FIGS. 50A and 50B except that it uses a tubular spring structure 5704 comprising slots 5706 to create a plurality of cantilever springs. The springs 5704 can be pre-formed outward as illustrated in FIG. 50A. The anchors 5708 are configured to be held against the bony tissue or other vertebral structures to retain the anchoring function no matter what the spacing of the vertebrae. The tubular spring structure 5704 can be fabricated from materials such as, but not limited to, superelastic nitinol, shape memory nitinol, cobalt nickel alloy, titanium, stainless steel, and the like. The anchors 5708 can be semi-spherical, semi-elliptical, squared off, or comprise barbs, hooks, or other features that facilitate effective engagement of tissue.

FIG. 57B illustrates a front, lateral view of the implant 5700 showing the anchors 5708, the spring elements 5704, and the slots 5706. Although four are shown in the illustrated example, the number of slots 5706, spring elements 5704, and anchors 5708 can range from two to 20, for example from 3 to 10. The anchors can be fabricated from PEEK, polysulfone, polycarbonate, polyester, polyamide, polyamide, or the like. The central region inside the spring elements 5704 can be filled, in part, or in whole, with elastomeric materials such as, but not limited to, polyurethane, polycarbonate urethane, silicone elastomer, thermoplastic elastomer, hydrophilic hydrogel, and the like.

FIG. 58A illustrates a side cross-sectional view of an annular implant 5800 configured to treat a defect in an intervertebral disc (not shown). The implant 5800 comprises a tail flange 5802, an adjustment screw 5804 further comprising a threaded section 5806 and a wedge-shaped expander 5812, a body 5816 further comprising an internal threaded section 5814, the spring elements 5808, and the anchors 5810. The implant 5800 is illustrated in its radially compressed, minimum cross-sectional profile suitable for introduction into an annular defect of an intervertebral disc.

The tail flange 5802 can be affixed to, or integrally formed with, the body 5816. The internal threaded section 5814 can be integrally formed with the body 5816. The anchors 5810 can be integrally formed with, or affixed to, the spring elements 5808. The spring elements 5808 can be affixed to, or formed integrally with, the body 5814. The adjustment screw 5804 is captured by the body 5816 and radially restrained. The adjustment screw 5804 can travel axially within the body 5816 in response to rotation resulting from an interaction between the adjustment screw 5804 and the internal threaded section 5814. The wedge-shaped expander 5812 can be affixed to, or integrally formed with, the adjustment screw 5804, and either rotates therewith or comprises a rotary bearing (not shown) that limits rotation of the expander 5812 while it is being advanced, or retracted, by the adjustment screw 5804. In some embodiments, the angle of the distal end of the expander 5812 can range from about 10 degrees to about 80 degrees (one side), and in some embodiments, from about 20 degrees to about 60 degrees.

FIG. 58B illustrates a longitudinal cross-sectional view of the annular implant 5800 of FIG. 58A in its radially expanded configuration. In some embodiments, the inner surface of the anchors 5810 can be tapered inward moving distally. In some embodiments, the inward taper of the anchors 5810 can comprise an inwardly projecting ridge or bump. The adjustment screw 5804 can be advanced distally resulting in the wedge-shaped expander 5812 forcing the anchors 5810 radially and outward to engage the vertebrae, or their end plates, thus effective to limit the risk of the implant being expelled from site of the annular defect. The body 5816 can be fabricated from PEEK, polycarbonate, polyamide, polyamide, stainless steel, titanium, polyester, nitinol, or other high-strength biocompatible material.

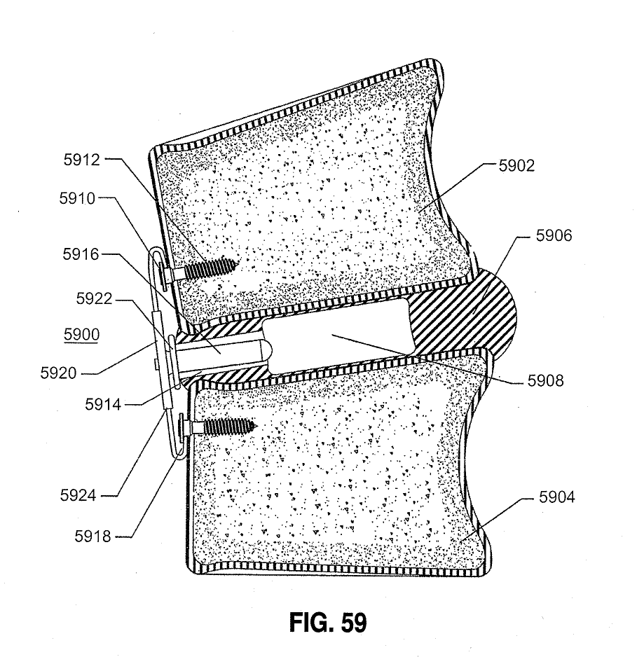

FIG. 59 illustrates a side cross-sectional view of an annular implant 5900 positioned within an annular defect 5914 of an intervertebral disc comprising an annulus 5906 and a nucleus 5908, and sandwiched between an upper vertebra 5902 and a lower vertebra 5904. The implant 5900 comprises a tail 5916, a tail flange 5922, a restraining member 5920, a plurality of vertebral fasteners 5912, and a plurality of fastener quick-connects 5918. The restraining member 5920 can comprise length changing elements 5924 to permit the restraining member to shorten or lengthen, as required by variable intervertebral spacing, without allowing the restraining member 5920 to move further proximal (posterior) away from the spine. These length-changing elements can be of the type including, but not limited to, telescoping members as shown in the illustrated embodiment, resilient bending members, hinged members, and the like.

The tail flange 5922 can be affixed, or integral, to the tail 5916. The restraining member 5920 can be affixed, or integral, to the tail 5916. The length changing elements 5924 can be received within the restraining member 5920, such that the length changing elements 5924 can move axially relative to the restraining member 5920, but are otherwise restrained from moving or bending laterally. The quick-connects 5918 can be affixed to the length changing elements 5924. The quick-connects 5918 can be configured with a fork-shape, hook, or other shape. The fasteners 5912 can be separate and can be affixed to the bone prior to attachment of the quick-connects 5918. The fasteners 5912 can also be pre-attached through the quick-connects 5918 and made free to rotate but restrained from axial relative motion therethrough. The tail 5916 can be coated with a water-swellable hydrophilic hydrogel to enhance filling and sealing of the annular defect 5914.

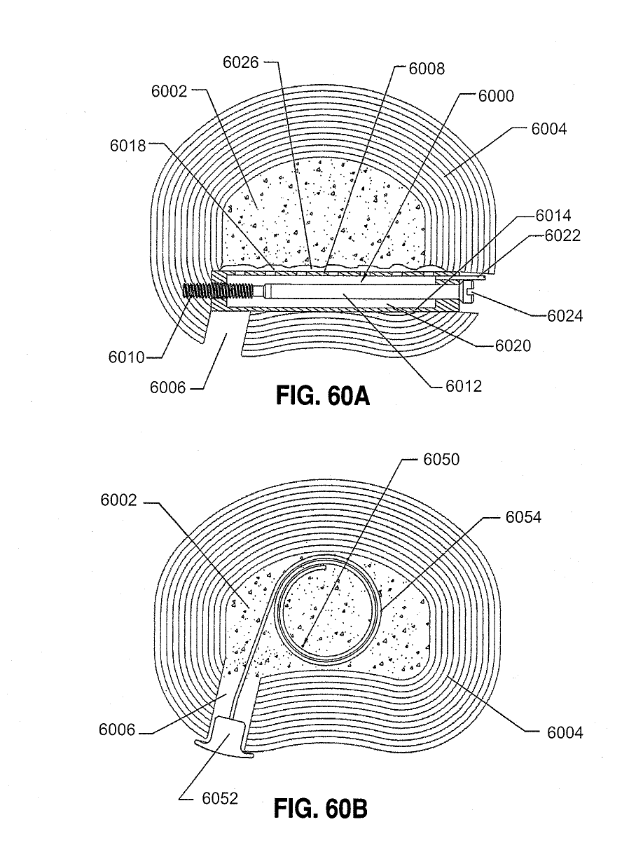

FIG. 60A illustrates a cross-sectional view of an intervertebral disc, wherein an implant 6000 has been placed within the disc. The intervertebral disc comprises an annulus 6004, a nucleus 6002, and an annular defect 6006. The implant 6000 comprises an outer shell 6008 further comprising a central lumen 6020, a fluid injection port 6022, and a plurality of purge ports 6018, a fixation screw 6012 further comprising a head 6024 and a threaded end 6010. The lumen 6020 can be filled with material comprising a pharmaceutical, hydrophilic hydrogel, and the like. Water injected into the fluid injection port 6022 can be used to hydrate a dried hydrogel, such that it swells and extrudes through the ports 6018 to form the exterior layer 6026.

The outer shell 6008 surrounds and restricts the fixation screw 6012 from lateral and longitudinal motion, but permits rotary motion of the fixation screw 6012. The fluid injection port 6022 can be integral, or affixed to, the outer shell 6008. A lumen of the fluid injection port 6022 can be operably connected to the inner lumen 6020 of the outer shell 6008. The purge ports 6018 can be formed integrally into the outer shell 6008 and operably connect the inner lumen 6020 of the outer shell 6008 to the environment outside the outer shell 6008.

In the illustrated example, the implant 6000 is placed across the annular defect 6006 via a posterior lateral approach, thus avoiding potential entanglements with spinal nerves. The implant 6000 can be axially elongate and can have a circular, rectangular, oval, triangular, or any other suitable cross-sectional configuration. The position of the implant 600 is not affected by the extent of annulus 6004 encroachment into the nucleus 6002. The implant can be placed using a flexible delivery system including a sheath, a plunger, a rotary driver drill that reversibly engages the head 6024, and appropriate steering mechanisms.

FIG. 60B illustrates a cross-sectional view of an intervertebral disc, wherein an implant 6050 is positioned to occlude an annular defect 6006. The intervertebral disc comprises an annulus 6004, a nucleus 6002, and an annular defect 6006. The implant 6050 comprises a tail flange 6052 and a coil retainer 6054. The implant 6050 is placed through the annular defect 6006.

The tail flange 6052 is affixed to the coil retainer 6054. The coil retainer 6054 can be formed from shape-set nitinol that is either superelastic or shape memory in characteristics. An austenite finish temperature (A_{f}) from about 28° C. to about 32° C. can permit the coil retainer 6054 to be inserted relatively straight, and then be configured to form a coil as it equilibrates to body temperature, which is above the austenite finish temperature. In certain embodiments, other forms of activation energy can be used. In certain embodiments, the coil retainer 6054 can be inserted in a relatively straight configuration with the use of a restraining member, e.g., a removable sheath (not shown).

The coil retainer 6054 can be formed from round or flat wire having a first lateral dimension ranging from about 0.010 inches to about 0.050 inches and a second lateral dimension ranging from about 0.010 to about 0.050 inches. An introducer (not shown) can also be used to move the coil retainer 6054 through the annular defect 6006 and into the intervertebral disc where the coil retainer 6054 will form a circular coil or in some embodiments, a coil of complex three-dimensional shape. The coil retainer 6054 can be configured to form at least a single complete coil. In some embodiments, the coil retainer 6054 is configured to form more than one coil.

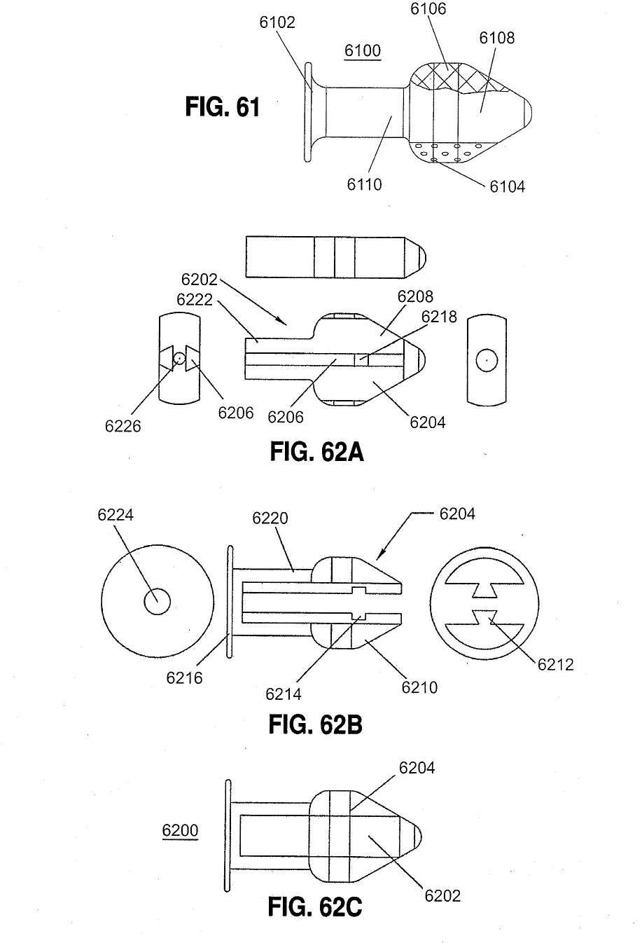

FIG. 61 illustrates a side view of an annular implant 6100 comprising a head 6108, a tail flange 6102, and a tail 6110. The implant 6100 can further comprise a layer of bone growth factor 6106 applied to the top or the bottom of the head 6108. In some embodiments, the bone growth factor 6106 is applied to one of the top or bottom of the head 6108. In some embodiments, the surface of the head 6108 can be configured to comprise holes, wells, dimples, or protrusions 6104 capable of improving affixation of the bone growth material 6106.

The tail flange 6102 can be affixed to the tail 6110, which can be affixed to the head 6108, or the parts can be integrally formed. The bone growth factor 6106 can be pre-applied to the head 6018, either during manufacturing or by the implanting medical personnel. Where applied to one surface of the head 6108, the bone growth factor 6106 results in the head 6108 attaching to either the upper or the lower vertebrae but not both, thus allowing for motion preservation while still maximizing anchoring within the vertebral structures.

FIG. 62A illustrates side, top, and two end views of the inner part 6202 of a two-part annular implant 6200. The inner part 6202 comprises the center of the two-part implant 6200, and provides the major function of restraining or anchoring the implant 6200 within an annular defect. The inner part comprises a head or anchor 6208, a tail 6222, an engagement groove 6206, a longitudinal lock mechanism 6218, and an introducer coupler 6226. The anchor 6208 can be formed integrally to, or is affixed to, the tail 6222. The engagement groove 6206 and the longitudinal lock mechanism 6218 can affixed to, or integrally formed within, the anchor 6208 and the tail 6222. The engagement groove 6206 can comprise a dovetail slot or it can comprise a T-slot other functional equivalent.

The anchor head 6208 of the inner implant 6202 can be configured to be higher than it is wide so that it can be turned sideways for insertion between the vertebral lips. Once the head 6208 is inside and past the vertebral lip, the inner part 6202 can be rotated about 90° to maximize interference with the lip. The tail 6222 of the inner implant 6202 can be, as shown in the illustrated embodiment, the same width or slightly narrower than the narrow width of the inner part implant 6202. The introducer coupler 6226 can be integral to the tail 6222 or it can be affixed thereto.

In some embodiments, the tail 6222 can comprise an attachment feature (not shown) on its proximal end to facilitate connection with an introducing tool or instrument (not shown). The attachment feature permits connection with the introducing tool or instrument such that rotation of the instrument also rotates the inner implant 6222, but also permits release of the introducing tool or instrument when desired. The inner implant 6202 can be formed from PEEK, titanium, cobalt nickel alloy, polysulfone, polyester, and the like and can further comprise radiopaque markers fabricated from materials such as, but not limited to, tantalum, platinum, iridium, gold, barium sulfate filler, bismuth sulfate filler, and the like, to enhance visibility under fluoroscopy or X-ray imaging.

The introducer coupler 6226 can be a threaded hole, a bayonet mount, an undercut hole, or any other type of reversible locking mechanism suitable for selectively affixing or decoupling the inner implant 6202 to the distal end of an introducer (not shown). The introducer coupler 6226 can advantageously provide torque coupling between the introducer (not shown) and the inner implant 6202 so that the inner implant 6202 can be inserted into an annular defect and then be rotated into a position of maximum interference with the vertebrae. In some embodiments of a threaded or bayonet mount type introducer coupler 6226, the implant 6202 can be rotated clockwise by the introducer and then decoupled from the introducer by rotating the introducer counterclockwise to disengage the two parts.

FIG. 62B illustrates a top and two end views of the outer part 6204 of the annular implant 6200. The outer part 6204 further comprises the coupler 6206, an engagement projection 6212, a lock detent 6214, a tail flange 6216 further comprising a holder attachment 6224, a tail structure 6220, and one or more anchor heads 6210.

The tail structure 6220 can be affixed, or formed integrally, to the tail flange 6216 and the anchor heads 6210. The engagement projection 6212, in some embodiments one affixed to each tail structure 6220 and anchor head 6210 can comprise a dovetail shape, a T-shaped cross-section, or other shape that corresponds with the engagement groove 6206 on the inner implant 6202. The engagement projection 6212 can have dimensions that permit it to fit within the engagement groove 6206 of the inner implant 6202 with sufficient clearance to slide smoothly, but still be retained from coming apart laterally.

The holder attachment 6224 can be a round or irregularly shaped hole in the tail flange 6216 that permits passage of an introducer (not shown). The irregularly shaped hole, such as a rectangular, keyed, or slotted hole, can index on a rectangular cross-sectional holder shaft to not permit the holder shaft to rotate within the hole, until the tail flange 6216 has been completely, or almost completely, advanced against and locked to the inner implant 6202. Rotation within the holder attachment 6224 can be beneficial after the outer part 6204 has been advanced substantially completely onto the inner implant 6202, by allowing, for example, the introducer (not shown) to be rotated counterclockwise to disengage the introducer from the inner implant 6202.

The outer part 6204 can be fabricated from the same or similar materials as those used for the inner implant 6202. The tail flange 6216 can be round (as illustrated), rectangular, elliptical, oval, or other shape suitable for closing the annular defect.

FIG. 62C illustrates the inner part 6202 with an outer part 6204 inserted over it, and with the engagement projection 6212 of FIG. 62B slidably restrained within the engagement groove 6206 of FIG. 62A. The lock mechanism 6218 of FIG. 62A is irreversibly engaged within the lock detent 6214 of FIG. 62B. The inner implant 6202 and the outer part 6204 can be pre-positioned in a staged position on an implantation instrument so that they are restrained from improper relative motion, and so that they are aligned for connection. The embodiment illustrated in FIG. 62C shows a bottom or top view, with the widest projection illustrated. However, the inner implant 6202 comprises a much greater height (in and out of the plane of the page) than would be possible with a single piece implant. In an exemplary embodiment, the inner implant 6202 can be inserted into an annulus sideways such that the height profile ranges from about 4-mm to about 5-mm. The inner implant 6202 can be rotated approximately 90° to have a profile height within the annulus from about 9-mm to about 10-mm. The outer part 6204 can be inserted with a height of about 4-mm to about 5-mm and locked in place around the inner implant 6202 to create a single implant 6200 that ranges from about 9-mm to about 10-mm high and from about 11-mm to about 12-mm wide. Having a final width greater than the height for the implant 6200 further enhances its stability within the annulus under the compressive forces of the vertebrae, thus preventing inadvertent rotation.

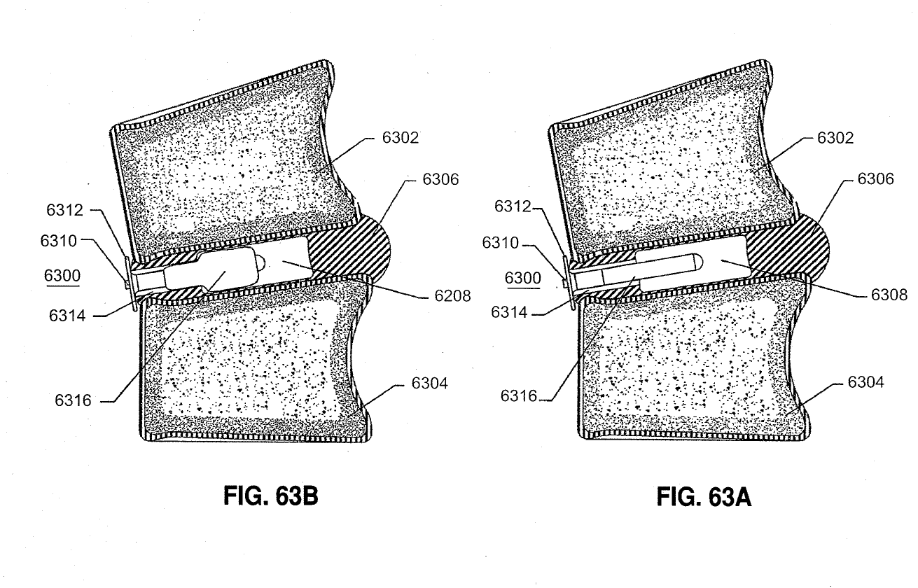

FIG. 63A illustrates an annular implant 6300 placed within an annular defect 6314 of an intervertebral disc further comprising an annulus 6306, and a nucleus 6308. The disc is sandwiched between an upper vertebra 6302 and a lower vertebra 6304. The annular implant 6300 comprises a tail flange 6312, an expandable anchor 6316, illustrated in a non-expanded state, and an anchor inflation port 6310. The tail flange 6312 can be affixed to the expandable anchor 6316. The anchor inflation port 6310 can be affixed to, or integral to, the tail flange 6312. The anchor inflation port 6310 comprises a lumen and valve (not shown) that are operably connected to the interior of the expandable anchor 6316. An inflation device (not shown), such as a syringe, angioplasty balloon inflation device, or similar can be temporarily and reversibly affixed to the anchor inflation port 6310 and used to inject fluid therethrough to fill the expandable anchor 6316.

The valve (not shown) in the inflation port 6310 can be configured to automatically seal the lumen of the expandable anchor 6316 from losing fluid or fluid pressure to the ambient environment. Such a valve can comprise, but is not limited to, a duckbill valve, a membrane valve, a slit in a sheet of elastomer, a Tuohy-Borst valve, a stopcock, or the like. The expandable anchor 6316 can be fabricated from elastomeric materials such as silicone elastomer, thermoplastic elastomer, polyurethane, latex rubber, or the like. In another embodiment, the expandable anchor 6316 can be fabricated from non-elastomeric materials such as, but not limited to, polyester, polyamide, polyamide, cross-linked polyethylene, or the like. The expandable anchor 6316 in the non-elastomeric embodiment is analogous to a non-stretchable bag that when filled with fluid becomes very rigid and exerts very high forces on surrounding structures.

FIG. 63B illustrates the annular implant 6300 of FIG. 63A, wherein the expandable anchor 6316 has been expanded by filling with fluid, gas, or other material through the anchor inflation port 6310. The expandable anchor 6316 can be a structure such as an angioplasty balloon, essentially an inelastic bag filled with fluid, or it can be a diaphragm, bellows, or like structures that have little or no resiliency under expansive pressure. The fluid used to fill the expandable anchor 6316 can comprise, but is not limited to, water, saline, hydrogel, cellulose, two part epoxy, or the like. The expandable anchor 6316 can be filled at pressures ranging between about 0.1 psi and about 500 psi.

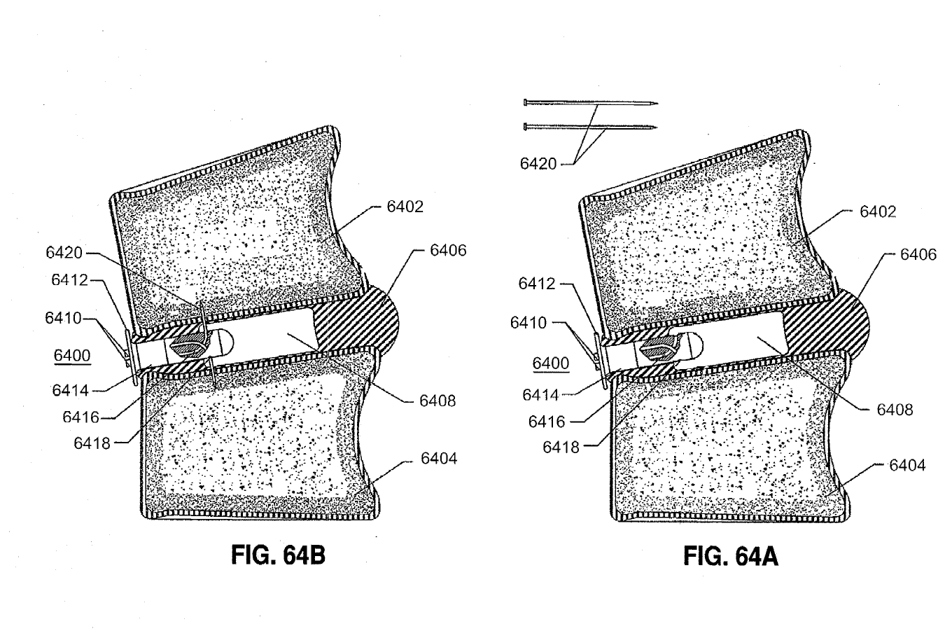

FIG. 64A illustrates an annular implant 6400 placed within a defect in an intervertebral disc. The intervertebral disc comprises the annulus 6406 and the nucleus 6408. The implant 6400 comprises a tail flange 6412, a plurality of anchor ports 6410, a body 6414, one or more anchor lumens 6416, and one or more anchor exit ports 6418. The implant 6400 can also comprise one or more anchors 6420, which in the illustration are shown not yet inserted into the implant 6400.

The tail flange 6412 is affixed to, or integrally formed with, the body 6414. The anchor ports 6410 are entry ports affixed to the tail flange 6412 and operably connected to the anchor lumens 6416. The anchor ports 6410 can further comprise locking couplers such as external or internal threads, bayonet mounts, snap locks, and the like for permanent connection with the proximal ends of the anchors 6420. The body 6414 can be configured to have as large in diameter as possible, for a given annulus size, to permit gradual bending of the anchor lumens 6414. The anchor lumens 6416 are terminated at their distal ends, and operably connected to the anchor exit ports 6418, which are integral to the body 6414. In some embodiments, the body 6414 is of sufficient caliber to abut the bony or fibrous tissue of adjacent vertebrae.

The anchors 6420, which can range in number from one to 20, in some embodiments between two and 10, can be sharpened at their distal end and flexible, and are constructed to generate significant column strength. The distal ends of the anchors 6420 can optionally comprise threads configured to be screwed into bony or cartilaginous tissue. The proximal ends of the anchors 6420 can comprise locks configured to mate with the locking couplers on the anchor ports 6410. The proximal ends of the anchors 6420 can further comprise keys, such as slots, hex heads, Phillips screwdriver heads, and the like, to permit rotation from an instrument (not shown) operated by the implanting surgeon. The shafts of the anchors 6420 are capable of rotation and bending and thus can move in a manner analogous to a speedometer/odometer drive cable. The construction of the anchor shafts can be spring wire fabricated from materials such as, but not limited to, nitinol, stainless steel, titanium, cobalt nickel alloy, and the like. The anchor shafts can also comprise braided or coiled structures capable of transmitting torque and having column strength while permitting bending and rotation. The anchor shafts can be configured to resist shear such that axial force applied to the implant 6400 will be resisted by the flexible anchors. This will result in little or no axial motion of the implant 6400 in response to these forces.

FIG. 64B illustrates the annular implant 6400 of FIG. 64A, wherein the anchors 6420 have been inserted into the anchor ports 6410, and advanced through the anchor lumens 6416 and the anchor exit ports 6418 into the vertebrae 6402 and 6404. In certain embodiments, the anchors 6420 may be at least partially inserted into the annular implant 6400 while the annular implant 6400 is inserted into the intervertebral disc. In certain embodiments, the anchors 6420 may be inserted into the annular implant 6400 after the annular implant 6400 is inserted into the intervertebral disc. In the illustrated embodiment, there are two anchors 6420 advanced through two anchor lumens 6416, which direct the flexible anchors 6420 toward the side exit ports 6418 and into the bone where they achieve substantial holding capability. The anchors 6420 are capable of bending, but resist shear, thus preventing retrograde, or antegrade, movement of the implant 6400 even when subjected to forces exerted by the spinal system. In some embodiments, the closer the side exit ports 6418 are to vertebrae 6402, 6404, the less will be the effect of bending on the anchors 6420. This results in better securement of the implant 6400 between adjacent vertebrae 6402, 6404.

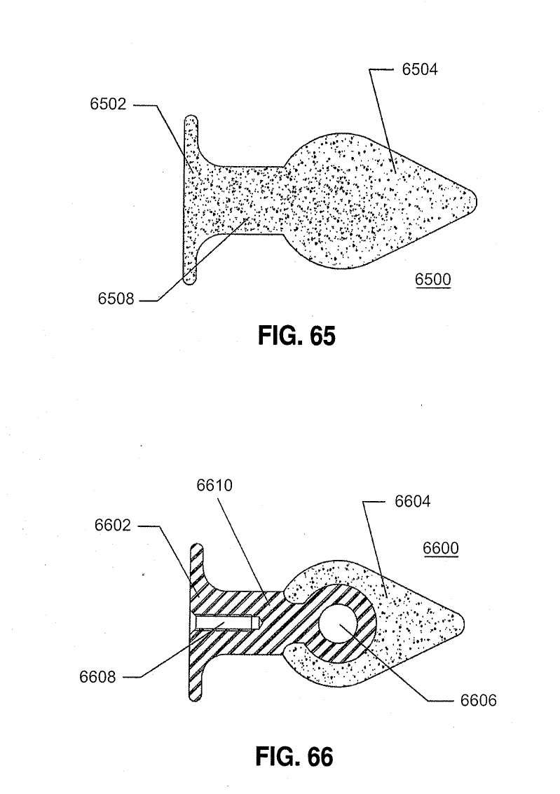

FIG. 65 illustrates an annular implant 6500 comprising a tail flange 6502, a tail 6508, and a head, or anchor, 6504. The body 6504, the tail 6508, and the tail flange 6502 are fabricated from soft resilient polymer such as, but not limited to, C-Flex, silicone elastomer, polyurethane, polycarbonate urethane, and the like.

The tail flange 6502 can be affixed to, or integrally formed with, the tail 6508, which can be affixed to, or integrally formed with, the head 6504. The hardness of the polymer can range from about 20 A to about 100 A, and in some embodiments, from about 40 A to about 85 A. The implant 6500 can further comprise radiopaque markers (not shown) embedded therein, wherein the radiopaque markers are fabricated from tantalum, gold, platinum, iridium, and the like. The implant 6500 can also comprise radiopaque materials such as barium or bismuth sulfate formulated with the polymer in percentages ranging from about 10% to about 50%.

FIG. 66 illustrates an annular implant 6600 comprising a tail flange 6602, an engagement feature 6608, a tail 6610, an anchor 6604, and a tail to head coupling feature 6606. The head 6604 of the illustrated embodiment can be fabricated from elastomeric, polymer with a hardness level much lower than that of the tail 6610 or the tail flange 6602. Suitable manufacturing techniques for fabricating the implant 6600 include insert molding, dip molding, and injection molding. The soft material used in the head 6604 may be advantageous during implantation of the device within an intervertebral disc.

The head 6604 can be fabricated from materials such as those suitable for the implant 6500 illustrated in FIG. 65 and having the same relative hardness. The tail 6610 and tail flange 6602 can be fabricated from harder materials such as, but not limited to, PEEK, polycarbonate, polysulfone, polyester, polyamide, polyamide, stainless steel, titanium, cobalt nickel alloys, and the like. The engagement feature 6608 can be integrally formed with, or affixed to, the tail 6610. The head or anchor 6604 can be insert-molded around, bonded to, or fastened to, the tail 6610, with the head-coupling feature 6606 facilitating a firm mechanical connection.

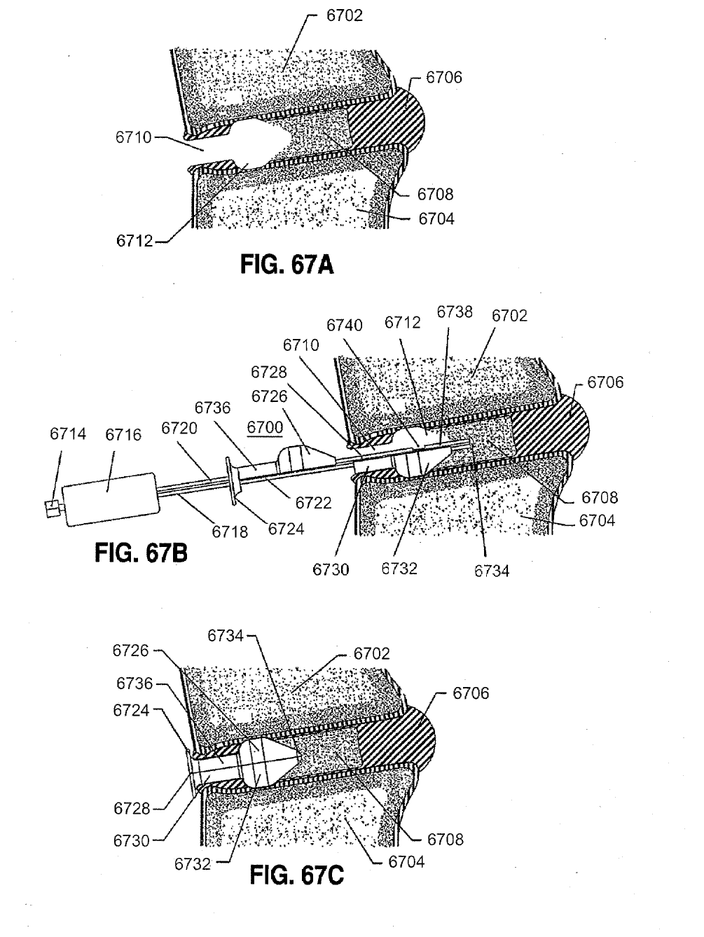

FIG. 67A illustrates a side cross-sectional view of a vertebral segment further comprising an upper vertebra 6702, a lower vertebra 6704, a disc annulus 6706, a disc nucleus 6708, an annular defect 6710, and a reamed region 6712 within the annulus 6706, the nucleus 6708, and the vertebrae 6702, 6704.

The reamed region 6712 can be created using a reamer (not shown). The reamer can have between two and eight flutes and the flutes can be either helical or straight. In some embodiments, the reamer comprises cross-sectional dimensions that permit it to be inserted through a small annulus height, and still be able to ream an adequately large cavity within the intervertebral space, into which an implant can be inserted. Such a reamer can comprise two flutes, it can comprise two flutes with lateral stabilizers, or it can comprise four flutes that fold together for insertion, and then open up to generate a larger dimension. The shape of the void created by the reamer can be configured to be similar to the shape of the head or anchor of an implant. The dimension of material removed from the annulus between the vertebral lips can reach to the bone, or it can retain some soft or softer tissue.

FIG. 67B illustrates the vertebral segment of FIG. 67A, wherein an annular implant 6700 is being advanced sequentially into the annular defect 6710. The implant 6700 comprises a forward head 6732, a forward tail 6730, a follow-up head 6728, a follow-up tail 6736, a follow-up tail flange 6724, a deployment rail 6720 further comprising an implant rail 6738, an implant lock detent 6740, an implant stop 6734, and an implant rail coupler 6728, an introducer handle 6716, and an implant rail coupler control 6714. In certain embodiments, the annular implant 6700 can be composed of more than two pieces, such as three pieces, four pieces, eight pieces, and so on.

The forward head 6732 is integrally formed with, or affixed to, the forward tail 6730. The follow-up head 6728 is integrally formed with, or affixed to, the follow-up tail 6736, which is integrally formed with, or affixed to, the follow-up tail flange 6724. In another embodiment, the forward tail 6730 can be affixed to, or integrally formed with, half of the tail flange 6724 while the follow-up tail 6736 is affixed to, or integrally formed with, the other half of the tail flange 6724. The implant 6700 is formed integral to the introducer which comprises the handle 6716 and the deployment rail 6720. The deployment rail 6720 is reversibly coupled to the implant rail 6738 which is affixed to or integrally formed with the implant stop 6734. The implant rail 6738 and the implant stop 6734 remain as part of the implant following detachment of the deployment rail 6720. The deployment rail 6720 has the same or similar cross-section as the implant rail 6738 and retains rotational alignment of the forward head 6732 and forward tail 6730 and the follow-up head 6728, follow-up tail 6736, and the tail flange 6724. The forward head 6732 and its tail 6730 and the follow-up head 6726 and its attached components are configured to slide longitudinally over the deployment rail 6720 but not separate laterally.

The cross-sectional shape of the deployment rail can be similar to that of the engagement projection 6212 of FIG. 62B. The cross-sectional shape of the slot (not shown) in the implant heads, tails, and tail flanges, can be the same or similar to that of the engagement slot 6206 in FIG. 62A. In the illustrated example, the implant rail coupler control 6714 has been activated to release the implant rail coupler 6728 so that the deployment rail 6720 and the handle 6716 have become disconnected from the implant rail 6738 and removed from the figure, leaving the implant within the reamed out region 6712. The implant rail coupler control 6714 can be a knob connected to a rotating linkage (not shown) extending through the length of the deployment rail 6720 to a screw or bayonet mount at the distal end of the deployment rail 6720. Counterclockwise rotation, for example, of the implant rail coupler control 6714 can unscrew or detach the implant rail 6738 from the deployment rail 6720.

FIG. 67C illustrates the implant 6700 of FIG. 67B, wherein the follow-up head 6728, the follow-up tail 6736, and the follow-up tail flange 6724 have been advanced over the deployment rail 6720 until they are aligned with and locked into the forward head 6732, the forward tail 6730, the implant rail 6738, and the implant stop 6734. This configuration of implant 6700 allows for linear sequenced implantation of the implant 6700 with a larger head structure 6726 and 6732 through a narrow annulus 6710 than could be achieved with a one-piece implant.

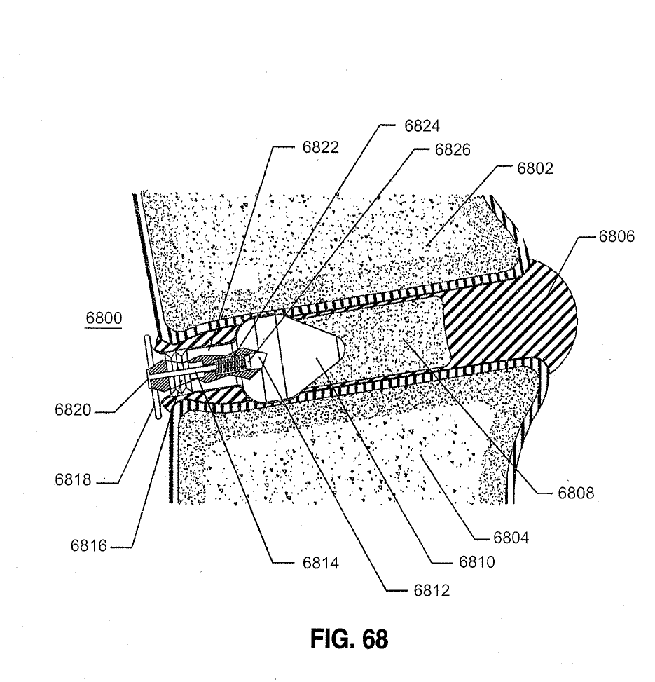

FIG. 68 illustrates a partial breakaway, side view of an annular implant 6800 implanted within an annular defect within the annulus 6806 of an intervertebral disc also comprising a nucleus 6808. The intervertebral disc is sandwiched between an upper vertebra 6802 and a lower vertebra 6804. The implant 6800 comprises a tail flange 6818, a head 6810, a tail shaft 6814, a spring 6824, a tail 6822, a collapsible region 6816 in the tail 6822, a tail shaft stop 6826, and a tail shaft coupler 6820.

In the illustrated embodiment, the tail flange 6818 is shown affixed to the tail shaft 6814 by the tail shaft coupler 6820. The tail shaft 6814 is affixed to, or integral to, the tail shaft stop 6826. The spring 6824 is radially constrained around the tail shaft 6814 and linearly constrained by an area of reduced diameter in the tail 6822 at its proximal end and by the tail shaft stop 6826 at its distal end. The tail 6822 is affixed, or integral, to the head 6810. The collapsible region 6816 is affixed between the tail flange 6818 and the tail 6822 and permits axial movement therebetween while preventing tissue encroachment therein. The collapsible region 6816 can be fabricated from elastomeric polymers or it can be fabricated from accordion folded polymeric materials. The collapsible region 6816 can comprise a telescoping structure, a hinged structure, or the like. The spring 6824 biases the tail shaft stop 6826 distally to keep the tail flange 6818 biased toward the intervertebral disc. The tail flange 6818 can comprise porous materials on its proximal side, distal side, or both, for the purpose of encouraging tissue ingrowth. The tail 6822 can further comprise porous materials configured to encourage tissue ingrowth. The porous materials can be affixed to the tail flange 6818 or the tail 6822 or they can be integral. Suitable porous materials include, but are not limited to, polyester woven or knitted fabric, polytetrafluoroethylene woven or knitted fabric, holes formed in the surface of the implant, and the like.

The spring-loaded tail flange 6818 is effective in maintaining a seal against the annular defect that prevents additional annulus 6806 or nucleus 6808 from being expelled and impinging on a nerve following a discectomy procedure. Such spring bias is desirable because while motion in the intervertebral disc is preserved, the anchor head 6810 can shift slightly proximally or distally. Thus, maintaining the seal is important no matter what the location of the head 6810. The spring 6824 can comprise a coil of wire, or it can be configured as a cantilever spring, leaf spring, and the like. The spring 6824 can be fabricated from metallic materials such as nitinol, stainless steel, cobalt nickel alloy, and the like. The spring 6824 can, in another embodiment, comprise polymeric spring materials such as, but not limited to, silicone elastomer, thermoplastic elastomer, polyurethane elastomer, and the like. The spring-loaded tail flange 6818 and the elements of the implant 6800 can beneficially be applied to any of the implants disclosed herein.

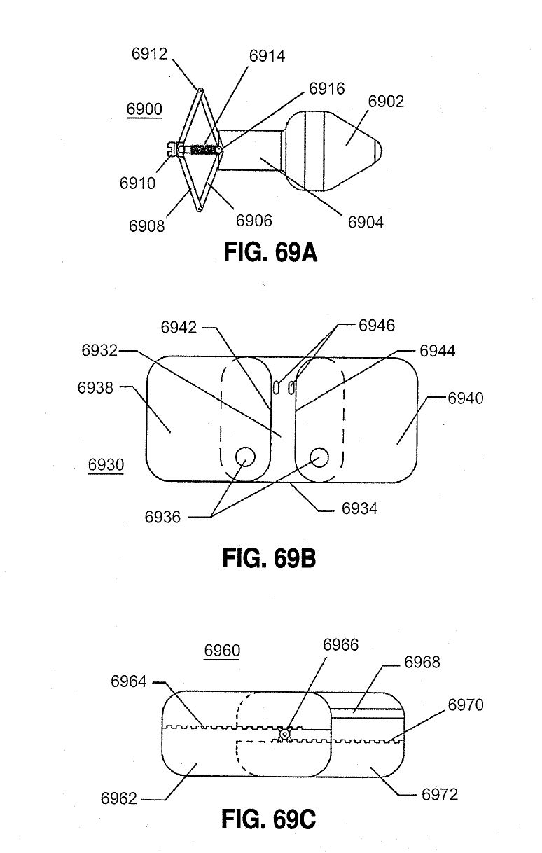

FIG. 69A illustrates a side view of an annular implant 6900 comprising an anchor head 6902, a tail 6904, and a radially expandable tail flange comprising a plurality of distal tail segments 6906, a plurality of proximal tail segments 6908, an adjustment screw 6910 comprising a threaded section 6914, a plurality of outer hinge joints 6912, a hinged tail flange connector 6916. The implant 6900 can be configured to permit tail flange elements 6906 and 6908 to expand to a lateral dimension greater than that of the anchor head 6902 while still being advanceable through a small diameter access port (not shown). The anchor head 6902 is affixed to, or integral with, the tail 6904. The tail 6904 is affixed to the tail flange connector 6916. The distal tail segments 6906 are rotatably affixed to the tail flange connector 6916, which serves as a hinge point for the rotation. The proximal tail segments 6908 are affixed to the distal tail segments 6906 by the outer hinge points 6912, about which they are rotatably connected. The adjustment screw 6910 is threaded into the tail 6904 by the threaded section 6914, which engages inner threads within the tail 6904. The head of the adjustment screw 6910 is enlarged and exerts axial force on the proximal tail segments 6908 as it is threaded into, or out of, the tail 6904. As with other embodiments discussed herein, the adjustment screw 6910 can be at least partially inserted in the annular implant 6900 while the annular implant 6900 is inserted into the intervertebral disc space, or, alternatively, the adjustment screw 6910 can be inserted in the annular implant 6900 after the annular implant 6900 is inserted into the intervertebral disc space.

Rotation of the adjustment screw 6910 can be accomplished with a tool somewhat like a screwdriver, Phillips screwdriver, hex wrench, or the like. The vertical dimension of the tail flanges 6906 and 6908 can be very small when the adjustment screw 6910 is unscrewed axially proximally away from the tail 6904, with a projection ranging in length from about 2-mm to about 10-mm. When the adjustment screw 6910 is fully advanced distally toward the tail 6904, the maximum projection of the tail flanges 6906 and 6908 can be increased to between about 3-mm and about 25-mm. The lateral dimension of the tail flanges 6906 and 6908 into and out of the plane of the page, can range between about 4-mm and about 25-mm or greater. The accordion-type tail flange embodiment of the implant 6900 can be incorporated into the embodiments of the annular implant disclosed herein.

The materials suitable for construction of the adjustable tail segments 6906 and 6908 include, but are not limited to, polysulfone, PEEK, titanium, polycarbonate, polyester, polyamide, polyamide, nitinol, silicone elastomer, thermoplastic elastomer, polyurethane, polycarbonate urethane, and the like. The hinges 6912 and 6916 can be fabricated from metallic or polymeric components.

FIG. 69B illustrates a view looking distally at the tail flange 6930 along the longitudinal axis of an annular implant. The tail flange 6930 comprises a central region 6932, a right foldout region 6940, a left foldout region 6938, a plurality of hinges 6936, and a plurality of locks 6946. The central region 6932 comprises a bottom edge 6934. The right foldout region 6940 comprises a left edge 6944, and the left foldout region 6938 comprises a right edge 6942. The tail flange 6930 is configured with a lateral collapsed profile not substantially larger than that of the central region 6932 during insertion through an access port. The right and left fold-out regions 6940 and 6938 can be unfolded about hinges 6936 to generate a tail flange 6930 substantially wider than that of the central region 6932. Once folded outward, the locks 6946 prevent the right and left foldout regions 6940 and 6938 from retracting.

The materials suitable for fabricating the tail flange 6930 can be the same or similar to those used in fabricating the tail flange 6906 and 6908 of FIG. 69A. The materials suitable for fabricating the hinges 6936 can be the same or similar to those used to fabricate the hinges 6912 and 6916 of FIG. 69A. The open and closed dimensions of the expandable tail flange 6930 can be similar to those of the tail flange of the implant 6900 of FIG. 69A. An advantage is that the system 6930 can be implanted with a relatively square, or rounded, tail flange no larger than that of the central region 6932 and then the right and left fold-out regions 6940 and 6938 expand laterally and locking at approximately the same height but a much larger width than the central region 6932. The height and width of the central region 6932 can be configured to permit introduction through a minimally invasive port access device with inner diameters ranging, for example, between about 10-mm and about 25-mm, and in some embodiments between about 15-mm and about 20-mm. The rotatably outward folding tail flange embodiment 6930 can be incorporated into the embodiments of the annular implant disclosed herein.

FIG. 69C illustrates a tail flange 6960 of an annular implant looking distally along the axis of the implant. The tail flange 6960 comprises a right part 6972, a left part 6962, and a gear wheel 6966. The right part 6972 further comprises the integral engagement groove 6968 that slidably couples with an integral or affixed engagement projection (not shown) on the distal side of the left part 6962.

As shown in the illustrated embodiment, the gear wheel 6966 can be affixed to the tail of an annular implant, such as the implant 6900 of FIG. 69A, and can further comprise a control knob (not shown) that can be actuated by the person implanting the device. The right part 6972 comprises a linear gear 6970 that is configured to engage the gear wheel 6966. The left part 6962 further comprises a linear gear 6964 that is configured to engage the gear wheel 6966. When the gear 6966 is rotated counterclockwise as viewed in FIG. 69C, the left part 6962 moves further left and the right part 6972 moves further right to generate the configuration shown in FIG. 69C. When the gear wheel 6966 is rotated clockwise, the right part 6972 moves left or inward and the left part 6962 moves right or inward to reduce the width of the tail flange 6960. The tail flange 6960 can further comprise a lock (not shown) to maintain the tail flange 6960 in its fully expanded configuration, once so positioned.

The materials suitable for fabricating the tail flange 6960 can be the same or similar to those used in fabricating the tail flange 6906 and 6908 of FIG. 69A. The tail flange 6960 comprises an approximately rectangular configuration with rounded corners. The tail flange 6960 can be sized to be advanced through a port access device similar to that described for the tail flange 6930 of FIG. 69B. The jackscrew type outwardly driven tail flange embodiment 6960 can be incorporated into the embodiments of the annular implant disclosed herein.

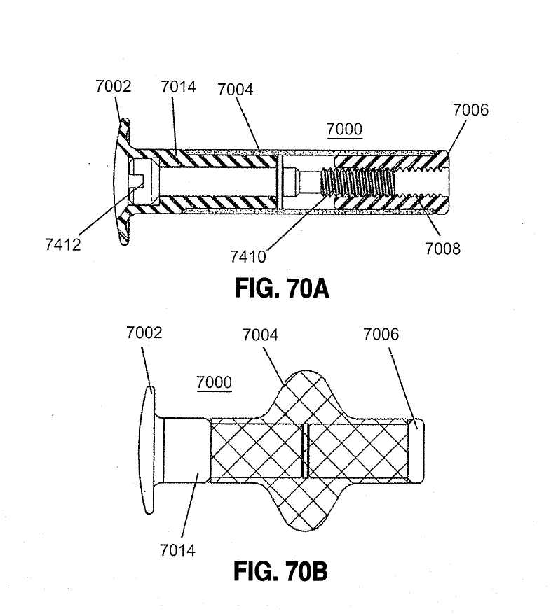

FIG. 70A illustrates a side cross-sectional view of a radially collapsed, expandable annular implant 7000 comprising a tail flange 7002, a tail 7014, an adjustment screw 7412 further comprising a threaded region 7410, an expandable mesh anchor 7004, and a distal end 7006 further comprising internal threads 7008. As shown in the illustration, the tail flange 7002 can be affixed to the tail 7014. The adjustment screw 7012 rotates within and is radially and longitudinally constrained by the tail 7014. The distal end 7006 is constrained to move longitudinally but not rotate relative to the tail 7014. Thus, the tail 4616 and the distal end 7006 telescope relative to each other, the relative position being controlled by the adjustment screw 7012. The distal end 7006 and the tail 7014 comprise features that constrain the ends of the expandable mesh anchor 7004 and capture the expandable mesh anchor 7004 from migrating axially or radially.

When the adjustment screw 7012 is turned to compress the distance between the tail 7014 and the distal end 7006, the expandable mesh anchor 7004 compresses in length and expands in diameter. Conversely, turning the adjustment screw 7012 in the other direction results in the tail 7014 moving away from the distal end 7006, lengthening the expandable mesh anchor 7004 and reducing its diameter. The expandable mesh anchor 7004 can comprise a braid, a weave, and the like. The expandable mesh anchor 7004 can be shape-set from, for example, nitinol, in its fully expanded configuration so that axial stretching of the ends of the expandable mesh anchor 7004 can cause it to axially lengthen and constrict radially. The nitinol can be martensite, superelastic and austenitic at body temperature, room temperature, or both, or it can have shape memory characteristics that are affected by heating or cooling.

FIG. 70B illustrates a side view of the annular implant 7000 of FIG. 70B, wherein the distal end 7006 has been compressed axially toward the tail flange 7002 and the tail 7014, resulting in radial expansion of the mesh anchor 7004.

The anchor elements 7004 can be configured to expand to a maximum diameter in a range from about 1.1 to about 5 times their unexpanded diameter. The expandable mesh anchor 7004 can be configured to expand with various longitudinal cross-sectional shapes. For the purposes of illustration, the space between the proximal end of the compression head 7006 and the distal end of the tail 7014 has been reduced to a minimum distance in FIG. 70B. The outside of the tail 7014, the compression head 7006, or both, can be coated with a dried, hydrophilic, water-swellable hydrogel that increases its volume upon exposure to the moisture of the body, to fill the region interior to the expandable mesh anchor 7004.

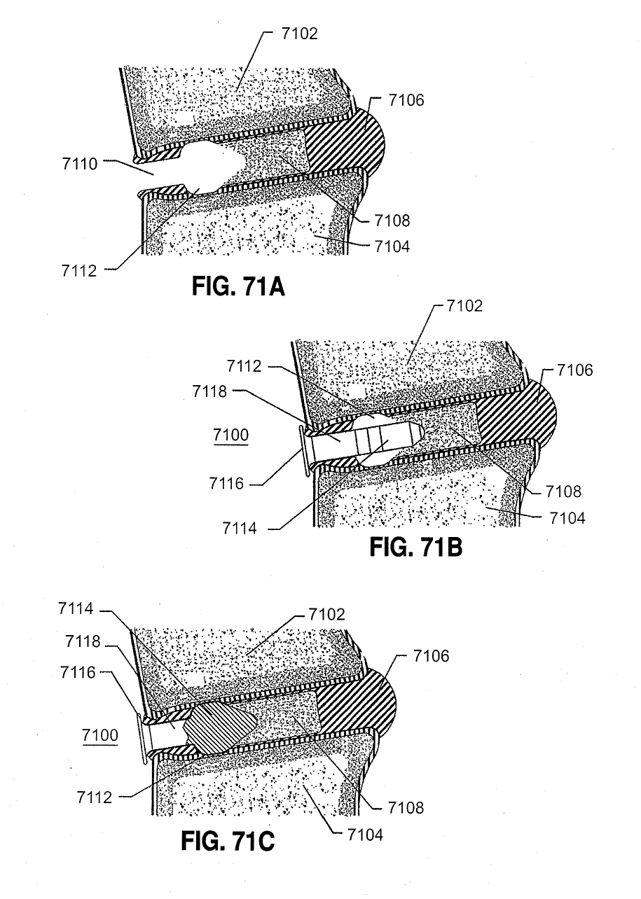

FIG. 71A illustrates a vertebral segment comprising an upper vertebra 7102, a lower vertebra 7104, disc annulus 7106, a disc nucleus 7108, an annular defect 7110, and a prepared region 7112 within the nucleus 7108, the annulus 7106, the upper vertebra 7102, and the lower vertebra 7104. In certain embodiments, the prepared region is cut into the bony structures 7102 and 7104 to maximize anchoring of another implant (see FIGS. 71B and 71C). A surgical reamer as disclosed for earlier embodiments herein can be used to generate the prepared region 7112.

FIG. 71B illustrates an annular implant 7100 inserted into the annular defect 7110. The implant 7100 has been turned so that its small dimension runs laterally and fits between the lip of the upper vertebra 7102 and the lip of the lower vertebra 7104. The implant 7100 comprises a tail flange 7116, a tail 7118, and a head 7114. The head 7114 is turned so that its wide dimension is oriented laterally and does not project into the prepared region 7112.

The tail flange 7116 can be affixed, or integral, to the tail 7118, which can be affixed, or integral, to the head 7114. The cross-sectional shape of the head 7114 can be rectangular or it can be rounded, oval or elliptical and truncated in the vertical direction as illustrated. The truncated dimension of the implant 7100 can range from about 2-mm to about 8-mm, in some embodiment ranging from about 3-mm to about 6-mm. The implant 7100 can be fabricated from materials such as, but not limited to, PEEK, polysulfone, polycarbonate, polyurethane, titanium, cobalt nickel alloy, polyester, and the like. A coupling indent (not shown) in the tail flange 7116 can be a keyed slot suitable for engagement with an implantation tool which can rotate the part about its longitudinal axis.

FIG. 71C illustrates a partial breakaway view of the annular implant 7100 of FIG. 71B, wherein the implant 7100 has been rotated about 90° to maximally engage the head 7114 within the prepared region 7112. In certain embodiments, the implant can be rotated greater than 90° or less than 90° to achieve various positions within the intervertebral disc space.

The wide dimension, shown in the vertical direction of FIG. 71C, can range from about 4-mm to about 25-mm, and in some embodiments, from about 5-mm to about 20-mm. The tail 7118 is configured to be wider horizontally than vertically, in lateral cross-section, to improve the stability of the implant following placement. The tail flange 7116 can be round, oval, rectangular, or similar. The tail flange 7116 can be symmetric or asymmetric and project laterally more to one side than the other side.

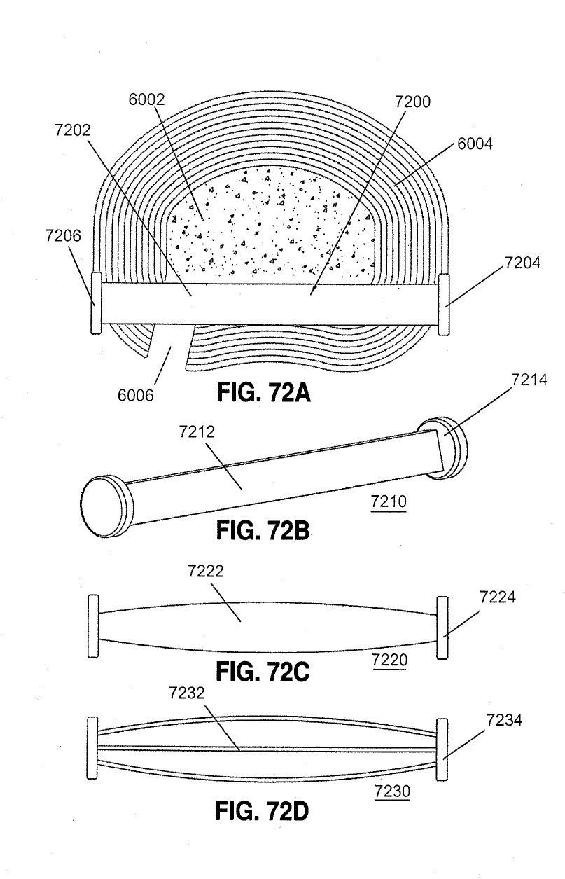

FIG. 72A illustrates an implant 7200 implanted within an intervertebral disc comprising a nucleus 6002, an annulus 6004, and an annular defect 6006. The implant 7200 comprises an axially elongate central connector 7202, a first end plate 7204 and a second end plate 7206. As illustrated, the end plates 7204 and 7206 can be affixed, or integral to, the connector 7202. The central connector 7202 comprises an axially elongate structure having a round, oval, elliptical, rectangular, triangular, or other geometric cross-section. The end plates 7204 and 7206 can be circular, but could have other shapes such as rectangular, triangular, and the like.

The implant 7200 can be fabricated from materials such as, but not limited to, polymers, metals, resorbable polymers, hydrophilic hydrogels, and the like. Suitable metals include stainless steel, cobalt nickel alloys, nickel titanium alloys, gold, platinum, and the like. Suitable polymeric materials for the implant 7200 include, but are not limited to, PEEK, polyester, polysulfone, silicone elastomer, thermoplastic elastomer, PTFE, and the like. Resorbable materials can include, without limitation, polyglycolic acid and polylactic acid as well as certain sugar and collagen structures. The implant 7200 can be coated on its outer surface with porous materials such as woven or knitted fabrics of polyester, polyamide, polyamide, PTFE, or the like. The implant 7200 can comprise radiopaque markers (not shown) to enhance its visibility under fluoroscopy. The end plates 7204 and 7206, as well as the connector 7202 can comprise a central lumen (not illustrated) having a diameter of between 0.010 and 0.100 inches suitable for tracking over a guidewire or other guiding device. One or both end plates 7204 and 7206 can be detachable or expandable structures to facilitate insertion of the implant 7200 through tissue and then expand, for example, after the implant 7200 is in its final desired location.

FIG. 72B illustrates an embodiment of the implant 7210 wherein the connector 7212 is substantially flat and ribbon-like in lateral cross-section. In some embodiments, the cross-section can be similar to an I-beam with somewhat wider edges designed to minimize tissue trauma. The end plates 7214 are affixed to each end of the connector 7212.

FIG. 72C illustrates an embodiment of the implant 7220 wherein the connector 7222 comprises a central bulge. The connector 7222 can have any cross-sectional configuration along its length and could have a central depression with the bulges at the ends, for example. The connector 7222 is affixed, or integral, to the end plates 7224.

FIG. 72D illustrates an embodiment of the implant 7230 wherein the central connector 7232 comprises a plurality of outwardly expandable structures. The outwardly expandable central connector 7232 can be a plurality of resilient metallic or polymeric bars, or it can be configured like a stent that is either balloon expandable or self-expanding in nature.

Any of the implant embodiments shown in FIGS. 72A-72D can be configured so that they can be inserted with a minimum dimension oriented along the axis of the patient to minimize interference with vertebral lip spacing. Following insertion, the implants can be rotated or expanded to maximize interference to a reduction in vertebral lip spacing. The implants can comprise bone growth factors or other pharmaceutical agents such as anti-infective compounds.

Certain embodiments include instruments or tools to prepare the site for the implant and instruments to deliver the implant to the treatment site. The preparation instruments include, but are not limited to, lip sizers to determine the spacing between the vertebral lips, trial units to determine the size of the area reamed out inside the intervertebral space, reamers to enlarge the spacing between the vertebral lips at the implant location, reamers to remove material within the intervertebral space, annulus cutters to remove annulus in the target region, and the like.

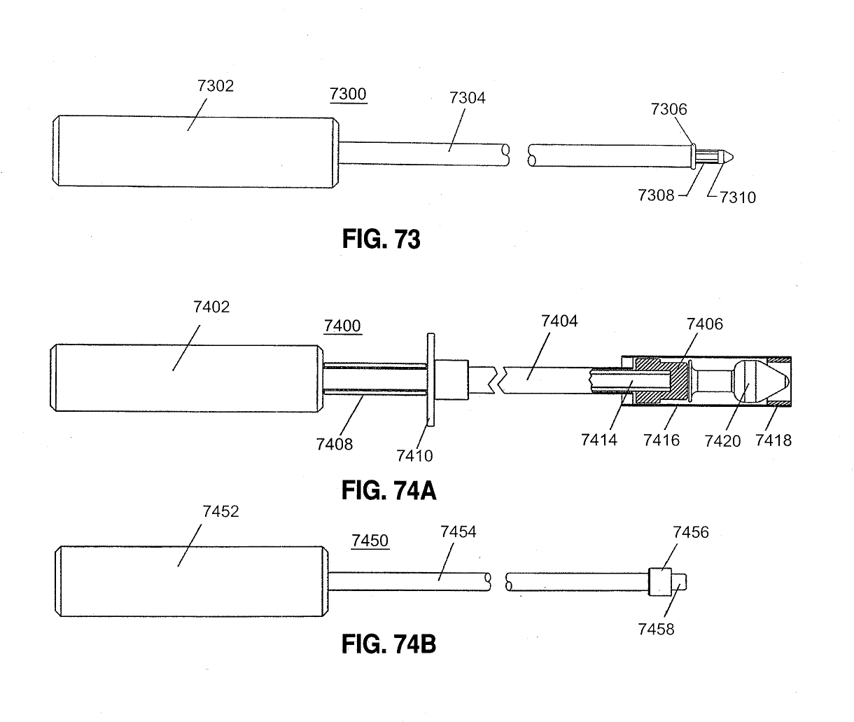

Various embodiments of lip reamers can be used to remove bone, cartilage, and soft tissue in the outermost region of vertebra, otherwise known as the vertebral lip. The vertebral lip generally is the location of the narrowest gap in between the vertebrae. FIG. 73 illustrates an embodiment of a lip reamer 7300. As illustrated, the lip reamer 7300 can comprise a handle 7302, a shaft 7304, and a cutting blade 7308. The lip reamer 7300 can also comprise an optional tail flange 7306 to limit the depth of penetration into the annulus or space between the vertebral lips. In some embodiments, the lip reamer 7300 can comprise a nose cone 7310 to distract the vertebral lips during insertion of the lip reamer 7300 into the annulus. In some embodiments, the nose cone 7310 can comprise a reverse taper on its proximal end to facilitate removal of the lip reamer 7300 from the annulus following use. The lip reamers 7300 can come in the same sizes as lip sizers. The lip reamers 7300 can be fabricated from the same materials as used for lip sizers, standard reamers, or other spinal instruments. The cutting blade 7308 of the lip reamer 7300 can comprise a plurality of flutes with either a straight or helical pattern. Conveniently, a large, deep space between the flutes can permit rapid removal of substantial amounts of material from the annulus. The lip reamer 7300 can be used following the discectomy and either before or after a lip sizer is used.

In certain embodiments, implants configured to treat defects in the annulus of a spinal disc can be placed using minimally invasive techniques. Typical minimally invasive implantation methodology includes port access devices. Such port access devices can include trocars, axially elongate tubular sheaths, radially expandable tubular sheaths, or the like. The implant can be inserted through such port access systems and such insertion can be facilitated by use of an insertion or delivery system. FIG. 74A illustrates an embodiment of a delivery system 7400 for an annular implant 7420. The delivery system 7400 comprises a handle 7402, an axially elongate outer shaft 7404, an implant coupler 7406, an alignment shroud 7416, a linkage 7414, an optional lock 7408, and an optional retainer 7418.

The proximal region of the delivery system 7400 can comprise a release mechanism 7410 operably coupled to the alignment shroud 7416, by the outer shaft 7404. The implant coupler 7406 can be affixed, slidably movable relative, rotatably movable relative, or integral, to the distal end of the linkage 7414, while the handle 7402 can be affixed or integral to the proximal end of the linkage 7414. Coupling of the implant coupler 7406 to the release mechanism 7410 can be through a mechanical linkage, electronic linkage, hydraulic linkage, electromechanical linkage, or the like. The lock 7408 is a removable structure that separates the release mechanism 7410 from the handle 7402. The lock 7408 is an axially elongate tubular structure with a window or gap cut out of the side to create a “C” shaped cross-section that can be removed from the central linkage 7414.

FIG. 74B illustrates an embodiment of the delivery system 7450. In some embodiments, the delivery system 7450 can be configured to permit axial forces, both compression and tension, to be applied to an annular implant (not shown). The delivery system 7450 can comprise a handle 7452, an axially elongate shaft 7454, a compression flange 7456, and an implant coupler 7458. In some embodiments, the delivery system 7450 can be configured to permit rotational forces to be applied to the implant. The implant coupler 7458 can be configured to grasp the implant (not shown) at or near the tail or tail flange of the implant, such that actuation of the release mechanism results in detachment of the implant coupler 7458, and delivery system 7450, from the implant.

In the illustrated embodiment, the implant coupler 7458 is a rectangular structure, similar to a flat bladed screwdriver, but can be of any other shape such as a hex driver, a Phillips head screwdriver, and the like, capable of applying rotational forces to the implant. Application of rotational forces to the implant are important so that the implant can be inserted in one orientation to minimize engagement and interference with spinal structures, and then be rotated in a roughly orthogonal direction (approximately 90°) to maximally engage the spinal structures.

In some embodiments, the delivery system can be configured to permit a first part of an implant to be delivered to the target region. The delivery system can then serve to track one or more follow-up parts of the implant so that they remain aligned with and lock to the first part of the implant. Such tracking can include a groove T-slot, dovetail, rectilinear cross-section, asymmetrical cross-section, and the like, over which a complimentary or mating hole in the second part of the implant is able to slide. Thus, when the handle of the delivery system is rotated about its longitudinal axis, the shaft rotates, as does both the first and subsequent parts of the implant, such that implant alignment is retained.

In some embodiments, the implant coupler can be configured as a retractable pin, bayonet mount, threaded region, latch, and the like. The implant can comprise an undercut, bayonet engaging pin, threaded region, latch undercut, or the like, respectively, which are complimentary to the implant coupler. The implant coupler can also be a can with a reduced diameter exit port which interferes slightly with the outer diameter of the implant, as illustrated in FIG. 74A.

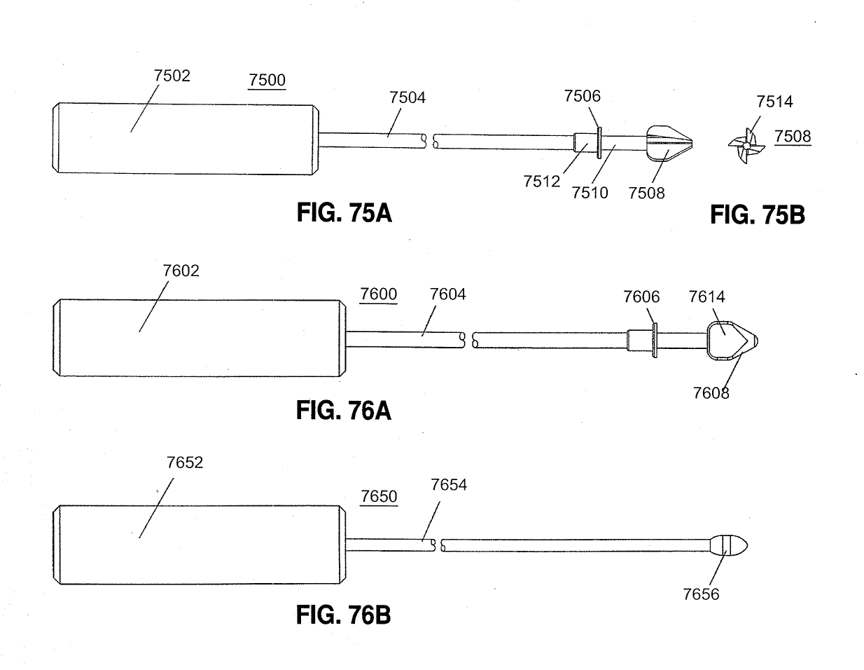

FIG. 75 illustrates a reamer 7500 configured for an annular implant. The reamer 7500 can comprise a handle 7502, a shaft 7504, and a cutting blade 7508. In some embodiments, the cutting blade 7508 can comprise a longitudinal cross-section that approximates that of the implant (not shown). The reamer 7500 can further comprise a tail flange 7506 to control or limit the penetration of the reamer into the annular space. The tail flange 7506 can be immovable and pre-set relative to the shaft 7504, or it can be adjustable, optionally comprising index lines or detents to assist with correct positioning of the tail flange 7506. The tail flange 7506 can be affixed to the shaft 7504 by the collar 7512 to which the tail flange 7506 is affixed. The cutting blade 7508 can be fabricated from stainless steel, cobalt nickel alloy, titanium, carbide steel, or other metals. The cutting blade 7508 can be fabricated from metals that can be hardened to maximize their durability.

FIG. 75B illustrates a front view of an embodiment of a reamer cutting blade 7508 comprising a plurality of flutes 7514. The space and depth of the groove between the flutes 7514 of the reamer can be made deep to permit entrapment of a maximum amount of tissue. The reamer cutting blade 7508 can comprise between 2 and 25 flutes 7514, in some embodiments between 2 and 8 flutes. The flutes 7514 can be straight or helical. In an embodiment, the reamer can be rotated manually. In another embodiment, the reamer 7500 can be rotated by a motor drive, using electrical power, for example, controlled by the user. In the illustrated embodiment, the reamer 7500 cuts when rotated clockwise. In some embodiments, the reamer can be configured to cut when rotated counterclockwise.

The reamer flutes 7514 can be of substantially different height or width to facilitate insertion into the annulus. In some embodiments, the reamer 7500 can comprise four flutes 7514 oriented roughly orthogonally to each other. The flutes 7514 can be turned approximately 45° sideways to reduce the spacing distance between the vertebral lips through which the reamer can be inserted. In some embodiments, the reamer 7500 can comprise four flutes 7514, which can be rotated relative to each other to permit insertion through a narrow slit. In some embodiments, two of the flutes 7514 can be cut off at the back while the other two, roughly orthogonally oriented flutes 7514, can be cut off at the front so that the first two flutes can be inserted through a narrow annulus and then the reamer turned 90° so that the second two flutes can be inserted through the annulus. In some embodiments, the reamer 7500 can comprise two immovable flutes 7514, and two slidable flutes 7514 that are capable of being advanced into alignment with the first two flutes 7514 after the first two flutes 7514 are completely through the annulus and turned vertically. In another embodiment, the reamer 7500 comprises two flutes 7514 that are relatively wide to provide balance during reaming but still narrow enough to facilitate insertion through the annulus.

FIG. 76A illustrates a trial unit 7600. The trial units 7600 can be provided with heads 7608 configured as duplicates or approximate duplicates of the implant, which are affixed, or integral to, the distal end of a shaft 7604, which can be itself affixed, at its proximal end, to an optional handle 7602. In an embodiment, the trial units 7600 can have approximately the same longitudinal cross-section as the implant. The trial units, in an embodiment, can have, approximately the same lateral cross section as the implant. In an embodiment, the trial units 7600 can have part of their lateral extent reduced to facilitate removing the trial unit from the annulus. This cut off lateral extent is illustrated in FIG. 76A as a face 7614. By rotating the trial unit 7600 about its longitudinal axis, the reduced lateral extent, or face 7614, of the trial unit 7600 can be aligned in the same direction as the lip spacing and thus the trial unit can be more easily removed from the annulus than if its orientation was such that the larger dimension spanned the vertebral lips. The trial units 7600 can be fabricated from the same materials as the lip sizers illustrated in FIG. 76B.

In some embodiments, a method of use of the trial units 7600 comprises inserting the head 7608 of the trial unit 7600 into an annular defect after the defect and the intervertebral space has been prepared using reamers, coring tools, rongeurs, etc. The trial unit 7600 can be inserted in its normal orientation or turned sideways to reduce lip interference. The trial unit 7600 can then be turned, approximately 90°, for example, to maximize its interference with the vertebrae. Proper fit of the trial unit 7600 can be determined by ensuring the vertebral spacing is not adversely affected by the trial unit 7600, and that sufficient interference exists to prevent expulsion of the implant. Following determination of correct size, the trial unit 7600 can be removed from the annulus in the reverse of the way it was inserted into the annulus. The handle 7602 or other part of the trial unit 7600 can comprise a label containing information regarding the trial unit size, etc. The trial units 7600 can be provided in a kit or set comprising anticipated sizes needed for use. The trial units 7600 and certain other devices disclosed herein are provided in a range of sizes and pre-sterilized by generally accepted methods.

FIG. 76B illustrates a lip sizer 7650. The lip sizers 7650 can be used prior to placement of the annular implant. The lip sizers 7650 are axially elongate tapered structures 7656 affixed to the distal end of a shaft 7654. The proximal end of the shaft 7654 is affixed to a handle 7652 to facilitate grasping the instrument. The axially elongate tapered structures 7656 can come in diameters ranging from about 2-mm to about 25-mm, in some embodiments in a range from about 3-mm to about 12-mm, in increments of about 0.5-mm. Conveniently, the lip sizers 7650 can have the size designation imprinted, etched, or stamped onto the handle to permit easy determination of the size.

The axially elongate tapered structures 7656 can appear in longitudinal cross-section as pear shaped, oval, elliptical, triangular, or the like. The proximal end of the axially elongate structure 7656 can be slightly tapered or rounded to facilitate removal of the lip sizer from the annulus. The distal end of the lip sizer 7656 can be tapered inward moving distally to facilitate insertion into the annulus. The lateral cross-sectional shape of the head 7656 can be round, oval, elliptical, or rectangular. The shaft 7654 length can range from about 1-cm to about 50-cm. The lip sizers 7650 can be fabricated from metals such as, but not limited to, stainless steel, titanium, nickel chrome alloy, and the like, or polymers such as, but not limited to, polysulfone, polycarbonate, PEEK, polyester, polyamide, polyamide, and the like. The lip sizers can be used following a discectomy by inserting them into the annulus through the intervertebral space to measure the height of the lip opening. The sizers head 7656 should pass easily into and be removed from the annulus. A lateral dimension of the implant can be determined from the dimension of the lip by using a multiplier such as 2×, 3×, 4×, etc. This sizing can be used to ensure proper interference fit between the implant and the annulus. The lip sizers 7650 can be provided in a set or a kit spanning the useful range of sizes.

The annulus cutter (not shown) can comprise a handle, a shaft, a cutting element, a central shaft, a central shaft handle, and a nose cone. The cutting element can comprise a cylindrical saw. The central shaft, nose cone, and central shaft handle are optional but, in some embodiments, can be used to distract the vertebral lips and to entrap annulus tissue following excision by the annulus cutter. The annulus cutter can be used to completely remove annulus tissue, rather than crushing and tearing the tissue but not removing it, as can happen with other removal devices. The annulus cutter can comprise calibration marks to assist with penetration depth determination, or it can comprise a flange to limit the depth of penetration.

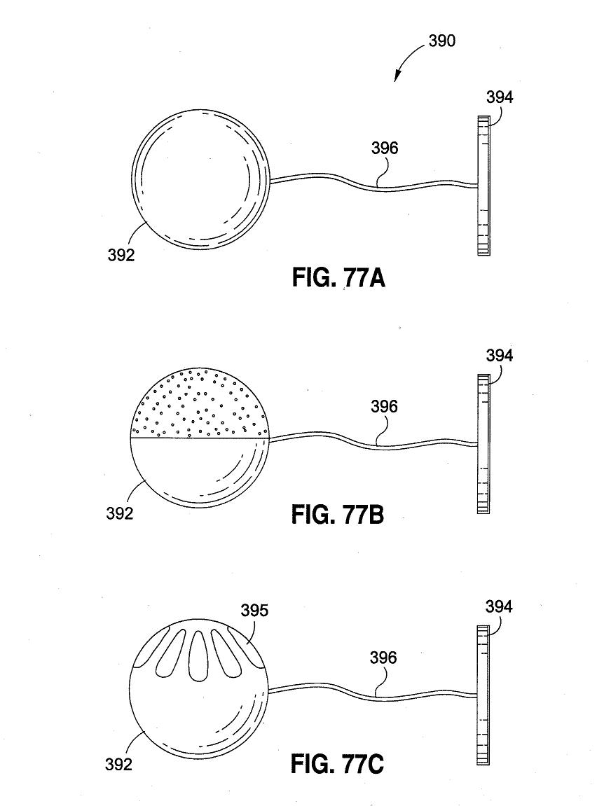

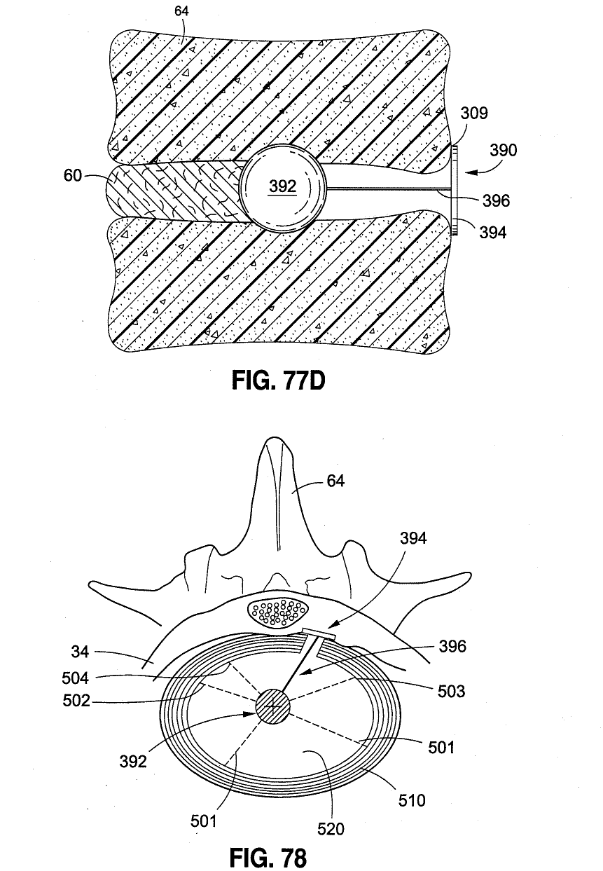

In some embodiments, as illustrated in FIG. 77A, the spinal implant 390 can comprise a head portion 392 and a barrier portion 394, coupled by a flexible tether 396. The head portion 392 can be constructed of more than material as shown in FIG. 77B, or may have bone-compaction holes 395 as in FIG. 77C. Having a flexible tether permits movement of the barrier portion and the head portion relative to each other and yet provides that the head portion and barrier portion each remain substantially located in a stable position relative to the intervertebral disc, the adjacent vertebrae, and the repair site, as illustrated in FIG. 77D. The illustration in FIG. 77D is but one embodiment of an implant with a flexible and is thus not limiting. A variety of shapes, sizes, and compositions of head and barrier portions are possible and will be readily apparent to those skilled in the art. Furthermore, the tether can be any of a number of flexible substances including monofilaments, braided lines, and the like. The size, shape and length of the tether and the materials from which it is constructed are not limiting.

Providing a flexible tether can enhance mobility of the spine without compromising the function of each portion of the implant. Thus the head portion remains effective as a spacer, effectively supporting the adjacent vertebrae, and the barrier portion remains effective to prevent substantial extrusion of material from the intervertebral disc, for example nucleus pulposus.

Providing a tether further increases the functional flexibility of the spinal implant with respect to implantation locations. For example, as shown in FIG. 78, where the barrier portion 394 has been placed at a site of herniation to effectively close it off and prevent extrusion of nucleus from the damaged area, the head portion 392 can conveniently be placed at any one of a number of desired locations, 500, 501, 502, 503,504 within the intervertebral disc. The dashed lines in FIG. 78 represent the fact that with a flexible tether 396 the head portion 392 can be placed in any one of a plurality of locations along points whose distance from the barrier portion 394 is related to the length of the flexible tether 396. Alternatively, as with previously described embodiments, the head portion can be placed within the region of the annulus if desired. The choice of a desired site will be made by the surgeon. If desired, with a flexible tether, the head portion can be located in the annulus 510, or in the nucleus 520, while still maintaining the barrier portion 394 in contact with an exterior surface of the intervertebral disc.

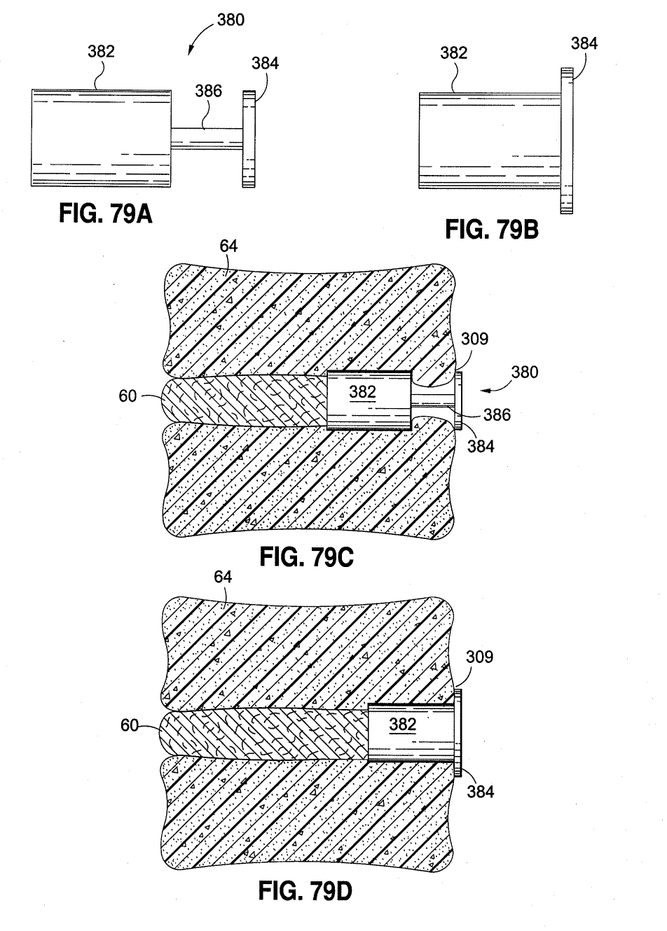

It is also contemplated within the scope of the disclosure to provide in some embodiments, a spinal implant 380 in which none of the segments comprise a taper. As illustrated in FIGS. 79A and B, an implant 380 that is substantially rectilinear along its longitudinal axis can still provide a head portion 382 and barrier portion 384 that is effective in the repair of an annular defect. The implant 382 can optionally include a tail segment 386 that couples the head portion 382 to the barrier portion 384. Alternatively, as illustrated in FIG. 79B, it is also not essential that there be an intervening segment between the head portion 382 and barrier portion 384, and these two domains can be directly coupled of the spinal implant in order for the implant to function as described herein. Placement of a non-tapered implant is analogous to placement of a tapered implant, as is illustrated in FIGS. 80C and D.

In some embodiments, as shown in FIG. 80A-C, there is provided a spinal implant 400, comprising a head portion 402, a barrier portion 404, with the implant further comprising a first portion 405 having bone-compaction holes 406, and a second portion lacking holes 407. The bone-compaction holes 406 are located around a portion of the circumference of the implant, in contrast to FIG. 31A, where bones compaction holes are located substantially around the entire circumference of the implant. Compaction holes 406 can be located, without limitation, in either the head portion 402, the barrier portion 404, or in both portions. Bones compaction holes 406 provide for ingrowth of bone material from the adjacent vertebrae and are thus operative to permit in situ “fusion” of the implant with at least a portion of the adjacent vertebrae.

In some embodiments, as shown in FIG. 80B, the implant can be made such that the portion comprising bone-compaction holes is formed from a first material 410, with the remainder of the implant made from a second material 412. In some embodiments, a plurality of different materials can be used depending on the structural and functional characteristics to be imparted. Thus, materials used to make the implant could be selected to provide both for the fusion and fixation of one portion (i.e. the region comprising holes), while providing a relatively smooth bearing surface in another portion (i.e. the region lacking holes), and may also provide for resilience or compliance of the implant.

As shown in FIG. 80C, when implanted between adjacent vertebrae at a site needing repair in the annulus, the implant can be placed such that the holes 406 are accessible for growth of bone into the hole. This will result in increased stability of the implant placement, due to the contact of a vertebra with the holes 406, and ingrowth of bone material into the holes 406. The region lacking holes 407 provides a relatively smooth surface. The implant therefore provides both a “fusion” region 411, and non-fusion region 413, in the implant. The fixed region 411 is effective to provide for “fusion” of the implant to at least one of the adjacent vertebrae, while the non-fixed region 413 allows a degree of motion of an adjacent vertebra relative to the implant, potentially improving spinal mobility.

In some embodiments there can also be provided a compliant implant, as depicted in FIG. 81A-C. Here compliance of the implant 420 is provided by a split 426 included in at least a part of the head portion 422. The split 426 creates a space between an upper portion 425 and a lower portion 427 of the implant, and permits flexion of the implant such the upper portion 425 and lower portion 427 can be flexibly moved relative to each other owing to compressive forces imposed by the adjacent vertebrae when the implant is situated in a patient. In some embodiments, more than one split could be provided, for example, two splits placed at right angles to each other can provide additional compliance along more than one axis.

As shown in FIG. 81C, the split 426 is configured to run substantially the length of the head portion. However, the precise start and end points, length, and placement of the split are not limiting. For example, it would be equally possible to have the split begin at the barrier portion 424 end of the implant. This configuration can be effective to provide a compliant implant able to flexibly resist forces imposed by loading of the adjacent vertebrae. Compression of the implant by the adjacent vertebrae 64 will thus result in flexion of the implant at, or near, a flex region 429. The degree of flexion will depend on the material comprising the implant, as well as the length of the split 426, the width of the split 426, and the location of the flex region 429. Using this disclosure, those skilled in the art will be able to readily design an implant to provide the desired flexibility. Conveniently, the particular materials chosen to manufacture the implant can be such that they effectively mimic the normal compliance of the natural intervertebral disc material.

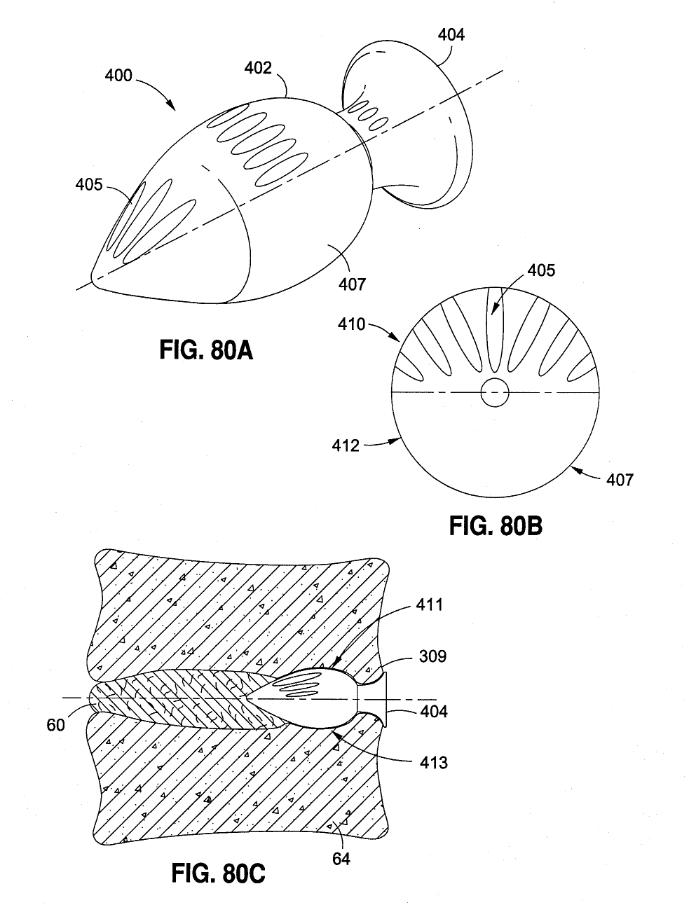

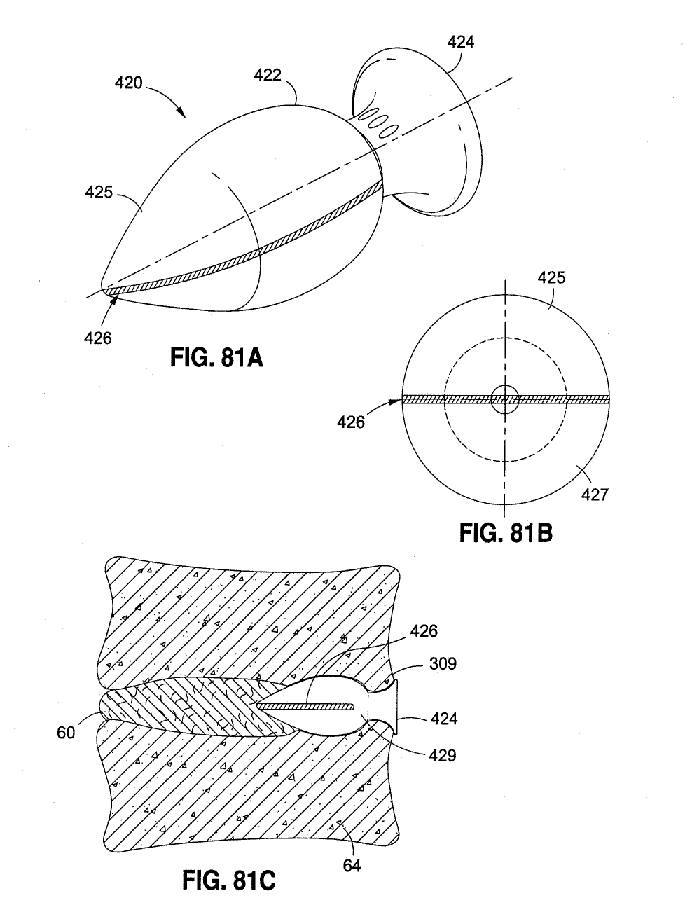

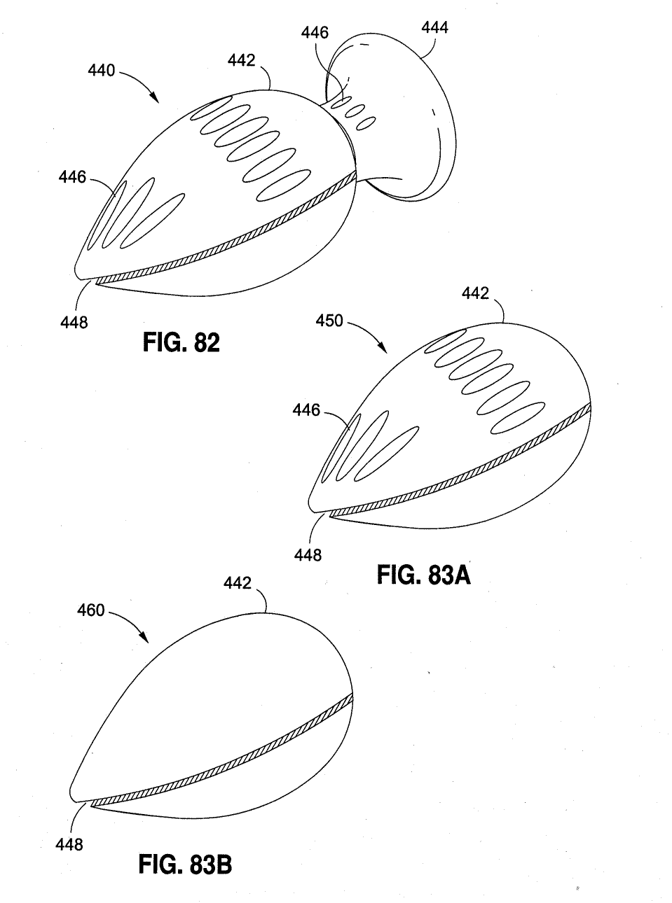

As shown in FIG. 82, in some embodiments a spinal implant can combine the features of those depicted in FIGS. 82A-C, and 82A-C, to provide a compliant implant 440. The compliant implant 440 comprises a split 448, and also includes bone-compaction holes 446. The compliant implant 440, embodiments includes a head portion 442 and a barrier portion 444. The compaction holes 446 may be present in the head portion 442, the barrier portion 444, both portions of the implant, and any combinations thereof. In addition, holes can be provided in one part of the implant, as shown in FIG. 82, or holes may be present around substantially the entire circumference of the implant, for example, as shown in FIGS. 31A and B.

In some embodiments, as shown in FIGS. 83A and B, there is provided a compliant implant 450 that includes a split 448, but which comprises solely a head portion 442 that when positioned between adjacent vertebrae spans a distance between and contacts the vertebrae. At least a portion of the implant is compliant such that it flexibly resists compressive forces imposed by the adjacent vertebrae. In some embodiments, the implant may comprise a head portion having bone-compaction holes 446, as shown in FIG. 83A, or may lack bone-compaction holes, as shown in FIG. 83B. As with other compliant embodiments, the start and end point of the split 448, the length, or location are not limiting to the scope of the disclosure.

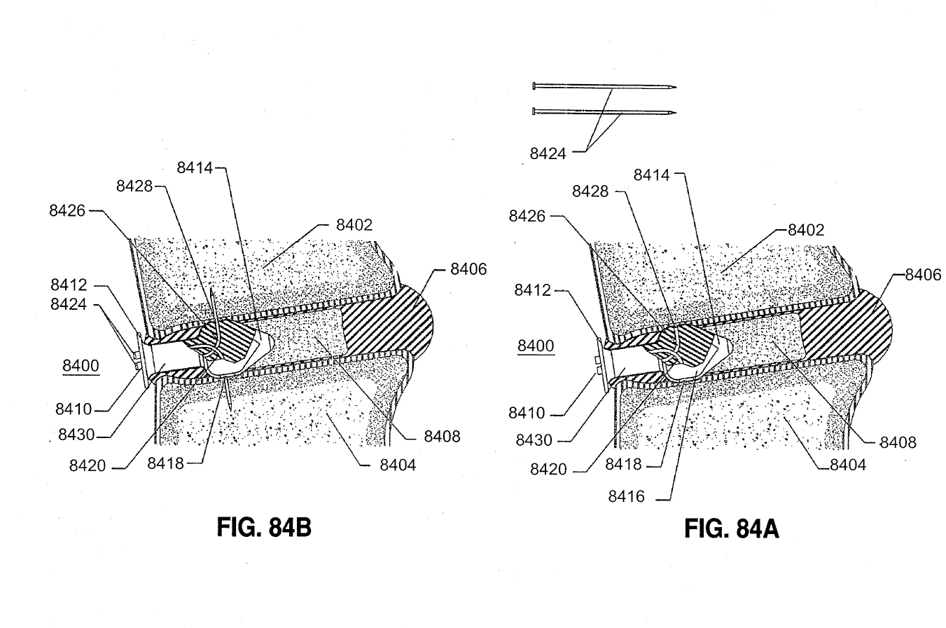

FIG. 84A illustrates an embodiment of an annular implant 8400 placed within a defect in an intervertebral disc. The intervertebral disc comprises the annulus 8406 and the nucleus 8408. The implant 8400 comprises a tail flange 8412, a tail 8430, a plurality of anchor ports 8410, a body 8414, one or more anchor lumens 8420 and 8426, and one or more anchor exit ports 8418 and 8428. The body 8414 has flats or regions of reduced width 8416 disposed laterally within the plane of the intervertebral disc annulus 8406. The implant 8400 also comprises one or more anchors 6420, which are shown not yet inserted into the implant 8400.

The tail flange 8412 can be affixed to, or integrally formed with, the tail 8430, which can be integrally formed with, or affixed to, the body 8414. The anchor ports 8410 are entry ports integral, or affixed, to the tail flange 8412 and operably connected to the anchor lumens 8420 and 8426. The anchor ports 8410 can further comprise locking couplers such as external or internal threads, bayonet mounts, snap locks, and the like for permanent connection with the proximal ends of the anchors 6420.

The body 8414 is as large in diameter as possible for a given annulus size to permit gradual bending of the anchor lumens 8420 and 8426. The body 8414 is large enough to directly abut the hard, bony or fibrous tissue of adjacent vertebrae or related structures. The anchor lumens 8420 and 8426 terminate at their distal ends, and can be operably connected to the anchor exit ports 8418 and 8428, respectively, which are integral to the body 6414. The anchor lumens 8420 and 8426 can be separate or share the same lumen when running generally axially, as through the tail 8430. The anchor lumens 8420 and 8426 can comprise a gentle curve or deflection from the axial direction to a more radially oriented direction, to facilitate guiding the anchors 6420 from being axially disposed to being more radially or laterally disposed.

The anchors 6420, are sharpened at their distal end and flexible, but are constructed to generate significant column strength. In some embodiments from one to about 20 anchors can be used. In some embodiments from about two to about 10 anchors can be used. The anchors 6420, if more than one is used, can be affixed to each other at their proximal ends, for example by welding, fastening, or by other methods well known in the art, to facilitate control. The distal ends of the anchors 6420 can optionally comprise threads configured to engage bony or cartilaginous tissue. The proximal ends of the anchors 6420 can comprise locks configured to mate with the locking couplers on the anchor ports 8410. The proximal ends of the anchors 6420 can further comprise keys, such as slots, hex heads, Phillips screwdriver heads, and the like, to permit rotation by an instrument (not shown) operated by the implanting surgeon.

The shafts of the anchors 6420 are configured to rotate and bend and thus can operate analogously to a speedometer cable. The construction of the anchor shafts can be spring wire fabricated from materials such as, but not limited to, nitinol, stainless steel, titanium, cobalt nickel alloy, and the like. The anchor shafts can also comprise braided or coiled structures capable of transmitting torque and having column strength while permitting bending and rotation. The anchor shafts can be configured to resist shear such no substantial axial motion of the implant 8400 occurs in response to an axial force applied to the implant 8400.

The flat 8416 is configured to reduce the width of the head 8414 so that it can be inserted into the annulus between the vertebral lips with minimum distraction. Once in place, or advanced fully within the annulus, the implant 8400 can be rotated, for example by about 90°, to maximize engagement with the vertebral lips. In some embodiments, the head 8414 has a generally round lateral cross-section with one or both sides truncated by the flats 8416. In some embodiments, the width of the head 8414 from flat 8416 to flat 8416 can range between about 1-mm and 10-mm smaller than the height of the head undistorted by the flats 8416. In some embodiments, the height difference can range from about 2-mm to about 6-mm. In some embodiments, the height difference can range from about 3-mm to about 6-mm.

In some embodiments, the height (or width) of the head 8414 undistorted by the flats 8416 can be about 3 times or more the height of the tail 8430 taken in the same direction. In some embodiments, the height of the undistorted head 8414 can be from about 4-mm to about 8-mm greater than the height of the tail 8430 taken in the same direction, and in some embodiments, from about 5-mm to about 7-mm greater. The width difference between the head 8414 and the tail 8430 is beneficial since the curvature of a vertebra does not change even though the intervertebral disc may degenerate and compress significantly. Thus, in some cases a fixed height differential may be indicated as opposed to the use of a simple ratio of heights.

FIG. 84B illustrates an embodiment of an annular implant 8400 like that shown in FIG. 64A, where the anchors 6420 have been inserted into the anchor ports 8410, advanced through the anchor lumens 8420 and 8426, out the anchor exit ports 8418 and 8428, and into the vertebrae 8402 and 8404. In the illustrated embodiment, there are two anchors 6420 advanced through two anchor lumens 8420 and 8426, which direct the flexible anchors 6420 toward the side exit ports 6418 and into the bone where they achieve substantial holding capability. The anchors 6420 are capable of bending, but resist shear, and thus are configured to limit or prevent retrograde or antegrade movement of the implant 8400 under the forces exerted by the spinal system. The closer the side exit ports 8418 are to the vertebrae 8402 and 8404, the less will be any effect of bending on the anchors 6420, thus the implant 8400 will be better secured within the vertebrae 8402 and 8404.

In some embodiments the anchors are fashioned from wire that can be round or flattened. Orienting the small cross-sectional dimension of a flat wire in the direction of bending permits easier deflection of the flat wire anchor within the body of the implant. In some embodiments, a wire will have dimensions ranging from about 0.05-mm to about 0.65-mm in one dimension, and from about 0.50-mm to about 1.25-mm in another dimension. In embodiments where a round wire is used, the dimensions of the wire can range from about 0.10-mm to about 1.25-mm, and in some embodiments from about 0.25-mm to about 0.65-mm. The distal end of an anchor can be formed in the shape of a taper, a wedge, a barb, and other useful shapes that will be readily apparent to those of skill in the art. Lumens through which the anchors are advanced can be configured to have in internal diameter that is slightly larger than the diameter of the wire used to prevent binding or jamming of a spike within a channel.

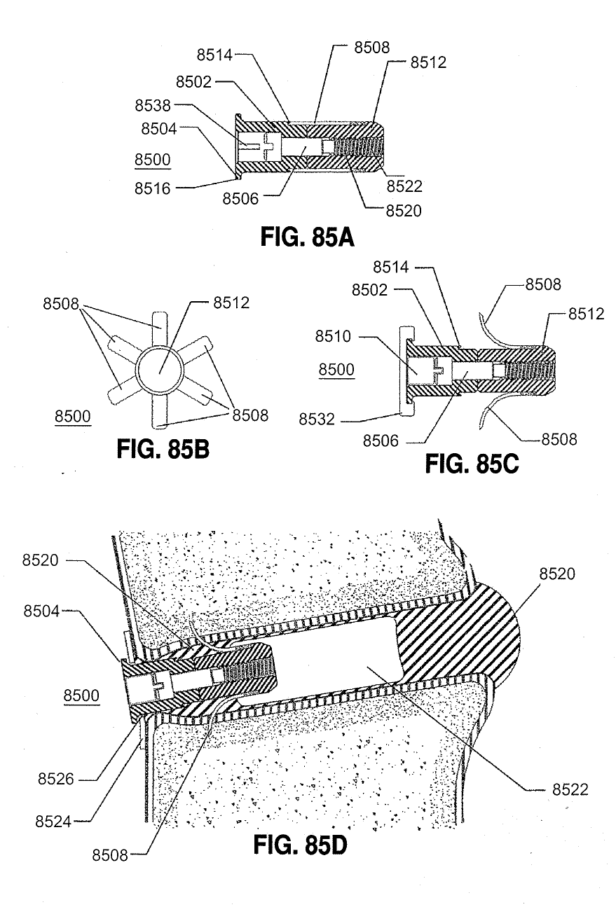

FIG. 85A illustrates an embodiment of an implant 8500 wherein spikes, anchors, feet, pads, or retention structures, collectively termed anchors, are provided which can be advanced radially outward to become affixed in the vertebral structures. The anchors 8508 are forced radially outward or lateral to the axis of the implant 8500 by retrograde or proximal motion of a traveler or anchor connector 8512. The implant 8500, shown with its anchors 8508 retracted, comprises a main body 8502, a tail flange connector 8504, an adjustment screw 8506 further comprising external threads 8522, and a plurality of anchors 8508, an anchor connector 8512 further comprising internal threads 8520, optional anchor deflectors 8536 (not shown), optional anchor retainers 8514, and anti-rotation features 8516 on the main body 8502 or the tail flange connector 8504. The implant 8500 can further comprise an optional tail flange 8524, which can be permanently affixed, or releasably attachable, to the tail flange connector 8504 and it can optionally comprise a rotation lock 8510 (not shown) that comprises protrusions that engage longitudinally running grooves 8538 in the main body 8502.

With regard to FIG. 85A, the main body 8502 can be permanently affixed, or integral, to the tail flange 8504 or the tail flange 8504 can be separately attached to the main body 8502 as a separate procedure after implantation of the main body 8502. The adjustment screw 8506 is axially and radially constrained within the main body 8502 but is able to rotate when forced to do so. The main body 8502 can further comprise an optional rotation lock 8510. The plurality of anchors 8508 can be affixed or integral to each other, or they can be affixed to the separate anchor connector 8512. The anchor connector 8512 can comprise an internal threaded lumen 8520 that engages the threads 8522 on the adjustment screw 8506 such that when the adjustment screw 8506 is rotated, the connector 8512 moves in an axial direction, either forward (distally) or backward (proximally). The anchor connector 8512 is rotationally and laterally constrained to prevent rotation and lateral motion, although longitudinal motion, either smooth or ratcheted is facilitated. Backward, or proximal, motion of the anchor connector 8512 forces the anchors 8508 to be advanced proximally. The main body 8502 can further comprise the deflectors 8536 (not shown) which direct the proximally moving anchors 8508 superiorly (toward the patient's head), inferiorly (toward the patient's feet), or both. The tail flange connector 8504 can comprise the anti-rotation features 8516, affixed or integral to the tail flange connector 8504, which engage a delivery instrument and prevent the tail flange 8504 from rotating while the adjustment screw 8506 is being rotated. The adjustment screw 8506 can be rotated by a tool (not shown) having a handle, an axially elongate shaft, and an engagement portion that cooperates with an engagement portion on the proximally oriented face of the adjustment screw 8506.

The main body 8502 can have a cross-sectional configuration that is round, oval, elliptical, rectangular, triangular, rectangular with rounded edges, or the like. The main body 8502 can be sized for insertion between the vertebral lips either following reaming, following coring with a hole-saw, or following an incision with a scalpel or other sharp instrument. The main body 8502 can be sized and configured for placement using noninvasive or minimally invasive techniques using diagnostic imaging such as magnetic resonance imaging, fluoroscopy, ultrasound, and the like.

FIG. 85B illustrates a frontal view of the implant 8500 wherein the implant 8500 comprises the plurality of expanded anchors 8508 and the anchor connector 8512. FIG. 85B shows six anchors 8508 but the number of anchors can range between two and 20. The anchors 8508 are shown evenly distributed about the circumference of the implant 8500.

FIG. 85C illustrates the implant 8500 wherein the spikes or anchors 8508 have been released from the anchor retainers 8514 and advanced and deflected radially outward in both the superior and inferior directions so as to engage the bony structures of the vertebrae near the outside of the vertebrae and in the area of the intervertebral disc annulus. A detachable, separate tail flange 8532 has been affixed to the tail flange connector 8504. The implant 8500, in the illustrated embodiment, comprises an optional anti-rotation lock 8510, which prevents the adjustment screw 8506 from turning and is, in the illustrated embodiment, held in place by keyed features 8530 and the tail flange 8532, which is releasably affixed to the main body 8502 at the tail flange connector 8504 or the anti-rotation feature 8516. The anchor connector 8512 has been advanced distally to release the anchors 8508 from the anchor retainers 8514 and then withdrawn proximally by rotation of the adjustment screw 8506 and the anchors 8508 have likewise moved proximally with the anchors 8508 having been directed radially outward by their biased, pre-curved shape, so that they can be forced into the superior and inferior vertebrae. The anchors 8508 can be fabricated from wire, either round or flat wire with the tips either sharpened, tipped, blunted, or bent back on itself to form a thicker, blunter end. The anchors 8508 can be fabricated from materials such as, but not limited to, stainless steel, titanium, nitinol, cobalt nickel alloy, PEEK, polyester, polyethylene, polycarbonate, or the like. The anchors 8508 can be tipped with blunt bumpers 8534 (not shown) fabricated from, for example, PEEK, polycarbonate urethane, polyester, polysulfone, silicone elastomer, or the like. The anchors 8508 in the illustrated embodiment are fabricated from shape-set nitinol and are biased toward a radially outwardly curved configuration to engage the vertebral structures but they could also be deflected outward with anchor deflectors 8536 (not shown) affixed to the main body 8502. The bumpers 8534 can beneficially distribute the force of the anchors against the bony structures to prevent penetration so that the bumpers 8534 ride against the bone and optionally against facets, or bone seats, cut into the bone by, for example, a prior reaming process. By this configuration, the anchors 8508 are advanced outward very close to the tail flange connector 8504 such that expansion occurs outside any subannular space, defined as where the nucleus might reside, and within the annulus itself.

FIG. 85D illustrates the implant 8500 implanted with the annulus 8520 of an intervertebral disc. The expandable anchors 8508 are expanded fully within the annulus 8520 while a portion of the anchor connector 8512 resides within the annulus 8520 and another portion resides within the nucleus 8522. The implant 8500 further comprises a separate tail flange 8524 which further comprises a central orifice 8526 through which the main body 8502 is passed and against which the tail flange connector 8504 is advanced to hold the tail flange 8524 securely against the annulus 8520.

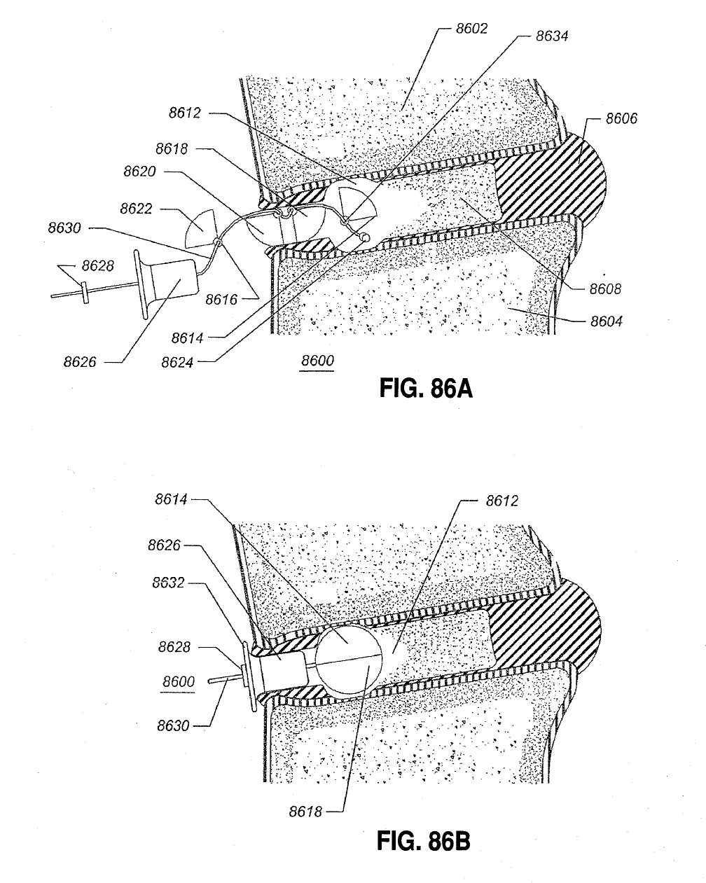

FIG. 86A illustrates an annular implant 8600 comprising a plurality of geometric shapes configured to be passed through an annular defect 8612 into a volume wherein intervertebral disc material, either annulus 8606 or nucleus 8608, has been removed. The annular implant 8600 comprises a tail 8626, a first geometric solid 8614, a second geometric solid 8618, a third geometric solid 8620, and a fourth geometric solid 8622. The annular implant 8600 comprises a tail strand 8630, a tip retainer 8624, and a tail lock 8628. Each of the geometric solids 8614, 8618, 8620, and 8622 comprises an eyelet 8616 further comprising a central through-hole 8634. Each of the geometric solids 8614, 8618, 8620, and 8622 are configured to be passed through an annular defect and under applied tension on the tail strand 8620 terminated by the tip retainer 8624, self-align, or forcibly align, into a single geometric solid capable of serving as an anchor for the tail 8626. The annular implant 8600 is shown being placed within a spine cross-section comprising a superior vertebra 8602, an inferior vertebra 8604, an annulus 8606, and a nucleus 8608.

Referring to FIG. 86A, the tip retainer 8624 is affixed, or integral, to the tail strand 8630 and the tip retainer 8624 is larger in diameter than the hole 8634 in the eyelets 8616. The hole 8634 is sufficiently large that the strand 8630 is slidably constrained within the hole 8634 so that the geometric solids 8614, 8618, 8620, and 8622 can move axially along the strand 8630. The geometric solids 8614, 8618, 8620, and 8622, which can be solid, hollow, layered with hard and soft layers, or the like, are affixed, or integral, to the eyelets 8616. The strand 8630 is slidably constrained within the tail 8626 generally in the same direction as the central axis of the tail 8626.

The geometric solids 8614, 8618, 8620, and 8622 can be quarters of a sphere, a pear, an egg, a rectangle, a pyramid, another polygonal solid or polyhedron, or the like. Further, the geometric solids 8614, 8618, 8620, and 8622, while shown as being four in number, can, in certain embodiments, number between two and twenty, and between three and ten. In certain other embodiments, another number of geometric solids can be used. The central region of the geometric solids 8614, 8618, 8620, and 8622 can be cored or hollowed out to allow for the eyelets 8616 to pass through during the alignment process into a single structure. Each eyelet 8616 is disposed at a different axial location on the geometric solids 8614, 8618, 8620, and 8622 and they are sequenced to permit self-alignment and non-interference. The final geometric shape can also be three-dimensional and irregular, comprising one or more central void. The final geometric shape can, for example form a general sphere, egg, pear, mushroom, or other structure having a lateral dimension ranging between 5 and 20-mm and large enough that the composite structure cannot pass through the distracted lips of the vertebrae 8602 and 8604. In the illustrated embodiment, the final geometric shape will be a sphere with a diameter of 12 mm while the width dimension of the quarter-sphere geometric solids 8614, 8618, 8620, and 8622 is approximately 6 mm, a size that can be delivered to an annular defect through a minimally invasive port access approach and pass through the access window past the retracted nerve and between the vertebral lips. The relative flexibility of the strand 8630 permits lateral displacement of the geometric solids 8614, 8618, 8620, and 8622 to facilitate implantation through the window. The tail lock 8628 is advanced distally to permit tightening of the system over the strand 8630. Calibration marks (not shown) on the strand 8630 can be used to ensure proper alignment of the components. The tail lock 8628 can engage features on the strand 8630, such features including ratchet teeth, bumps, ridges, circumferential grooves, and the like. The tail lock 8628 can be configured to advance distally but not release proximally.

FIG. 86B illustrates the annular implant 8600 of FIG. 86A wherein the implant 8600 has been installed and the tail lock 8628 fully tightened around the strand 8630. The final spherical shape of the anchor structure is complete and cannot be withdrawn through the annulus even under the conditions of significant intradiscal pressure and complex vertebral motion which could include vertebral flexion, torsion, and the like. The tail 8626 is illustrated near the visible geometric components 8614 and 8618 but it could also be configured to touch these components. The tail 8626 can comprise a tail flange 8632. The delivery procedure for the implant 8600 can be facilitated by use of a delivery system, not shown, which allows for retention and control of the components of the implant 8600. The same delivery system, or a secondary instrument, can be used to tighten the tail lock 8628 over the strand 8630. The strand 8630 can be fabricated from polyimide, polyamide, polyester, stainless steel, titanium, nitinol, poly-paraphenylene terephthalamide or the like. The strand 8630 can be multifilament or monofilament in construction.

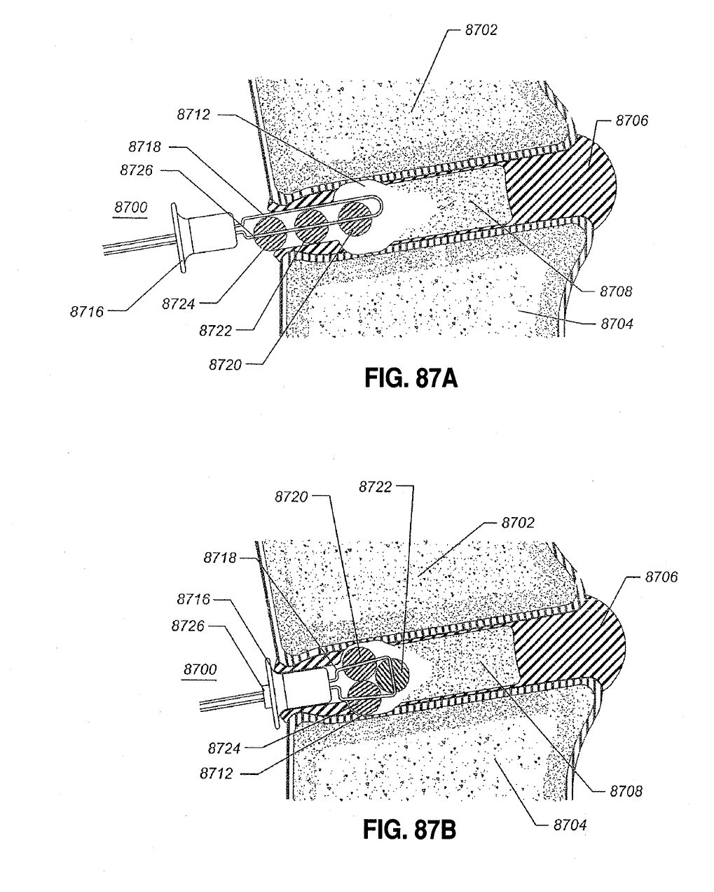

FIG. 87 illustrates an annular implant 8700 comprising a tail 8716, a strand 8718, a first geometric solid 8720, a second geometric solid 8722, and a third geometric solid 8724. Each of the geometric solids 8720, 8722, and 8724 comprise a through lumen 8726, through which the strand 8718 is slidably constrained.

Referring to FIG. 87, the annular implant 8700 is passed through an annular defect into a volume 8712 which has been surgically created in the annulus 8706 and the nucleus 8708 of an intervertebral disc, which is sandwiched between a superior vertebra 8702 and an inferior vertebra 8704. The geometric solids 8720, 8722, and 8724 are sized to fit into the annular defect between the lips of the vertebrae 8702 and 8704. The geometric solids 8720, 8722, and 8724 can be spherical, polyhedral solids, egg-shaped, rounded rectangular solids, or the like. The geometric solids 8720, 8722, and 8724 can be either solid, hollow, or comprise layers of soft and hard material. The materials used in the construction of the implant 8700 can comprise stainless steel, titanium, nitinol, cobalt nickel alloy, PEEK, polyester, polyethylene, polycarbonate, silicone elastomer, polycarbonate urethane, water-swellable hydrophilic hydrogels, or the like. The geometric solids 8720, 8722, and 8724 can further comprise indents or detents on their surface to assist with self-alignment. The number of geometric solids in the illustrated embodiment is three but the number can range between two and 20, or, in certain embodiments, can between three and seven. In certain embodiments, another number of geometric solids can be used. A single strand 8718 can be used, as illustrated, where the strand 8718 is folded back into a loop and passed twice through lumens (not shown) in the tail 8716. In another embodiment, each geometric solid 8720, 8722, and 8724 can comprise a permanently affixed strand 8718. In yet another embodiment, a portion, less than 100% of the geometric solids can be strung together by a strand 8718 while other portions, less than 100% can be strung together by another strand 8718. It is beneficial that the strands 8718 be slidably disposed through lumens 8726 in the geometric solids 8720, 8722, and 8724. In another embodiment, the implant 8700 comprises three (or four) geometric solids affixed by a flexible strand 8718 while a cap geometric solid, which is implanted first, comprises a relatively inflexible strand 8718 and is used to control the geometry of the final self-aligning structure. The cap geometric solid (not shown) can be shaped or configured as a mushroom cap with optional detents to facilitate capturing the geometric solids 8720, 8722, and 8724 against the tail 8716.

FIG. 87B illustrates the implant 8700 of FIG. 87A, wherein the tail has been tightened up against the annular defect, and the geometric solids 8720, 8722, and 8724 have been tightened by tension on the strand 8718. A tail lock 8726 has been installed and advanced distally to tighten the strand 8718 and prevent further relative motion between the strand 8718, the tail 8716, and the geometric solids 8720, 8722, and 8724, which have formed into a composite structure larger in lateral dimension than can pass through the annular defect. The tail lock 8726 and the strand 8718 can be fabricated using methodology and configurations similar to those outlined for the tail lock 8628 and strand 8630 of FIGS. 86A and 86B.

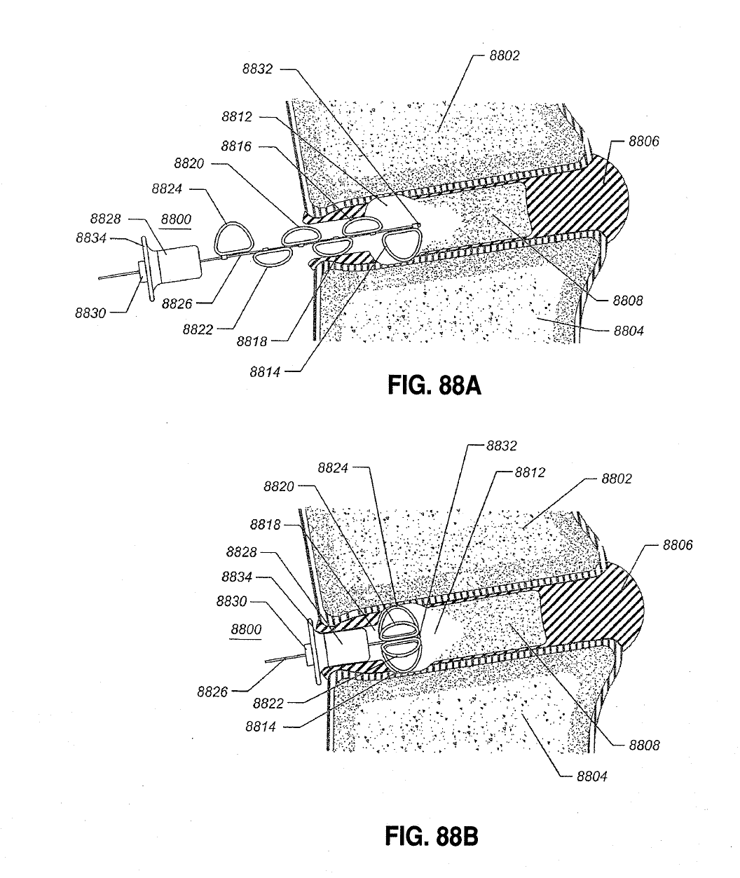

FIG. 88A illustrates an annular implant 8800 comprising a tail 8828, a tail lock 8830, a strand 8826, a tip retainer 8832, a tail flange 8834, and a plurality of hoops 8814, 8816, 8818, 8820, 8822, and 8824. Each hoop 8814, 8816, 8818, 8820, 8822, and 8824 comprises an eyelet 8836, through which the strand 8826 is slidably constrained. The annular implant 8800 is passed through an annular defect into a volume 8812 which has been surgically created in the annulus 8806 and the nucleus 8808 of an intervertebral disc, which is sandwiched between a superior vertebra 8802 and an inferior vertebra 8804. The volume 8812 can be surgically created with a reamer, an expandable reamer, a coring tool, or the like. Preparation or creation of the space or volume 8812 is beneficial for many of the concepts and embodiments described herein because the nucleus of the disc is very undefined or nonexistent and the wall dividing the annulus and the nucleus is a blended structure comprising no clear boundary. Since the nucleus, or subannular space, is not clearly defined, fibrous tissue exists therein which would prevent proper expansion of a device without creating the void or volume 8812. The embodiments described for FIG. 88A and elsewhere in this document are configured to expand or be placed within annulus and not within the subannular space. Due to the fibrous nature of the annulus and its expanded nature as the patient ages, removal of this material and possibly some of the bone and end plate facilitate placement of annular implants.

Referring to FIG. 88A, the tip retainer 8832 is affixed to the distal end of the strand 8826. The proximal end of the strand 8826 is slidably inserted through and radially constrained by, a lumen (not shown) in the tail 8828 and the tail flange 8834. The eyelets 8836 are affixed, or integral, to the hoops 8814, 8816, 8818, 8820, 8822, and 8824 and the eyelets 8836 further comprise a central through hole (not shown), which is slightly larger in diameter than the strand 8826. The strand 8826 passes through the central through hole of the eyelets 8836. The eyelets 8836 are positioned at unique, sequential locations on the hoops 8814, 8816, 8818, 8820, 8822, and 8824 so that the eyelets do not interfere with each other and cause the hoops 8814, 8816, 8818, 8820, 8822, and 8824 to self-align. The tail lock 8830 can engage features on the strand 8826, such features including ratchet teeth, bumps, ridges, circumferential grooves, and the like. The tail lock 8830 can be configured to advance distally but not release proximally. The delivery procedure for the implant 8800 can be facilitated by use of a delivery system, not shown, which allows for retention and control of the components of the implant 8800. The same delivery system, or a secondary instrument (not shown), can be used to tighten the tail lock 8830 over the strand 8826. The strand 8826 can be fabricated from polyimide, poly amide, polyester, stainless steel, titanium, nitinol, or the like. The strand 8826 can be multifilament or monofilament in construction.

FIG. 88B illustrates the annular implant 8800 in its fully assembled shape within the annular defect 8812. The hoops 8814, 8816, 8818, 8820, 8822, and 8824 can be configured to have a round, rectangular, oval, flat, triangular, polygonal, or other suitable cross-section. The hoops 8814, 8816, 8818, 8820, 8822, and 8824 can be configured to be shaped round or circular, oval, D-shaped as in the illustrated embodiment, pear shaped, rectangular, or in any other suitable geometric two-dimensional shape. The width of the hoops 8814, 8816, 8818, 8820, 8822, and 8824 is beneficially such that when the hoops are pulled together as shown, they will self-align circumferentially and index against each other near the central axis with sufficient spacing for clearance but not enough spacing so as to allow the hoops to individually rotate substantially out of the desired three-dimensional shape, which is a flattened sphere in the illustrated embodiment. The width of the hoops 8814, 8816, 8818, 8820, 8822, and 8824 can be increased on the most outward extent to distribute stress on the vertebrae, end plates, etc., thus, the width of the hoops 8814, 8816, 8818, 8820, 8822, and 8824 need not be constant throughout their circumference. The hoops 8814, 8816, 8818, 8820, 8822, and 8824 can further be coated with hydrophilic hydrogel, silicone elastomer, thermoplastic elastomer, or the like, to reduce trauma to bony structures and minimize the risks of bone subsidence. The tail 8828 has been advanced distally into close proximity or even touching the proximal ends of the hoops 8814, 8816, 8818, 8820, 8822, and 8824. The tail lock 8830 has been advanced over the strand 8826 and tightened to generate the illustrated final device. The excess strand 8826 can be cut off or left long as desired. The hoops 8814, 8816, 8818, 8820, 8822, and 8824 can be fabricated from elastomeric materials such as nitinol, polyester, cobalt nickel alloy, stainless steel, or the like. They can also be fabricated from rigid materials such as PEEK, polysulfone, or the like, although elastomeric materials may provide for better biocompatibility and resistance to bone subsidence. The ability of the hoops 8814, 8816, 8818, 8820, 8822, and 8824 to deform under stress can allow the implant to follow spinal compression but then expand to retain their engagement with the vertebrae 8802 and 8804 or the other structures within the annulus 8806.

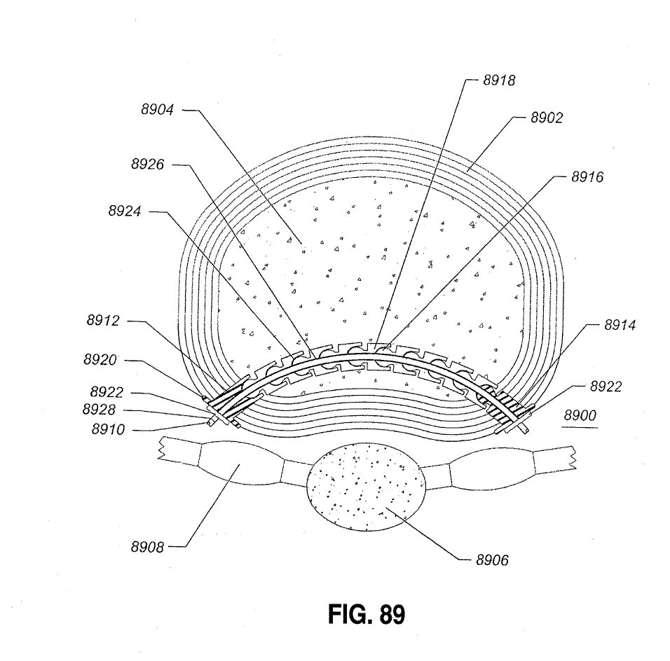

FIG. 89 illustrates an annular implant 8900 for the treatment of posterior disc herniation or for spinal height preservation in a degenerated disc. The implant 8900 comprises an articulating structure that is placed either using open surgery or minimally invasive techniques. The implant 8900 comprises two end caps 8912, 8914, each comprising a tail flange 8922 and a central lumen 8926, and a plurality of articulating connector members 8918, each of which further comprises a ball 8916, a socket 8924, and a central lumen 8926. The implant 8900 further comprises a central core wire 8910 and a plurality of end locks 8922 with the core wire 8910 comprising optional detachment regions 8928. The implant 8900 is illustrated within the cross-sectional view of an intervertebral disc further comprising an annulus 8902, a nucleus 8904. The spinal cord 8906 is illustrated in cross-section and the nerve roots 8908 are shown projecting laterally from the spinal cord 8906.

Referring to FIG. 89, the core wire 8910 is slidably constrained within the central lumen 8926 of the connector members 8918 and the end caps 8912. The ball of one connector member 8918 is constrained from axial motion by the socket 8924 of its adjacent connector member 8918. In another embodiment, the ball and socket junctures between the end caps 8912, 8914, and the junction between the connector members 8918 can be replaced by hinges (not shown) in the same direction, or a portion of the hinges are oriented in a direction different than that of the other hinges. In the illustrated embodiment, the connector members 8918 are, however, free to rotate about the axis of the ball 8916 with some rotational constraint being maintained by the core wire 8910. The core wire 8910 can comprise the optional detachment areas 8928 at which point the excess length can be broken, cut, or otherwise removed from the implant 8900 once the end locks 8922 are tightened and secured against the end caps 8912, 8914. In another embodiment, the core wire 8910 can be removed once the implant 8900 is placed since the implant 8900 is axially locked into a fixed length by the ball 8916 and socket 8924 connectors. The end locks 8922 can be separate, as shown, or they can be integral or affixed to the end caps 8912, 8914. The end locks 8922 can be ratchet-type, threaded type, or fastener-type locks. The entire structure of the implant 8900 can be coated with water-swellable hydrophilic hydrogel to assist with maintenance of a seal with the intervertebral disc structure. The entire implant 8900 can further comprise an outer layer of woven, or knitted material, such as polyester, polyimide, polytetrafluoroethylene, or the like, which can encourage tissue ingrowth.

The core wire 8910 can be a separate device or it can be a guidewire. The implant 8900 can be placed through minimally invasive techniques such as port access. The implant 8900 can be placed from a posterior-lateral approach, as illustrated, it can be placed from a direct lateral approach, it can be placed from a posterior approach wherein the device is formed into a U shape, or it can be placed from a double sided posterior approach where two devices are inserted and interconnected to each other within the nucleus 8904 or the annulus 8902 of the intervertebral disc. The implant 8900 can comprise steering elements, such as pull wires actuated from the proximal end of the device, to force a given curve that varies as the implant 8900 is being advanced into an incision in the intervertebral disc. Access to the intervertebral disc can be gained by a port access procedure using an 18 mm ID access port, for example, it can be gained over a guidewire placed percutaneously, or a combination of both.

The implant 8900 can beneficially be used to prevent migration of nucleus or annulus from a compromised intervertebral disc into the posterior space near the nerve root where it could cause compression, pain, numbness, loss of body function, and the like. The advantage of this very wide device is that, when a disc herniation occurs, the region of compromised annulus may be very wide and a single-point annular repair device may be inadequate to treat the entire posterior region of the intervertebral disc. However, the embodiment shown in FIG. 89 can treat the entire posterior portion of the intervertebral disc.

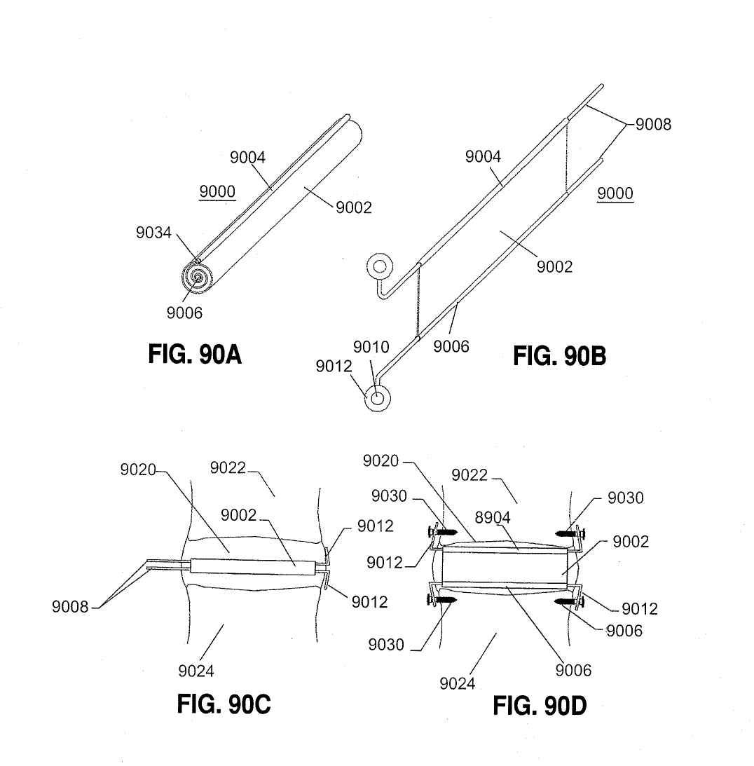

FIG. 90A illustrates an annular implant 9000 in its rolled-up first, smaller diameter, comprising a first tubular guide 9004, a second tubular guide 9006, and an interconnecting membrane 9002. The first tubular guide 9004 is affixed or integral to one end of the interconnecting membrane 9002 while the second tubular guide 9006 is affixed or integral to the other end of the interconnecting membrane 9002. Each of the tubular guides 9004 and 9006 comprise a through lumen 9034 capable of receiving a fixation wire (not shown). The interconnecting membrane 9002 can be fabricated from elastomeric or inelastic materials such as, but not limited to, polyester, polytetrafluoroethylene, silicone elastomer, nitinol, stainless steel, titanium, polyethylene, polyurethane, or the like. The tubular guides 9004, 9006 can be rigid or flexible but beneficially exhibit column strength and freedom from kinking. The tubular guides 9004, 9006 can be reinforced with a mesh, braid, or coil fabricated from metals such as, but not limited to, stainless steel, cobalt nickel alloy, titanium, nitinol, and the like. The rolled up diameter of the implant 9000 can range between 1-mm and 15-mm, and in certain embodiments, the implant 9000 can range between about 3-mm to about 10-mm. The length of the implant 9000 should approximate the width of the intervertebral disc and can range between 2-cm and 10-cm.

FIG. 90B illustrates the annular implant 9000 of FIG. 90A in it's stretched out, expanded configuration. The annular implant 9000 comprises the interconnecting membrane 9002, the first tubular guide 9004 and the second tubular guide 9006 through which fixation wires 9008 have been inserted. The fixation wires 9008 can further comprise the optional eyelets 9012 with through holes 9010. The length of the annular implant 9000 is substantially unchanged from its compressed, smaller configuration as shown in FIG. 90A. The fixation wires 9008 can be fabricated from materials such as, but not limited to, stainless steel, cobalt nickel alloy, titanium, nitinol, polyester, polyimide, polyamide, and the like. The diameter of the fixation wires 9008 can range between 0.025-inches and 0.250-inches, and, in certain embodiments, ranging between 0.050 and 0.187-inches.

FIG. 90C illustrates a view of an intervertebral disc 9020 sandwiched between an upper vertebra 9022 and a lower vertebra 9024. The compressed implant 9000 has been inserted through the intervertebral disc 9020 from the right side to the left side with general positioning toward the posterior side of the disc 9020. The eyelets 9012 are oriented on the right side of the implant 9000 while straight wires 9008 protrude out the left side of the implant 9000. The view of FIG. 90C is from the posterior side of the intervertebral disc looking anteriorly.

FIG. 90D illustrates a view of an intervertebral disc 9020 from the posterior side looking anteriorly. The implant 9000 has been expanded vertically and the interconnecting membrane 9002 forms a barrier against migration of nucleus or annular tissue posteriorly. The interconnecting membrane 9002 is affixed to the first tubular guide 9004 and the second tubular guide 9006, through which the fixation wires 9008 have been inserted and affixed to the upper vertebra 9022 and the lower vertebra 9024 by fixation screws 9030. The implant 9000 can be place by an open surgical procedure or by minimally invasive bilateral port access. The fixation screws 9030 can be inserted through the eyelets 9012 or the screws 9030 can comprise lateral through holes (not shown) through which the wires of 9008 can be passed, after first bending upward or downward. The wires 9008 can be tightened into the holes in the fixation screws 9030 using clamps or locks (not shown).

The tail flange, which can be a radially enlarged region that rests against the outside of the annulus and seals an annular defect against the retrograde herniation of annular or nuclear tissue, can be a separate component from the body of the implant. The tail flange can be inserted first against the intervertebral disc either alone or over a guidewire, through a port access device, or using a specialized implantation instrument. A hole or passageway through the tail flange can accept the annular implant therethrough. A small diameter flange, larger in outside diameter than the outside diameter of the hole through the tail flange, can be positioned on the proximal end of the annular implant can engage the hole through the tail flange and force the tail flange against the annulus and seal the annulus against future herniation. The tail flange can be fabricated from rigid, semi-flexible, or flexible materials so that it can be folded to decrease its profile during insertion or placement.

In many of the embodiments disclosed herein, the annular plug is configured with an anchor, a tail flange, and a connector between the anchor and the tail flange. The anchor is intended to keep the device in place against the forces imposed by postural changes and mechanical loading and to permit the motion of that spine segment to be preserved to provide maximum clinical benefit. Such motion preservation is important because reduction in spine segment mobility can result in adjacent spine segments bearing excessive loads and, therefore, becoming damaged, degraded, or diseased. The motion preservation can occur about one axis or about two axes. For example, the implant 7200, illustrated in FIG. 72A is a cylindrical rod with its axis disposed laterally relative to normal patient anatomy and substantially completely spans the width of the intervertebral disc. The device can provide for vertebral spacing preservation or disc height preservation, or even a modest increase therein to unload the facet joints. Motion or bending in the anterior-posterior (flexion-extension, respectively) direction is preserved or maintained but lateral bending is impeded by the presence of this structure. Alternatively, the anchors of implant 6800, illustrated in FIG. 68, or implant 7100, illustrated in FIG. 71C are substantially rounded, or near round, and thus is able to function while the spine flexes both in the anterior-posterior direction, and in the lateral directions, both left and right. The anchor is the primary height preservation structure of these annular repair devices and rides against or near to the vertebrae. Thus, the anchor determines to a large extent, how much, and in what direction, motion, especially bending, within the spine segment will be preserved. In other embodiments, the connector, herein sometimes termed a tail, between the anchor and the tail flange can provide vertical height preservation support to the vertebrae depending on how close the vertebral lips are disposed relative to said connector. The connector can be configured to ride very close to, or touching, the vertebral lips. In this embodiment, the connector can reduce, minimize, or prevent bending in extension because the vertebral lips cannot move closer together than the height of the connector. Such motion restriction can be beneficial in certain clinical cases. Otherwise, the distance between the connector and the vertebral lips can be increased such that annular tissue resides between the connector and the vertebral lips, thus permitting greater bending in extension for that motion segment of the spine.

The annular implant can be configured, in certain embodiments, to generate distraction or decompression of the vertebrae surrounding the disc within which the device is implanted. For example, the height, or diameter, of the implant 7200, as illustrated in FIG. 72A can be configured to be equal to the vertebral spacing, or it can have a height or diameter that is between 0.5 and 12-mm greater than the unstressed vertebral spacing, or lip height. The benefits of using an implant with a greater height or diameter is that the vertebrae can be distracted and the intervertebral disc can be decompressed. In some embodiments, the maximally distracted vertebral lip height, or spacing, can be used to determine the approximate width of the implant head, tail, or both. In some embodiments, the head height can be configured to be a fixed distance greater than the maximum distracted vertebral lip height. In certain embodiments, if the maximum distracted lip height is about 6 mm, the implant width can be about 6 mm while the implant head height can be about 9-mm, a fixed about 3-mm larger than the maximum distracted disc lip height. The range of implant head height increase over the maximum distracted lip height can range from about 1-mm to about 6-mm, and, in certain embodiments, a range of about 2-mm to about 4-mm. The tail height can be set at approximately 50% of the maximum distracted lip height so in the cited example of about 6-mm maximum lip distraction, the tail height would be about 3-mm. In another embodiment, the implant head height can be set to a proportion of the maximally distracted lip height. For example, the head height can be calculated as between about 20% and about 100% greater than the maximum distracted lip height, and, in certain embodiments, a height increase ranging between about 33% and about 75%. In other embodiments, the tail height can be set at between 0 mm (tail lip contact) and about 4-mm smaller than the resting vertebral lip height, and, in certain embodiments, a tail height of about 1-mm to about 2-mm smaller than the resting lip height. The tail height is generally measured in an orientation perpendicular to the width of the implant but parallel to the head height of the implant. The purpose of such dimensional relationships is to ensure that sufficient interference between the head height and the vertebral lip spacing exists to prevent device expulsion from the intervertebral space under physiological or supra-physiological circumstances of spinal loading. These dimensions apply to implants with rounded, or arcuate, head cross-sections, truncated rounded head cross-sections, or rectangular head cross-sections. The rectangular head cross-sections can further comprise rounded corners with radii ranging from about 0.010-inches to about 0.125-inches, and, in certain embodiments, a radius of about 0.030 to about 0.080-inches.

The implant 7200 can be fabricated from permanently implantable materials such as, but not limited to, PEEK, polycarbonate urethane, titanium, or the like. It can also be fabricated from biodegradable materials such as, but not limited to, polylactic acid, polyglycolic acid, sugar, collagen, or the like. The implant 7200 or many of the other implants described herein, can be coated on their exterior with porous materials, irregularities, or surface structures such as, but not limited to, polyester, polytetrafluoroethylene, porous metal, holes, or fenestrations in any of the materials described herein, to encourage tissue ingrowth, mechanical attachment to tissue, and the promotion of scar or other tissue formation to assist in stabilization of the implant and prevention of intervertebral material extrusion or expulsion from an annular defect. The embodiments that comprise biodegradable materials can be used for temporary disc height increase to allow the body to rejuvenate the intervertebral disc naturally, or with augmentative procedures such as nuclear material injections. Bilateral placement of implants such as the device 6800, illustrated in FIG. 68 can perform the same function of decompression or distraction as can the implant 7200, cited earlier in this section, and maintain vertebral spacing evenly. A unilateral implant of the type in FIG. 68 could result in uneven loading on the vertebrae and the potential for mechanical imbalance, or it could be used to correct for an imbalance, such as found in scoliosis patients to restore a more natural spinal configuration.

In certain embodiments, the intervertebral disc implants, also termed annular implants, can act as facet unloading devices. Nerve compression by the facets in some clinical situations can lead to pain and dysfunction. In certain medical pathologies, the facet joints, which are the projections located on the posterior side of the spine, can endure significant excess force loading, sometimes leading to fracture, failure, nerve compression, tissue extrusion, or the like. An annular implant can be placed in the posterior region of the spine to relieve excess loading on the facet joints and prevent, or reduce, the risk of facet damage. It can be beneficial to implant the device as near to the posterior region of the intervertebral disc as possible to maximize the unloading effect on the facets. Thus, a plurality of devices, for example one each, placed on each side of the spine within the intervertebral disc annulus in a bilateral fashion, can beneficially reduce the forces on the facets. Many of the embodiments described herein can be used for this purpose. The methodology of use would involve measuring the intravertebral spacing, distracting the vertebrae, and placing an implant with a height greater than that of the intervertebral spacing, and locking the device or devices in place so that they cannot become expelled. The additional height can range from 0.5-mm to 12-mm and the precise amount will be chosen by the implanting physician to maximize clinical benefit.

In other embodiments, many of the devices described herein can be used as a plug to seal an access port in the intervertebral disc annulus through which a nucleus replacement was inserted. The use of nucleus replacement devices may see widespread increased use and it would be beneficial to close an annular defect that was created or enlarged in order to allow for implantation of such a device. The placement of nucleus replacement devices can require fairly large access ports within the disc annulus and closure of such defects can prevent or minimize future loss of disc material into the posterior spinal column where it could impinge on nerves and cause pain, loss of tactile sensation, and loss of function. Nucleus replacement technologies can be found, for example, in U.S. Pat. No. 6,482,235, to Lambrecht et al., the entirety of which is hereby incorporated herein by reference. The use of a multiple piece implant for nucleus replacement, as described herein, which allows for assembly in place, provides a less invasive methodology for insertion and construction of appropriately sized devices.

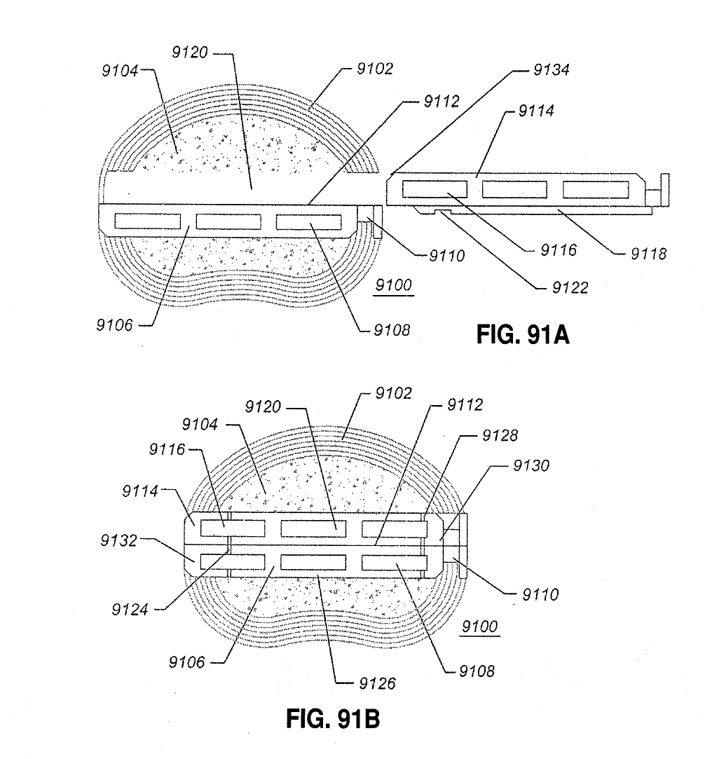

FIG. 91A illustrates a vertebral body replacement 9100 comprising a plurality of components which are assembled in situ. The vertebral body replacement 9100 comprises a first part 9106 and a second part 9114. The second part 9114 comprises a plurality of fenestrations or openings 9116, a tail 9110, and an interlock projection 9118 further comprising a locking detent 9122 and a distal ramp 9134. The first part 9106 comprises a plurality of fenestrations, holes or openings 9108, an interlock groove (not shown), a lock prong (not shown), and a tail 9110. The vertebral body replacement 9100 is illustrated looking in the anatomically axial direction as it is placed into an intervertebral disc comprising an annulus 9102, a nucleus 9104, and a surgically created void 9120.

The first part 9106 and the second part 9114 can be fabricated from metals such as, but not limited to, titanium, nitinol, tantalum, stainless steel, cobalt nickel alloy, and the like. The first and second parts 9106 and 9114 can also be fabricated from polymeric materials such as, but not limited to, PEEK, polycarbonate, polysulfone, polyester, and the like. The holes 9108 and 9116 are integrally formed in the first part and the second part, respectively. The interlocking groove (not shown), the lock projection (not shown), and the interlock projection 9118 are integrally formed within the first part 9106 and the second part 9114, respectively.

The first part 9106 can be inserted through a port access device under direct vision using an introducer that is reversibly affixed to the tail 9110. Following placement through the annulus 9102, the first part 9106 can be indexed anatomically posteriorly to allow room for the second part 9114 to be inserted through the surgically created void 9120 and into the intervertebral disc between the vertebrae (not shown). The second part 9114 can be inserted riding with its interlock projection 9118 riding within the interlocking groove (not shown) of the first part 9106 in order to maintain alignment. The beveled leading edge 9134 of the interlock projection 9118 is configured to deflect the lock prong (not shown) back into the first part 9106 under spring tension. The lock prong (not shown) can be biased toward the second part 9114 by a coil spring, leaf spring, or the like. The spring (not shown) can be integral to the first part 9106 or it can be trapped or affixed thereto. The spring (not shown) in its integral form can be a projection of polymeric material that elastically flexes toward or away from the first part 9106.

The holes 9108 and 9116 are configured to permit ingrowth of tissue within their void, or to permit the first part 9106 and the second part 9114, respectively, to be loaded with bone growth factor or other bioactive substance such as biological cement or adhesive, antimicrobial agent, or the like. The holes 9108 and 9116 are oriented anatomically axially so that the bioactive substance comes into contact with the vertebrae between which the implant 9100 is placed. The number of holes 9108 and 9116 can range between 1 and 20 and, in certain embodiments, a range between about two and about ten on either the first part 9106 or the second part 9114.

FIG. 91B illustrates the vertebral body replacement 9100 with the first part 9106 aligned with the second part 9114 and the lock prong (not shown) on the first part 9106 advanced or biased into the locking detent 9122 of the second part 9114 such that the first part 9106 and the second part 9114 are permanently and irreversibly connected together to form a single implant. The vertebral body replacement 9100 comprises the proximal transition zone 9128 which steps down from the central region toward the lower height tail. The transition zone 9124 steps down between the higher central region and the lower distal region 9132. Note that the vertebral body replacement or spacer 9100 resides with its lower height regions near the periphery of the vertebrae, with in the region of the vertebral lips.

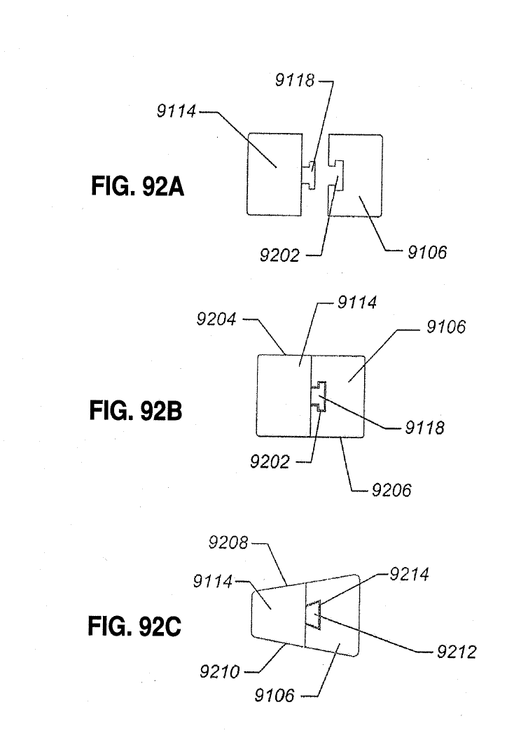

FIG. 92A illustrates a rear view of the vertebral body replacement first part 9106 and second part 9114. The second part comprises a T-shaped interlock projection 9118 and the first part 9106 comprises a slightly larger T-shaped interlock groove 9202. The cross-sectional areas of the first part 9106 and the second part 9114 are individually smaller than that of an assembled device and therefore the first part 9106 and the second part 9114 can be individually placed down a port access device using minimally invasive techniques where a larger, fully assembled unit might not fit.

FIG. 92B illustrates a rear view, looking from the proximal end toward the distal end, of the vertebral body replacement of FIG. 92 A, whereby the first part 9106 is fitted against the second part 9114. The first part 9106 and the second part 9114, when assembled comprise a top surface 9204 and a bottom surface 9206. In the illustrated embodiment, the top surface 9204 is substantially parallel and aligned with the bottom surface 9206. The top surface 9204 or the bottom surface 9206, or both, can be oriented in a single plane or they can be curvilinear in a convex or concave fashion. The top and bottom surfaces 9204 and 9206 can also be flat but the top surface 9204 of the first part 9106 can reside in a plane not the same as the top surface 9204 of the second part. For instance, the top surfaces 9204 can form a peak or a valley or even have a serrated edge. The bottom surfaces 9206 can have configurations similar to those defined for the top surfaces 9204. The interlock projection 9118 is fitted to be slidably retained within the interlock groove 9202 such that axially oriented motion is substantially permitted, substantially defining the small amount of gap between the sides of the interlock projection 9118 and the interlock groove 9202, which is present to prevent binding.

FIG. 92C illustrates a rear view looking distally of the first part 9106 and the second part 9114 wherein the interlocking projection 9212 and the interlock groove 9214 are of a dovetail shape rather than a T-shape. The cross-sectional shapes of the interlocking projection 9212 and the interlocking groove 9214 can also comprise any other geometry including an undercut such as a circle at the end of a rectangle wherein the circle has a larger diameter than the width of the rectangle. The top surface 9208, in the illustrated embodiment, is disposed at an angle relative to the central axis of the implanted parts 9106 and 9114. The bottom surface 9210 is likewise disposed at an angle relative to the central axis of the implanted parts 9106 and 9114. In the illustrated embodiment, the top surface 9208 and the bottom surface 9210 are angled relative to each other so as to form a trapezoid or blunted wedge shape. The top surface 9208 and the bottom surface 9210 can be smooth, rough, deeply serrated, grooved, drilled with holes, or the like.

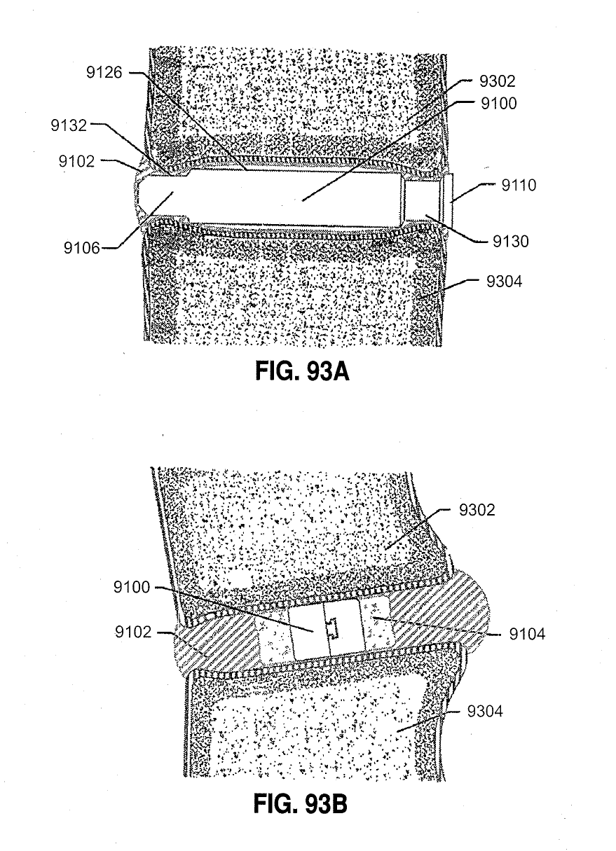

FIG. 93A illustrates a cross-sectional view of an intervertebral disc annulus 9102 and adjacent vertebrae 9302, 9304 with a first part 9106 of a vertebral body spacer 9100 implanted therein. The vertebral body spacer 9100 comprises a central region 9126 having an enlarged height, a tail 9110, a tail recess 9130, and a distal region of reduced height 9132. The central region 9126 is configured to fit within the concavity of the vertebrae 9302, 9304 while the distal region of reduced height 9132 and the tail recess 9130 are configured to capture the vertebral lips near the periphery of the vertebrae 9302, 9304. The tail 9110 resides generally at the periphery, or outside, of the intervertebral disc annulus 9132.

FIG. 93B illustrates a laterally directed view of two vertebrae 9302 and 9304 sandwiching the annulus 9102 and the nucleus 9104 of an intervertebral disc. Referring to FIGS. 93A and B, the vertebral body spacer 9100 is illustrated looking at its tail 9110. The vertebral body spacer 9100 is illustrated being placed approximately along the lateral centerline of the disc and residing within a significant portion of the annulus 9104. Note that the parallel alignment of the top and bottom surfaces of the vertebral body spacer 9100 distributes the load and maximally support the vertebrae 9302 and 9304.

In other embodiments, many of the annular implants described herein can be used as intervertebral spacers which can be placed using minimally invasive techniques. These intervertebral spacers can be used with associated spinal fusion procedures to provide for early spinal segment stabilization while the fusion procedure heals and takes full effect. The spinal fusion procedures generally entail placing vertebral connectors against the posterior part of the spine and affixing said vertebral connectors to the vertebrae using pedicle screws and the like. Spinal fusion devices can be found, for example, in U.S. Pat. No. 7,118,571 by Kumar et al. and U.S. Pat. No. 5,947,966 to Drewry et al., the entirety of which are hereby incorporated herein by reference. The vertebral connectors can comprise rods and brackets, wherein the brackets comprise holes through which the pedicle screws can be passed to secure the brackets to the vertebrae. The brackets can also comprise receivers and locks which allow the rods to be affixed to the brackets.

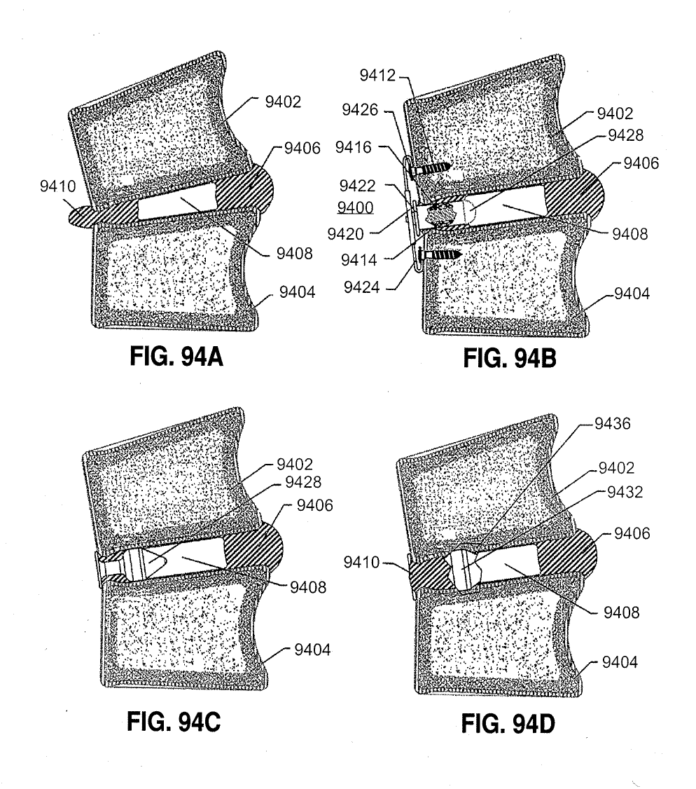

FIG. 94A illustrates a cross-sectional view of a segment of the spine comprising an upper vertebra 9402, a lower vertebra 9404 and an intervertebral disc comprising an annulus 9406 and a nucleus 9408. In this illustration, the posterior portion of the intervertebral disc 9410 has become pathologic, having degenerated and lost height such that the posterior portion of the intervertebral disc 9410 has herniated outward. The upper vertebra 9402 has rotated posteriorly due to the loss of posterior disc height.

FIG. 94B illustrates a cross-sectional view of the spine segment illustrated in FIG. 94A comprising the upper vertebra 9402, the lower vertebra 9404, the intervertebral disc annulus 9406 and the intervertebral disc nucleus 9408. The posterior aspect of the intervertebral disc 9410 has expanded to restore the original height and angle of the upper vertebra 9402. This expansion is generated and maintained as a result of implantation of the spacer 9400. The spacer 9400 comprises a nose 9428, a body 9416, an optional bumper layer 9414, and a tail flange 9422. The spacer 9400 further comprises a tail attachment 9420, a plurality of struts 9424, one or more eyelet 9426, and one or more threaded fasteners 9412. Placement of the spacer 9400 causes one or more of the therapies of restoration of the normal spinal geometry, distraction of the vertebrae 9402, 9494, facet unloading, motion preservation, height preservation, height restoration, nerve decompression or fusion support. The spacer 9400 can be used in the lumbar spine, the thoracic spine, or the cervical spine.

Referring to FIG. 94B, the tail attachment 9420 is affixed to the tail flange 9422, or integrally formed therewith. The tail flange 9422 is affixed or integral to the body 9416, which is integral or affixed to the nose cone 9428. The body 9416 can be coated or surrounded with a resilient or conformable material bumper 9414 to pad or soften the interaction between the body 9416 and the vertebrae 9402 and 9404. The threaded fasteners or screws 9412 can be pre-placed in the vertebrae 9402, 9404, the facets (not shown), pedicles (not shown) or other suitable bony structures of the vertebrae. The threaded fasteners 9412 can be placed through the eyelets 9426, which can have circular, U-shaped, slotted, or other suitable shape of opening within a structural support that is affixed to the struts 9424, which are, in turn, affixed to the tail attachment 9420.

The tail attachment 9420 can be configured to allow the struts 9424 to slide up and down but not posteriorly, laterally, or laterally left or right, with respect to the spinal axis, thus providing a system that maintains spinal segment mobility. The struts 9424 can be affixed to the upper vertebra 9402, the lower vertebra 9404, or both. In certain embodiments, there is one strut 9424 that is affixed to the upper or lower vertebra 9402 and 9404 respectively, depending on the surgical access. The struts 9424 can be rigid or they can be somewhat flexible to encourage spinal mobility. The body 9416, the tail flange 9422, the nose cone 9428, the tail attachment 9420, the struts 9424, the eyelets 9426, and the screws 9412 can be fabricated from metals such as, but not limited to, titanium, cobalt nickel alloy, nitinol, stainless steel, and the like. The body 9416, the tail flange 9422, and the nose cone 9428 can, in certain embodiments, be fabricated from polymers such as, but not limited to, PEEK, polysulfone, polyester, polyimide, polyamide, reinforced polymer, or the like. The bumper material 9414, which is comprised by an optional embodiment, can be fabricated from soft polymers such as, but not limited to, polyurethane, polycarbonate urethane, silicone elastomer, thermoplastic elastomer, or the like. The hardness of the bumper material 9414 can range from a 5 A to 90 A, and, in certain embodiments, a range of 30 A to 72 A. The bumper material 9414 can also comprise one or more layer of woven, knitted, or braided fabric fabricated from materials such as, but not limited to, polyester and PTFE. These fabric layers can use porosity to encourage tissue ingrowth and scar tissue healing, thus assisting with sealing of any annular defect caused by implantation of the spacer 9400. The fabric layers can be used alone or as an outer layer over the soft resilient bumper materials described herein. The tail flange 9422 is optional and may not be required in certain embodiments.

FIG. 94C illustrates the side cross-sectional view, looking laterally, at the spine segment of FIG. 94A, wherein an intradiscal implant 9428 has been placed for the purpose of restoration of the normal spinal geometry, distraction of the vertebrae 9402, 9494, facet unloading, motion preservation, height preservation, height restoration, nerve decompression or fusion support. The implant 9428 can be used in the lumbar spine, the thoracic spine, or the cervical spine. The annulus 9406 and the nucleus 9408 are undistorted and fully expanded, especially in the posterior region, as a result of placement of the implant 9428. The enlarged head of the implant 9428 is configured to fit within the undercut on the discal surfaces of the vertebrae 9402, 9404 and prevent expulsion of the implant 9428. The implant 9428 can be placed without the need for reaming or removing any bone from the vertebrae 9402, 9404, although removal of some annular tissue 9406 may be beneficial. Note that the implant 9428 can be one piece or multiple piece devices such as those illustrated in FIGS. 85 through 88.

FIG. 94D illustrates a side cross-sectional view, looking laterally, at the spine segment of FIG. 94A, wherein a spinal implant 9432 has been placed for the purpose of restoration of the normal spinal geometry, distraction of the vertebrae 9402, 9494, facet unloading, motion preservation, height preservation, height restoration, nerve decompression or fusion support. The implant 9432 can be used in the lumbar spine, the thoracic spine, or the cervical spine. In this illustration, the implant 9432 is illustrated behind the spinal cross-section and a window 9436 has been created to show the head of the implant 9432. FIG. 94D clearly illustrates how the posterior portion of the annulus 9410 has been rendered normal in curvature with the herniated bulge of FIG. 94A being eliminated by placement of the implant 9432. The implant 9432 differs from the implant 9428 of FIG. 94C in that the implant 9432 is larger in diameter relative to the vertebral spacing and, thus, requires reaming or removal of bone material from the upper vertebra 9402 and the lower vertebra 9404, prior to device placement. Note that the implant 9432 can be one piece or multiple piece devices such as those illustrated in FIG. 85, 86, 87, or 88.

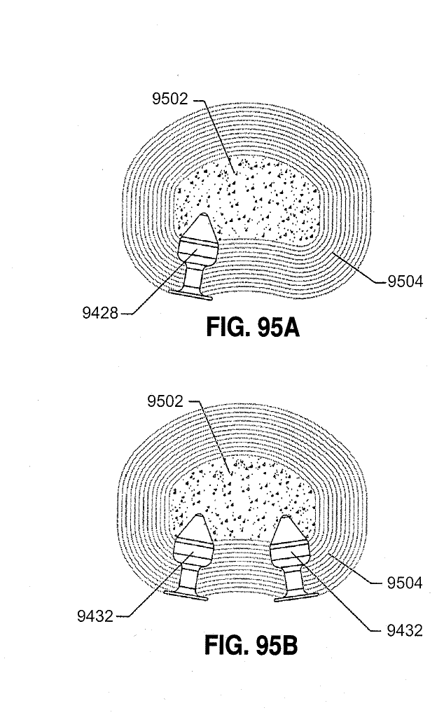

FIG. 95A illustrates the implant 9428 of FIG. 94C as viewed looking caudally, along the axis of the spine, at a cross-sectional view of the intervertebral disc annulus 9504 and nucleus 9502. A single implant 9428 is placed unilaterally placed on the anatomical right side of the posterior spine.

FIG. 95B illustrates two implants 9432 of the type illustrated in FIG. 94D as viewed looking caudally along the long axis of the spine, at a cross-sectional view of the intervertebral disc annulus 9504 and nucleus 9502. The two implants 9432 are placed, one on each side of the posterior spine, to provide a balanced distraction to the spinal column. The tail flanges of the implants 9432, the heads of the implants 9432, or both, are configured to engage the vertebral apophyseal ring, which comprises one or more vertebral lips. In other embodiments, for example, the implant 9428 of FIG. 95A can likewise engage one or both apophyseal rings of the vertebrae.

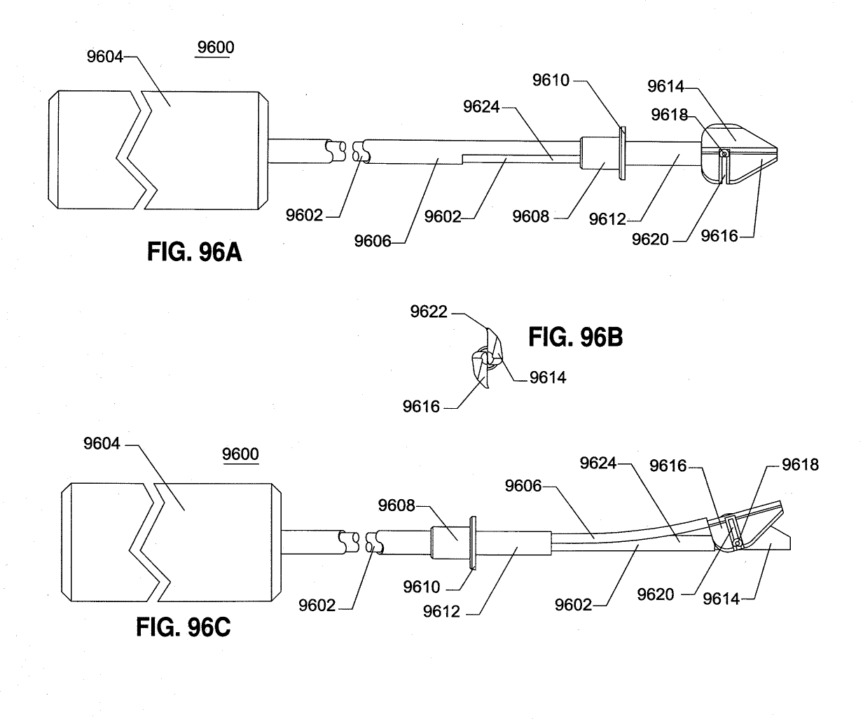

FIG. 96A illustrates a side view of an expandable reamer 9600 with its reamer bit in its second, laterally expanded configuration. The expandable reamer 9600 comprises a handle 9604, a central shaft 9602, an outer shaft 9606 further comprising a sidecut 9624, a tail boss 9608, a tail flange 9610, a tail standoff 9612, a first cutter blade 9614, a second cutter blade 9616 further comprising a slot 9620, and a slot retainer 9618.

The handle 9604 is affixed to the inner shaft 9602 and the outer shaft 9606. The tail boss 9608, the tail flange 9610, and the tail standoff 9612 are affixed, or integral, to each other. The tail flange 9610, the tail standoff 9612, and the tail boss 9608 comprise a central lumen (not shown) permitting them to slidably constrain the outer shaft 9606 and the inner shaft 9602. The first cutter blade 9614 is affixed, or integral, to the inner shaft 9602 while the second cutter blade 9616 is affixed, or integral to, the outer shaft 9606. The outer shaft 9606 comprises the cutout 9624, which is integral thereto. The outer shaft 9606 is spring biased to arc away from the inner shaft 9602 at its distal end but is constrained not to move apart by the slider comprising the tail flange 9610, the tail standoff 9612, and the tail boss 9608 when the slider is advanced distally, as illustrated in FIG. 96A. In this configuration, the cutter blades 9614 and 9616 are at their maximum separation distance or their expanded condition.

FIG. 96B illustrates a front view of the distal end of the expandable reamer 9600 in the expanded configuration. The distal end of the expandable reamer 9600 comprises the first cutter blade 9614 further comprising the cutting edge 9622, and the second cutter blade 9616.

The cutting edge 9622 is integral to the first cutter blade 9614 as illustrated and a similar cutting edge 9622 can optionally be affixed, or integral, to the second cutter blade 9616. The cutting edges 9622 operate when the first cutter blade 9614 and the second cutter blade 9616 are rotated clockwise as viewed from the proximal end of the device. In another embodiment, the cutting edges 9622 can be reversed so the first cutter blade 9614 and the second cutter blade 9616 are rotated in the counterclockwise direction.

FIG. 96C illustrates a side view of the expandable reamer 9600 in its reamer head in its first, unexpanded configuration. The expandable reamer 9600 comprises the handle 9604, the central shaft 9602, the outer shaft 9606 further comprising the cutout 9624, the tail boss 9608, the tail flange 9610, the tail standoff 9612, the first cutter blade 9614, the second cutter blade 9616 further comprising the slot 9620, and the slot retainer 9618.

Referring to FIG. 96C, the tail flange 9610, the tail standoff 9612, and the tail boss 9608 are retracted proximally to permit the outer shaft 9606 to fully deflect and permit the second cutter blade 9616 to align with the first cutter blade 9614 in the most compact, non-expanded configuration. Manual application of force, in the proximal direction, on the tail flange 9610 or the tail boss 9608 will retract tail flange assembly permitting the spring biased outer shaft 9606 to deflect out of the longitudinal axis with the inner shaft 9602 clearing the outer shaft through the cutout 9624 or window. The slot retainer 9618, which is affixed to the first cutter blade 9614, projects through the slot 9620, which is integral to the second cuter blade 9616. A head or cap on the slot retainer 9618, which is affixed or integral thereto, prevents the first cutter blade 9614 from moving away from the second cutter blade 9616 in a direction normal to the plane in which the slot 9620 resides. The head or cap on the slot retainer 9618 is wider than the width of the slot, thus preventing motion other than sliding along the longitudinal axis of the slot 9620

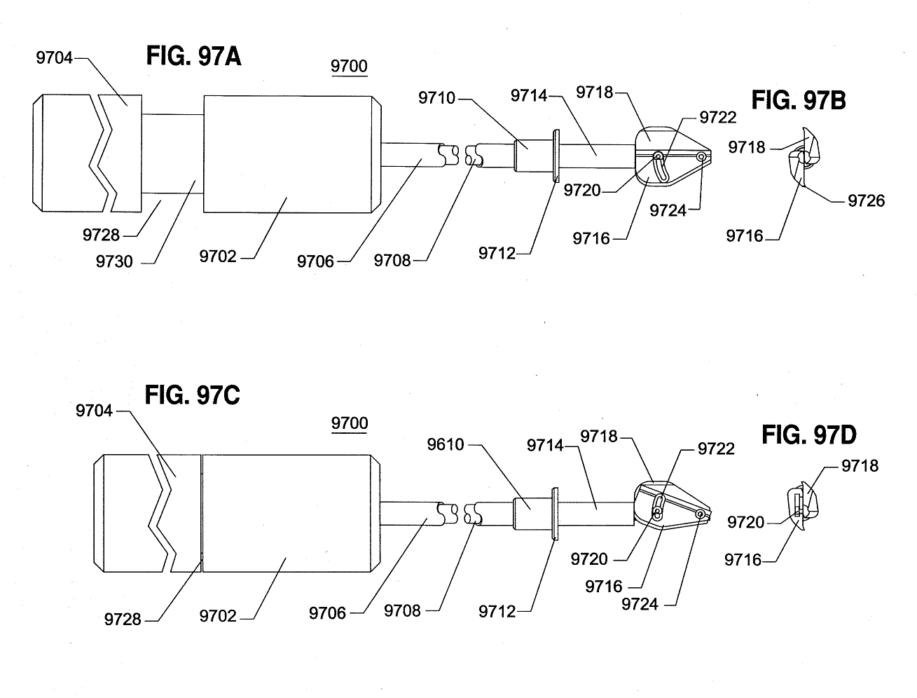

FIG. 97A illustrates a side view of an expandable reamer 9700 comprising pivoting cutter blades, in its second, fully expanded state. The expandable reamer 9700 comprises a rear handle 9704, a front handle 9702, a rear handle step-down 9730, a handle gap 9728, an outer shaft 9706, an inner shaft 9708, a tail boss 9710, a tail flange 9712, a tail standoff 9714, a first cutter blade 9718, a second cutter blade 9616 further comprising a slot 9722, a slot retainer 9720, and a pivot 9724.

Referring to FIG. 97A, the rear handle 9704 is constrained to move along the longitudinal axis, or a rotational axis, of the reamer 9700. The rear handle step-down 9730 is slidably retained within a lumen of the front handle 9702 and is affixed, or integral, to the rear handle 9704. The distal end of the rear handle step-down 9730 is affixed to the central shaft 9708. The central shaft 9708 is slidably retained within a lumen of the outer shaft 9706 and can move in the longitudinal axis or a rotational axis. The tail flange 9712, the tail standoff 9714, and the tail boss 9710 are affixed, or integral to, the outer shaft 9606. The first cutter blade 9718 is affixed to the distal end of the outer shaft 9706. The second cutter blade 9716 is affixed to a linkage (not shown), which is affixed to the central shaft 9708. In an embodiment, longitudinal motion of the central shaft 9708, caused by movement of the rear handle 9704 relative to the front handle 9702, causes the second cutter blade 9716 to rotate about its pivot 9724 and constrained by the slot 9722 and the slot retainer 9720. The gap 9728 provides potential space for movement of the rear handle 9704 relative to the front handle 9702 and it also provides a positive stop against over-displacement. Once the second cutter blade 9716 has been advanced to its fully expanded configuration, it can be locked in place by rotating the rear handle 9704 about its axis to engage a lock (not shown). The slot retainer 9720 slidably moves along the axis (either straight or arcuate as illustrated) of the slot 9722. A head or cap, integral, or affixed, to the slot retainer 9720 prevents separation of the first cutter blade 9714 from the second cutter blade 9716. In another embodiment, rotation of the rear handle 9704 about its longitudinal axis can turn a jackscrew (not shown) which moves the second cutter blade 9716 with significant mechanical advantage. Once the second cutter blade has been moved to its fully expanded condition, as illustrated in FIG. 97A, the second cutter blade can be locked in position by movement of the rear handle 9704 along its longitudinal axis to engage a lock (not shown).

The components of the expandable reamers 9600, 9700, and 9800 can comprise materials such as, but not limited to, stainless steel, cobalt nickel alloy, titanium, nitinol, or the like. The handle components of these reamers can be fabricated from metals, as described, or polymers such as, but not limited to, polycarbonate, acrylonitrile butadiene styrene (ABS), polyester, polysulfone, PVC, or the like. The reamers 9600, 9700, 9800 are beneficially configured to be sterilizable using steam, gamma irradiation, ethylene oxide gas, electron beam irradiation, and the like. In certain embodiments, these devices are disposable and are packaged appropriately for single use.

FIG. 97B illustrates a front view of the distal end of the reamer bit of the expandable reamer 9700 in the expanded configuration. The reamer bit at the distal end of the expandable reamer 9700 comprises the first cutter blade 9718 and the second cutter blade 9616 further comprising a cutting edge 9726. The cutting edge 9720 is illustrated on the second cutter blade 9616 but in an exemplary embodiment, both the second cutter blade 9616 and the first cutter blade comprise cutting edges 9726.

FIG. 97C illustrates a side view of an expandable reamer 9700 comprising pivoting cutter blades, in its first, unexpanded state. The expandable reamer 9700 comprises a rear handle 9704, a front handle 9702, a handle gap 9728, an outer shaft 9706, an inner shaft 9708, a tail boss 9710, a tail flange 9712, a tail standoff 9714, a first cutter blade 9718, a second cutter blade 9716 further comprising a slot 9722, a slot retainer 9720, and a pivot 9724.

Referring to FIG. 97C, the rear handle 9704 has been advanced distally relative to the front handle 9702 causing the inner shaft 9708 to advance distally relative to the outer shaft 9706. Distal movement of the inner shaft 9708 causes the linkage connecting the inner shaft 9708 to the second cutter blade 9716 to move the second cutter blade 9716 to rotate about the pivot 9724 as constrained by the slot 9722 and the slot retainer 9720. The pivoting motion of the second cutter blade 9716 can be accomplished with a lever, a cam, a jackscrew, a wedge, or other motion transfer device operatively connecting the inner shaft 9708 and the second cutter blade 9716. A spring return (not shown) can assist or dominate return of the second cutter blade 9716 to its fully expanded state when desired.

FIG. 97D illustrates a front view of the distal end of the reamer bit of the expandable reamer 9700 in its unexpanded configuration. The reamer bit at the distal end of the expandable reamer 9700 comprises the first cutter blade 9718, the second cutter blade 9616, and the slot retainer 9720. The slot retainer 9720 can be seen in cross-section to visualize the cap or enlargement.

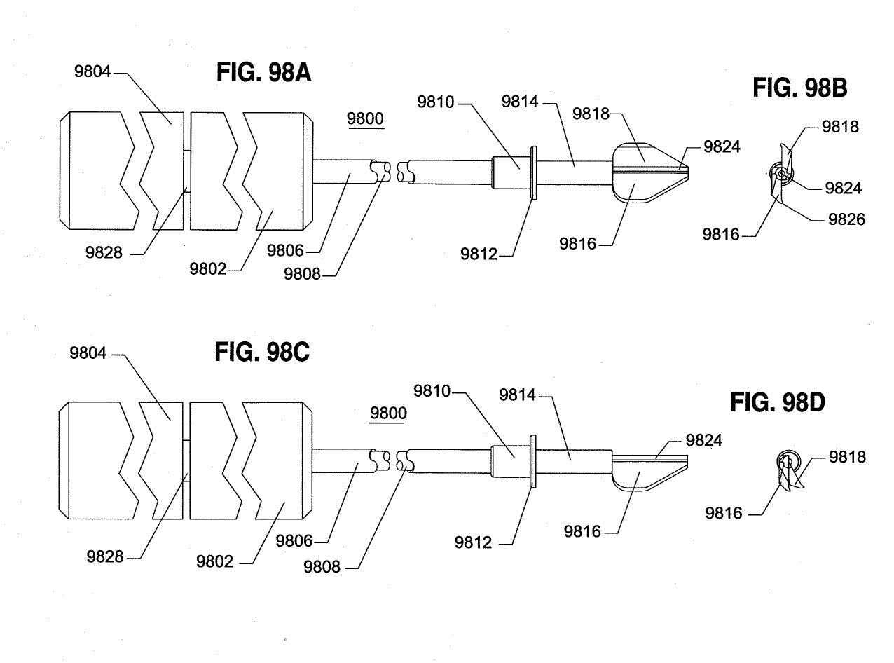

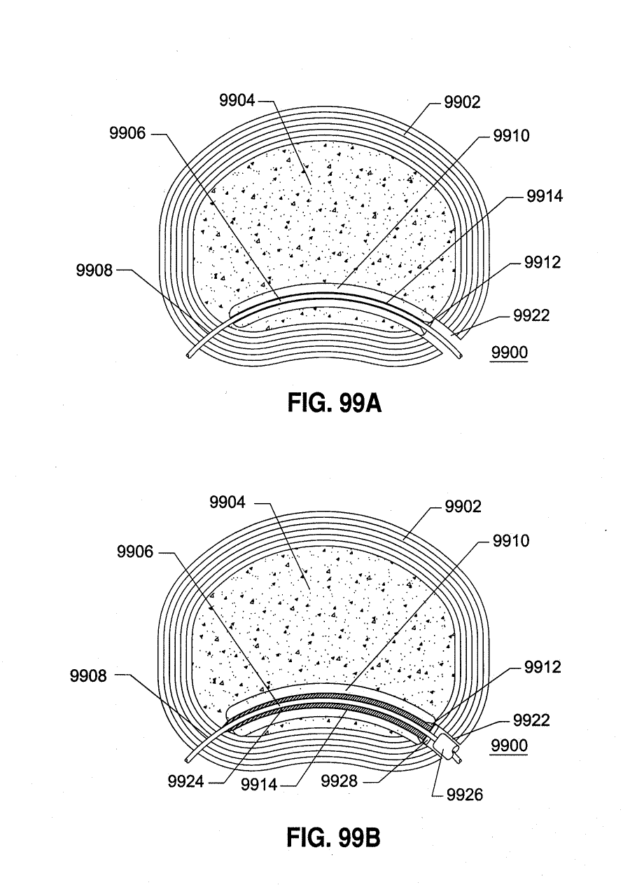

FIG. 98A illustrates an expandable reamer 9800 in its second, fully expanded state. The expandable reamer 9800 comprises a rear handle 9804, a front handle 9802, a handle shaft 9828, an outer shaft 9806, an inner shaft 9808, a tail boss 9810, a tail flange 9812, a tail standoff 9814, a first cutter blade 9818, a second cutter blade 9816, and a cutter pivot 9824.

Referring to FIG. 98A, the rear handle 9804 is constrained to rotate about its longitudinal axis. The rear handle 9804 is affixed, or integral, to the proximal end of the handle connector 9828. The handle connector 9828 is constrained to rotate about its longitudinal axis with a portion of the handle connector 9828 extending into a lumen of the front handle 9802. The distal end of the handle connector 9828 is affixed to the inner shaft 9808. The front handle 9802 is affixed, at its distal end, to the proximal end of the outer shaft 9806. A protrusion (not shown) affixed to the front handle 9802, riding in a groove (not shown), integral to the handle connector 9828 prevents longitudinal relative motion between the handle connector 9828 and the front handle. The tail boss 9810, the tail flange 9812, and the tail standoff 9814 are integral, or affixed, to each other and the assembly is affixed, or integral, to the outer shaft 9808. The second cutter blade 9816 is affixed to the distal end of the outer shaft 9806. The first cutter blade 9818 is affixed to the distal end of the inner shaft 9808. Rotation of the inner shaft 9808 about its longitudinal axis causes the first cutter blade 9818 to rotate about the cutter pivot 9824. A lock (not shown) can optionally be provided in the handle to restrain the rear handle 9804 from rotating relative to the front handle 9802 unless the lock is unlocked. Marks, scribes, or indices can also be printed or engraved in the rear handle 9804, the front handle 9802, or both, to provide a visual indication of the position of the second cutter blade 9816 relative to the first cutter blade 9818.

FIG. 98B illustrates a front view of an expandable reamer bit of the expandable reamer 9800, comprising the first cutter blade 9818, the second cuter blade 9816 further comprising a cutting edge 9826, and the cutter pivot 9824. The cutting edge 9826 is shown integral to the second cutter blade 9816 but it can, in another embodiment, be integral to the first cutter blade 9818, or both cutter blades 9816 and 9818.

FIG. 98C illustrates the expandable reamer 9800 in its first, unexpanded state. The expandable reamer 9800 comprises the rear handle 9804, the front handle 9802, the handle shaft 9828, the outer shaft 9806, the inner shaft 9808, the tail boss 9810, the tail flange 9812, the tail standoff 9814, the second cutter blade 9816, and a cutter pivot 9824. The first cutter blade 9818, as illustrated in FIGS. 98A and 98B is rotated out of view and is not visible in this illustration.

Referring to FIG. 98C, the rear handle 9804 has been rotated counterclockwise relative to the front handle 9802 causing the inner shaft 9808 and the first cutter blade 9818 to rotate counterclockwise to a minimum profile configuration. In this configuration, the reamer 9800 is not suitable for reaming, but rather for insertion or removal from the annular space. Thus, following a reaming procedure, the reamer 9800 can be returned to the configuration shown in FIGS. 98C and 98D to facilitate removal from the body.

FIG. 98D illustrates a front view of the expandable reamer bit of the expandable reamer 9800, comprising the first cutter blade 9818, the cutter pivot 9824, and the second cutter blade 9816 wherein the first cutter blade 9818 has been rotated about the cutter pivot 9824 to a minimum profile configuration.

FIG. 99A illustrates an intervertebral disc looking inferiorly and shown in cross-section. The intervertebral disc comprises an annulus 9902 and a sub-annular space, or nucleus 9904. An implant 9900, further comprising an inner lumen 9914 with a proximal internal flare 9912, has been routed into the intervertebral disc over a guidewire 9906, which is routed through the annulus 9902 through the puncture 9908. The implant 9900 has been routed through the annulus 9902 through the access tunnel 9922.

Referring to FIG. 99A, the implant 9900 is expandable and can comprise longitudinal slits (not shown), expandable linkages, or it can comprise elastomeric or plastically deformable materials to permit the expansion in a direction lateral to the longitudinal axis of the implant 9900. In certain embodiments where the implant 9900 is elastomerically expandable, the implant 9900 can be fabricated from silicone elastomer, polyurethane elastomer, polycarbonate urethane, thermoplastic elastomer, or the like. In certain embodiments where the implant comprises longitudinal disconnections, slits, slots, expandable linkages, or the like. The expandable linkages can comprise malleable metal such as titanium, tantalum, gold, platinum, stainless steel, or the like. The longitudinal slits can comprise thin areas or disconnections between circumferentially adjacent segments that are capable of moving apart circumferentially. The central lumen 9914 tracks over the guidewire 9906 and slidably constrains the implant 9900 to follow the guidewire 9906 when the implant 9900 is advanced distally.

FIG. 99B illustrates the implant 9900 placed within the intervertebral disc and further wherein the implant 9900 has been expanded diametrically, laterally, radially, circumferentially, or the like. The implant 9900 is expanded because of the introduction of a dilator 9924 through the flared proximal end 9912 of the implant 9900 and into the central lumen 9914. The implant 9900 can expand circularly, elliptically, or in an inferior-superior direction. The amount and direction of expansion can be controlled by the cross-sectional geometry of the dilator 9924. The dilator 9924 further comprises an optional proximal head 9928 which can be configured to lock into the implant 9900 or to limit distal motion of the dilator 9924, to prevent proximal motion of the dilator following placement, or both. The dilator proximal head 9928 can, in certain embodiments, lock into the proximal end of the implant 9900. The dilator 9924 can be coerced into position by the dilator pusher 9926, illustrated placed over the guidewire 9906. In other embodiments, the implant 9900 can be made to expand by use of water swellable materials such as hydrogels, polymethyl cellulose, or the like. An outer, porous coating (not shown) surrounding the implant 9900 can permit water intake but prevent loss of water swellable material from the environs of the implant 9900.

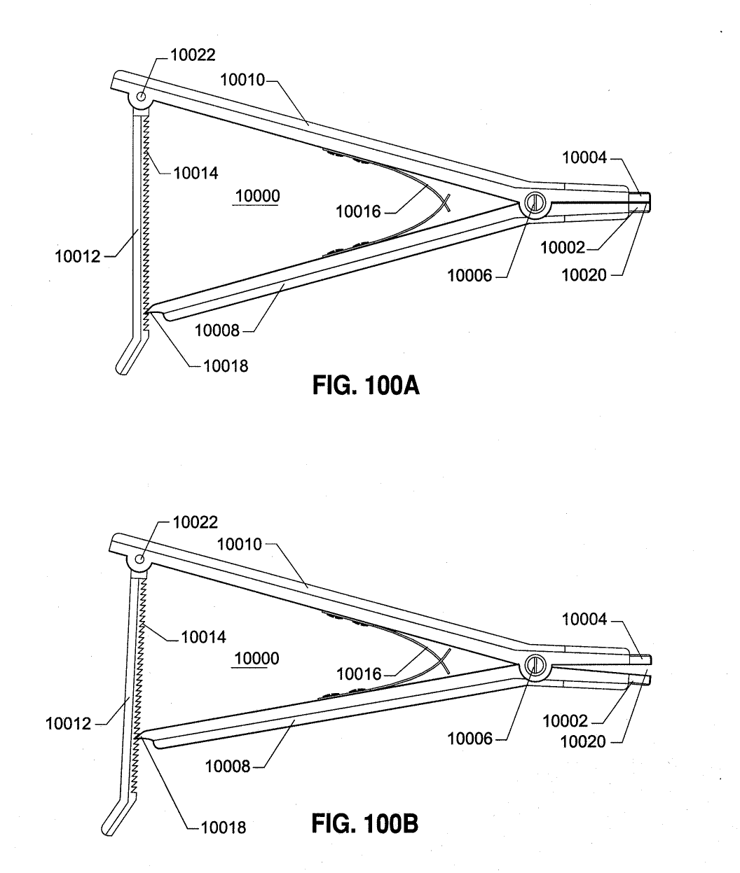

FIG. 100A illustrates a distraction instrument 10000 in side view with the jaws 10004 and 10002 in their closed position. The distraction instrument 10000 comprises the upper jaw 10004, the lower jaw 10002, a jaw division 10020, a pivot 10006, an upper handle 10010, a lower handle 10008 further comprising a ratchet engagement 10018, a bias spring 10016, and a ratchet rod 10012 further comprising a plurality of ratchet teeth 10014.

Referring to FIG. 100A, the upper handle 10010 is rotatably connected to the lower handle 10008 by the pivot 10006. The upper handle 10010 is integral, or affixed to, the upper jaw 10004. The lower handle 10008 is integral, or affixed to, the lower jaw 10002. The ratchet rod 10012 is affixed, or integral, to the ratchet teeth 10014 and is rotatably connected to the upper handle 10010 about the ratchet rod pivot 10022. The ratchet engagement 10018, integral, or affixed to, the lower handle 10008 can be engaged or disengaged with the ratchet teeth 10014 at a plurality of discreet locations. The bias spring 10016 is affixed to the upper handle 10010 and the lower handle 10016 such that the bias spring 10016 forces the handles 10010 and 10008 apart with some pre-determined, or adjustable, force or spring constant.

The entire distraction instrument 10000 can be fabricated from stainless steel, cobalt nickel alloy, titanium, nitinol, or alloys thereof. High strength stainless steel and integral construction with attention to minimizing high stress areas can beneficially be employed to fabricate the distraction instrument 10000. In certain embodiments, the bias spring 10016, which can comprise one or more elements, is fabricated from spring-temper stainless steel, nitinol, or a cold rolled cobalt nickel alloy such as Elgiloy®.

The jaw portion of the distraction instrument 10000 is beneficially of constant height moving distally to the pivot 10006. In this way, the profile is minimized so that the jaws 10004 and 10006 can be inserted into a port access device. In other embodiments, a plurality of pivots 10006 and linkages can be utilized to maintain a small profile through a long port access system.

FIG. 100B illustrates a distraction instrument 10000 in side view with the jaws 10004 and 10002 in their open position. The distraction instrument 10000 comprises the upper jaw 10004, the lower jaw 10002, the jaw division 10020 which is now open, the pivot 10006, the upper handle 10010, the lower handle 10008 further comprising the ratchet engagement 10018, the bias spring 10016, and the ratchet rod 10012 further comprising the plurality of ratchet teeth 10014.

The handles 10010 and 10018 have been rotated slightly together causing the jaws 10004 and 10006 to pivot open about the pivot 10006. The distance between the outside of the open jaws 10004 and 10006 near the distal end can range between about 1-mm to 20-mm, and, in certain embodiments, with a range of about 5-mm to 15-mm. Engagement of the ratchet engagement 10018 with the ratchet teeth 10014 prevents the jaws from re-closing until it is desired to do so. Disengagement of the ratchet engagement 10018 with the ratchet teeth 10014 can be accomplished by pulling the ratchet rod 10012 proximally to disengage the teeth 10014.

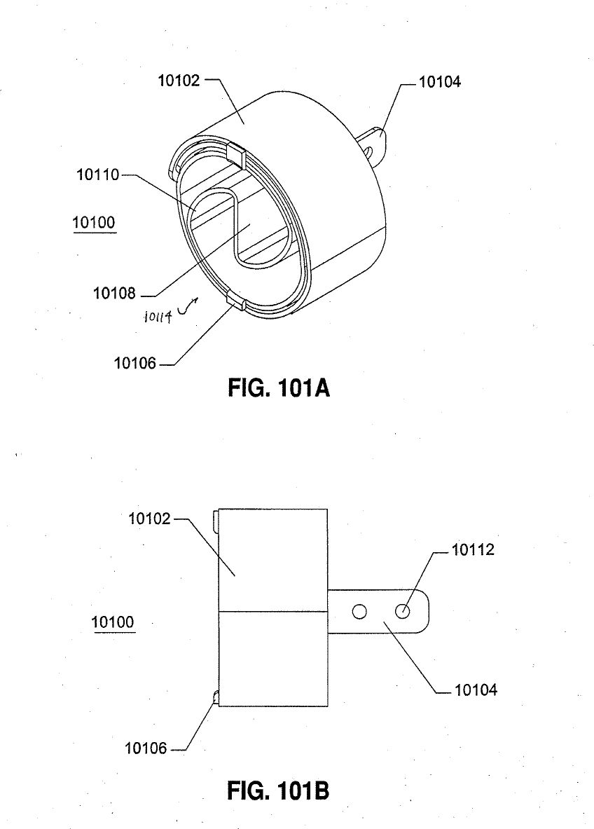

FIG. 101A illustrates an expandable spiral reamer 10100 in oblique view. The expandable spiral reamer 10100 comprises a contact surface member 10102 further comprising at least one free edge 10114, an attachment tab 10104, a stabilizer tab 10106, a torque application member 10108, and a radial transition zone 10110.

Referring to FIG. 101A, the spiral reamer 10100 is configured to be gripped by an instrument or handle at the attachment tab 10104. The attachment tab 10104 is affixed, or integral to, the torque application member 10108. The torque application member 10108 is affixed, or integral to, the radial transition zones 10110. The radial transition zones 10110 are affixed, or integral to, the surface contact member 10102, which forms the outermost surface of the reamer 10100. The stabilizer tabs 10106 are affixed, or integral to, at least one region of the surface contact member 10102. The stabilizer tabs 10106, provide guidance to the plurality of layers comprising the surface contact member 10102, thus preventing longitudinal dislocation of the surface contact member 10102. The reamer 10100 can comprise between 1 and 10 stabilizer tabs 10106. In certain embodiments, the stabilizer tabs 10106 can also prevent, or limit, radial separation of the layers of the surface contact member 10102 by comprising caps or protrusions that grip the outer surface of the surface contact member 10102 but allow circumferential sliding of one layer of the surface contact member 10102 relative to another.

The spiral reamer 10100, in certain embodiments, can be used to create rotary cuts in the tissue of the intervertebral disc and neighboring vertebrae, when inserted therein and rotated in the correct direction. Cutting will occur when the spiral reamer 10110 is rotated such that the free edge, or end, 10114 of the surface contact member 10102 is advanced first so as to become the leading edge 10114. When cutting occurs, tissue will fill in the spaces within the spiral reamer 10100. In some embodiments, the cutting action also can cause the layers of the surface contact member 10102 to move radially apart and expand diametrically. Reverse motion of the spiral reamer 10100 will generally not cause cutting and may generate reduced diameter, however, tissue that has become entrapped between the layers of the surface contact member 10102 or even the central area surrounding the torque application member 10108 and the radial transition zones 10110 may not be expelled sufficiently to allow a diameter reduction.

FIG. 101B illustrates a side view of the spiral reamer 10100. The spiral reamer comprises the surface contact member 10102, the plurality of stabilizer tabs 10106, and the attachment tab 10104, further comprising a plurality of instrument attachment features 10112.

Referring to FIG. 101B, the instrument attachment features 10112 are holes, protrusions, or fenestrations, formed integral, or attached, to the attachment tab 10104. Instruments used to grip the attachment tab 10104 can be reversibly locked to the attachment tab 10104 by means of the instrument attachment features 10112. Materials used for fabrication of the spiral reamer 10100 can include, but are not limited to, titanium, nitinol, stainless steel, cobalt nickel alloy, PEEK, polycarbonate, reinforced polymers, or the like. The spiral reamer 10100 can comprise a spiral of material having a thickness ranging from about 0.003 to 0.050 inches, and, in certain embodiments, with a range of about 0.005 to 0.030 inches. The axial length of the reamer 10100, excluding the attachment tab 10104 can range from about 0.050 inches to about 1.0 inches, and, in certain embodiments, with a range of about 0.100 to 0.500 inches.

In certain embodiments, the spiral reamer 10100 is an instrument that can be advanced into a defect in an intervertebral disc and then be rotated to remove tissue. In other embodiments, the spiral reamer 10100 is an implant that can be advanced into a defect in an intervertebral disc and expanded to fill the space. In certain embodiments, the spiral reamer 10100 can be expanded and then released to remain behind as an implant. The spiral reamer implant 10100 can be detached by releasable locking mechanisms on a handle or other delivery system. Tissue that remains behind within the interstices of the spiral reamer 10100 can support the structure of the spiral reamer 10100 to form a structurally solid implant.

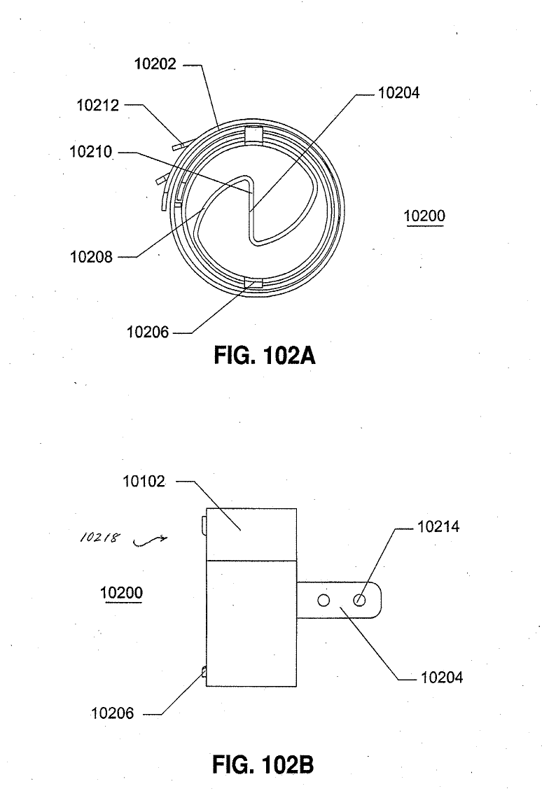

FIG. 102A illustrates another embodiment of an expandable reamer 10200 in end view. The expandable spiral reamer 10200 comprises a contact surface member 10202 further comprising at least one free edge 10218, at least one stabilizer tab 10206, a torque application member 10204, a plurality of radial transition zones 10208, and at least one reaming feature 10212.

Referring to FIG. 102A, the construction of the expandable reamer 10200 is essentially similar to that of the expandable reamer 10100, with the exception that additional layers can exist within the surface contact member 10202 and a plurality of reaming features or burrs 10212 are provided either integral to, or affixed to, the surface contact member 10202. The reaming features or burrs 10212 can comprise sharpened exposed edges. The reaming features or burrs 10200 can be affixed or integral to inner layers of the surface contact member 10202 and project through holes or fenestrations (not shown) in outer layers of the surface contact member 10202. The reaming features or burrs 10200 can serve the additional purpose of preventing axial relative motion of one layer of the surface contact member 10202 relative to another layer thereof.

The expandable reamer 10200 can serve as an expandable or collapsible reamer, or, in other embodiments, it can serve as an expandable reamer and an expandable implant. The implant can entrain spinal tissue into its interstices to create a composite tissue and prosthetic implant structure.

FIG. 102B illustrates a side view of the expandable reamer 10200. The expandable reamer 10200 comprises the attachment tab 10216 further comprising the attachment features 10214, the plurality of stability tabs 10206, and the surface contact member 10202.

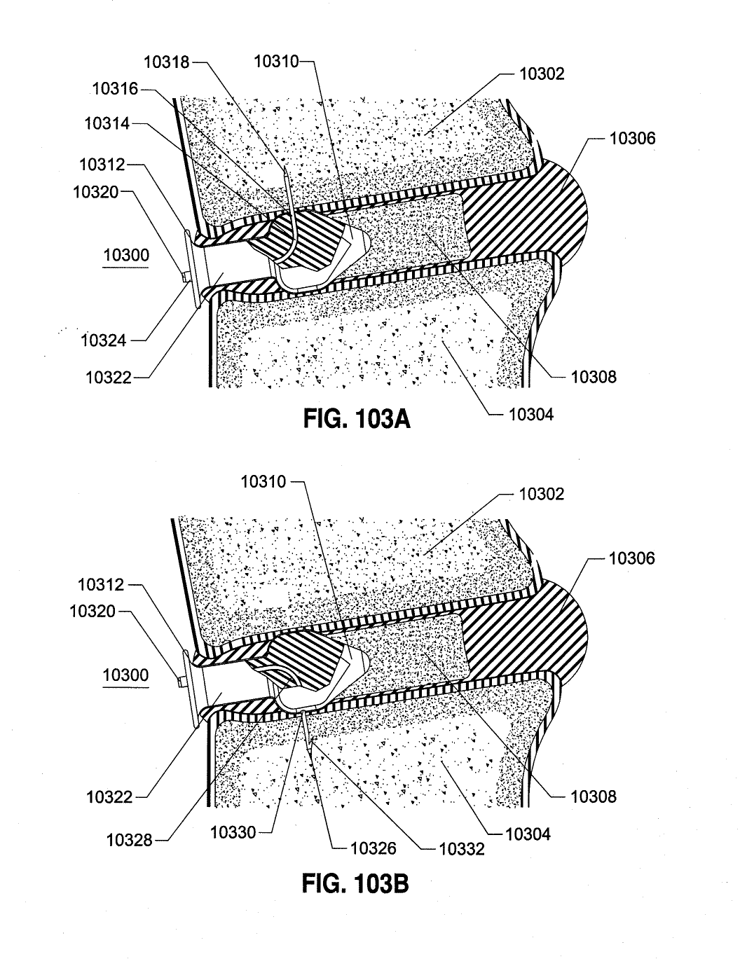

FIG. 103A illustrates a cross-sectional view of a spine segment comprising a superior vertebra 10302, an inferior vertebra 10304, an intervertebral disc annulus 10306, and an intervertebral disc nucleus 10308. An implant 10300 is placed from the posterior direction through the annulus 10306 and extending into the nucleus 10308. The implant 10300 comprises a head 10310, a tail 10322, a tail flange 10312, an inferiorly directed, deflecting spike lumen 10314 further comprising an exit port 10316 and an inlet port 10324, and a spike 10318 further comprising a proximal head 10320. The spike 10318 is oriented to be affixed into the superior vertebra 10302.

Referring to FIG. 103A, the implant 10300 is placed in the manner of other intervertebral disc implants described herein. The spike 10318, which can be pre-placed such that it does not project out beyond the exit port 10316, or not placed within the implant 10300, is advanced under mechanical advantage, being deflected by the lumen 10314 and embedded within the superior vertebra 10302. The spike 10318 can be tapped in place with a mallet, rotated and screwed in place using distal threads (not shown) and a screwdriver type arrangement at the proximal end, or forced therein using a specialized delivery system that advances the spike 10318 relative to the tail flange 10312. Once in place, the proximal spike head 10320 can be affixed or locked to the inlet port 10324 of the interior deflecting lumen 10314 using means such as a bayonet mount, screw threads, locking detent, or the like. The spike 10318 is advantageously fabricated from flexible materials exhibiting high strength. The spike 10318 can be fabricated from nitinol, cobalt nickel alloy, titanium, or the like. By embedding the spike 10318 in the superior vertebra 10302, some motion preservation is maintained while ensuring that the implant 10300 cannot be expelled from its implant location.

FIG. 103B illustrates a cross-sectional view of a spine segment comprising a superior vertebra 10302, an inferior vertebra 10304, an intervertebral disc annulus 10306, and an intervertebral disc nucleus 10308. An implant 10300 is placed from the posterior direction through the annulus 10306 and extending into the nucleus 10308. The implant 10300 comprises a head 10310, a tail 10322, a tail flange 10312, a superiorly directed, deflecting spike lumen 10328 further comprising an exit port 10330 and an inlet port 10324, and a spike 10326 further comprising a proximal head 10320 and a barb 10332. The spike 10326 is oriented to be affixed into the inferior vertebra 10304.

Referring to FIG. 103B, the function of the implant 10300 is identical to that of the implant 10300 in FIG. 103A, with the exception that the spike 10326 is directed inferiorly in the anatomically downward direction and into the inferior vertebra 10304. Another difference is that the spike 10326 further comprises a barb 10332 to prevent or minimize the risk of the barb 10332 becoming disengaged from the vertebra 10304.

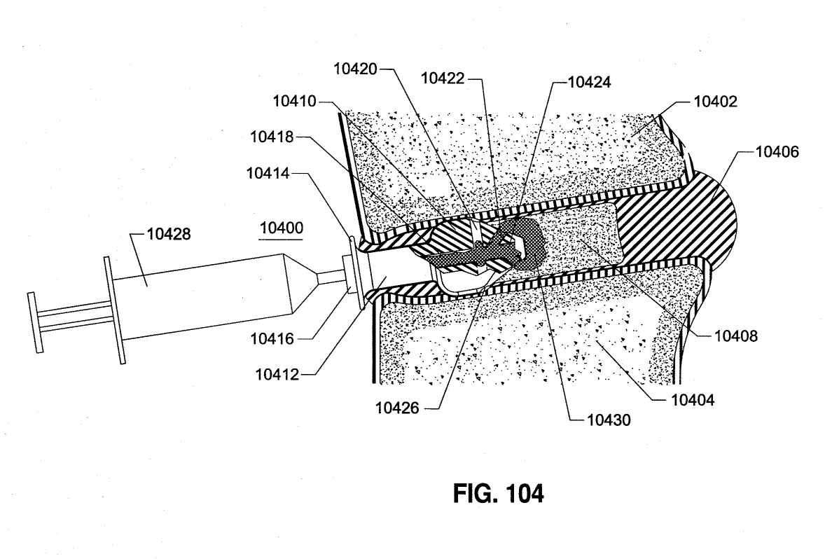

FIG. 104 illustrates a spinal implant 10400 placed within a spine segment. The spine segment comprises a superior vertebra 10402, an inferior vertebra 10404, an intervertebral disc annulus 10406, and an intervertebral disc nucleus pulposus 10408. The implant 10400 comprises a head 10410, a tail 10412, a tail flange 10414, an injection port 10416, a main injection lumen 10418, a plurality of side lumens 10420, a forward directed lumen 10424, a plurality of oblique lumens 10422, an injection device 10428, and a volume of injectable material 10430. Each side lumen 10420, forward lumen 10424 and oblique lumen 10422 comprises an exit port or vent 10426.

The side lumens 10420, forward lumen 10424, and oblique lumens 10422 are operably connected to the main injection lumen 10418, which is operably connected to the injection port 10416. The injection port 10416 is reversibly connected to the injection device 10428, which can be a syringe having a Luer-lock fitting, a Luer fitting, a threaded fitting, a bayonet mount, or the like. The injection device 10428 can further comprise a jackscrew mechanism to provide mechanical advantage for injecting its contents. The contents 10430 of the injection device 10428 are illustrated flowing through the main lumen 10418, the forward directed lumen 10424, and the oblique lumens 10422, such that the material 10430 flows into the nucleus 10408. Material 10430 does not flow through the side lumens 10420 because the exit ports 10426 of the side lumens 10420 are blocked by bone. Lumens 10418, 10420, 10424, and 10422 are integral to the head 10410 while the main injection lumen 10418 passes through the tail 10412 and extends to the proximal end of the tail flange 10414.

The material 10430 can comprise bone growth factors, nucleus replacement elements, hydrophilic hydrogel, collagen, cross-linked collagen, and the like. One or more of the lumens 10420, 10424, and 10422 can be eliminated or blocked selectively to route material to the appropriate location. The injection port 10416 can advantageously comprise a one way valve, or other backflow prevention device, such as a pinhole valve, duckbill valve, iris valve, slit valve, stopcock, and the like, to prevent fluid from leaking out of the device and disc nucleus following injection.

With respect to the foregoing embodiments, it will be readily apparent to those skilled in the art that various combinations of the embodiment depicted are possible in order to combine features as disclosed herein. For example, spinal implants may include bone-compaction holes or not. Where present the holes may be placed in the head portion, the barrier portion or in both portions. Likewise, where holes are present they may be present substantially around the entire circumference of the implant or may be in a region of the implant.

Further, each of the embodiments also provides that the implant may be fashioned from a single piece of material or from more than one material where different properties are required in different functional regions of the implant. Similarly, embodiments of the implants described can be provided in multiple parts, for example, separate head and barrier portions that are either lockably connected or reversibly connected.

Moreover, in some embodiments the spinal implant is at least partially biodegradable. A biodegradable implant can be fashioned of natural substances such as collagen, or artificial polymers many of which are well known in the art. In addition, it can be useful to provide an implant which is remodelable, e.g., that the material would be subject to natural biological tissue remodeling processes that occur in vivo. For example, this can include, without limitation, the use of natural or synthetically produced bone or cartilage, either as autograft or allograft material. In some embodiments, synthetic materials that simulate the properties of bone or cartilage can be used.

Using an implant fashioned from a relatively permeable matrix material, such as cartilage, permits the inclusion of additional factors to promote healing of the disc. For example, an artificial cartilage implant can include growth factors for specific cell types to promote healing and/or remodeling of the damaged disc and surrounding tissues, or inhibitory substances to reduce inflammation in response to the surgical procedure at the site where the implant is located.

The skilled artisan will recognize the interchangeability of various features from different embodiments. Similarly, the various features and steps discussed above, as well as other known equivalents for each such feature or step, can be mixed and matched by one of ordinary skill in this art to perform compositions or methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

1.-119. (canceled)

120. A method, for at least one of (i) treating a defect in an intervertebral disc between adjacent vertebrae, and (ii) maintaining a separation between adjacent vertebrae, comprising:
positioning a spacer in an intervertebral disc space between the adjacent vertebrae;
inserting a dilator into a lumen in the spacer, thereby expanding the spacer from a first configuration to a second configuration and thereby securing the implant in the intervertebral disc space.

121. A method according to claim 120, wherein the positioning step comprises inserting the spacer through a defect in the annulus fibrosus of an intervertebral disc between the adjacent vertebrae.

122. A method according to claim 120, wherein the positioning step comprises inserting the spacer transversely, from one lateral aspect of the intervertebral disc space toward an opposite lateral aspect of the intervertebral disc space.

123. A method according to claim 120, further comprising locking the spacer in the second configuration.

124. A method according to claim 120, further comprising locking the dilator in the spacer, such that the spacer is in the second configuration after the locking.

125. A method according to claim 120, further comprising interacting the dilator with the spacer to result in at least one of locking the dilator in the spacer and limiting axial movement of the dilator within the spacer.

126. A method according to claim 120, further comprising:
inserting a guidewire into the lumen; and
advancing a pusher along the guidewire, thereby pushing the dilator into the lumen and expanding the spacer.

127. A method according to claim 120, further comprising:
entering, with a guidewire, into the intervertebral disc at a first location;
exiting, with the guidewire, from the intervertebral disc at a second location;
advancing the spacer along the guidewire into the intervertebral disc space; and
advancing the dilator along the guidewire into the lumen, thereby expanding the spacer.

128. A method according to claim 120, wherein, when the inserting step results in the spacer expanding primarily in an inferior-superior direction with respect to the adjacent vertebrae, as the spacer expands from the first configuration to the second configuration.

129. A method according to claim 120, further comprising:
moving the dilator axially within the lumen; and
controlling at least one of an amount and a direction of expansion of the spacer based on a cross-sectional geometry of the dilator.

130. A method, for maintaining a height between adjacent vertebrae of a patient, comprising:
inserting an implant between the adjacent vertebrae;
after the inserting, and with a movable portion of the implant, penetrating an endplate of at least one of the adjacent vertebrae, thereby securing the implant between the adjacent vertebrae.

131. A method according to claim 130, wherein the inserting is performed through a minimally invasive surgical opening in the skin of the patient.

132. A method according to claim 130, wherein the anchor member comprises is a screw.

133. A method according to claim 130, wherein the anchor member comprises at least one of a hook and a barb.

134. A method according to claim 130, wherein the implant comprises:
a head;
a tail coupled to the head; and
a flange coupled to the tail.

135. A method according to claim 134 wherein the tail and flange of the implant are configured to form a barrier that prevents substantial expulsion of material from the intervertebral disc.
